(19) United States
(12) Patent Application Publication
(10) Pub. No.: US 2025/0195855 A1
(43) Pub. Date: Jun. 19, 2025

(54) INGESTIBLE DEVICE FOR DELIVERY OF THERAPEUTIC AGENT TO THE GASTROINTESTINAL TRACT

(71) Applicant: BIORA THERAPEUTICS, INC., San Diego, CA (US)

(72) Inventors: Nelson QUINTANA, Temecula, CA (US); Jeffrey A. SHIMIZU, Poway, CA (US); Christopher Loren WAHL, San Diego, CA (US); Sharat SINGH, Rancho Santa Fe, CA (US); Bryan Jacob Zachary SMITH, Dallas, TX (US); Jeffrey Allan SMITH, Earlysville, VA (US); Pejman RAHIMIAN, Colleyville, TX (US); Stuart Robert ABERCROMBIE, Cambridge, Cambridgeshire (GB); Edward MUDGE, Cambridge, Cambridgeshire (GB); Nicholas Mark SALT, Cambridge, Cambridgeshire (GB); Nia Eleri STEVENS, Cambridge, Cambridgeshire (GB); Christopher lan BUNCE, Cambridge, Cambridgeshire (GB); Aruna PERERA, Alameda, CA (US); Thotsaphon VONGASAVARIT, San Diego, CA (US); Jack AULD, Irvine, CA (US); Natalie JASSO, Irvine, CA (US); Pete BLISS, Irvine, CA (US); Cho Kin CHIU, Irvine, CA (US); Eric ANDERFASS, Irvine, CA (US); Michelle Miyako WALKER, San Diego, CA (US); Rene Octavio VALENZUELA-RIVAS, Chula Vista, CA (US)

(21) Appl. No.: 18/847,875

(22) PCT Filed: Mar. 20, 2023
(86) PCT No.: PCT/US2023/064716

§ 371 Date: Sep. 17, 2024

Related U.S. Application Data
(60) Provisional application No. 63/321,537, filed on Mar. 18, 2022.

(51) Int. Cl. A61M 31/00; A61M 5/30

(52) U.S. Cl. CPC A61M 31/002; A61M 5/30; A61M 2205/8225; A61M 2210/1042

(57) ABSTRACT

Ingestible devices can directly deliver therapeutic agents to desired tissue(s) of the GI tract of a subject, such as the submucosa, the mucosa, and/or the mucus layer of the GI tract, and methods of using the same. The ingestible devices can deliver therapeutic agents in a safe, effective, and reliable manner. The disclosure also provides pharmaceutical compositions for use in methods of treating a disease or condition.

### FIELD

The field of the invention is ingestible devices capable of delivering a dispensable substance, such as a therapeutic agent, as well as related components, systems and methods.

### BACKGROUND

The gastrointestinal (GI) tract generally provides a therapeutic medium for an individual's body. At times, it is desirable to dispense therapeutic agents to the GI tract to treat a medical condition.

### SUMMARY

Ingestible devices can directly deliver therapeutic agents to desired tissue(s) of the GI tract of a subject, such as the submucosa, the mucosa, and/or the mucus layer of the GI tract. The ingestible devices can deliver therapeutic agents in a safe, effective, and reliable manner. Pharmaceutical compositions for use in methods of treating a disease or condition in humans and animals, and methods of use, are also described.

Ingestible devices are configured to provide at least three different modes of direct delivery of therapeutic agents to the GI tract of a subject, referred to herein as trans-epithelial, epithelial, and topical delivery. Direct delivery, as used herein, refers to a force-driven delivery mechanism.

Thus, in one aspect, a trans-epithelial delivery of a therapeutic agent is delivered to the GI tract of a patient. An ingestible device can directly deliver a therapeutic agent past the epithelial cell layer of the mucosa of the GI tract to yield systemic exposure of the therapeutic agent. The ingestible device may be configured to directly deliver the therapeutic agent past the epithelial cell layer of the mucosa and into the submucosa and/or into a region of the mucosa beneath the epithelial layer (e.g., into the lamina propria) of the GI tract, where it is available for systemic uptake. This can be particularly relevant when the oral bioavailability of the therapeutic agent is otherwise low. In some embodiments, systemic exposure of the therapeutic agent is achieved by trans-epithelial delivery of the therapeutic agent into the submucosa and/or into a region of the mucosa beneath the epithelial layer (e.g., into the lamina propria) of the small intestine, for example, in the duodenum, the jejunum, and/or the ileum. In further embodiments, the trans-epithelial delivery directly delivers the therapeutic agent into the submucosa and/or into a region of the mucosa beneath the epithelial layer (e.g., into the lamina propria) of the GI tract such that the percent systemic uptake of the trans-epithelial delivery relative to intravenous or subcutaneous administration is at least about 10% (e.g., at least about 15%, at least about 20%, at least about 25% %, at least about 30%, at least about 35%, at least about 40% or more).

In another aspect, epithelial delivery of a therapeutic agent to the GI tract is provided. An ingestible device may be configured to directly deliver the therapeutic agent into the mucus and/or onto the epithelial layer, but not past the epithelial layer of the mucosa, of the small or large intestine, from which it can act locally, and in some cases away from the site of direct delivery. The device may be configured so that the therapeutic agent is delivered from the device with sufficient force to provide the epithelial delivery, the force being lower than that required for trans-epithelial delivery.

In yet another aspect, an ingestible device can provide topical delivery of a therapeutic agent to the GI tract. The ingestible device is then configured to deliver the therapeutic agent into the lumen and/or onto the mucus or other surface of the GI tract facing the lumen of the small or large intestine, from which it can act locally, and in some cases away from the site of delivery. In some embodiments, the device is configured so that the therapeutic agent is delivered from the device with sufficient force so that the therapeutic agent is delivered topically, the force being lower than that required for the epithelial or the trans-epithelial delivery.

The ingestible device, whether configured for trans-epithelial, epithelial or topical delivery, can have a streamlined and/or relatively simple mechanical design, be relatively small, and/or be inexpensive to manufacture. In general, the device protects a dispensable substance (e.g., a therapeutic agent, or a pharmaceutical formulation comprising the therapeutic agent) until the device reaches a desired location of the subject. As an example, the device can be designed to deliver dispensable substance to a desired location in the GI tract of a subject, and the device can be designed so that the dispensable substance is not subject to constituents of the GI tract (e.g., acids, enzymes) prior to reaching the desired location in the GI tract. As another example, the device can be designed to deliver dispensable substance such that the therapeutic properties of the dispensable substance are not altered during delivery (e.g., the dispensable substance is a therapeutic agent that binds its therapeutic target after delivery).

The ingestible devices described can directly deliver therapeutic agents to desired tissue(s) of the GI tract of a subject (such as the submucosa, the mucosa, and/or the mucus layer of the GI tract), e.g., to treat a particular class of disease, or a specific disease. Relatedly, methods of using the device to deliver the therapeutic agents to desired tissue(s) of the GI tract, e.g., to treat a particular class of disease, or a specific disease, are disclosed. These disclosures also inherently provide disclosures of corresponding medical uses-that is, disclosures of the recited therapeutic agents for use in a method of treating the recited class of disease, or specific disease, by using the device to deliver the recited agents to desired tissue(s) of the GI tract of a subject or patient.

In another aspect, an ingestible device includes a housing configured to contain a dispensable substance comprising a therapeutic agent. One or more openings in the housing are configured to allow the dispensable substance to move out of the housing. The ingestible device is configured to deliver the dispensable substance to the GI tract as a jet with a peak jet power of 3 Watts to 8 Watts, and/or at a peak jet velocity of from 40 m/s to 50 m/s, and/or a peak jet force of 150 mN to 310 mN. The ingestible device may include a gas container in the housing containing a compressed gas. A piercer may be used for piercing the gas cylinder to release the compressed gas from the gas container into the housing. In some designs, no piercer is used.

In another aspect, an ingestible device includes a housing, openings and a gas container, as described above, further including a first piston and a second piston both slidable longitudinally within the housing, and the therapeutic agent in a storage reservoir formed by and between the first piston, the second piston, and the housing. The second piston, if used, may include an internal breakaway cap having a breakaway section which lodges into a housing recess after it is displaced by movement of the second piston. The storage reservoir may be formed by the inner walls of the housing itself, or by a separate element positioned in the housing.

In embodiments including a piercer, a spring may apply a spring force on the piercer in a first direction towards the gas container, with a release component or trigger holding the piercer in place against the spring force. In this case, a trigger support having a conical surface may be held against the trigger by the spring, the conical surface having an angle of 10 to 25 degrees. With or without this trigger support, the trigger may hold arms of a piercer in a first position, wherein the arms hold the piercer in place against the spring force, with the arms movable to second position wherein the piercer is released and moves under the spring force to pierce the gas container, when the trigger is partially or fully dissolved, degraded and/or eroded.

In any of the embodiments described that include a piercer, a breakaway piercer base or a breakaway piercer flange may be used to hold the piercer in place until a threshold force is overcome wherein the piercer is released and moves under the spring force to pierce the gas container, when the trigger is partially or fully dissolved, degraded and/or eroded. Alternatively, shear pins may be used to hold the piercer in place until the threshold force by the force of pressurized gas released from the gas container. A trigger crown in between the trigger and an end of the housing to better allow for catastrophic single step destruction of the trigger, so that the device actuates quickly and smoothly.

In a design having no piercer, a pin is threaded into a container cap. A first O-ring on the pin seals against the gas container. A second O-ring on the container cap seals against the device housing. A trigger housing containing a release component is attached to the device housing. The container cap is rotatable from a first position wherein the gas container is substantially sealed, to a second position wherein the gas container is unsealed. The container cap is displaced from the gas container to release gas from the pressurized container when the release component releases.

In another design having no piercer, seals or O-rings are displaced from a receiver to release gas from the gas container.

A gas container used as an energy source for the device can be housed in any kind of housing, cannister, container, etc. in the housing, the gas container having a breakable seal; a spring, a piston, and a piercer in the interior of the housing; a retainer; and a trigger exposed to an environment external to the housing. In a first state of the ingestible device: the trigger holds the retainer in a first position; the retainer holds the piercer in a first position in which the piercer does not break the breakable seal of the gas container; and the interior of the ingestible device is configured to contain a dispensable substance without the dispensable substance being delivered from the ingestible device via the opening in the housing.

In a second state of the ingestible device: the trigger is partially or fully dissolved, degraded and/or eroded so that the trigger is unable to hold the retainer in its first position; and the retainer is unable to hold piercer in its first position.

In the second state of the ingestible device: the spring applies a force to the piercer to move the piercer so that the piercer breaks the breakable seal of the gas container; a gas is released from the gas container; the gas applies a force to the piston so that the piston applies a force to the dispensable substance; and the dispensable substance is delivered out of the ingestible device via the opening in the housing.

The ingestible device can further include a seal between the piston and the housing, and/or a seal between the piercer and the housing.

The ingestible device may include: a housing configured to contain a dispensable substance comprising a therapeutic agent in an interior of the housing; a gas cylinder, a spring, and a piston in the interior of the housing; a seal between the piston and the housing; a piercer in the interior of the housing; a retainer; and a release component or trigger exposed to an environment external to the housing. A second seal between the retainer and the housing may also be used. An ingestible device can be a 00 sized device. The release component or trigger can include an enteric material. The housing can include first and second housing parts, with the piston and the dispensable substance inside the first housing part, and the spring and the retainer inside the second housing part. The opening of an ingestible device can be a nozzle, e.g., having a diameter of from about 325 μm to 375 μm. The dispensable substance may be a solution or a suspension. The one or more openings can be arranged radially around the ingestible device.

In some embodiments, at least one of the following holds: the ingestible device is configured to deliver the dispensable substance to tissue of the GI tract of a subject as a jet with a peak jet power from 3 Watts to 8 Watts, or, in some embodiments, greater than 4.2 Watts and less than 7.8 Watts, or, in some embodiments, greater than 5.0 Watts and less than 6.3 Watts; the ingestible device is configured to deliver the dispensable substance at an average jet velocity of, in some embodiments, from 28 meters per second to 44 meters per second; the ingestible device is configured to deliver the dispensable substance to tissue of the GI tract of a subject at a peak jet force of, in some embodiments, from 150 mN to 310 mN, or, in some embodiments, from 195 mN to 285 mN, or, in some embodiments, from 215 mN to 250 mN; the ingestible device is configured to deliver the dispensable substance as a jet having jet stable length of at least 5 millimeters; the ingestible device is configured to provide an internal pressure of, in some embodiments, from 250 psig to 400 psig, or, in some embodiments, from 300 psig to 350 psig; and the ingestible device is configured to contain the dispensable substance at a peak fluid pressure of from, in some embodiments, from 230 psig to 380 psig, and in some embodiments, from 280 psig to 330 psig.

Some embodiments may include one or more of: at least one component which includes a cylic olefin polymer; the breakable seal is scored; the breakable seal has a varying thickness; and/or the gas container has a burst pressure of from about 2,800 psig to about 4,500 psig, with the gas comprising at least one gas selected from the group consisting of air, nitrogen, oxygen, carbon dioxide, hydrofluorocarbon gases and noble gases, or a mixture of them.

In some embodiments, an ingestible device further includes an element having a first state in which the element at least partially covers the opening in the housing and a second state in which the element does not cover the opening in the housing, wherein the ingestible device is configured so that, when the piston moves, the element moves from its first state to its second state. The element can move synchronously with the piston. When the piston moves a distance, the element can move the same distance. The ingestible device can further include a seal mechanically coupled with the piston and element. The seal can be configured to cause the movement of the piston to result in the movement of the element. The element can conform to an inner radius of the housing.

In some embodiments, the ingestible device further includes a covering over the opening in the housing. The covering can be removable from the ingestible device. The covering can be configured to be removed from the housing due to pressure applied by the dispensable substance. The covering can include an enteric material. The covering can erode or dissolve-either partially or completely. The covering can be a film, a foil, a band, a plug, or a patch. In some embodiments, the covering has a burst pressure of at most about 440 psi.

The ingestible device may further include a second piston configured so that, when the first piston applies the force on the dispensable substance, the dispensable substance applies a force on the second piston to slide the second piston to expose the openings and the dispensable substance is forced out of the ingestible device via the openings.

In some embodiments, the ingestible device further includes a removable cap affixed to the ingestible device and configured so that, when the piston moves to apply the force on the dispensable substance, the dispensable substance applies a force on the cap to slide the cap to expose the opening in the housing.

The ingestible device may also have an inflated membrane volume covering the opening and configured so that, when the piston moves to apply force on the dispensable substance, the dispensable substance applies force on the inflated membrane volume and the inflated membrane volume is compressed to expose the opening in the housing.

In an aspect, the disclosure provides a method that includes using an ingestible device according to the disclosure to deliver a dispensable substance to the GI tract of a subject.

The details of one or more embodiments of the device and methods are set forth in the accompanying drawings and the description below. Other features and advantages will be apparent from the description and drawings, and from the claims.

### BRIEF DESCRIPTION OF THE DRAWINGS

FIG. 1A is a schematic cross section of the different regions of healthy intestinal tissue.

FIG. 1B is a schematic cross section corresponding to FIG. 1A but for diseased intestinal tissue.

FIG. 2 is a cross section of an ingestible device.

FIG. 3 is a cross section of another ingestible device.

FIG. 4 shows an exemplary process flow chart for use of an ingestible device in which pressure is not applied to the dispensable substance before the subject swallows the ingestible device.

FIGS. 5A-5C show another ingestible device.

FIGS. 6A-6C shows an ingestible device with aspects similar to those shown in FIGS. 4 and 5.

FIGS. 7-13 show an ingestible device having multiple chambers for one or more dispensable substances.

FIGS. 14-17 show another ingestible device.

FIG. 18 shows certain elements of an ingestible device of FIG. 16.

FIGS. 19 and 20 show states of an ingestible device.

FIGS. 21 and 22 show states of an ingestible device.

FIGS. 23 and 24 show states of an ingestible device.

FIGS. 25-27 show ingestible devices.

FIGS. 28-30 show states of an ingestible device.

FIGS. 31-34 show states of an ingestible device.

FIGS. 35-40 show additional ingestible devices.

FIGS. 41A-41C show aspects of an ingestible device.

FIGS. 42A-47C show aspects of an ingestible device.

FIGS. 48A and 48B show states of an ingestible device.

FIGS. 49A-49E show second pistons for use in ingestible devices.

FIGS. 50A and 50B show exploded views of an ingestible device.

FIGS. 51A and 51B show exploded views of an ingestible device.

FIGS. 52A-52D show views of an ingestible device.

FIGS. 53A and 53B show views of a portion of an ingestible device.

FIGS. 54A and 54B show views of a portion of an ingestible device.

FIG. 55 shows a view of a portion of an ingestible device.

FIG. 56 is an exemplary graph of liquid pressure versus time for experiment(s) run with a variety of pressures and/or nozzle diameters.

FIG. 57 is an exemplary graph of jet impact force versus time for experiment(s) run with a variety of pressures and/or nozzle diameters.

FIG. 58 is an exemplary graph of jet power versus time for experiment(s) run with a variety of pressures and/or nozzle diameters.

FIG. 59 is an exemplary graph of jet velocity versus time for experiment(s) run with a variety of pressures and/or nozzle diameters.

FIG. 60 is an exemplary graph of liquid pressure versus time for experiment(s) run with a variety of pressures and/or nozzle diameters.

FIG. 61 is an exemplary graph of jet impact force versus time for experiment(s) run with a variety of pressures and/or nozzle diameters.

FIG. 62 is an exemplary graph of jet power versus time for experiment(s) run with a variety of pressures and/or nozzle diameters.

FIG. 63 is an exemplary graph of jet velocity versus time for experiment(s) run with a variety of pressures and/or nozzle diameters.

FIG. 64 is an exemplary graph of liquid pressure versus delivered volume for experiment(s) run with a variety of pressures and/or nozzle diameters.

FIG. 65 is an exemplary graph of jet impact force versus delivered volume for experiment(s) run with a variety of pressures and/or nozzle diameters.

FIG. 66 is an exemplary graph of jet power versus delivered volume for experiment(s) run with a variety of pressures and/or nozzle diameters.

FIG. 67 is an exemplary graph of jet velocity versus delivered volume for experiment(s) run with a variety of pressures and/or nozzle diameters.

FIG. 68 is an exemplary graph of jet impact force versus time for experiment(s) run at 320 psi.

FIG. 69 is an exemplary graph of jet impact force versus time for additional experiment(s) run at 320 psi.

FIG. 70 is an exemplary graph of PGN-OB1 PK in swine.

FIG. 71 are exemplary graphs of bioavailability versus deployment time.

FIG. 72A is an exemplary graph of jet impact force versus time for experiment(s) run at 320 psi.

FIG. 72A is an exemplary graph of jet impact force versus time for experiment(s) run with 14% Kollicoat nozzle covers at 320 psi.

FIG. 72B is an exemplary graph of jet impact force versus time for experiment(s) run with 20% Kollicoat nozzle covers at 320 psi.

FIGS. 73A and 73B show a dispensable substance container and a piston.

FIGS. 74A-74C show an alternative trigger housing.

FIGS. 75A-75D show alternative trigger supports.

FIGS. 76A-76F show alternative piercer designs.

FIGS. 77A-77D show breakaway piercer bases.

FIGS. 78A-78F show breakaway piercer flanges.

FIGS. 78G1-78G4 show detail options for the piecer flanges of FIGS. 78A-78F.

FIGS. 78H-78L show additional piercer flange designs.

FIGS. 79A-79C show a trigger having shear pins.

FIGS. 80A-80J show devices having a trigger crown.

FIGS. 81A-81F show devices having a gas container cap.

FIGS. 82A-821 show devices with a piercer coupled to a gas container.

FIGS. 83A-83E show devices with a breakaway cap.

FIGS. 84A-84B show devices using a two-piece gas container.

FIGS. 84C AND 84D show a device having a release component holding a cylinder cap in sealed engagement with a gas container.

FIGS. 85A-85D show devices having a release component including O-rings.

FIGS. 85E1-85E4 are schematic diagrams of O-ring movement.

FIG. 85F shows graphs of percentage of gas released over time.

FIG. 86 are graphs of adalimumab plasma concentration over time.

FIG. 87 are graphs of semaglutide plasma concentration over time.

FIG. 88 are graphs of jet force over time.

FIG. 89 is a graph of jet impact force over time for 400 psi.

FIG. 90 is a graph of jet impact force over time for 350 psi.

FIG. 91 is a graph of jet impact force over time for 320 psi.

FIG. 92 is a graph of jet impact force over time for 280 psi.

FIG. 93 are graphs of jet force over time.

FIG. 94 is a section view of an ingestible device having external gas release capability or point-of-use gas release capability.

FIG. 95 is a perspective view of the ingestible device of FIG. 94 in a holder.

FIG. 96 is a partial section view of the ingestible device as shown in FIG. 95.

FIG. 97 is a section view of the ingestible device of FIG. 96 during initial gas release.

FIG. 98 is a section view of the ingestible device of FIGS. 94 and 97, now shown after completion of actuation.

FIGS. 99 and 100 show an alternative second piston configured to make it easier to fill the drug module and provide a higher peak jet force.

FIGS. 100 and 101 are graphs of trigger data.

FIGS. 101 and 102 are charts showing the time to piercer movement after dissolution or degradation of a release component or trigger.

### DETAILED DESCRIPTION

### Definitions

“Ingestible,” as used herein in reference to the device, means that the device can be swallowed whole.

“Dispensable” as used herein with reference to any substance, refers to any substance that may be released from an ingestible device as disclosed herein, or from a component of the device such as a reservoir. For example, a dispensable substance may be a therapeutic agent as disclosed herein, and/or a formulation that includes a therapeutic agent as disclosed herein. A dispensable substance may be a fluid, such as a liquid, a suspension or a semi-solid. In such embodiments, the substance may be converted to a fluid prior to being delivered from the ingestible device. In some embodiments, the therapeutic agent is a small molecule. In other embodiments, the therapeutic agent is a large molecule, such as a biologic drug. In some embodiments, a dispensable substance delivered as described herein is particularly well-suited for treatment of diseases and conditions of the endoderm, for example, it may be more efficacious in gut-associated lymphoid tissue (GALT) or the hepatic system as compared to subcutaneous or intravenous administration. The dispensable substance may have a viscosity of at least about 0.5 centipoise (cP) (to about 100 cP, 2 cP to about 50 cP or about 5 cP to about 25 cP.

As used herein, the term “enteric” refers a material that permits transition to a desired location in the GI tract (e.g., through the stomach to the intestine) before being dissolved/degraded/eroded due to exposure of certain conditions (e.g., pH, temperature, enzymes) of the GI tract. An enteric material may prevent a drug from degradation by gastric fluid and enzymes. In some embodiments, an enteric composition (e.g., when formed as a coating on the housing of an ingestible device) is selected from mixtures of fats and fatty acids; shellac and shellac derivatives; and cellulose acetate phthalates. An enteric material can be an enteric polymer. An enteric polymer can remain insoluble in the stomach, but dissolve at the higher pH of the intestine (e.g., small intestine or large intestine), and are used to deliver drugs to the intestine. Examples include-Eudragit L 100-55 Mixture Methacrylic copolymers); Evonik's Eudragit L 100-55 Methacrylic copolymers, Eudragit L 30 D-55 Methacrylic copolymers (30%), Eudragit L 100 Methacrylic copolymers, Eudragit L 12.5 Methacrylic copolymers (12.5%), Eudragit S 100 Methacrylic copolymers, Eudragit S 12.5 Methacrylic copolymers (12.5%), Eudragit FS 30 D Methacrylic copolymers (30%); Kerry's SheffCoat ENT Cellulose Acetate Phthalate, Acrylate copolymer, HPMC-P; Eastman's C-A-P NF Cellulose Acetate Phthalate; Sensient's PROTECT™ ENTERIC Shellac & Sodium Alginate.

In certain embodiments, an enteric material dissolves in the small intestine and is suitable for small intestine release. Examples of such enteric materials include, but are not limited to, cellulose derivatives, e.g., cellulose acetate phthalate, hydroxypropylmethylcellulose phthalate (HPMCP), hydroxypropylmethylcellulose acetate succinate (HPMCAS) and RL100 (e.g., HP-55), malic acid-propane 1,2-diol, polyvinyl acetate phthalate, anionic polymers of methacrylic acid and methyl methacrylate, hydroxypropylcellulose acetate phthalate, polyvinyl acetate phthalate, methacrylate-methacrylic acid copolymers, styrol, maleic acid copolymers, shellac, and others. Another suitable enteric material is a water emulsion of ethylacrylate methylacrylic acid copolymer, or hydroxypropyl methyl cellulose acetate succinate (HPMAS). In some embodiments, an enteric material dissolves in the large intestine and is suitable for colonic release. Enteric materials suitable for large intestine (e.g., colonic) release are known to one of skill in the art. In some embodiments, degradation of the coating is microbially triggered, e.g., the bacteria in the colon enzymatically trigger degradation of the coating In some embodiments, the coating is a pH-dependent polymer that is insoluble at low pH but becomes increasingly soluble as pH increases. In some embodiments, the coating is a polymethacrylates with a pH-dependent dissolution threshold of about pH 6.0 to about 7.0. Examples of suitable enteric materials include, but are not limited to, chitosan, alginates (e.g., as calcium salts), Eudragit® L (e.g., Eudragit® 100), Eudragit® S (e.g., Eudragit® S 100), Eudragit® L (e.g., Eudragit® L-30D), Eudragit® FS (e.g., Eudragit® FS 30D), hydroxypropylmethylcellulose phthalate 50, hydroxypropylmethylcellulose phthalate 55, and cellulose acetate trimellate. In some embodiments, the colon-specific degradation of an enteric material can be based on the presence of microorganisms that reside only in the colon, more particularly, biodegradable enzymes produced by these microorganisms. In general, such microorganisms are anaerobic bacteria, e.g., Bacteroides, Bifidobacteria, Enterobacteria, Eubacteria, Clostridia, Enterococci, and Ruminococcus, etc. These micro floras fulfill their energy needs by fermenting various types of substrates that have been left undigested in the small intestine, e.g., polysaccharides, di- and tri-saccharides, etc. These polymers are stable in the environments of the stomach and small intestine. On reaching the colon, the polymers undergo degradation by the enzyme or break down of the polymer backbone leads to a subsequent reduction in their molecular weight and thereby loss of the mechanical strength.

The term “jet,” as used herein, refers to a collimated stream of fluid, e.g., liquid or suspension, that is stable without breaking up into a spray. A jet may be formed by forcing the fluid, e.g., liquid or suspension, through an opening in an ingestible device. Generally, a jet maintains a stable form and is capable of achieving its intended purpose by maintaining appropriate properties (e.g., to penetrate a surface), such as its diameter and/or velocity.

As used herein, “jet diameter” is the cross-sectional diameter of a jet at a given location.

As used herein, “average jet diameter” refers to the average cross-sectional diameter of a jet between the location where the jet is formed (e.g., a nozzle opening through which the dispensable substance is delivered from the ingestible device) and the location where the jet impacts the GI tissue of the subject.

“Jet stable length,” as used herein, refers to the distance from an opening (e.g., nozzle opening) of an ingestible device that a dispensable substance delivered through the opening remains in the form of a jet.

“Jet velocity,” as used herein is the average fluid velocity across the cross-section of a jet at a given point in time.

As used herein, “peak jet velocity,” refers to the maximum jet velocity of a jet at the interface of the lumen and the surface of the GI tract facing the lumen. In general, the peak jet velocity is achieved at the time of initial delivery of the dispensable substance from the ingestible device.

As used herein, “minimum jet velocity,” refers to the minimum velocity of a jet at the interface of the lumen and the surface of the GI tract facing the lumen. In general, the minimum jet velocity is achieved at the end of delivery of the dispensable substance from the ingestible device.

“Mean jet velocity” and “average jet velocity,” as used herein, refer to the average velocity of a jet at the interface of the lumen and the surface of the GI tract facing the lumen as determined over the time that the ingestible device delivers the dispensable substance.

As used herein, “peak jet power” refers to the maximum power of a jet at the interface of the lumen and the surface of the GI tract facing the lumen. In general, the peak jet power is achieved at the time of initial delivery of the dispensable substance from the ingestible device.

As used herein, “minimum jet power,” refers to the minimum power of a jet at the interface of the lumen and the surface of the GI tract facing the lumen. In general, the minimum jet power is achieved at the end of delivery of the dispensable substance from the ingestible device.

“Mean jet power” and “average jet power,” as used herein, refer to the average power of a jet at the interface of the lumen and the surface of the GI tract facing the lumen as determined over the time that the ingestible device delivers the dispensable substance.

“Jet power during delivery,” as used herein, refers to the power of a jet at the interface of the lumen and the mucosa of the GI tract of a subject.

“Jet pressure,” as used herein, refers to the pressure of a jet at the interface of the lumen and the surface of the GI tract facing the lumen. As an example, the jet pressure can be the pressure by the jet measured at the intestinal wall. In some embodiments, jet pressure is referred to herein as “impact pressure.”

“Peak jet pressure,” as used herein, refers to the maximum pressure of a jet at the interface of the lumen and the surface of the GI tract facing the lumen. In general, the peak jet pressure is achieved at the time of initial delivery of the dispensable substance from the ingestible device.

As used herein, “minimum jet pressure,” refers to the minimum pressure of a jet at the interface of the lumen and the surface of the GI tract facing the lumen. In general, the minimum jet pressure is achieved at the end of delivery of the dispensable substance from the ingestible device.

“Mean jet pressure” and “average jet pressure,” as used herein, refer to the average pressure of a jet at the interface of the lumen and the surface of the GI tract facing the lumen as determined over the time that the ingestible device delivers the dispensable substance.

“Jet force,” as used herein, refers to the force of a jet at the interface of the lumen and the surface of the GI tract facing the lumen. In some embodiments, jet force is referred to herein as “impact force.”

“Peak jet force,” as used herein, refers to the maximum force of a jet at the interface of the lumen and the surface of the GI tract facing the lumen. In general, the peak jet force is achieved at the time of initial delivery of the dispensable substance from the ingestible device. In some embodiments, peak jet force is referred to herein as “impact force.”

As used herein, “minimum jet force,” refers to the minimum force of a jet at the interface of the lumen and the mucosa of the GI tract of a subject. In general, the minimum jet force is achieved at the end of delivery of the dispensable substance from the ingestible device.

“Mean jet force” and “average jet force,” as used herein, refer to the average pressure of a jet at the interface of the lumen and the surface of the GI tract facing the lumen as determined over the time that the ingestible device delivers the dispensable substance.

As used herein, “fluid volume” refers to the volume of the dispensable substance contained in the ingestible device.

“Initial fluid volume,” as used herein, refers to the volume of the dispensable substance contained in the ingestible device just prior to delivery of the dispensable substance from the ingestible device.

“Final fluid volume,” as used herein, refers to the volume of the dispensable substance contained in the ingestible device just after delivery of the dispensable substance from the ingestible device has ended.

As herein, “delivered fluid volume” refers to the volume of dispensable substance delivered from the ingestible device. In some embodiments, the delivered fluid volume is less than the fluid volume.

“End round” as used herein is the radius on the curve at the end of the housing of the ingestible device.

“Fluid pressure” as used herein refers to the pressure in the fluid volume.

As used herein, “peak fluid pressure” refers to maximum pressure generated in the fluid volume. Generally, the peak fluid pressure is achieved at initial delivery of the dispensable substance from the ingestible device. In some embodiments, peak fluid pressure is referred to herein as “internal pressure on the pharmaceutical formulation in the device, prior to release from the device.”

As used herein, “minimum fluid pressure” refers to minimum pressure generated in the fluid volume. Generally, the minimum fluid pressure is achieved at the end of delivery of the dispensable substance from the ingestible device.

“Fluid pressure during delivery,” as used herein, refers to the pressure in the fluid volume as it decreases during the delivery process.

As used herein, “nozzle” refers to a channel between a fluid reservoir space and an external environment. Generally, in embodiments in which a nozzle is used, pressure in the fluid volume generates a high speed flow of fluid through the nozzle to produce a fluid jet at the opening of the nozzle through which the dispensable substance leaves the ingestible device and enters an environment exterior to the ingestible device.

“Nozzle diameter,” as used herein, refers to the diameter of the opening of the nozzle at the opening of the nozzle through which the dispensable substance leaves the ingestible device and enters an environment exterior to the ingestible device.

As used herein, “nozzle length” refers to the length of the opening of the nozzle.

“Nozzle stand-off distance,” as used herein, refers to the distance between: 1) the opening of the nozzle through which the dispensable substance leaves the ingestible device and enters an environment exterior to the ingestible device; and 2) the interface of the lumen and the surface of the GI tract facing the lumen.

As used herein, the “internal pressure” of an ingestible device refers to the pressure applied to a dispensable substance, such as a therapeutic agent, or a formulation containing a therapeutic agent, contained in the ingestible device prior to delivery of the dispensable substance from the ingestible device. In some embodiments, the internal pressure is provided by the drive force generator of the ingestible device. In certain embodiments, the internal pressure is greater than the fluid pressure. This may be due, for example, to friction, such as O-ring friction, acting on the drive coupling of the ingestible device. This friction is referred to herein as the “piston friction.”

“Nozzle pressure” as used herein refers to the pressure of a dispensable substance at a nozzle opening as measured at the surface facing the interior of the nozzle as the dispensable substance is delivered from the ingestible device. In general, for a given ingestible device at a given point in time, the nozzle pressure is approximately the same as the fluid pressure.

“Topical delivery” or “topical administration,” as used herein, refers to a route of administration of a dispensable substance (for example, a therapeutic agent or a pharmaceutical formulation containing a therapeutic agent) where the dispensable substance is delivered to a localized area of the body or to the surface of a body part, regardless of the location of the effect; more particularly, the topical administration of the dispensable substance comprises releasing the dispensable substance to the lumen of the GI tract, a surface of the GI tract facing the lumen, a mucous membrane and/or a lining of the gastrointestinal tract of a subject, including, but not limited to, a surface, mucous membrane or lining containing one or more disease sites, such as gastrointestinal mucosal lesions. The effect of the topical delivery or topical administration of the dispensable substance may be local to, or away from (e.g., distal to), the site of the topical administration.

“Epithelial delivery” or “epithelial administration,” as used herein, refers to a route of administration of a dispensable substance (for example, a therapeutic agent or a pharmaceutical formulation containing a therapeutic agent) where the dispensable substance is directly delivered into the mucus or onto the epithelium, but not past the epithelial layer, of the GI tract of a subject, such as the small or large intestine, from which the dispensable substance can act locally or peripherally. In some embodiments of epithelial delivery or epithelial administration, the therapeutic agent can move deeper into the GI tissue (i.e., past the epithelial layer) away from the site of direct delivery, such as, for example, via diffusion or active transport.

“Trans-epithelial delivery” or “trans-epithelial administration,” as used herein, refers to a route of administration of a dispensable substance (for example, a therapeutic agent or a pharmaceutical formulation containing a therapeutic agent) where the dispensable substance is directly delivered through the epithelial layer of the mucosa of the GI tract to the submucosa of the GI tract of a subject; optionally, at least a portion of the dispensable substance is directly delivered past the epithelial layer to a region of the mucosa beneath the epithelial layer. In embodiments of trans-epithelial delivery in which a portion of the dispensable substance is directly delivered to a region of the mucosa beneath the epithelial layer, at least some (e.g., all) of the portion of the dispensable substance is directly delivered to the lamina propria. Once the therapeutic agent or a pharmaceutical formulation containing a therapeutic agent is directly delivered past the epithelial layer of the GI tract, it is available for systemic exposure of the therapeutic agent to the subject.

“Triggering” or a “triggering event”, or “actuation” as used here, means a change in condition or position of a component which results in a movement within the device causing the device to deliver a dispensable substance. A trigger or triggering component or element, also referred to as a release component, is an element that provides or contributes to triggering.

As used herein, “about” means the number disclosed, plus or minus 10% of that number.

### General Introduction

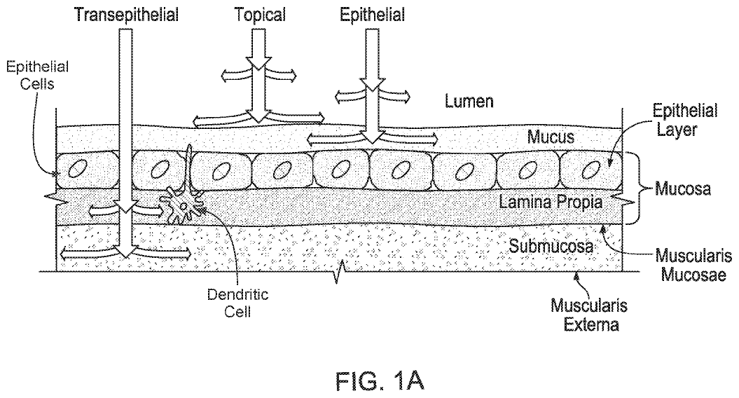

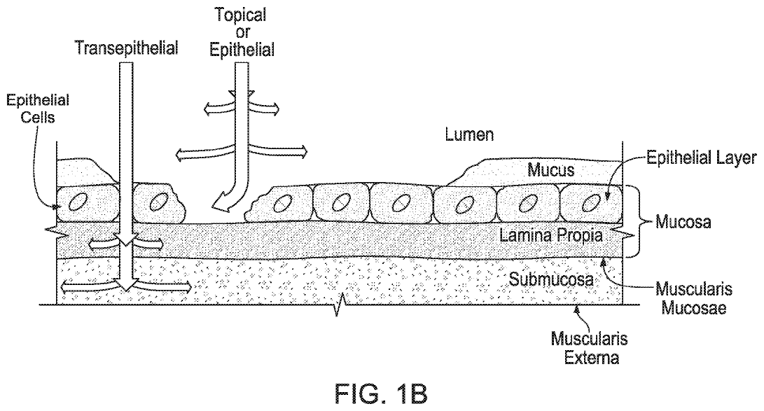

FIG. 1A schematically describes the different regions of healthy intestinal tissue, presented in a cross section. The regions include the lumen of the GI tract, the mucus of the GI tissue, the mucosa of the GI tissue and the submucosa of the GI tissue. The mucosa of the GI tissue includes the epithelial layer and the lamina propria. The muscularis mucosae separates the mucosa from the submucosa. The muscularis extrema is below the submucosa. FIG. 1B schematically describes corresponding regions of diseased intestinal tissue, presented in a cross section.

An ingestible device described herein can deliver a therapeutic agent via topical delivery (without being directly delivered to the mucus, mucosa or submucosa), epithelial delivery (directly delivered to the mucus or epithelium without being directly delivered past the epithelial layer to the mucosa or submucosa) or trans-epithelial delivery (directly delivered to the submucosa and/or into a region of the mucosa beneath the epithelial layer, such as the lamina propria.

In general, the form of delivery may depend on the design of the ingestible device and parameters used with the device (e.g., internal pressure, fluid pressure, number of nozzles, design of nozzles). Holding other parameters constant, at relatively low fluid pressures and/or internal pressures, the therapeutic agent may be topically delivered, while higher fluid pressures and/or internal pressures may result in epithelial delivery, and still higher fluid pressures and/or internal pressure may result in trans-epithelial delivery. During trans-epithelial delivery, a bolus of the therapeutic agent initially contained in the dispensable substance may form within the submucosa and/or into a region of the mucosa beneath the epithelial layer, such as the lamina propria.

In some embodiments, the following holds. The ingestible device is designed to deliver a dispensable substance, for example, a therapeutic agent or a pharmaceutical formulation containing a therapeutic agent through the epithelial layer of the mucosa of the GI tract. In some embodiments, the dispensable substance is a solution formulation; optionally, a suspension. In some embodiments, the dispensable substance enters the submucosa and/or into a region of the mucosa beneath the epithelial layer, such as the lamina propria, of the small intestine, where it can be absorbed systemically. After the patient swallows the device, it passes through the GI tract and eventually reaches the small intestine. The device includes a restraining mechanism, an optionally a triggering mechanism (e.g., a degradable and/or erodible coating, such as an enteric coating, that partially or completely degrades and/or erodes when the device reaches the desired location in the GI tract). The desired location can be the small intestine or the large intestine. When the device is configured for trans-epithelial GI tract delivery to the submucosa and/or into a region of the mucosa beneath the epithelial layer, such as the lamina propria, the preferred location can be the small intestine. With the restraining element is removed, relative movement between certain components (e.g., sliding of a component) occurs such that one or more openings in the ingestible device (e.g., in a compartment containing the dispensable substance, such as a reservoir, sometimes referred to herein as the “drug reservoir,” “storage reservoir” or “substance reservoir”) become aligned with one or more additional openings (e.g., one or more nozzles) in the ingestible device (e.g., in the housing). With the ingestible device now in this open position, a force (e.g., generated by a force generator and/or transferred by a drive coupling, such as a membrane or a piston) forces the dispensable substance from the drug reservoir out of the device via the one or more openings (e.g., the one or more nozzles). The dispensable substance is delivered as a jet of fluid (e.g., liquid) through the epithelial layer of the mucosa and directly into the submucosa and/or into a region of the mucosa beneath the epithelial layer, such as the lamina propria, of the GI tract in the form of single or multiple boluses. After swallowing the device, the device travels through the GI tract (mouth, esophagus, stomach, duodenum, jejunum, ileum, cecum and colon), ultimately exiting the GI tract via the anus.

Thus, in general, the ingestible devices disclosed herein provide delivery of therapeutic agent to the GI tract of a subject. In one aspect, the disclosure relates to trans-epithelial delivery of a dispensable substance (e.g., a therapeutic agent or a formulation comprising a therapeutic agent) to the GI tract of a subject. Accordingly, the disclosure provides an ingestible device that can directly deliver a dispensable substance (e.g., a therapeutic agent or a formulation comprising a therapeutic agent) to the submucosa and/or into a region of the mucosa beneath the epithelial layer, such as the lamina propria, of the GI tract of a subject, which may result in systemic exposure of the therapeutic agent to the subject. In such embodiments, the ingestible device is configured to directly deliver the dispensable substance past the epithelial cell layer of the mucosa and into the submucosa and/or into a region of the mucosa beneath the epithelial layer, such as the lamina propria, of the GI tract, where the therapeutic agent so delivered is available for systemic uptake. In some embodiments, systemic exposure of the therapeutic agent is achieved by trans-epithelial delivery of the dispensable substance into the submucosa and/or into a region of the mucosa beneath the epithelial layer, such as the lamina propria, of the small intestine, for example, in the duodenum, the jejunum, and/or the ileum. In some further embodiments, the trans-epithelial delivery directly delivers the dispensable substance into the submucosa and/or into a region of the mucosa beneath the epithelial layer, such as the lamina propria, of the GI tract such that the percent systemic uptake of the therapeutic agent via the trans-epithelial delivery relative to intravenous or subcutaneous administration is at least about 10% (e.g., at least about 15%, at least about 20%, at least about 25% or more).

The direct delivery of the therapeutic agent to the submucosa and/or into a region of the mucosa beneath the epithelial layer, such as the lamina propria, via trans-epithelial delivery may also or alternatively provide therapeutic effects locally and/or away from (e.g., distal to) the site of the direct delivery.

Trans-epithelial delivery may directly deliver a first portion of the dispensable substance to the submucosa of the GI tract, and a second portion of the dispensable substance to the mucosa, all or a further portion of which may be directly delivered to the lamina propria. In some embodiments, the second portion of the dispensable substance delivered to the mucosa, such as the lamina propria, of the GI tract via the trans-epithelial delivery may provide therapeutic effects locally and/or away from (e.g., distal to) the site of the direct delivery.

In another aspect, the disclosure relates to epithelial delivery of a dispensable substance (e.g., a therapeutic agent or a formulation comprising a therapeutic agent) to the GI tract of a subject. Accordingly, the disclosure provides an ingestible device configured to directly deliver a dispensable substance (e.g., a therapeutic agent or a formulation comprising a therapeutic agent) into the mucus, but not past the epithelial layer of the mucosa, of the small or large intestine, from which it may provide therapeutic effects locally and/or away from (e.g., distal to) the site of the direct delivery. In some further embodiments, the ingestible device directly delivers the dispensable substance such that it contacts the surface of the epithelial cell layer of the mucosa facing the lumen, but as previously noted, the epithelial delivery does not directly delivery the dispensable substance past the epithelial layer of the mucosa. In some embodiments, the device is configured so that the dispensable substance is delivered from the device with sufficient force to provide the epithelial delivery, the force being lower than that required for trans-epithelial delivery to the GI tract. In some further embodiments, the epithelial delivery directly delivers the dispensable substance into the mucus of the GI tract such that the percent systemic uptake of the therapeutic agent via the epithelial delivery relative to intravenous or subcutaneous administration is greater than that for topical delivery, but less than for trans-epithelial delivery. In other embodiments, the epithelial delivery directly delivers the dispensable substance into the mucus of the GI tract such that the percent systemic uptake of the therapeutic agent via the epithelial delivery relative to intravenous or subcutaneous administration is about 0.5% to about 10% or more (e.g., about 0.5%, about 2%, about 3%, about 4%, about 5%, about 6%, about 7%, about 8%, about 9%, about 10%, or more).

Accordingly, provided herein are new systemic delivery devices and methods that deliver therapeutic agents into the small intestinal mucosa and/or submucosa by jet injection. Current methods of administration for most large molecule therapeutic agents are subcutaneous (SC), intramuscular (IM), or bolus intravenous (IV) injection targeting the systemic circulation. The devices and methods described herein provide an alternative route of administration to current injectable medications, which can lead to greater convenience and compliance since they minimize or avoid the logistical challenges, patient compliance and adherence challenges, pain, and discomfort associated with traditional routes of administration.

Also, by providing a higher concentration of therapeutic in GI tissue, the devices and methods described herein are particularly well-suited for treatment of diseases and conditions of the endoderm, including the liver.

In some embodiments of epithelial delivery, the therapeutic agent directly delivered into the mucus of the GI tract via the epithelial delivery may undergo active or passive transport or diffusion past the epithelial layer. Once past the epithelial layer, the therapeutic agent may provide therapeutic effects locally and/or away from (e.g., distal to) the site of the direct delivery. In some embodiments, the therapeutic agent binds to a therapeutic target present in the GI epithelial layer or elicits other pharmacodynamic effects locally or away from the site of delivery via immune cells or tissue in the GI tract (e.g., dendritic cells, lymphocytes, mucosa-associated lymphoid tissue).

In yet another aspect, this disclosure relates to topical delivery of a dispensable substance (e.g., a therapeutic agent or a formulation comprising a therapeutic agent) to the GI tract of a subject. Accordingly, the disclosure provides an ingestible device configured to deliver the dispensable substance (e.g., a therapeutic agent or a formulation comprising a therapeutic agent) into the lumen and/or onto the mucus or other surface (e.g., a diseased surface) of the GI tract facing the lumen of the small or large intestine, from which it may provide therapeutic effects locally and/or away from (e.g., distal to) the site of delivery. In some embodiments, the device is configured so that the dispensable substance is delivered from the device with sufficient force so that the dispensable substance is delivered topically, the force being lower than that required for the epithelial or the trans-epithelial delivery to the GI tract. In some embodiments, the topical delivery to the GI tract results in reduced systemic uptake of the therapeutic agent compared to trans-epithelial delivery to the GI tract, intravenous or subcutaneous delivery.

In some further embodiments, topical delivery delivers the dispensable substance into the lumen and/or onto the mucus or the other surface facing the lumen of the GI tract such that the percent systemic uptake of the therapeutic agent via the topical delivery relative to intravenous or subcutaneous administration is less than about 20%, less than about 10%, less than about 5%, less than about 4%, less than about 3%, less than about 2% or less than about 1%. In some embodiments, the topical delivery to the GI tract results in negligible or no systemic uptake of the therapeutic agent compared to trans-epithelial delivery to the GI tract, intravenous or subcutaneous delivery.

The topically delivered dispensable substance may spread over the mucus or other surface facing the lumen of the GI tract, thereby coating the surface of the GI tract at or away from (e.g., distal to) the site of delivery. In some embodiments, upon or after the dispensable substance has been topically delivered, the therapeutic agent may undergo transport (e.g., diffusion) from the surface of the mucus into the mucus, and optionally, active or passive transport or diffusion past the epithelial layer of the mucosa.

The mucus and/or epithelial layer of the mucosa may be disrupted or even absent, such as in a patient having a disease or condition of the GI tract. In such embodiments, the topical delivery of the dispensable substance to the GI tract of the patient may provide direct delivery of the dispensable substance to the surface of the GI tract facing the lumen, such as mucosal tissue exposed by the disruption and/or absence (e.g., both the mucus layer and/or epithelial layer are completely or partially absent or compromised in portions of the GI tract due to a disease or condition). For example, in some embodiments, the topical delivery of the dispensable substance to the GI tract of the patient may provide topical delivery to one or more lesions of the GI tract. In some embodiments, the disease or condition is an inflammatory bowel disease. In some further embodiments, the inflammatory bowel disease is ulcerative colitis. In some other embodiments, the inflammatory bowel disease is Crohn's disease.

Accordingly, provided herein are new systemic delivery devices and methods that deliver therapeutic agents into the small intestinal mucosa and/or submucosa by jet injection. Current methods of administration for most large molecule therapeutic agents are subcutaneous (SC), intramuscular (IM), or bolus intravenous (IV) injection targeting the systemic circulation. The devices and methods described herein provide an alternative route of administration to current injectable medications, which can lead to greater convenience and compliance since they minimize or avoid the logistical challenges, patient compliance and adherence challenges, pain, and discomfort associated with traditional routes of administration.

Also, by providing a higher concentration of therapeutic in GI tissue, the devices and methods described herein are particularly well-suited for treatment of diseases and conditions of the endoderm, including the liver.

### Device Description

### General

In general, the ingestible device is suitable for swallowing by a patient and for safely and effectively passing through the GI tract of the patient. Generally, the device can be in the shape of a capsule, a pill or any other swallowable form that may be orally consumed by the subject. In some embodiments, the ingestible device can be swallowed voluntarily under medical supervision or in a home use environment with instruction provided ahead of subsequent ingestion. Generally, ingestible devices are intended for single subject, single use. The ingestible device can have a density high enough to cause the ingestible device to sink within human stomach fluid, e.g., the unfilled ingestible device can have a density of greater than 1.01 g/cm3. The ingestible device can have maximum dimensions that allow the ingestible device to pass through an average human GI tract. In some embodiments, the ingestible device is configured to prevent tumbling in the small intestine of a human. For example, the ingestible device is of sufficient length whereby it will not tumble in the small intestine of a human before, during, or after the dispensable substance is released. Generally, the ingestible device is configured to deliver a sufficient amount of therapeutic agent contained in the dispensable substance to be effective for its intended purpose. In general, the ingestible device's patient-contacting portions (e.g., exterior surface) and dispensable substance-contacting portions are biocompatible. Preferably, the device can withstand an indirect bite force without damaging the housing damage or resulting in leakage. As an example, when containing the dispensable substance, the ingestible device can withstand a bite force of at least about 60 Newtons (N). Generally, unless otherwise intended (see discussion below) components of the ingestible device can withstand exposure to a pH range expected in the human GI tract without substantial loss of functionality, substantial structural damage, or substantial leakage. As an example, in some embodiments, the ingestible device can withstand submersion in a pH 1.5+0.5 fluid environment for at least about 24 hours without substantial loss of functionality, substantial structural damage, or substantial leakage. In general, the ingestible device can maintain an external fluid barrier between the inside of the ingestible device and the GI tract of the subject during transit therethrough. Generally, the ingestible device can withstand external fluid pressures to which it is exposed during use without substantial loss of functionality, substantial structural damage, or substantial leakage. As an example, in some embodiments, the ingestible device undergoes no substantial loss of functionality, substantial structural damage, or substantial leakage when exposed to a sustained pressure of at least about 2 psig for at least about 24 hours and/or when exposed to a momentary pressure of at least about 5 psig momentary pressure for at least about 1 minute.

In general, an ingestible device disclosed herein includes the following features.

### Housing

In some embodiments, the ingestible device comprises a housing configured to maintain its mechanical integrity during use of the ingestible device. In some embodiments, the housing has a first portion and a second portion. In some further embodiments, the housing has a first actuation component on the housing, and a second actuation component within the housing. In some embodiments, a storage reservoir is located within the housing, wherein the storage reservoir is configured to store a dispensable substance. In some embodiments, the housing has an opening in fluid communication with the storage reservoir. In some embodiments, the ingestible device employs an electrolytic mechanism for creating one or more openings in the ingestible device, wherein a substance can be dispensed through the opening as described in WO2019178071. For example, the housing may comprise an external electrolytic circuit (electrolytically erodible surface being on the exterior of the device), whereby the surrounding gastric fluids are the electrolyte that completes an electrolytic circuit between anode and cathode. With sufficient bias voltage (e.g., 1.5-15 volts, such as 3-5 volts), the anode will dissolve or erode electrolytically and thus create an opening in the housing within a desired time interval. In some embodiments, the one or more openings created by an electrolytic mechanism are coupled to one or more nozzles, thereby allowing for trans-epithelial, epithelial, or topical delivery as described herein. In some embodiments an ingestible device includes an enteric coating on the housing. In certain embodiments, the enteric coating covers only certain regions of the housing. The housing may be designed to withstand the chemical and mechanical environment of the GI tract (e.g., effects of muscle contractile forces and concentrated hydrochloric acid in the stomach). A broad range of materials that may be used for the housing. Examples of these materials include, but are not limited to, thermoplastics, fluoropolymers (such as fluorinated ethylene propylene (FEP) and perfluoroalkoxy (PFA)), elastomers, stainless steel, and glass complying with ISO 10993 and USP Class VI specifications for biocompatibility; and any other suitable materials and combinations thereof. In certain embodiments, these materials may further include liquid silicone rubber material with a hardness level of 10 to 90 as determined using a durometer (e.g., MED-4942™ manufactured by NuSil™), a soft biocompatible polymer material such as, but not limited to, polyvinyl chloride (PVC), polyethersulfone (PES), polyethylene (PE), polyurethane (PU) or polytetrafluoroethylene (PTFE), and a rigid polymer material coated with a biocompatible material that is soft or pliable (e.g., a poly(methyl methacrylate) (PMMA) material coated with silicone polymer). Use of different materials for different components may enable functionalization of certain surfaces for interaction with proteins, antibodies, and other biomarkers. For example, Teflon® may be used as a material in the ingestible device for movable components in order to reduce friction between these components. Other example materials may include other materials commonly used in micro-fabrication, such as polydimethylsiloxane (PDMS), borosilicate glass, and/or silicon. Although specific materials may be referred to herein as being used to construct the device for illustrative purposes, the materials recited are not intended to be limiting, and one skilled in the art may easily adapt the device to use any number of different materials without affecting the overall operation or functionality of the device. In some embodiments, the housing of the ingestible device may be manufactured from a type of plastic, such as a photosensitive acrylic polymer material or an inert polycarbonate material. The housing may also be formed using material that can be sterilized by chemicals. In some embodiments, the wall of the housing may have a thickness of, for example, from about 0.5 millimeter to about 1 millimeter. In some embodiments, in addition to being biocompatible, the material from which the housing is made is non-ferric and non-magnetic. Such materials include various plastics (e.g., PVC, or polycarbonate). Optionally, the housing can include a metal-based material, such as an alloy, stainless steel or a substantially pure metal. Such materials can be sterilized without affecting the mechanical workings of the ingestible device or the exterior surface of the ingestible device. In some embodiments, the metal-based material is compatible with the dispensable substance over long duration of storage. A wide variety of stainless steel alloys satisfy these criteria, including SAE grades 303, 304, 304L, 316, 316L, 440. In consideration of nickel content, purity, and/or traceability, in some embodiments, the stainless steel grade is approved for use as a surgical implant material, such as ASTM grades F138, F1314, F1586, F2229, or F2581. The walls of the housing of the ingestible device generally are sufficiently thick to withstand internal and external pressures to which they are exposed without substantial loss of functionality, substantial structural damage, or substantial leakage. In general, the walls of the housing are desirably as thin as possible to enhance the volume available for containing dispensable substance. As an example, in some embodiments, the walls are from about 0.05 mm to about 0.5 mm thick (e.g., if made of metal-based material, such as stainless steel) or from about 0.1 to about 1 mm thick (e.g., if made of plastic, such as polycarbonate). In general, the housing is made of material with a thermal expansion coefficient low enough that the device does not substantially deform at temperatures encountered during shipping and storage, or within the GI tract. In some embodiments, the walls of the housing are made of an electrolytically erodible surface as described in WO2019178071. For example, in some embodiments, the housing includes an electrolytically erodible valve coupled to a nozzle for exposing the liquid volume to its surrounding environment. The exposed metal anode material acting as valve can include a metal alloy or substantially pure metal that is acceptable for human ingestion from consideration of its biocompatibility in the amounts electrolyzed during opening of the valve. It can be desirable to have the thickness of metal in the valve area be small (e.g., to reduce the time and amount of current used to open the valve). For example, the metal portion of the drug container can be 0.025 mm thick across a diameter that matches or slightly exceeds the diameter of the coupled nozzle (e.g., 0.60 mm). In general, the thickness of the metal in the valve area can be in the range 0.002 mm to 0.200 mm.

The housing of an ingestible device may be assembled from multiple modules. For example, in some embodiments, the housing is assembled from two modules. In such embodiments, one of the modules can contain the dispensable substance (“drug module”), and the other module can contain the drive force generator and the drive coupling (“drive module”). Typically, the drug module includes a housing part of appropriate size, shape and material(s) as discussed herein. Usually, the housing part is sterilized, and dispensable substance is subsequently disposed within the housing under aseptic conditions. Optionally a sterile seal (e.g., a sterile foil seal) is incorporated into the drug module. The components of the drug module (e.g., a housing part, a drive force generator, a drive coupling) are assembled in a clean environment. The drug module and the drive module are subsequently combined to form the ingestible device. Representative examples of modules, their separate assembly, and their combination to form an ingestible device, are provide elsewhere herein.

Generally, an ingestible device is sized and shaped for relatively safe and effective movement and intended use within the GI tract of the subject. In certain embodiments, an ingestible device is a capsule having an industry standard size. For example, in some embodiments, an ingestible device is configured as a 00 capsule or a 000 capsule.

In certain embodiments, the housing of an ingestible device has a length of at least about 20 mm (e.g., at least about 21 mm, at least about 22 mm, at least about 23 mm) and/or at most about 28 mm (e.g., at most about 27 mm, at most about 26 mm).

In some embodiments, the housing of an ingestible device has a diameter of at least about 7 mm (e.g., at least about 7.5 mm, at least about 8 mm, at least about 8.5 mm, at least about 9 mm, at least about 9.5 mm) and/or at most about 12 mm (e.g., at most about 11.5 mm, at most about 11 mm, at most about 10.5 mm, at most about 10 mm, at most about 9.5 mm, at most about 9 mm).

In certain embodiments, the housing of an ingestible device has an aspect ratio (ratio of length to width) of at least about 0.75 (e.g. at least about 1) and/or at most about 4 (e.g., at most about 3, at most about 2). In some embodiments, the housing of an ingestible device has an aspect ratio of from about 0.75 to 4 (e.g., from about 1 to about 3, from about 1 to about 2). For example, in some embodiments, the housing aspect ratio is about 1.5:1 (length: diameter). In some other embodiments, the housing aspect ratio is about 2:1 (length: diameter).

In certain embodiments, the housing of an ingestible device has a wall thickness of at least about 0.05 mm (e.g., at least about 0.5 mm, at least about 0.6 mm, at least about 0.7 mm) and/or at most about 1 mm (e.g., at most about 0.9 mm, at most about 0.8 mm). In certain embodiments, an ingestible device has a wall thickness of from about 0.05 mm to about 0.5 mm. In some embodiments, an ingestible device has a wall thickness of from about 0.1 mm to about 1 mm. In certain embodiments, one region of the housing of an ingestible device may have a wall thickness that is different from that of a different region of the housing of the ingestible device.

In some embodiments, the housing of an ingestible device has an end round that is spline-shaped or that is spherical. In certain embodiments, an ingestible device has an end round that is from about 1 mm to about 2 mm (e.g., about 1.5 mm). In some embodiments, an ingestible device has an end round that is from about 4 mm to about 4.5 mm (e.g., about 4.25 mm). In certain embodiments, an ingestible device has an end round that is from about 4.9 to about 5 mm (e.g., about 4.95 mm). In some embodiments, an ingestible device has an end round that is from about 5.4 mm to about 5.6 mm (e.g., about 5.5 mm).

In certain embodiments, the housing of an ingestible device has an internal volume of at least about 700 μL (e.g., at least about 750 μL, at least about 800 μL, at least about 850 μL) and/or most about 1700 μL (e.g., at most about 1650 μL, at most about 1600 μL, at most about 1500 μL, at most about 1400 μL, at most about 1300 UL, at most about 1200 μL).

In an exemplary embodiment, the housing of an ingestible device has a diameter of about 11 mm, a length of about 26 mm, a wall thickness of about 0.8 mm, an end round of about 1.5 mm, and an internal volume of about 1685 μL.

In another exemplary embodiment, the housing of an ingestible device has a diameter of about 11 mm, a length of about 26 mm, a wall thickness of about 0.8 mm, an end round of about 5.5 mm (spherical), and an internal volume of about 1475 μL.

In a further exemplary embodiment, the housing of an ingestible device has a diameter of about 9.9 mm, a length of about 26 mm, a wall thickness of about 0.8 mm, an end round of about 1.5 mm, and an internal volume of about 1315 μL.

In yet another exemplary embodiment, the housing of an ingestible device has a diameter of about 9.9 mm, a length of about 26 mm, a wall thickness of about 0.8 mm, an end round of about 4.95 mm (spherical), and an internal volume of about 1177 μL.

In a further exemplary embodiment, the housing of an ingestible device has a diameter of about 8.5 mm, a length of about 23.3 mm, a wall thickness of about 0.7 mm, an end round of about 1.5 mm, and an internal volume of about 861 μL.

In still a further exemplary embodiment, the housing of an ingestible device has a diameter of about 8.5 mm, a length of about 23.3 mm, a wall thickness of about 0.7 mm, an end round of about 4.25 mm (spherical), and an internal volume of about 773 μL.

In yet a further exemplary embodiment, the housing of an ingestible device has a diameter of about 8.5 mm, a length of about 23.3 mm, a wall thickness of about 0.7 mm, an end round that is spline-shaped, and an internal volume of about 820 μL.

In some embodiments, the ingestible device is Size 00.

### Fluid Volume

The ingestible device includes a fluid volume to contain a dispensable substance (e.g., a liquid, a suspension). In some embodiments, the fluid volume is completely disposed within the housing. Optionally, the fluid volume can be defined by a storage reservoir. Such a storage reservoir can be a component that can be prepared separately from the housing. In such a storage reservoir, the dispensable substance can be disposed in the storage reservoir before the storage reservoir is associated with the ingestible device.

### Dispensable Substance

The device may include one or more dispensable substances, with each dispensable substance including one or more therapeutic agents and/or one or more pharmaceutical formulations including one or more therapeutic agents.

### Nozzles

In some embodiments, an ingestible device includes one or more nozzles in fluid communication with the one or more openings in the ingestible device. The nozzle(s) is (are) configured so that the dispensable substance through the nozzle(s) when the dispensable substance is delivered from the ingestible device. In general, a nozzle can have any desired size and shape appropriate for the desired type of delivery of a dispensable substance from the ingestible device. In certain embodiments, a nozzle has a shape and/or size appropriate for trans-epithelial delivery, epithelial delivery or topical delivery. In some embodiments, an ingestible device includes more than one nozzle. In some embodiments, an ingestible device includes from 2 nozzles to 50 nozzles. In certain embodiments, an ingestible device includes 2 nozzles, three nozzles, four nozzles, five nozzles, six nozzles, seven nozzles, eight nozzles, 10 nozzles, 20 nozzles, 30 nozzles, 36 nozzles, 40 nozzles, 50 nozzles). In some embodiments, the nozzles are arranged at even intervals (optionally pair-wise if an even number of nozzles are used) around the circumference of the device.

### Restraining Mechanism

In some embodiments, the ingestible device comprises a restraining mechanism. Generally, a restraining mechanism has a first state in which it is configured to prevent the dispensable substance from exiting the ingestible device via the opening(s), and a second state in which it is configured so that it does not prevent the dispensable substance from exiting the ingestible device via the opening(s). The restraining mechanism can be configured to transition from its first state to its second state when it is exposed to a triggering condition. The restraining mechanism may be provided by one or more restraining elements. The restraining elements can have a first state in which they are configured to prevent the dispensable substance from exiting the ingestible device via the openings, and a second state in which they are configured to allow the dispensable substance to exit the ingestible device via the openings. The restraining elements can be configured to transition from the first state to the second state when the restraining elements are exposed to a triggering condition. In some embodiments, the restraining elements comprise a first type of restraining element and a second type of restraining element different from the first type of restraining element. The first type of restraining element can be configured to transition to its second state before the second type of restraining element transitions to its second state. In some embodiments, a restraining elements comprises a lid, a pin, a band, a plug, a dowel, a clasp, a clamp, a flange, a rivet, an annulus, a torus, a ring, a wafer, a cylinder, an asymmetric shape such as a partial annulus, a partial torus, a partial ring, a partial wafer, a partial cylinder, or any combination thereof (e.g., two partial tori). Optionally, a restraining element can have a filled interior (e.g., no hole). Optionally, a restraining element can have a varying thickness (e.g., a center region that is thinner than the edges). In some embodiments, the restraining elements comprise a plasticizer such as triethyl citrate (TEC). In some embodiments, the restraining elements comprise a degradable and/or erodible material, such as, for example, an enteric material. The enteric material may be degradable and/or erodible in the small intestine of the GI tract, or may be degradable and/or erodible in the large intestine of the GI tract, for example, the colon. In some embodiments, a restraining mechanism can be a mechanism that prevents the dispensable substance from being delivered from the ingestible device even when the drive force generator (or optionally the drive coupling) applies an internal force. For example, such a restraining can be an element (e.g., a pin, a band, a plug) in the opening (e.g., nozzle opening) through which the dispensable substance can be delivered from the ingestible device. Such a restraining element can be formed, for example, of a material that degrades and/or erodes as discussed above.

In general, a restraining mechanism includes a material that will lose a sufficient degree of its mechanical strength at the desired location to cause the ingestible device to deliver the dispensable substance. The material may undergo loss of mechanical strength to any appropriate mechanism or combination of mechanisms, including, for example, moisture ingress, solubility, swelling, leaching, eroding and/or the like.

In some embodiments, a restraining mechanism includes a degradable and/or erodible material such as a water soluble material, optionally with one or more coatings of one or more enteric materials. The degradable and/or erodible material is designed to lose its mechanical strength in the presence of moisture (e.g., liquid present in the GI tract or a “biological fluid”).

Generally, an enteric material erodes after being swallowed, e.g., in the small intestine or in the large intestine. In some embodiments, the degradable and/or erodible material is coated with an enteric material that limits the amount of moisture or fluid reaching the degradable and/or erodible material, whereby the degradable and/or erodible material is able to resist a trigger load, for example, for at least two hours at a pH of 1.1. In certain embodiments, the enteric material breaks down to release a trigger load after being exposed to a pH of 1.1 for two hours followed by exposure to a pH of 6.8 for 10, 20, 30, 40, 50, 60 or more minutes.

An enteric material can be in the form of one or more coatings at varying coating weights (e.g., one or more spray coatings and/or one or more dip coatings) on a degradable and/or erodible material such as a water soluble material. For example, in some embodiments, compared to the weight of the degradable and/or erodible material, the coating weight can be 5%, 10%, 15%, 20%, 25%, 30%, 35%, 40%, 45%, 50%, 55% or more. In general, the coating weight can be selected as desired, e.g., based on the intended use of the ingestible device. For example, the coating weight can be selected to select a desired location and/or time for the degradable and/or erodible material to degrade and/or erode to a sufficient extent to trigger delivery of the dispensable substance from the ingestible device.

Desirably, the degradable and/or erodible material is sufficiently strong enough to resist the trigger load when dry, but also capable of sufficiently weakening to release the trigger load when the degradable and/or erodible material is exposed to an aqueous environment for a desired period of time, such as, for example, at least two minutes, e.g., at least 5, 10, 30, 60, 120 or at least 160 minutes.

In some embodiments, a triggering mechanism has a density of from about one g/cm3 to about 3 grams/cm3 (e.g., from about 1.3 g/cm3 to about 2 g/cm3).

In certain embodiments, a triggering mechanism is from about 1 mm to about 5 mm thick (e.g., from about 1 mm to about 2 mm thick).

In some embodiments, a coating of enteric material has a density of from about 0.5 mg/cm2 to 20 mg/cm2 (e.g., from about 2 mg/cm2 to about 6 mg/cm2).

Examples of degradable and/or erodible materials include polyethylene glycol (PEG) and Isolmalt. In some embodiments, a degradable and/or erodible material includes one or more diluents/fillers, one or more binders, and/or or more disintegrants. Examples of diluents/fillers include lactose, starch, mannitol, microcrystalline cellulose, carboxymethyl cellulose, and dicalcium phosphate. Examples of binders include povidone, hypromellose, hydroxypropyl cellulose, copovidone, and microcrystalline cellulose. Examples of disintegrants include crospovidone, croscarmellose sodium (SD-711), sodium starch glycolate, and low-substituted hydroxypropyl cellulose. Optionally, a degradable and/or erodible material can include a lubricant, such as, for example, magnesium stearate.

As an example, a degradable and/or erodible material includes starch (e.g., StarTab grade from Colorcon, Starch 1500 grad from Colorcon), microcrystalline cellulose (e.g., Vivapur 102 grade from JRS Pharma), croscarmellose sodium (e.g., Ac-di-sol SD-711 grade from FMC Biopolymer), and magnesium stearate (e.g., Ligamed MF-2-V grade from Giusto Faravelli), and optionally further includes talc (e.g., PSD<75 μm grade from Acros), enteric methacrylate polymer (e.g., FL30 D-55 grade from Evonik), and HPMC polymer sub coat (e.g., Opadry 03K19229 grade from Colorcon). As an example, a degradable and/or erodible material can include Starch 1500 (e.g., 49.6% w/w), microcrystalline cellulose 102 Starch (e.g., 49.6% w/w); and croscarmellose sodium SD-711 (e.g., 0.5% w/w); and magnesium stearate (e.g., 0.26% w/w). As a further example, a degradable and/or erodible material can include Startab (e.g., 49.6% w/w), microcrystalline cellulose 102 Starch (e.g., 49.6% w/w); and croscarmellose sodium SD-711 (e.g., 0.5% w/w); and magnesium stearate (e.g., 0.26% w/w). As another example, a degradable and/or erodible material can include Starch 1500 (e.g., 48.9% w/w), microcrystalline cellulose 102 Starch (e.g., 48.9% w/w); croscarmellose sodium SD-711 (e.g., 2% w/w); and magnesium stearate (e.g., 0.26% w/w). As further example, a degradable and/or erodible material can include Startab (e.g., 49.6% w/w), microcrystalline cellulose 102 Starch (e.g., 49.6% w/w); croscarmellose sodium SD-711 (e.g., 2% w/w); and magnesium stearate (e.g., 0.26% w/w). As another example, a degradable and/or erodible material can include dicalcium phosphate (e.g., 48.9% w/w), microcrystalline cellulose 102 Starch (e.g., 48.9% w/w); croscarmellose sodium SD-711 (e.g., 2% w/w); and magnesium stearate (e.g., 0.25% w/w). As a further example, a degradable and/or erodible material can include dicalcium phosphate (e.g., 33.25% w/w), microcrystalline cellulose 102 Starch (e.g., 33.25% w/w); mannitol (e.g., 33.25% w/w); and magnesium stearate (e.g., 0.25% w/w).

Examples of enteric materials coated on a degradable and/or erodible material include: spray coated Eudragit FL 30 D-55 (e.g., 12 mg/cm2 direct spray coated Eudragit FL 30 D-55 on a water soluble material); dip coated Eudragit L 100 D-55 (e.g., 4 mg/cm2 Eudragit L 100 D-55 dip coated onto an HPMC capsule cap); and spray coated Eudragit FL 30 D-55 (e.g., 9 mg/cm2 Eudragit FL 30 D-55 direct spray coated on a water soluble material; 6 mg/cm2 Eudragit FL 30 D-55 direct spray coated on a water soluble material).

In some embodiment the entire release component comprises 80%, 85%, 90%, 95% or more of an enteric material (e.g., the release component is made entirely of Eudragit 100 or another enteric material).

### Release, Actuation or Triggering Mechanism

In some embodiments, the ingestible device comprises a triggering mechanism configured to cause the dispensable substance within the fluid volume to be released under one or more triggering conditions. The triggering mechanism, if used, initiates a drive force generator. A triggering mechanism may incorporate a mechanical feature like a restraining mechanism. As an example, one or more restraining elements degrade and/or erode in the presence of certain GI tract conditions (e.g., pH greater than 5), thereby triggering a drive force generator, such as a compressed spring. As another example, a spring may have a piercing element that pierces a cylinder with compressed gas, whereby the released gas acts as a force applied to a dispensable substance. In certain embodiments, a triggering mechanism incorporates an electrical feature. For example, an enteric coating degrades and/or erodes in the presence of certain GI tract conditions (e.g., pH greater than 5), thereby exposing conductors to intestinal fluid, which acts as a liquid conductor to triggering the drive force generator. In some embodiments, a triggering condition relates to a condition of the GI tract such as temperature, pH, presence of one or more enzymes, and time. In some more particular embodiments, the condition of the GI tract is a pH of greater than 5. In certain embodiments, the triggering mechanism is configured so that the release mechanism is autonomously triggered (e.g., due to degradation, dissolution and/or erosion of the restraining mechanism due to conditions in the GI tract).

A restraining element can include one or more small molecule therapeutic agents, e.g., one or more small molecule therapeutic agents as disclosed herein. In certain embodiments, a small molecule therapeutic agent contained in the restraining mechanism can be the same as a therapeutic agent contained in the dispensable substance. A small molecule therapeutic agent contained in the restraining mechanism can be different from a therapeutic agent contained in the dispensable substance. The restraining mechanism can include multiple small molecule therapeutic agents, with the dispensable substance containing the same therapeutic agents. In certain embodiments, the dispensable substance includes a therapeutic agent that is capable of treating a certain condition, and a small molecule therapeutic agent included in the restraining element is capable of treating the same condition. In some embodiments, the dispensable substance includes a therapeutic agent that is capable of treating a certain condition, and a small molecule therapeutic agent included in the restraining element is capable of treating a different condition. In certain embodiments, the dispensable substance includes a therapeutic agent that is capable of treating a certain condition, and the small molecule therapeutic agent included in the restraining element is capable of treating the same condition and at least one different condition. In some embodiments, a small molecule therapeutic agent included in the restraining element is capable of treating a certain condition, and the dispensable substance includes a therapeutic agent that is capable of treating at least one different condition. Other combinations are possible.

In general, the initial gas pressure within the gas cylinder (gas pressure before the gas cylinder is implemented as a force generator) is appropriate to provide the desired internal pressure. Typically, the initial gas pressure in the cylinder is at least about 500 psig (e.g., at least about 600 psig, at least about 700 psig, at least about 750 psig, at least about 800 psig, at least about 850 psig, at least about 900 psig) and/or at most about 1,200 psig (e.g., at most about 1,100 psig, at most about 1,000 psig, at most about 950 psig, at most about 900 psig). In some embodiments, the initial gas pressure within the gas cylinder is from about 500 psig to about 1,200 psig (e.g., from about 600 psig to about 1,100 psig, from about 700 psig to about 1,000 psig, from about 750 psig to about 950 psig, from about 800 psig to about 950 psig, from about 850 psig to about 950 psig).

The burst pressure of the gas cylinder (the minimum pressure at which the gas cylinder bursts) is usually based on the desired initial gas pressure within the gas cylinder. For initial gas pressures noted in the preceding paragraph, the burst pressure of the gas cylinder can be at least about 2,800 to 4500 psig

Generally, the gas within the gas container or cylinder can be a single gas or a mixture of two or more gases. Exemplary gases include air, nitrogen, oxygen, carbon dioxide, hydrofluorocarbon gases, and noble gases (e.g., helium, neon, argon, krypton, xenon). In some embodiments, the gas within the gas cylinder is a mixture of gases that include helium (e.g., nitrogen/helium mixture, argon/helium mixture). Optionally, such gas mixtures include at most about 5% helium. The presence of helium in a gas mixture can allow for leak checking the gas cylinder based on the presence of helium gas adjacent the exterior of the gas cylinder.

In general, the gas container may be made of any appropriate and/or desired material. Examples include metal, plastic, and/or composite materials. In some embodiments, the gas container is made of stainless steel or galvanized steel. In certain embodiments, the gas container may be made from a material which is itself prepared by a process that includes drawing, stamping, machining, casting, molding, and/or the like (e.g., deep drawing from sheet metal). In some embodiments, the gas container may be made of a ceramic, an alloy, aluminum and/or titanium.

In some embodiments the gas container includes a breakable seal (e.g., a membrane or septum) which is broken via an element (e.g., a piercer) when the gas container is being used as a force generator, as described in more detail below. Typically, the breakable seal is part of an end cap of the gas container. The end cap and/or the breakable seal can be formed of one or more of the materials noted in the preceding paragraph. Breaking the breakable seal may involve, for example, tearing a portion of the breakable seal and/or puncturing a portion of the breakable seal. More generally, breaking the breakable seal means to modify the seal in a manner such that the breakable seal is no longer able to confine the gas within the gas container. In general, the breakable seal is made of a material that has at least a region that is relatively thin and/or that is configured (e.g., scored) to break. Optionally, the entire barrier is relatively thin. As an example, the barrier may have a relatively thin perimeter with a relatively thick portion within the perimeter (e.g., central portion) so that, when the element (e.g., piercer) applies an appropriate force, the relatively thin portion of the breakable seal breaks.

As another example, the barrier may have an inner (e.g., central) portion surrounded by a portion that is scored so that, when the element (e.g., piercer) applies an appropriate force, the scored portion of the breakable seal breaks. In some embodiments, the breakable seal has a substantial constant thickness and has a portion that is configured (e.g., scored) to break when the element (e.g., piercer) applies an appropriate force. In general, such scoring can be configured as desired. As an example, scoring can be configured as a series of parallel lines. As another example, scoring can be configured as a grid (cross-hatched). As a further example, scoring can be configured as a plurality of dots (e.g., equally spaced dots).

In some embodiments, the element (e.g., piercer) has a contact point on the breakable seal. Optionally, the contact point is concentrated in a relatively small local area. For example, the piercer may be a needle or a thin rod element that is cut at an angle to initially generate a single point contact. Relative to the breakable seal, the point of initial contact may be on-center or off-center. Having the point of initial contact off-center relative to the breakable seal can result in a reduced force applied by the element (e.g., piercer). In embodiments where the modified (e.g., scored) region of the breakable seal is off-center, placing the element (e.g., piercer) off-center means that the contact point is closer to the modified (e.g., thinner scored region) of the breakable seal at the contact point of the element (e.g., piercer) with the breakable seal. In certain embodiments where the modified (e.g., scored) region of the breakable seal is a circle, the element (e.g., piercer) can be configures to that its contact point with the breakable seal is near one point on the circle. In general, the closer this contact point is to the modified region of the breakable seal, the lower the force of the element (e.g., piercer) used to break the breakable seal.

To create a relatively fast release, the modified (e.g., score) portion of the breakable seal desirably fails over substantially most of the modified region, e.g., the diameter of the circle when the modified region is a scored region shaped as a circle. In some embodiments, the closer the contact point is to the center of the scored circle, the more likely that the seal fails on the entire circumference of the scored circle. In such embodiments, it is typically desirable to have the contact point of the element (e.g., piercer) be near the circle but not on it. Optionally, the contact point can be move inwards to get fast release properties. Optionally, a wider footprint for the initial contact of the element (e.g., piercer) may be implemented in some embodiments. For example, the contact point can be a sector of an arc placed near a circular score of the breakable seal. This can encourage failure of the breakable seal over a larger sector of the score region, which can yield faster gas escape.

In some embodiments, before the gas container is used as a force generator, the element (e.g., piercer) is not in contact with the breakable seal. In certain embodiments, before the gas container is used as a force generator, the element (e.g., piercer) may be in contact with the breakable seal such that the element (e.g., piercer) applies a relatively low pressure to the breakable seal. This pressure may be, for example, at least about one Newton (e.g., at least about two Newtons, at least about three Newtons, at least about four Newtons, at least about five Newtons) and/or at most about 15 Newtons

Generally, to cause the gas in the gas container to be released from the gas container, the element (e.g., piercer) applies a relatively high pressure to the breakable seal. This relatively high pressure may be, for example, at least about five Newtons (e.g., at least eight Newtons, at least about 10 Newtons, at least about 15 Newtons) and/or at most about 40 Newtons (e.g., at most about 35 Newtons, at most about 30 Newtons, at most about 25 Newtons). In some embodiments, the relatively high pressure may be from about five Newtons about 35 Newtons. Examples of gas containers, including those with an end cap and/or breakable seal, are disclosed, for example, in US 2017/0258583.

In some embodiments, the element (e.g., the piercer) is coupled to an actuator in an actuator assembly. In some embodiments, the actuator assembly has a total length of less than about 2, 5 or 10 mm. The actuator may be provided in the form of a spring (e.g., a wave spring), e.g., having a compressed length of less than about 2.5 to 5 mm, and a stroke length of less than about 0.3 to 0.8 mm.

In some embodiments, the element (e.g., the piercer) is moved relatively quickly when applying the relatively high force to the breakable seal. In certain embodiments, the element (e.g., the piercer) is moved relatively slowly when applying the relatively high force to the breakable seal. In some embodiments, to break the breakable seal, using a lower speed for moving the element (e.g., piercer) allows for use of a lower force compared to the force used to break the breakable seal when the element (e.g., piercer) moves at a higher speed.

The element (e.g., piercer) may move relative to the gas container, or the gas container may move relative to the element (e.g., piercer). For example, the gas container can be coupled to an actuator which causes the gas container to move.

In some embodiments of any of the devices or methods described herein, the releasing of the therapeutic is triggered by one or more of: a pH in the jejunum of about 6.1 to about 7.2, a pH in the mid small bowel of about 7.0 to about 7.8, a pH in the ileum of about 7.0 to about 8.0, a pH in the right colon of about 5.7 to about 7.0, a pH in the mid colon of about 5.7 to about 7.4, or a pH in the left colon of about 6.3 to about 7.7, such as about 7.0.

### Drive Force Generator

The drive force generator is configured to provide the requisite force to the dispensable substance such that, when the restraining mechanism is removed, the dispensable substance is delivered from the ingestible device as desired. The drive force generator can apply force using different mechanisms, including, for example, a compressed gas, a gas generated by chemical reaction, a spring, a liquid-gas mixture, an impact ram, a sudden expansion caused by a controlled exothermic reaction, or the like. When the drive force generator is a spring, the spring can have one or more of the following properties: the outer diameter of the spring is smaller than the inner diameter of the ingestible device; the compressed length of the spring is minimized to leave more space for dispensable substance; the spring is of a conical shape, potentially with a reduction in the solid length of the spring; the free length of the spring is maximized and larger than the free length of the inner cavity of the ingestible device to ensure an acceptable driving pressure is provided throughout the entire time step of delivery; and the spring rate is large enough to provide acceptable pressure from the beginning until the end of delivery of the dispensable substance. Examples of springs include parallel springs, wave springs and conical springs. Examples of chemical reactants include an airbag inflator, a hydrogen cell (e.g., a Varta hydrogen cell), sodium bicarbonate and acid (e.g., alka seltzer and water on board the ingestible device, alka seltzer and GI tract fluid). Examples of compressed gas include a gas charged within the ingestible device, and a container (e.g., cylinder) of compressed gas. In some embodiments, the compressed gas is a gas container from Picocyl LLC, Golden, CO, USA. Exemplary gas cylinders are disclosed, for example, in US 2017-0258583. An example of a liquid-gas mixture is liquid nitrogen/HFA (hexafluoroacetone)/propane. An example of an impact ram is a two-phase spring/ram. Other examples of drive force generators include a wax actuator, heat generated by electric power (Peltier effect-based mechanism), and a mechanical puncture of tissue followed by delivery.

### Drive Coupling

In general, the drive force coupling transfers a force from the drive force generator to the dispensable substance. Examples of a drive coupling include a piston and a membrane. Examples of membranes include balloons and elastomeric materials. An example of a piston is an O-ring sealed piston. In some embodiments, a piston is provided by a gas container, e.g., with added O-rings or a custom housing. In some embodiments, a drive coupling is a vein, such as a rotating vein. In certain embodiments, a drive coupling is a double piston configured to counteract cap impact. In certain embodiments, a drive coupling is a collapsing bag, such as a collapsing foil bag. In some embodiments, a drive coupling is a collapsing bellows. In some embodiments, the drive force generator also serves as the drive coupling. For example, a gas container may not be secured in a fixed position such that, upon actuation, it moves longitudinally to displace drug payload from the device.

### Storage Reservoir

The ingestible device can include a storage reservoir configured to store a dispensable substance. The storage reservoir may be completely disposed within the housing. The ingestible device may be provided with or without the dispensable substance in the storage reservoir.

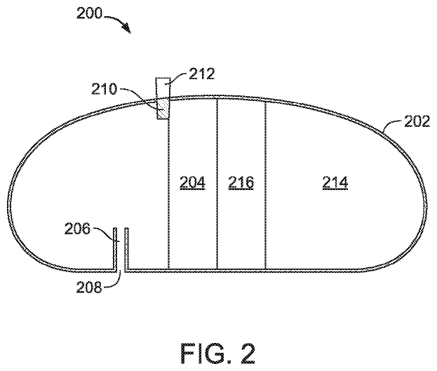

FIG. 2 is a schematic representation of an ingestible device 200 which includes a housing 202, a fluid volume 204 containing a dispensable substance, a nozzle 206 with a nozzle opening 208, a restraining mechanism 210, a triggering mechanism 212, a drive force generator 214 and drive coupling 216. During use, ingestible device 200 is swallowed by a subject and traverses the GI tract. At an appropriate location, the triggering mechanism 212 is triggered, allowing the drive force generator to apply pressure to the drive coupling 216, which then applies pressure to the fluid volume such that at least some of the dispensable substance is delivered out of fluid volume 204, through the nozzle 206, and out of the device 200 via the nozzle opening 208. In some embodiments, the internal pressure is applied, even before the triggering mechanism 212 is triggered. As an example, at an appropriate location, the triggering mechanism 212 is triggered, allowing the drive coupling 216 to apply pressure to the fluid volume 204. In certain embodiments, the internal pressure is not applied until the triggering mechanism 212 is triggered.

### Device for Trans-Epithelial Delivery

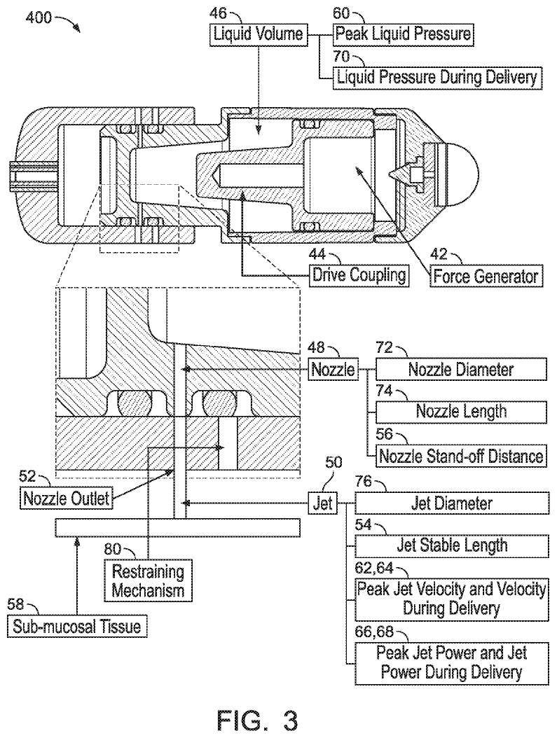

FIG. 3 shows cross sectional views of a representative ingestible device 400 for trans-epithelial delivery, schematically illustrating certain parameters and components of action for the device 400. These include a drive force generator 42 which applies a force (resulting in an internal pressure) to a drive coupling 44. The drive coupling 44 transfers force from the force generator 42 to a fluid volume 46 containing a dispensable substance (e.g., a liquid, a suspension). The force applied to the fluid volume 46 by the drive coupling 44 generates pressure in the fluid volume 46 (fluid pressure). The pressure in the fluid volume 46 generates high-speed flow through an open nozzle 48 to produce a jet 50 of fluid at the nozzle outlet 52 that has a nozzle diameter 72 and the nozzle has a nozzle length 74.

During trans-epithelial delivery, the fluid jet 50 has a jet stable length 54 that is sufficient for the fluid jet 50 to travel across a nozzle stand-off distance 56 to reach the interface of the lumen of the GI tract and the surface of the GI tract facing the lumen. Ultimately, the fluid (e.g., liquid, suspension) impacts the mucosal layer of the GI tract (e.g., the epithelial layer and any mucus that may be present on the epithelial layer) as a stable stream of fluid with little breakup into a spray and is deposited in the submucosal and/or the mucosal tissue 58. That is, between the nozzle outlet 52 and the site of impact at the mucosa, the jet 50 has a jet diameter 76 that can vary in the manner discussed above with respect to the average jet diameter.

The fluid volume 46 experiences a peak fluid pressure 60 that generates the fluid jet 50 that exits the device 40 with a peak jet velocity, and impacts the interface of the lumen of the GI tract and the surface of the GI tract facing the lumen with a peak jet power, peak jet pressure and peak jet force. One of ordinary skill in the art recognizes that these three parameters are interconnected.

The pressure in the fluid volume 46 decreases during delivery so that the fluid pressure during delivery 70 varies, as does the jet power, jet force, and jet pressure. The fluid pressure during delivery 70 maintains the fluid jet 50 at sufficient jet impact force during delivery to continue fluid (dispensable substance including one or more therapeutic agents) delivery from the fluid volume 46 into the submucosal and/or mucosal tissue 58. The surrounding tissue can then absorb the delivered therapeutic agents for systemic delivery of the therapeutic agent.

Even prior to when the subject swallows the ingestible device, the drive coupling 44 transmits force from the force generator 42 to the fluid volume 46. The drive coupling 44 is prevented from moving by a restraining mechanism 80 (e.g., a pin or plug that selectively degrades and/or selectively erodes) until movement of the drive coupling is triggered by a triggering mechanism, and/or an opening becomes open.

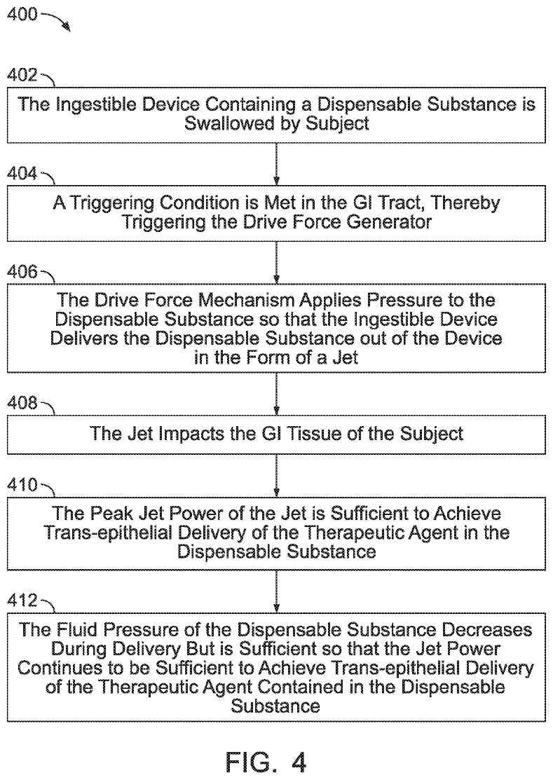

FIG. 4 shows an exemplary process flow chart 400 for use of an ingestible device in which pressure is not applied to the dispensable substance before the subject swallows the ingestible device. The process beings at step 402, when the patient swallows the ingestible device. In step 404, a triggering condition (e.g., pH, change in pH, presence of certain enzyme, concentration of certain enzyme) is met in the GI tract, thereby triggering the drive force generator. In step 406, the drive force mechanism applies pressure to the dispensable substance, resulting delivery of a jet of the dispensable substance from the ingestible device for each opening. In step 408, the jet has a sufficient jet stable length for the jet to impact the GI tissue of the subject. In step 410, the peak jet power of the jet is sufficient to achieve trans-epithelial delivery of the therapeutic agent contained in the dispensable substance. In step 412, the fluid pressure of the dispensable substance decreases during delivery but is sufficiently so that the peak jet power continues to be sufficient to achieve trans-epithelial delivery of the therapeutic agent contained in the dispensable substance.

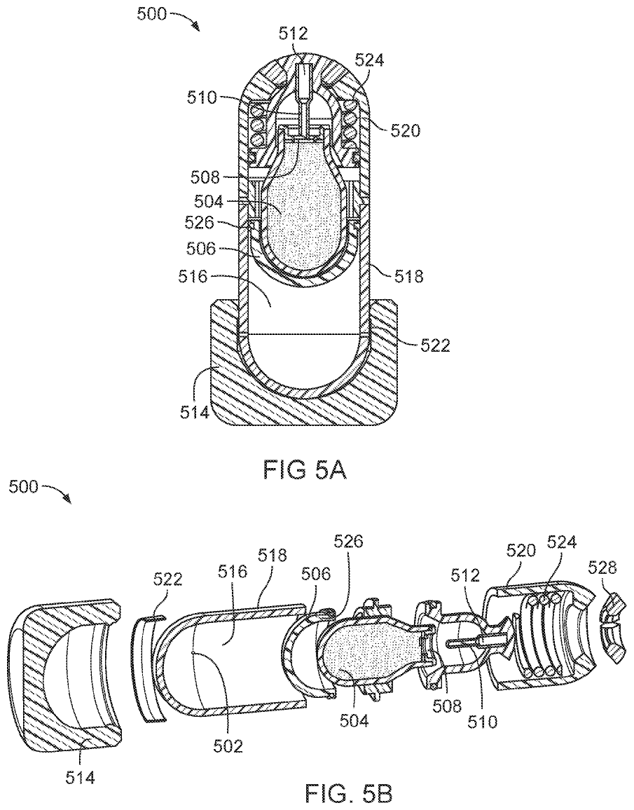

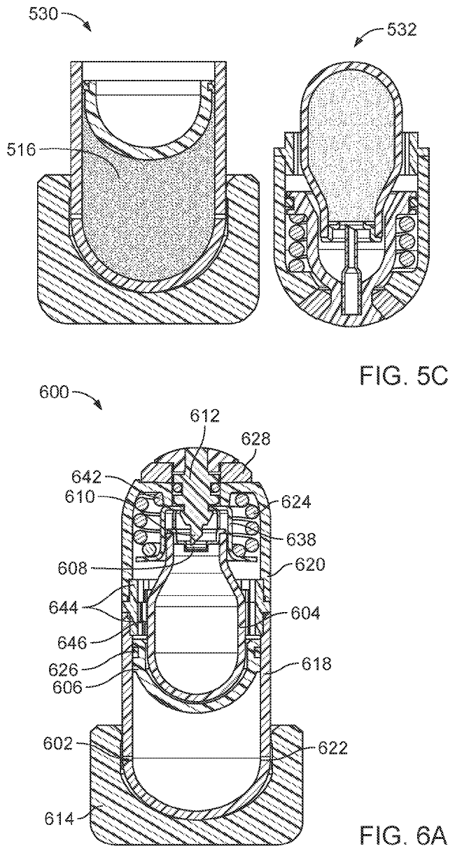

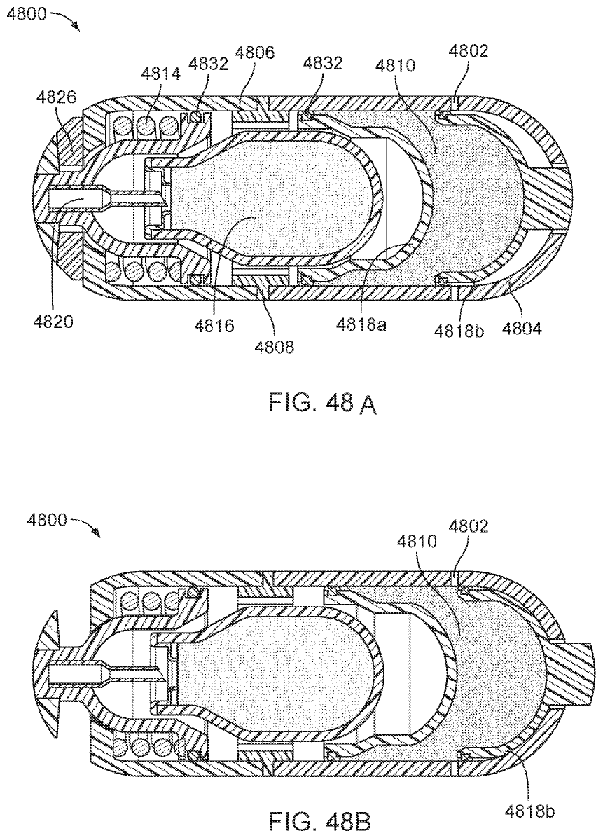

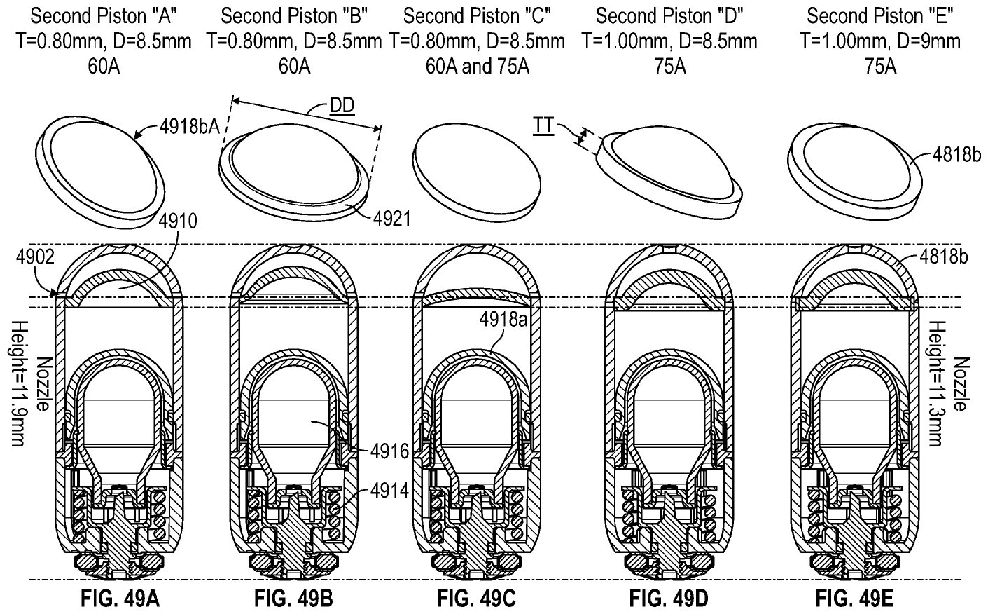

FIGS. 5A-5C show, respectively, a view of an ingestible device 500 as assembled, an exploded of the ingestible device, and aspects of a process of assembly for the ingestible device. Ingestible device 500 includes, a nozzle 502, gas container 504, piston 506, seal 508, pierce pin 510, and piercer 512. A removable cap 514 can be secured over a portion of the ingestible device 500 and removed prior to swallowing. The ingestible device 500 may be used for trans-epithelial delivery. The ingestible device 500 is configured so that a dispensable substance 516 retained within the device that is not under pressure when the subject swallows the ingestible device 500. The ingestible device has two housing parts, a primary container 518 and a secondary container 520. The primary container 518, which includes a fluid volume containing the dispensable substance, can be formed of a cyclic olefin copolymer (COC), such as a molded COC. The primary container 518 includes nozzles 502 with nozzle openings. In some embodiments, the nozzle lengths are about equal to the primary container wall thickness. In some embodiments, the nozzle length is from 0.5 mm to 0.7 mm, or, in some embodiments 0.6 mm. The ingestible device also includes coverings 522 over the nozzle openings, a spring 524, a gas container 504 having a breakable seal, a piston 506 (e.g., made of a COC), a piercer 512, and an O-ring 526. Nozzle covering 522 can be an integral nozzle cover for gastric protection and can be softened after the ingestible device 500 is ingested, e.g., by gastric fluids, such that the coverings 522 dissolve/degrade and expose the nozzles 502. In some embodiments, O-ring 526 may be lubricated. Similarly, any O-ring or piston (including the second pistons of FIGS. 48A-49B) can optionally be lubricated, for example, with parylene-C(chlorinated poly(para-xylylene) polymer.

The ingestible device 500 also includes a collar-shaped trigger element 528 which is the triggering mechanism. Although FIG. 5B shows the trigger element 528 as being collar-shaped, other shapes may be used. In general, the trigger element 528 can have any appropriate shape. Examples of shapes for the trigger element include a complete annulus, an annulus partitioned into two pieces. In some embodiments, the trigger element includes two or more sectors of an annulus with gaps between the sectors. This can increase surface exposure to the environment (e.g., water environment) to promote degradation. The annulus can have an outer diameter of about 5.5 to 8 mm or about 6.85 mm and an inner diameter of about 2.4 to 4.2 mm or about 3.25 mm, and a maximum height or thickness of about 1 to 2 mm or about 1.45 mm.

The ingestible device 500 can comprise two modules: a drug module and a drive module. For example, FIG. 5C shows separate assembly modules 530 (drug module) and 532 (drive module) which are assembled to form ingestible device 500.

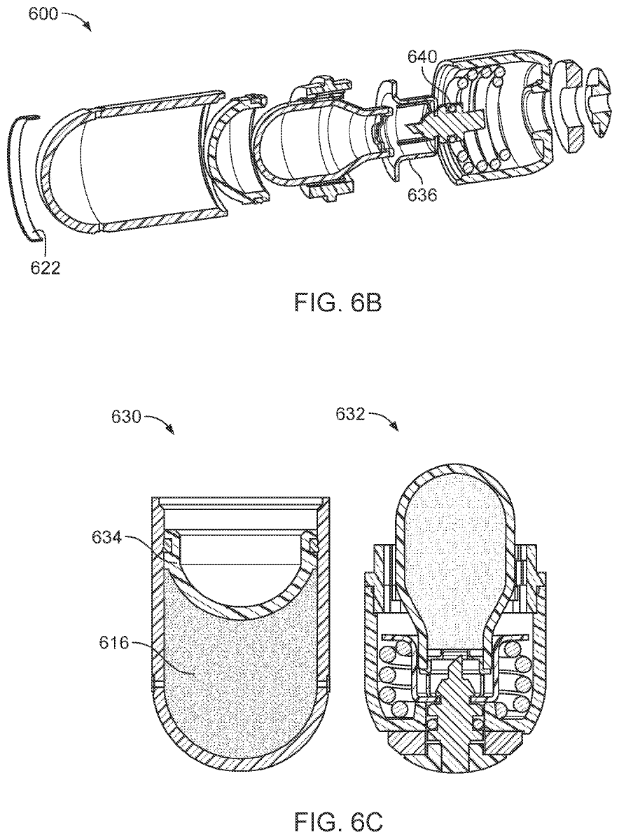

FIGS. 6A-6C, respectively, show view of an ingestible device 600 as assembled, an exploded of the ingestible device 600, and aspects of a process of assembly for the ingestible device 600. The ingestible device 600 may be used for trans-epithelial delivery or for other forms of delivery as appropriate as discussed elsewhere herein. Ingestible device 600 includes, a nozzle 602, gas container 604, piston 606, seal 608, pierce pin 610, and piercer 612. A removable cap 614 can be secured over a portion of the ingestible device 600 and removed prior to swallowing. The ingestible device is configured so that the dispensable substance 616 in the device is not under pressure when the subject swallows the ingestible device. The ingestible device has two housing parts, a primary container 618 and a secondary container 620. The primary container 618, which includes a fluid volume containing the dispensable substance, can be formed of a cyclic olefin copolymer (COC), such as a molded COC, or any other appropriate material as disclosed elsewhere herein. The primary container 618 includes nozzles 602 with nozzle openings. In some embodiments, the nozzle lengths are about equal to the primary container wall thickness. In some embodiments, the nozzle length is from 0.5 mm to 0.7 mm, or, in some embodiments 0.6 mm. The ingestible device 600 also includes coverings 622 over the nozzle openings, a spring 624, a gas container 604 having a breakable seal 608, a piston 606 (e.g., made of a COC, or another appropriate material), a two-part piercer, and an O-ring 626. The ingestible device also includes a collar-shaped trigger element 628 which is the triggering mechanism, which can be made of any appropriate material as discussed elsewhere herein. Although FIGS. 6A-6C depict the trigger element 628 as being collar-shaped, other shapes may be used, as disclosed elsewhere herein.

The device shown in FIGS. 6A-6C has an enhanced piston stabilization length 634 (e.g., about 2 millimeters). The device shown in FIGS. 6A-6C has a metal spring slider 636, which can enhance space efficiency. The device shown in FIGS. 6A-6C has a piercer slider 638 (e.g., a metal piercer slider) that bottoms out on the spring housing during assembly. This can enhance space efficiency. In the device shown in FIGS. 6A-6C, the two part piercer reduces (e.g., removes) tolerances when manufacturing the trigger element from the piercer clearance with the gas container. In the device shown in FIGS. 6A-6C, the piercer seal 640 (e.g., O-ring) has a relatively small diameter, which can enhance stability and/or reduce resistive pressure build up along its stroke length. In the device shown in FIGS. 6A-6C, the spring 624 has a tapered end coil 642, which can enhance the maximum force potential. In some embodiments, a wave spring may be used. FIGS. 6A-6C shows a gas tight seal 644 (e.g., an ultrasonic weld). FIGS. 6A-6C also shows a gas container retention feature 646.

In addition, the ingestible device includes a removable cap 614 which is removed (e.g., by the user) before the ingestible device is swallowed. When the device 600 is swallowed by the subject, the trigger element 628 prevents the dispensable substance 616 in the fluid volume from being under pressure by holding the spring 624 and the piercer 612 in place. When the device reaches the appropriate location in the GI tract, the trigger element 628 at least partially erodes, degrades and/or dissolves (e.g., due to pH, change in pH, presence of certain enzyme, and/or concentration of certain enzyme), and the trigger element 628 is no longer sufficient to hold back the pressure from the spring 624. In some embodiments, the trigger element 628 at least partially erodes, degrades and/or dissolves in the presence of water. In such embodiments, the trigger element may include a covering of a thin film of material that preferentially degrades due to, for example, a change in pH and/or presence of enzyme. The spring 624 forces the pierce pin 610 of piercer 612 into the breakable seal 608, causing the breakable seal to break. This causes gas at elevated pressure to leave the container 604, causing an elevated pressure to bear against the piston 606 and apply pressure to the fluid volume 616. This causes the coverings 622 of the nozzle openings, which are made of a relatively low mechanical strength material (e.g., a foil or a film), to break so that the dispensable substance is delivered out of the nozzle openings in the form of a jet. In certain embodiments, the covering 622 of the nozzle openings are made of a material that erodes, degrades and/or dissolves in the presence of, for example water or elevated pH (e.g., an enteric band or band of water soluble polymer material). The coverings may be partially or completely displaced from the capsule at the time the trigger element actuates. This results in in trans-epithelial delivery of the therapeutic agent contained in the dispensable substance.

FIG. 6C shows aspects of a process for the assembly of the ingestible device, e.g., separate assembly modules 630 and 632 which are assembled to form ingestible device 600. FIG. 6C shows that the primary container, combined with the cap and nozzle coverings, has the dispensable substance added thereto, followed by adding the piston. This may be done in aseptic environment or other environment appropriate for drug filling and separate from the environment where the mechanical drive assembly is constructed. The other housing part and its components are assembled in a clean environment with the piercer held in place by the trigger element. The gas container 604 is held in place by components of this assembly, including the assembly housing which includes features for locating the gas container in its proper position in the assembled ingestible device. Locating and mounting of the gas container may be aided by the formation of a mounting feature integral to the gas container component such as a flange.

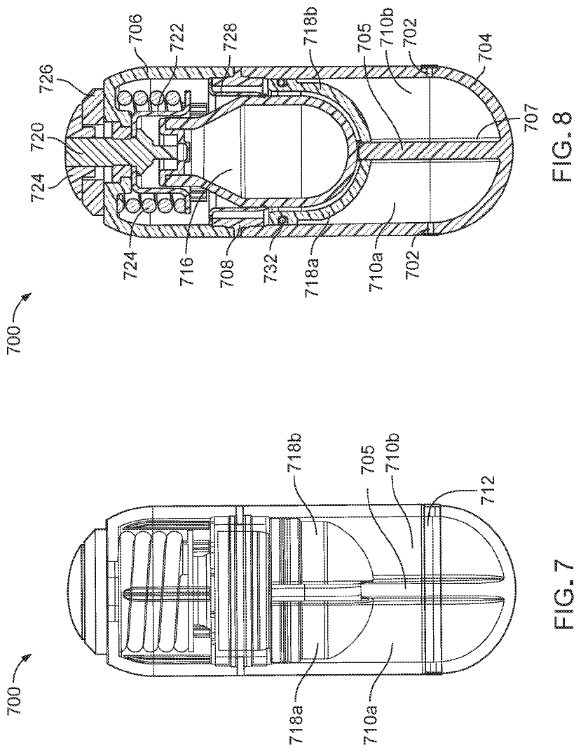

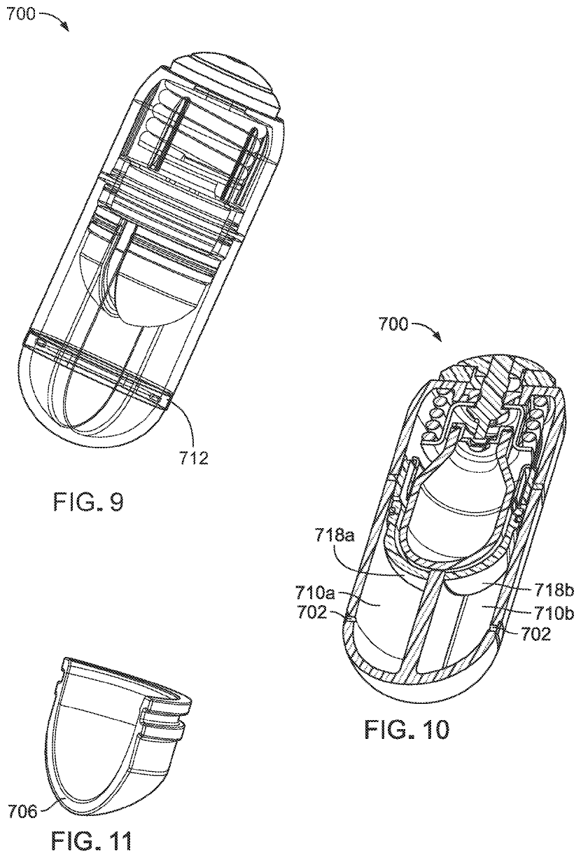

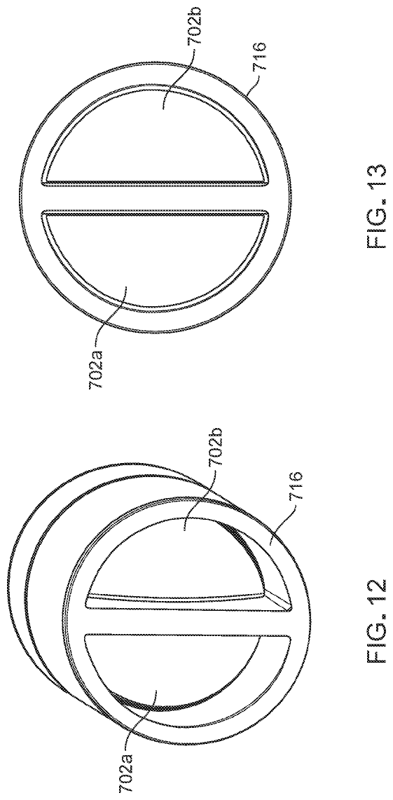

FIGS. 7-13 show various views of an ingestible device 700 and/or aspects of the ingestible device 700. As is apparent, the delivery mechanism of the ingestible device 700 is shown as having a design substantially similar to the device shown in FIG. 5, although, more generally, the ingestible device 700 shown in FIGS. 7-13 can have a delivery mechanism as described elsewhere herein.

Ingestible device 700 includes a gas container 716, a union ring 708, an O-ring 732, an enteric trigger 726, a piercer 720, a spring 724, a spring retention cup 722, a retention element 728, a drug housing 704, a drive housing 706, and a piercer retainer 724.

The ingestible device 700 has two chambers 710a, 710b, each containing a dispensable substance. The chambers are separated by a separator 705, such as a rib, which prevents the dispensable substance in one chamber from entering another chamber, e.g., from 710a to 710b and vice versa. In addition, the ingestible device 700 includes a face seal 707 that seals the separator. The ingestible device also has two pistons 718a, 718b, one for each chamber. Each chamber 710a, 710b has at least nozzle 702 for delivering the dispensable substance from the chamber to an exterior of the ingestible device 700. In general, the dispensable substance in one chamber, e.g., chamber 710a can be the same as or different from the dispensable substance in the other chamber, e.g., chamber 710b. While shown as having two chambers 710a, 710b, the disclosure is not limited in this sense. More generally, the ingestible device 700 can have as many chambers as desired (e.g., two chambers, three chambers, four chambers, five chambers, six chambers, seven chambers, eight chambers, nine chambers, 10 chambers, more than 10 chambers). In general, each chamber 710a, 710b will have a corresponding piston 718a, 718b, and there will be a separator 705 between adjacent chambers. In some embodiments, each chamber has the same internal volume. In certain embodiments, different chambers can have different volumes. Combinations of such embodiments are also possible.

The ingestible device may include an element 712 (e.g., covering) having a first state in which the element 712 at least partially covers the nozzle opening of nozzle 702 in the housing 704 and a second state in which the element 712 does not cover the nozzle opening in the housing 704, where the ingestible device 700 is configured so that, when the drive force coupling (e.g., piston 718a, 718b) moves, the element 712 moves from its first state to its second state. In certain embodiments, the element 712 conforms to an inner radius of the housing 704, is flexible and/or includes a cylindrical portion. In some embodiments, the element 712 is removable from the ingestible device 700 (e.g., when the element 712 is in its second state, the element 712 is removed from the ingestible device). Such a removable element 712 can be, for example, a cap. Optionally, the element 712 moves can move synchronously with the drive force coupling, e.g., pistons 718a, 718b. In some embodiments, when the drive force coupling moves a distance, the element 712 moves the same distance. The ingestible device can include a seal 718 (e.g., an O-ring) that mechanically coupled (e.g., sealed) with the drive force coupling and element 712. With this arrangement, the seal 718 can be configured to cause the movement of the drive force coupling to result in the movement of the element 712.

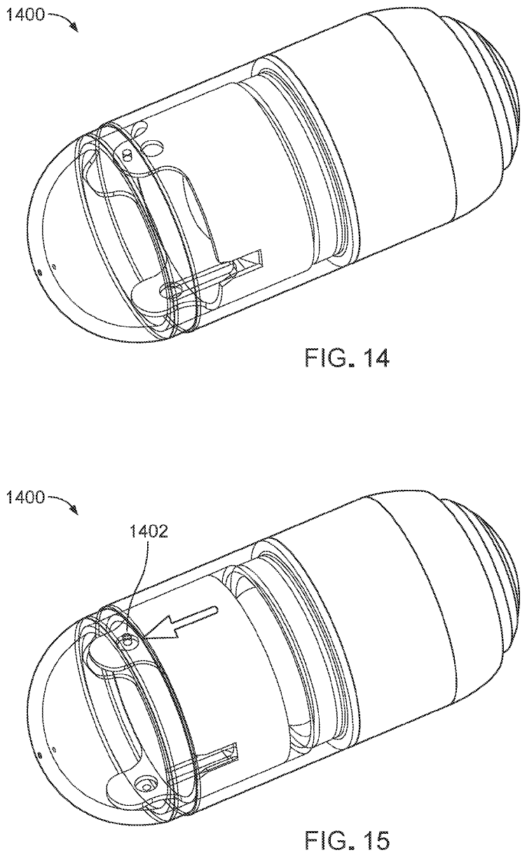

FIGS. 14-18 show an ingestible device 1400 which contains a dispensable substance that is not under pressure when the subject swallows the ingestible device. In FIG. 14, the jet opening 1402 is shown is covered, and in FIG. 15 the jet opening 1402 is uncovered.

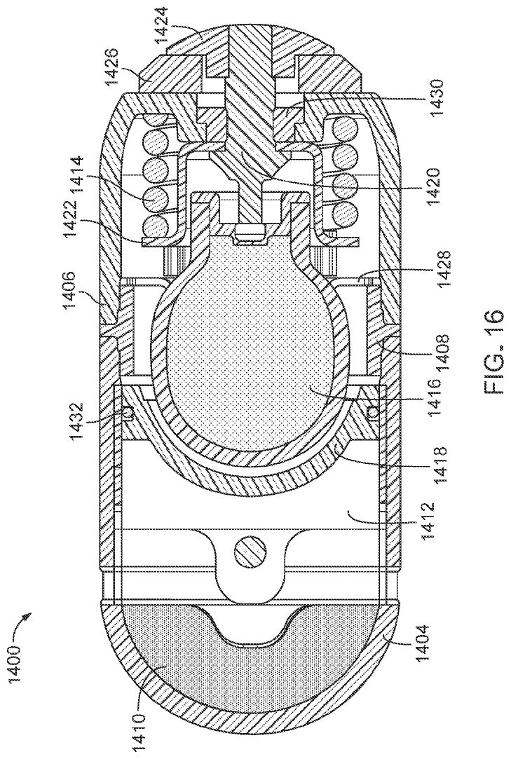

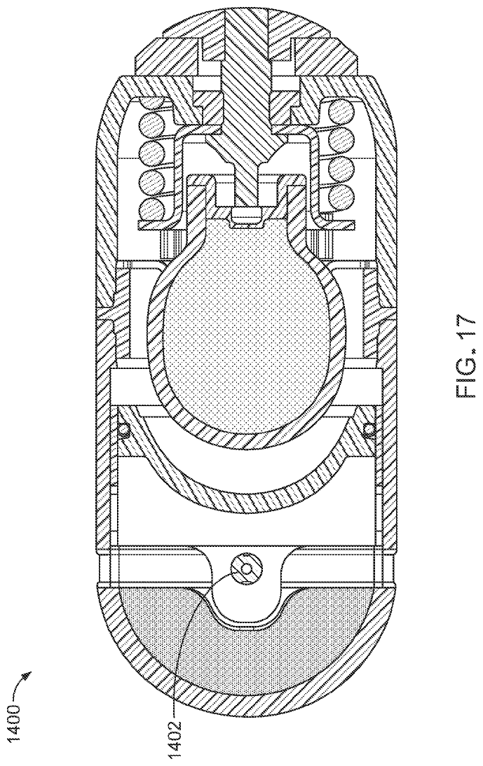

FIGS. 16 and 17 show the ingestible device 1400 in more detail. The ingestible device 1400 has housing parts 1404 and 1406 connected by a union ring 1408 and with a fluid volume 1410 containing a dispensable substance, opening 1402, and a jet covering 1412, e.g., a cylindrical sleeve made of a flexible material which is able to conform to an inside radius of the housing 1406 which slides to open or seal the opening 1402, a spring 1414, a gas container 1416, a piston 1418, a piercer 1420, and an O-ring 1432. Gas container 1416 is retained by retention element 1428. A seal 1430 forms a gas seal between the piercer 1420 and the housing 1404. A spring retention cup 1422 retains the spring-loaded piercer 1420. A piercer retainer 1424 holds the piercer 1420 in place with an enteric trigger 1426 that retains the piercer retainer in place until it dissolves and used as the triggering mechanism. When the device 1400 is swallowed by the subject, the enteric trigger 1426 prevents the dispensable substance in fluid volume 1410 from being under pressure by holding the spring 1414 and the piercer 1420 in place. When the device 1400 reaches the appropriate location in the GI tract, the enteric trigger 1426 degrades and/or dissolves (e.g., due to pH, change in pH, presence of certain enzyme, and/or concentration of certain enzyme) that the pierce pin retainer 1424 is no longer sufficient to hold back the pressure from the spring 1414. The spring 1414 forces the piercer 1420 into the gas container 1416, puncturing the gas container 1416 and causing gas at elevated pressure to leave the container 1416. This causes the gas container 1416 to press against the piston 1418 and apply pressure to the fluid volume 1410. The piston provides friction to slide the jet covering 1412 open exposing the jet openings 1402 such that the dispensable substance is delivered out of the jet opening 1402 in the form of a jet. This results in trans-epithelial delivery of the therapeutic agent contained in the dispensable substance. FIG. 17 shows the embodiment of the ingestible device 1400 in which the jet covering 1412 is slide open to expose the jet openings 1402.

Typically, the ingestible device 1400 is used in trans-epithelial delivery. However, the ingestible device 1400 may be used for either epithelial delivery or topical delivery. Appropriate parameters for the different types of delivery are provided elsewhere herein.

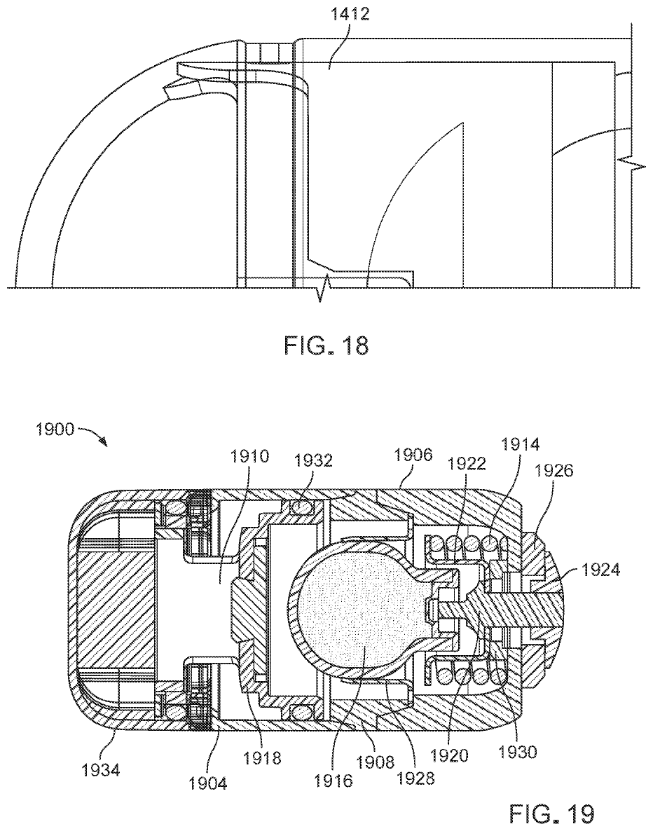

In some embodiments, the housing of the ingestible device 1400 has a diameter from about 9.5 mm to about 10.5 mm (e.g., from about 9.8 mm to about 10 mm), a length from about 23 mm to about 26.5 mm (e.g., from about 23.3 mm to about 26.1 mm), a wall thickness from about 0.4 mm to about 0.6 mm (e.g., about 0.5 mm), a fluid volume from about 425 μL to about 600 μL (e.g., from about 450 μL to about 585 μL), and/or a gas volume in the gas container 1416 from about 150 μL to about 175 μL (e.g., about 160 μL). In some embodiments, the housing of the ingestible device 1400 has a diameter of 9.9 mm, or, in some embodiments, 8.5 mm. In some embodiments, the length of an ingestible device is 23.3 mm, (Size 00) or, in some embodiments, 26 mm (Size 000). In some embodiments, the dispensable substance includes an initial fluid volume of 420 UL FIG. 18 shows the ingestible device 1400 where the jet covering 1412 conforms along a radius of the ingestible device 1400. Exemplary Size 00 devices are also provided in FIGS. 35-40.

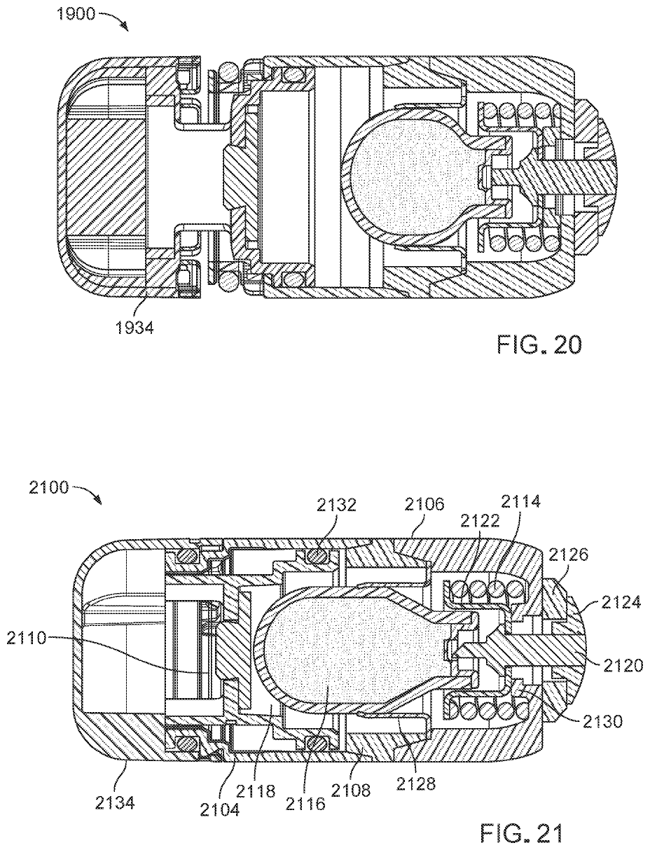

FIGS. 19 and 20 show an ingestible device 1900 in its closed and open states, respectively. The ingestible device 1900 contains a dispensable substance that is not under pressure when the subject swallows the ingestible device. The ingestible device 1900 has housing parts 1904 and 1906 connected by a union ring 1908 and with a fluid volume 1910 containing a dispensable substance, a spring 1914, a gas container 1916, a piston 1918, a piercer 1920, and an O-ring 1932. Gas container 1916 is retained by retention element 1928. A seal 1930 forms a gas seal between the piercer 1920 and the housing 1906. A spring retention cup 1922 retains the spring-loaded piercer 1920. A piercer retainer 1924 holds the piercer 1920 in place with an enteric trigger 1926 that retains the piercer retainer in place until it dissolves and used as the triggering mechanism. When the device 1900 is swallowed by the subject, the enteric trigger 1926 prevents the dispensable substance in fluid volume 1910 from being under pressure by holding the spring 1914 and the piercer 1920 in place. When the device 1900 reaches the appropriate location in the GI tract, the enteric trigger 1926 degrades and/or dissolves (e.g., due to pH, change in pH, presence of certain enzyme, and/or concentration of certain enzyme) that the piercer retainer 1924 is no longer sufficient to hold back the pressure from the spring 1914. The spring 1914 forces the piercer 1920 into the gas container 1916, puncturing the gas container 1916 and causing gas at elevated pressure to leave the container 1916. This causes the gas container 1916 to press against the piston 1918 and apply pressure to the fluid volume 1910. The piston provides friction to cause the cap 1934 to open/deploy such that the dispensable substance is delivered out of the volume 1910. This results in release of the therapeutic agent into the GI tract of the subject.

In some embodiments, the housing of the ingestible device 1900 has a diameter from about 10 mm to about 12 mm, a length from about 23 mm to about 26.5 mm, a wall thickness from about 0.4 mm to about 0.6 mm, a fluid volume from about 565 μL to about 630 μL and/or a gas volume in the gas container 1916 from about 150 μL to about 175 μL. The housing 1900 may have a diameter of 8.5 or 9.9 mm and a length of 23.3 (Size 00) or 26 mm, (Size 000). In some embodiments, the length of an ingestible device 1900 is 23.3 mm, (Size 00) or, in some embodiments, 26 mm (Size 000). In some embodiments, for example, for a Size 000 device, the dispensable substance includes an initial fluid volume of about 420 μL, and, in some embodiments, for a Size 00 device, the dispensable substance includes an initial fluid volume of about 180 μL to about 280 μL. In general, the ingestible device 1900 is used in topical delivery.

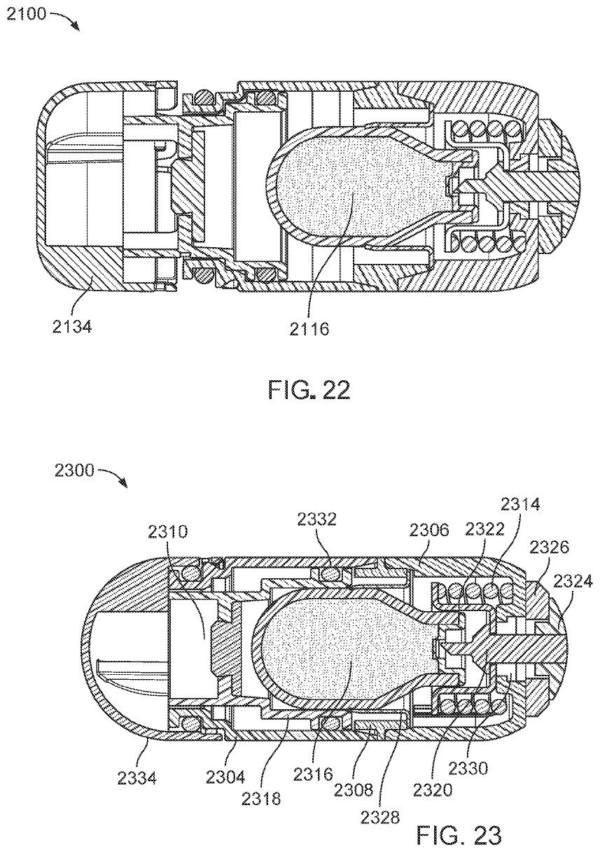

FIGS. 21 and 22 show an ingestible device 2100 in its closed and open states, respectively. The ingestible device 2100 contains a dispensable substance that is not under pressure when the subject swallows the ingestible device. The ingestible device 2100 has housing parts 2104 and 2106 connected by a union ring 2108 and with a fluid volume 2110 containing a dispensable substance, a spring 2114, a gas container 2116, a piston 2118, a piercer 2120, and an O-ring 2132. Gas container 2116 is retained by retention element 2128. A seal 2130 forms a gas seal between the piercer 2120 and the housing 2106. A spring retention cup 2122 retains the spring-loaded piercer 2120. A piercer retainer 2124 holds the piercer 2120 in place with an enteric trigger 2126 that retains the piercer retainer in place until it dissolves and used as the triggering mechanism. When the device 2100 is swallowed by the subject, the enteric trigger 2126 prevents the dispensable substance in fluid volume 2110 from being under pressure by holding the spring 2114 and the piercer 2120 in place. When the device 2100 reaches the appropriate location in the GI tract, the enteric trigger 2126 degrades and/or dissolves (e.g., due to pH, change in pH, presence of certain enzyme, and/or concentration of certain enzyme) that the piercer retainer 2124 is no longer sufficient to hold back the pressure from the spring 2114. The spring 2114 forces the piercer 2120 into the gas container 2116, puncturing the gas container 2116 and causing gas at elevated pressure to leave the container 2116. This causes the gas container 2116 to press against the piston 2118 and apply pressure to the fluid volume 2110. The piston provides friction to cause the cap 2134 to open/deploy such that the dispensable substance is delivered out of the volume 2110. This results in delivery (e.g., topical delivery) of the therapeutic agent contained in the dispensable substance.

In some embodiments, the housing of the ingestible device 2100 has a diameter from about 10 mm to about 12 mm (e.g., from about 11.3 mm to about 11.5 mm), a length from about 23 mm to about 26.5 mm (e.g., from about 23.3 mm to about 26.3 mm), a wall thickness from about 0.4 mm to about 0.6 mm (e.g., about 0.5 mm), a fluid volume from about 565 μL to about 630 μL (e.g., from about 574 μL to about 623 μL), and/or a gas volume in the gas cylinder 2116 from about 150 μL to about 175 μL (e.g., about 160 μL). In some embodiments, the housing of the ingestible device 2100 has a diameter of 9.9 mm, or, in some embodiments, 8.5 mm. In some embodiments, the length of an ingestible device 2100 is 23.3 mm, (Size 00) or, in some embodiments, 26 mm (Size 000). In some embodiments, for example, for a Size 000 device, the dispensable substance includes an initial fluid volume of about 420 μL, and, in some embodiments, for a Size 00 device, the dispensable substance includes an initial fluid volume of about 180 μL to about 280 μL.

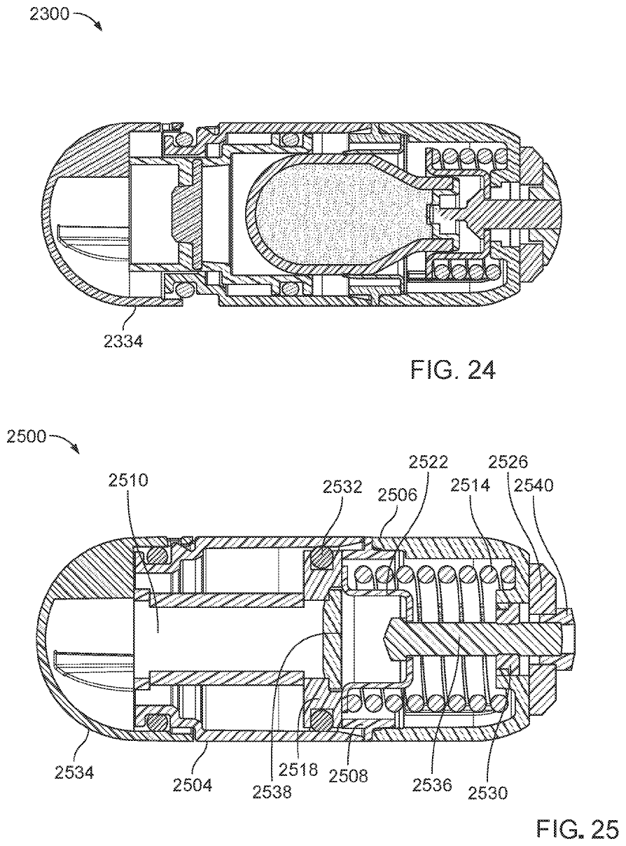

FIGS. 23 and 24 show an ingestible device 2300 in its closed and open states, respectively. The ingestible device 2300 contains a dispensable substance that is not under pressure when the subject swallows the ingestible device. The ingestible device 2300 has housing parts 2304 and 2306 connected by a union ring 2308 and with a fluid volume 2310 containing a dispensable substance, a spring 2314, a gas container 2316, a piston 2318, a piercer 2320, and an O-ring 2332. Gas container 2316 is retained by retention element 2328. A seal 2330 forms a gas seal between the piercer 2320 and the housing 2306. A spring retention cup 2322 retains the spring-loaded piercer 2320. A piercer retainer 2324 holds the piercer 2320 in place with an enteric trigger 2326 that retains the piercer retainer in place until it dissolves and used as the triggering mechanism. When the device 2300 is swallowed by the subject, the enteric trigger 2326 prevents the dispensable substance in fluid volume 2310 from being under pressure by holding the spring 2314 and the piercer 2320 in place. When the device 2300 reaches the appropriate location in the GI tract, the enteric trigger 2326 degrades and/or dissolves (e.g., due to pH, change in pH, presence of certain enzyme, and/or concentration of certain enzyme) that the piercer retainer 2324 is no longer sufficient to hold back the pressure from the spring 2314. The spring 2314 forces the piercer 2320 into the gas container 2316, puncturing the gas container 2316 and causing gas at elevated pressure to leave the container 2316. This causes the gas container 2316 to press against the piston 2318 and apply pressure to the fluid volume 2310. The piston provides friction to cause the cap 2334 to open/deploy such that the dispensable substance is delivered out of the volume 2310. This results in delivery (e.g., topical delivery) of the therapeutic agent contained in the dispensable substance.

The ingestible devices in the Figures may have has a diameter from about 8 mm to about 11 mm, a length from about 23 mm to about 26.5 mm, a wall thickness from about 0.4 mm to about 0.6 mm, a fluid volume from about 230 μL to about 355 μL, and/or a gas volume in the gas container 2316 from about 150 μL to about 175 μL.

FIG. 25 shows an embodiment of an ingestible device 2500, which contains a dispensable substance that is not under pressure when the subject swallows the ingestible device. The ingestible device 2500 has housing parts 2504 and 2506 connected by a union ring 2508 and with a fluid volume 2510 containing a dispensable substance, a spring 2514, a piston 2518, a spring retention pin 2536, and an O-ring 2532. A dispensable substance-containing cap 2538 seals in the dispensable substance (e.g., drug-containing liquid) after the housing part 2504 is filled with the dispensable substance (e.g., drug-containing liquid). A seal 2530 forms a gas seal between the spring retention pin 2536 and the housing part 2506. A spring retention cup 2522 retains the spring retention pin 2536. A pin retainer 2540 holds the spring retention pin 2536 in place with an enteric trigger 2526 that retains the pin retainer in place until it dissolves and used as the triggering mechanism. When the device 2500 is swallowed by the subject, the enteric trigger 2526 prevents the dispensable substance in fluid volume 2510 from being under pressure by holding the spring 2514 and the spring retention pin 2536 in place. When the device 2500 reaches the appropriate location in the GI tract, the enteric trigger 2526 degrades and/or dissolves (e.g., due to pH, change in pH, presence of certain enzyme, and/or concentration of certain enzyme) that the pin retainer 2540 is no longer sufficient to hold back the spring retention pin 2536, releasing spring 2514. The spring 2514 pushes against the piston 2518 such that the piston 2518 applies pressure to the fluid volume 2510. The piston provides friction to cause the cap 2534 to open/deploy such that the dispensable substance is delivered out of the volume 2510. This results in delivery (e.g., topical delivery) of the therapeutic agent out of the dispensable substance.

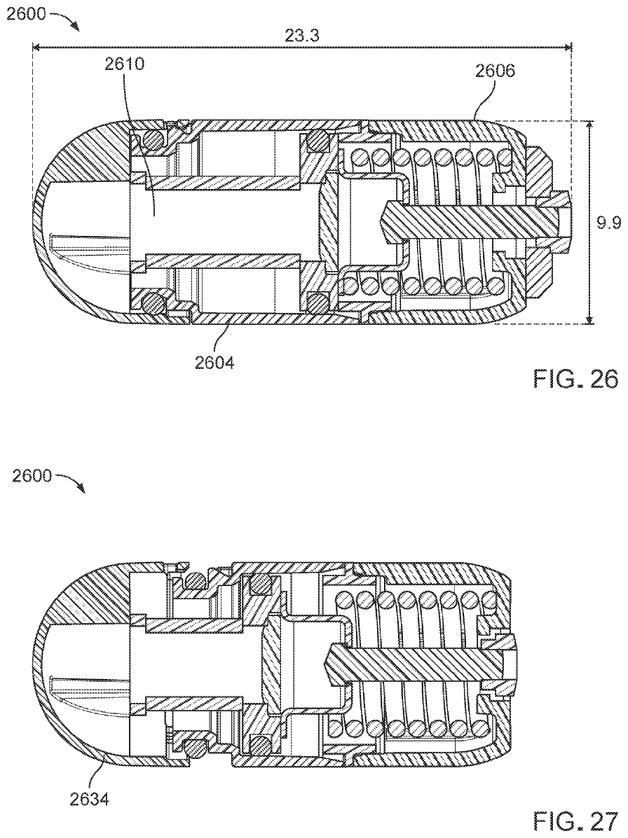

FIGS. 26 and 27 show an ingestible device 2600 in its closed and open states, respectively. The ingestible device 2600 is configured similarly to ingestible device 2500 and having housing components 2604 and 2606 with a smaller profile than the housing component of ingestible device 2500. Fluid volume 2610 of ingestible device 2600 can have a smaller capacity than fluid volume 2510 of ingestible device 2500.

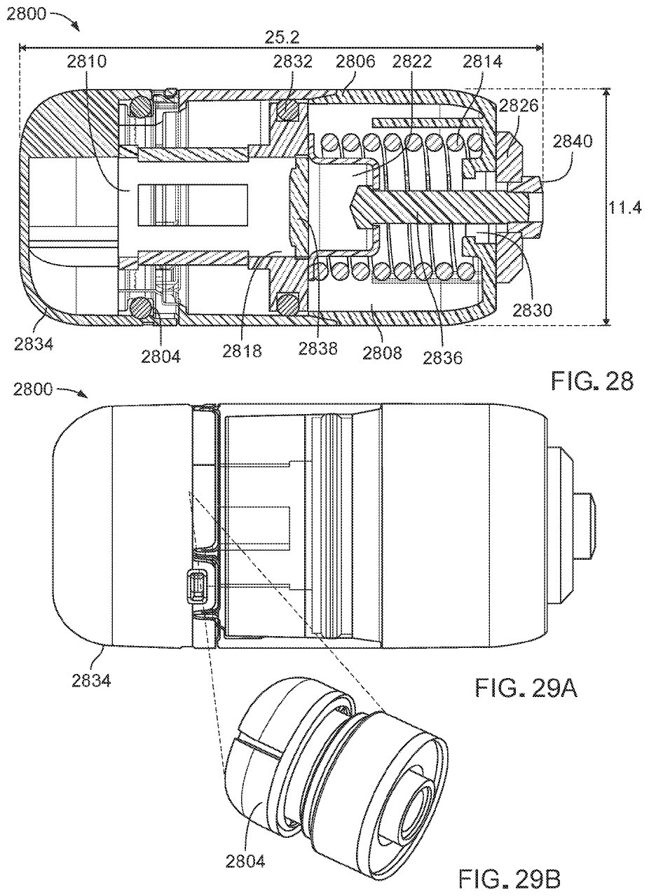

FIG. 28 shows an embodiment of an ingestible device 2800, which contains a dispensable substance that is not under pressure when the subject swallows the ingestible device. The ingestible device 2800 has housing parts 2804 and 2806 connected by a union ring 2808 and with a fluid volume 2810 containing a dispensable substance, a spring 2814, a piston 2818, a spring retention pin 2836, and an O-ring 2832. A dispensable substance-containing cap 2838 seals in the dispensable substance (e.g., drug-containing liquid) after the housing part 2804 is filled with the dispensable substance (e.g., drug-containing liquid). A seal 2830 forms a gas seal between the spring retention pin 2836 and the housing 2806. A spring retention cup 2822 retains the spring retention pin 2836. A pin retainer 2840 holds the spring retention pin 2836 in place with an enteric trigger 2826 that retains the pin retainer in place until it dissolves and used as the triggering mechanism. When the device 2800 is swallowed by the subject, the enteric trigger 2826 prevents the dispensable substance in fluid volume 2810 from being under pressure by holding the spring 2814 and the spring retention pin 2836 in place. When the device 2800 reaches the appropriate location in the GI tract, the enteric trigger 2826 degrades and/or dissolves (e.g., due to pH, change in pH, presence of certain enzyme, and/or concentration of certain enzyme) that the pin retainer 2840 is no longer sufficient to hold back the spring retention pin 2836, releasing spring 2814. The spring 2814 pushes against the piston 2818 such that the piston 2818 applies pressure to the fluid volume 2810. The piston provides friction to cause the cap 2834 to open/deploy such that the dispensable substance is delivered out of the volume 2810. This results in delivery (e.g., topical delivery) of the therapeutic agent contained in the dispensable substance.

FIG. 29A shows an outer view of the embodiment of ingestible device 2800, and FIG. 29B shows and an outer view of the housing component 2804 that retains a fluid volume 2810.

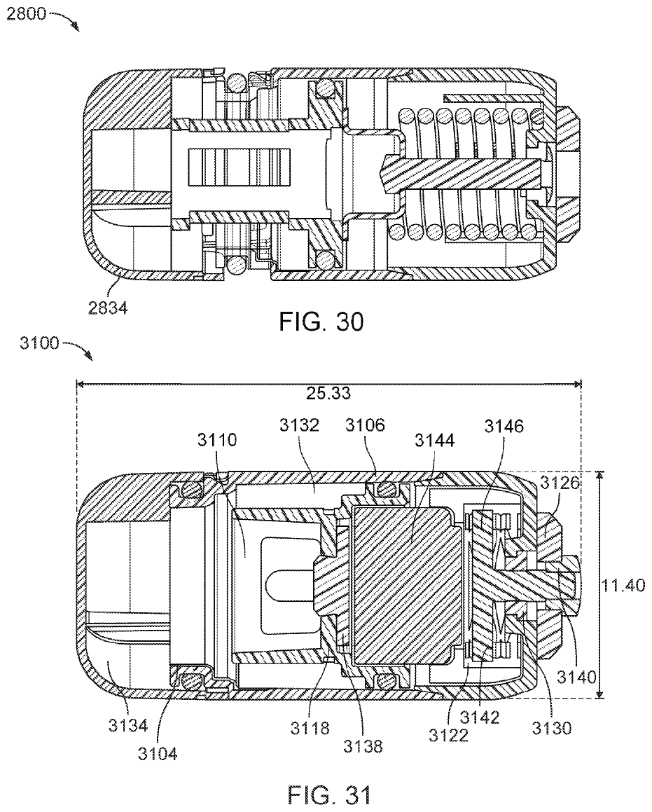

FIG. 30 shows the embodiment of the ingestible device 2800 in which the cap 2834 is opened/deployed.

FIG. 31 shows an embodiment of an ingestible device 3100, which contains a dispensable substance that is not under pressure when the subject swallows the ingestible device. The ingestible device 3100 has housing parts 3104 and 3106 and a fluid volume 3110 containing a dispensable substance, a piston 3118, a wave spring 3142, and an O-ring 3132. A dispensable substance-containing cap 3138 seals in the dispensable substance (e.g., drug-containing liquid) after the housing part 3104 is filled with the dispensable substance (e.g., the drug-containing liquid). A seal 3130 forms a gas seal between the switch 3146 and the housing 3106. A spring retention cup 3122 retains the wave spring 3142. A pin retainer 3140 holds the switch 3146 in place with an enteric trigger 3126 that retains the pin retainer in place until it dissolves and is used as the triggering mechanism. When the device 3100 is swallowed by the subject, the enteric trigger 3126 prevents the dispensable substance in fluid volume 3110 from being under pressure by holding the wave spring 3142 and the switch 3146 in place. When the device 3100 reaches the appropriate location in the GI tract, the enteric trigger 3126 degrades and/or dissolves (e.g., due to pH, change in pH, presence of certain enzyme, and/or concentration of certain enzyme) that the pin retainer 3140 is no longer sufficient to hold back the wave spring 3142 and releasing switch 3146. The switch 3146 completes a circuit with the gas cell 3144, which begins gas production. As pressure builds, piston 3118 slides along a trace and closes the circuit via a conductive O-ring 3132. The circuit opens when the trace ends at a defined travel distance to halt gas production by the gas cell 3144. The piston 3118 applies pressure to the fluid volume 3110 and provides friction to cause the cap 3134 to open/deploy such that the dispensable substance is delivered out of the volume 3110. This results in delivery (e.g., topical delivery) of the therapeutic agent contained in the dispensable substance.

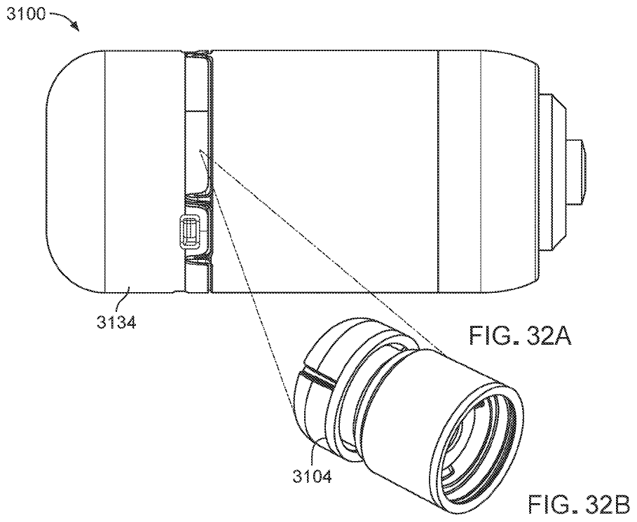

FIG. 32A shows an outer view of the embodiment of ingestible device 3100, and FIG. 32B shows an outer view of the housing component 3104 that retains a fluid volume 3110.

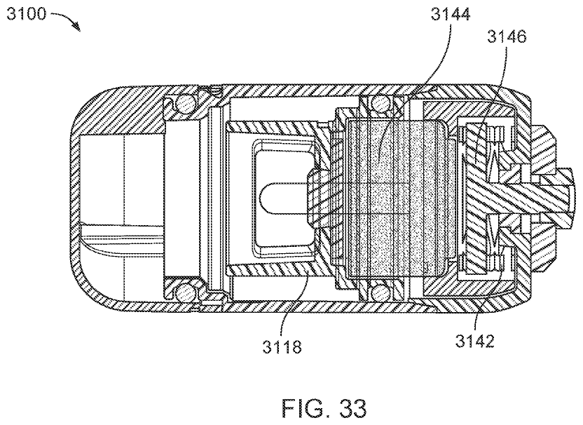

FIG. 33 shows another view of the embodiment of the ingestible device 3100.

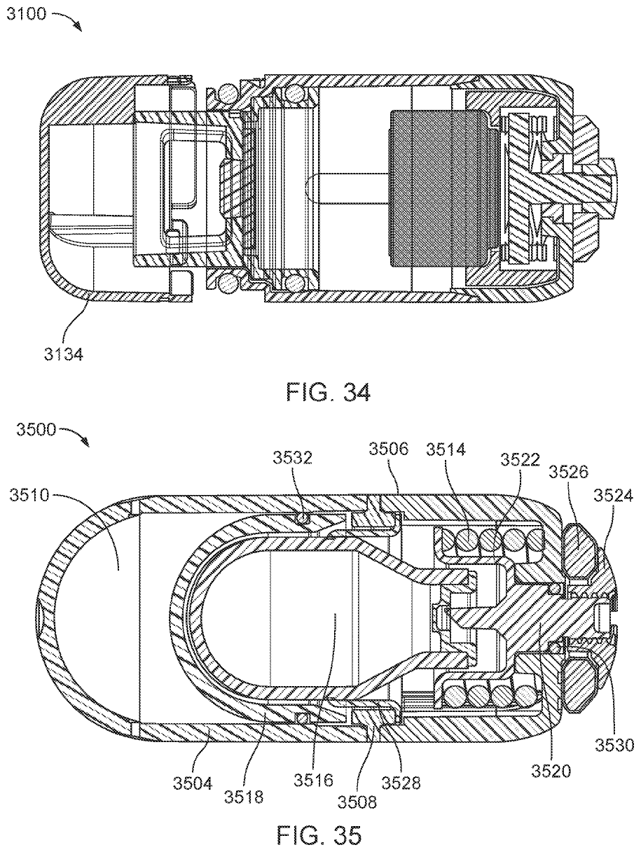

FIG. 34 shows the embodiment of the ingestible device 3100 in which the cap 3134 is opened/deployed.

In some embodiments, a length of an ingestible device can be reduced to achieve a modified 00 standardized length, e.g., approximately 23.3 mm in length, while maintaining a same diameter as a standard size 000. A reduced length of the ingestible device may result in a reduced volume available for the dispensable substance. Adjusting one or more dimensions of a gas container within the ingestible device and/or altering a position of a piston may be utilized to increase an available volume for the dispensable substance, while maintaining a threshold dispensable substance volume and/or pressure provided by the gas container for the ingestible device. In some embodiments, the length of an ingestible device is 23.3 mm, (Size 00) or, in some embodiments, 26 mm (Size 000). Example embodiments are described with reference to FIGS. 35-40 herein.

FIG. 35 shows an embodiment of an ingestible device 3500 for epithelial delivery in which a length of the ingestible device is reduced to achieve a modified size 00 submucosal device, and which contains a dispensable substance that is not under pressure when the subject swallows the ingestible device. The ingestible device 3500 has housing parts 3504 and 3506 connected by a union ring 3508 and with a fluid volume 3510 containing a dispensable substance, a spring 3514, a gas container 3516, a piston 3518, a piercer 3520, and an O-ring 3532. In some embodiments, there may be a gap between the internal housing parts (3504 and 3506) and the union ring 3508, for example, about 50 microns. Gas container 3516 is retained by retention element 3528. A seal 3530 forms a gas seal between the piercer 3520 and the housing 3506. A spring retention cup 3522 retains the spring-loaded piercer 3520. A piercer retainer 3524 holds the piercer 3520 in place with an enteric trigger 3526 that retains the piercer retainer in place until it dissolves and used as the triggering mechanism. When the device 3500 is swallowed by the subject, the enteric trigger 3526 prevents the dispensable substance in fluid volume 3510 from being under pressure by holding the spring 3514 and the piercer 3520 in place. When the device 3500 reaches the appropriate location in the GI tract, the enteric trigger 3526 degrades and/or dissolves (e.g., due to pH, change in pH, presence of certain enzyme, and/or concentration of certain enzyme) that the piercer retainer 3524 is no longer sufficient to hold back the pressure from the spring 3514. The spring 3514 forces the piercer 3520 into the gas container 3516, puncturing the gas container 3516 and causing gas at elevated pressure to 3534 leave the container 3516. This causes the gas container 3516 to press against the piston 3518 and apply pressure to the fluid volume 3510. The piston provides friction to cause the cap 3534 to open/deploy such that the dispensable substance is delivered out of the volume 3510. This results in epithelial delivery of the therapeutic agent contained in the dispensable substance.

In some embodiments, the ingestible device 3500 can retain a dispensable substance volume from about 250 μL to about 350 μL, can have an expansion volume from about 230 UL to about 260 μL, and can have a gas container fill volume from about 140 μL to about 150 μL.

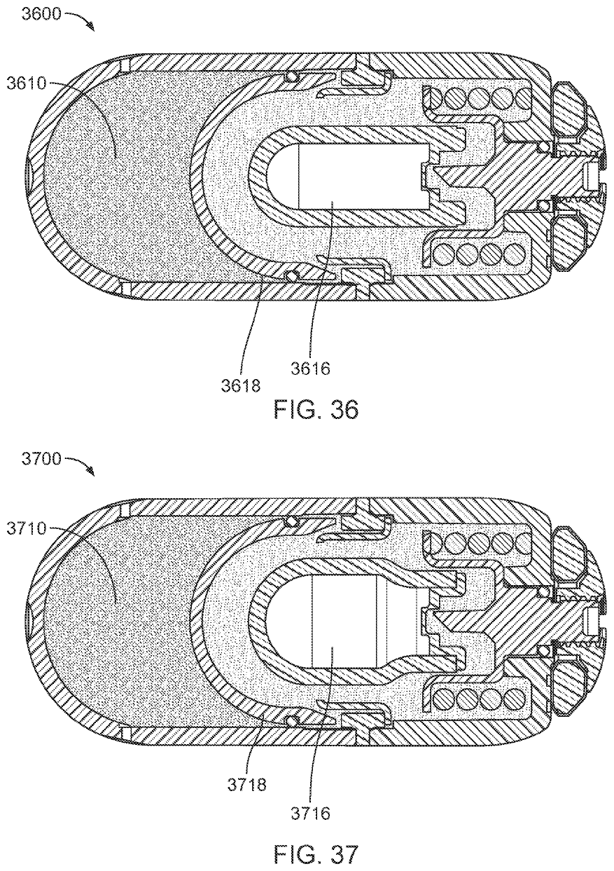

In some embodiments, one or more adjustments to a piston length and/or gas container dimensions can be modified for the ingestible device, e.g., ingestible device 3500. FIGS. 36-40 depict various modifications to piston length and/or gas container dimensions of the ingestible device structure described with reference to FIG. 35.

FIG. 36 shows an embodiment of an ingestible device 3600 in which a length of the ingestible device is reduced to achieve a modified size 00 submucosal device and a piston length is reduced. As shown in FIG. 36, ingestible device 3600 includes a piston 3618, a gas container 3616, and a fluid volume 3610. In some embodiments, the ingestible device 3600 can retain a dispensable substance volume from 300 μL to about 350 μL, can have an expansion volume from about 350 μL to about 380 μL, and can have a gas container fill volume from about 35 μL to about 45 μL. In some embodiments, a 280 PSIG fill pressure of the gas container corresponds to a drive pressure volume from about 70-80 μL. In some embodiments, a 240 PSIG fill pressure of the gas container corresponds to a drive pressure volume from about 90-100 μL.

FIG. 37 shows an embodiment of an ingestible device 3700 in which a length of the ingestible device is reduced to achieve a modified size 00 submucosal device and a piston length is reduced. In some embodiments, the length of the ingestible device 3700 is 23.3 mm, (Size 00) or, in some embodiments, 26 mm (Size 000). As shown in FIG. 37, ingestible device 3700 includes a piston 3718, a gas container 3716, and a fluid volume 3710. The ingestible device 3700 can retain a dispensable substance volume from 300 μL to about 350 μL, can have an expansion volume from about 320 μL to about 380 μL, and can have a gas container fill volume from about 65 μL to about 85 μL. A 280 PSIG fill pressure of the gas container corresponds to a drive pressure volume from about 140-150 μL A 240 PSIG fill pressure of the gas container corresponds to a drive pressure volume from about 170-190 μL.

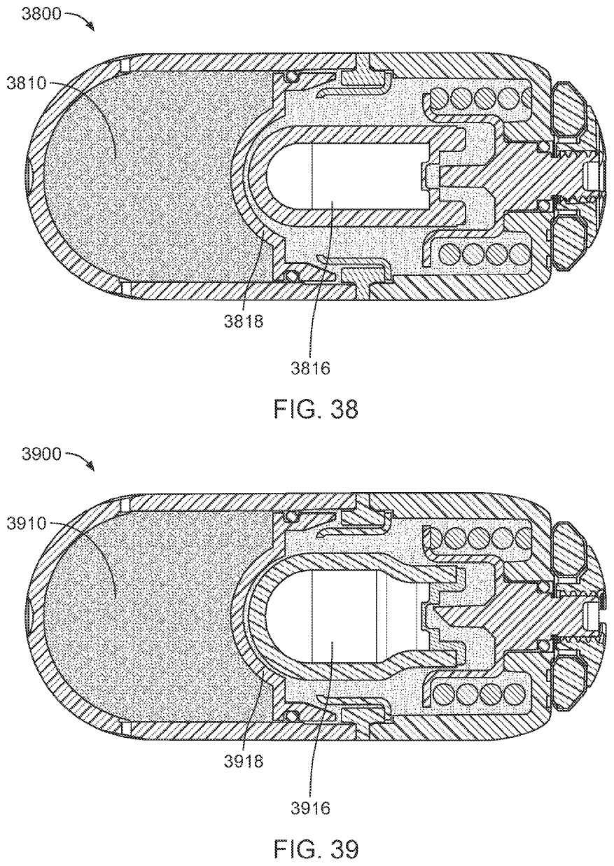

FIG. 38 shows an embodiment of an ingestible device 3800 in which a length of the ingestible device is reduced to achieve a modified size 00 submucosal device and a piston length is reduced. In some embodiments, the length of the ingestible device 3800 is 23.3 mm, (Size 00) or, in some embodiments, 26 mm (Size 000). As shown in FIG. 38, ingestible device 3800 includes a piston 3818, a gas container 3816, and a fluid volume 3810. In some embodiments, the ingestible device 3800 can retain a dispensable substance volume from 300 μL to about 350 μL, can have an expansion volume from about 300 μL to about 320 μL, and can have a gas container fill volume from about 35 μL to about 45 μL. Piston shape of piston 3818 may result in residual dispensable substance volume of about 80 μL (of a total of 335 μL delivered) within the dispensable substance housing after delivery.

FIG. 39 shows an embodiment of an ingestible device 3900 in which a length of the ingestible device is reduced to achieve a modified size 00 submucosal device and a gas container diameter is modified. The length of the ingestible device 3900 is 23.3 mm, (Size 00) or, in some embodiments, 26 mm (Size 000). As shown in FIG. 39, ingestible device 3900 includes a piston 3918, a gas container 3916, and a fluid volume 3910. The ingestible device 3900 can retain a dispensable substance volume from 300 μL to about 350 μL, can have an expansion volume from about 250 μL to about 290 μL, and can have a gas container fill volume from about 70 μL to about 80 μL. Piston shape of piston 19166 may result in residual dispensable substance volume from about 70-90 μL (e.g., about 80 μL) of a total amount of dispensable substance volume delivered within the housing after delivery.

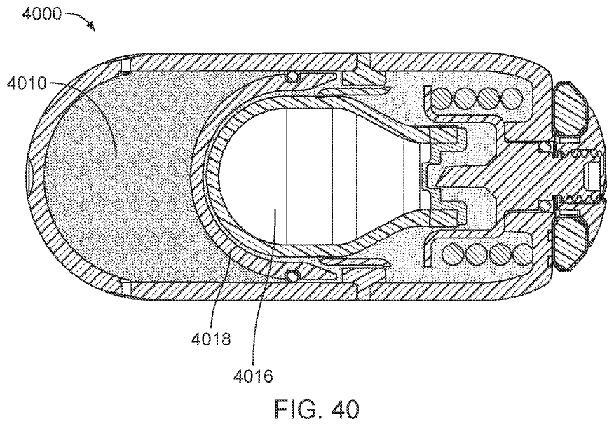

FIG. 40 shows an embodiment of an ingestible device 4000 in which a length of the ingestible device is reduced to achieve a modified size 00 submucosal device and a gas container diameter is modified. The length of the ingestible device 4000 is 23.3 mm, (Size 00) or, in some embodiments, 26 mm (Size 000). As shown in FIG. 40, ingestible device 4000 includes a piston 4018, a gas container 4016, and a fluid volume 4010.

In some embodiments, the ingestible device 4000 can retain a dispensable substance volume from 300 μL to about 350 μL (e.g., about 332 μL), can have an expansion volume from about 220 μL to about 270 μL (e.g., about 240 μL), and can have a gas cylinder fill volume from about 125 μL to about 145 μL (e.g., about 138 μL). In some embodiments, a 240 PSI drive pressure of the gas cylinder corresponds to a fill pressure from about 650-710 PSI (e.g., 684 PSI). In some embodiments, 280 PSI fill pressure of the gas cylinder corresponds to a drive pressure from about 770-830 PSI (e.g., about 798 PSI). In some embodiments, a 320 PSI drive pressure of the gas cylinder corresponds to a fill pressure from about 880-940 PSI (e.g., 912 PSI). Other gas cylinder fill pressures are provided in the Table 1 below.

TABLE 1
Gas Cylinder Fill Pressures

| Test Point | 1 | 2 | 3 | 4 | 5 | 6 |
|---|---|---|---|---|---|---|
| Gas Fill Mass (mg) | 11.8 | 13.8 | 15.7 | 17.2 | 19.7 | 28.5 |
| Drive Pressure at ref. temp (20deg C.) (PSI) | 240 | 280 | 320 | 350 | 400 | 580 |
| Drive Pressure at body temp (37deg C.) (PSI) | 254 | 296 | 339 | 370 | 423 | 614 |
| Fill Pressure at ref. temp (20deg C.) (PSI) | 684 | 798 | 912 | 997 | 1140 | 1653 |
| Fill Pressure at body temp (37deg C.) (PSI) | 724 | 844 | 965 | 1055 | 1206 | 1749 |

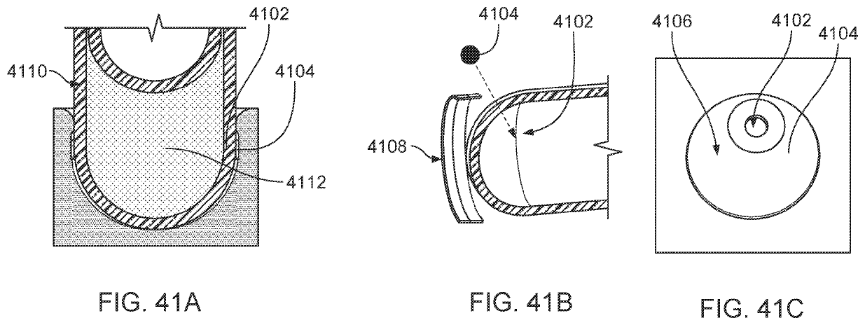

In FIGS. 41A-41C, a nozzle opening 4102 can be covered by a covering including a member, e.g., a patch 4104, that forms a barrier between a dispensable substance 4112 retained within the housing 4110 and an environment external to the ingestible device. A patch 4104 can be formed of a materials that is a degradable material, an erodible material and a dissolvable material. A patch can be a barrier film composed of various materials, for example, polyethylene (PE), polypropylene, cyclic olefin copolymer (COC), cyclo-olefin-polymer (COP), polycarbonate, polyvinyl chloride (PVC), polyurethane, or the like. A patch may be a multi-layer film, e.g., two or more layers of a same or different material, for enhanced barrier properties. Multi-layer construction of a patch 4104 can include, for example, PE/ethylene-vinyl alcohol copolymer (EVOH), ethylene-vinyl acetate (EVA)/EVOH/EVA, EVA/polyvinylidene chloride (PVDC)/EV, or the like. A multi-layer construction of a patch can include a metal layer.

A patch 4104 can have various shape profiles, for example, circular, rectangular, polygonal, or asymmetric profile. As shown in FIG. 41, a patch may be affixed off-center 4106 over the nozzle opening 4102 on an outside surface of the ingestible device such that a force of a jet expelled through the nozzle opening 4102, e.g., by pressurized release of the dispensable substance, may preferentially move the patch away from a direction of the formed jet.

A patch 4104 may be affixed loosely over a nozzle opening, e.g., using adhesive or another pressure sensitive method, or using static attraction. Adhesive to affix the patch may be utilized on a surface surrounding the nozzle but not directly on the nozzle.

A film, a coating, a foil, a band, or the like may be placed over the patch that is affixed over the nozzle opening, and may be composed on a dissolvable material, e.g., enteric material, such that the film, coating, foil, or band holds the patch in place over the nozzle opening during handling, storage, and ingestion of the ingestible device. In one example, a band 4108 is composed of a material that can dissolve upon entry into a body. The film or band may be composed of a material that is water soluble, e.g., hydroxypropyl methyl cellulose (HPMC), hydroxypropylmethylcellulose acetate succinate (HPMCAS), or gelatin. The film or band 4108 may be composed of a material that includes a pH-dependent solubility, e.g., composed of or including polymethacrylate, such that the material is more stable under acidic conditions, e.g., pH 1-4, and where a rate of dissolution increases when the material is exposed to higher pH, e.g., pH 5-7.

A band covering a nozzle opening can be composed of a heat shrink material that is heat shrunk to the housing such that it provides a nozzle covering. An example of a heat shrink material is polyethylene terephthalate (PET). Additional examples of heat shrink materials include polyolefin, polyethylene, LDPE, PTFE, FEP and COC. In general, such a heat shrink material does not operate by being dissolved. Instead, it is broken (e.g., punctured) by the pressure of the dispensable material applied to the heat shrink material. Such a heat shrunk band can have a thickness of, for example, from about 5 μm to about 100 μm

In general, a covering (e.g., a film, a coating, a foil, a band) of a nozzle opening can be scored, e.g., to make it easier for the seal to be broken when desired. Generally, such scoring can be configured as desired. As an example, scoring can be configured as a series of parallel lines. As another example, scoring can be configured as a grid (cross-hatched). As a further example, scoring can be configured as a plurality of dots (e.g., equally spaced dots). In some embodiments of a scored seal, the seal is composed of LDPE, for example of having a thickness of from 20 μm to 75 μm (e.g., 25 μm. 50 μm). For example, a seal composed of LDPE can be scored with stripes or a grid or a plurality of dots, with the LDPE having a thickness of 25 μm or 50 μm.

In some embodiments, covering (e.g., a coating, a film, a band, or a patch has a minimal burst pressure. In some embodiments, for example, the minimal burst pressure is less than 420, 220, 110 psig, Generally, the minimal burst pressure is more than 5 psig (e.g., more than 10 psig, more than 25 psig, more than 50 psig more than 80 psig).

A coating or film can be applied over a nozzle opening 4102 that may dissolve/degrade or otherwise become unstable after the ingestion of the ingestible device. In some embodiments, the coating or film is hydrophobic. The coating or film can be structurally weakened by drilling/scoring, e.g., using laser drilling, and/or can be composed of a material that weakens based on an environment surrounding the material, e.g., an enteric material within the body. In one example, laser microtoming can be utilized to thin a coating or film, e.g., a sanding/polishing process, to reduce the coating or film thickness. A coating or film of an enteric material can be applied over a nozzle opening 4102 and a portion of an outer surface of the ingestible device. A machining/polishing processes can be utilized to control a final thickness of the applied coating or film, e.g., centerless lapping or grinding. The coating or film can be further processed using a laser to drill, score, and/or perforate a portion of the coating or film to mechanically weaken the coating or film.

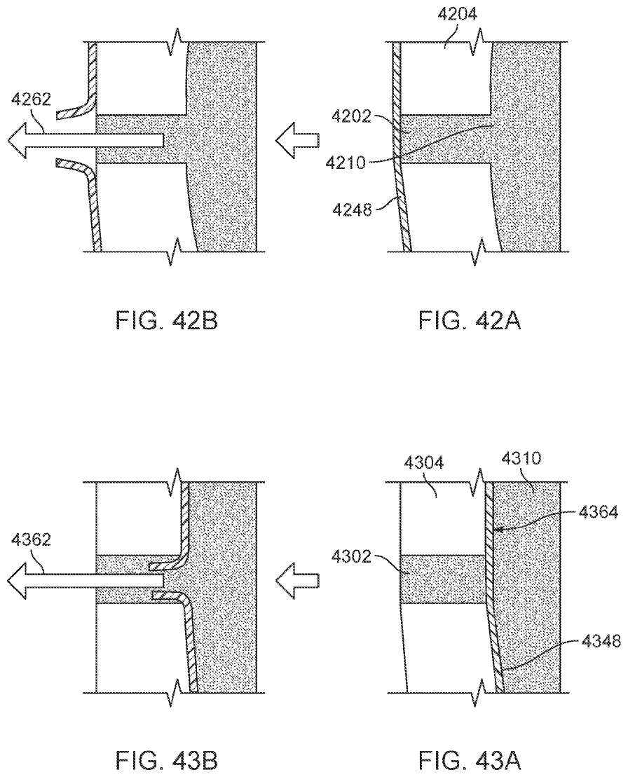

FIGS. 42A-47C depict embodiments of a patch, coating, film, foil and/or band that can be affixed to or in contact with the nozzle opening. While such embodiments are shown in these figures, the disclosure is not limited in this sense. In some embodiments, a combination of more than one (e.g., more than two, more than three) such approaches to covering a nozzle opening may be used in a given ingestible device. Further, variations on the approaches disclosed herein are available so long as they generally comport with the relevant function(s), such as, for example, providing a barrier between a dispensable substance (e.g., drug-containing liquid) retained within the drug housing and an environment external to the ingestible device.

As shown in FIGS. 42A and 42B, a nozzle opening 4202 can be covered by a covering including a member 4248, e.g., a patch, film, foil, band, or the like, that forms a barrier between a fluid volume 4210 including a dispensable substance (e.g., drug-containing liquid) retained within the housing and an environment external to the ingestible device. Certain embodiments including a nozzle covering member formed of a film, foil, patch, band, or the like are discussed, for example, with reference to FIGS. 17, 18, 19A-N of U.S. Pat. No. 11,007,356 B2, and optionally applicable to FIGS. 42A and 42B.

Internal pressure from a pressurized dispensable substance, e.g., during pressurized dispensable substance release, can cause the dispensable substance to puncture the covering member or partially peel/detach the covering member from the outer surface of the ingestible device to allow dispensable substance-containing jets 4262 to form. The covering member 4248 can be composed of various materials, e.g., PE, PP, PVC, cellulose acetate, hot blocking film, and the like. In some embodiments, the covering member can be composed of material that is intended to be insoluble in gastric media but may break down in the small intestine based on pH (e.g., enteric materials) or one or more enzymes, such as, for example, one or more pancreatic enzymes (e.g., lipid-based materials). The covering member 4248 can be composed of material that can hydrate and/or soften when exposed to gastric media without substantially dissolving. The covering member 4248 in this embodiment and others described herein can be composed on a gas-permeable membrane, e.g., which may help with de-gassing during a process of filing the ingestible device. The covering member can be applied, for example, in a post-molding operation, e.g., from a reel.

The covering member 4248 can be a thin shrink-fit film or adhesive label component applied to an external surface of the ingestible device to cover the nozzle openings. In certain embodiments, the thin film or adhesive label can be a thin barrier, e.g., having a thickness from 20 μm to 40 μm (e.g., from 25 μm to 35 μm, 30 μm).

The covering member 4248 can be an external band that is applied to cover the nozzle openings 4202. In certain embodiments, the band can be, for example, from 100 μm to 200 μm (e.g., from 125 μm to 175 μm, e.g., 150 μm) thick. Optionally, the band can be composed of materials such as gelatin, HPMC, or other materials that are soluble in gastric media, or can be composed of enteric material.

In some embodiments, the covering member 4248 can be a partial film or covering, e.g., an external cap, that is applied to an outside of the ingestible device to cover the nozzle openings 4202. The cap can be, for example, from 100 μm to 200 μm (e.g., from 125 μm to 175 μm, e.g., 150 μm) thick and/or cover less than the full exterior of the ingestible device.

As shown in FIGS. 43A and 43B, a covering member 4348, e.g., patch, film, foil, band, coating, or the like, that forms a barrier between a fluid volume 4310 including a dispensable substance (e.g., a drug-containing liquid) retained within the housing 4304 and an environment external to the ingestible device can be applied and/or affixed to an interior surface 4364 of the ingestible device. In some embodiments, the covering member 4348 is a thin film that is applied to an internal surface of a primary container 4304 of the ingestible device, e.g., during a molding process, to cover the nozzle openings 4302. Internal pressure from a pressurized dispensable substance, e.g., during pressurized dispensable substance release, can cause the dispensable substance to puncture the covering member or partially peel/detach the covering member from the outer surface of the ingestible device to allow dispensable substance-containing jets 4362 to form. The covering member 4348 can be composed of various materials. e.g., COC-based films such as COC+LLDPE laminate, and the like. In some embodiments, the covering member 4348 can be composed of material that is intended to be insoluble in gastric media but may break down in the small intestine based on pH (e.g., enteric materials) and/or or one or more enzymes, such as, for example, one or more pancreatic enzymes (e.g., lipid-based materials). The covering member 4348 can be composed of material that can hydrate and/or soften when exposed to gastric media without substantially dissolving. The covering member 4348 in this embodiment and others described herein can be composed on a gas-permeable membrane, e.g., which may help with de-gassing during a process of filing the ingestible device. The covering member 4348 can be applied, for example, using a molding process, e.g., based on an in-molded label or blow molded onto an interior surface 4364 of the ingestible device. The covering member can be, for example, from 20 μm to 40 μm (e.g., from 25 μm to 35 μm, 30 μm) thick. In some embodiments, the covering member can be applied/affixed without an adhesive, e.g., molded bond.

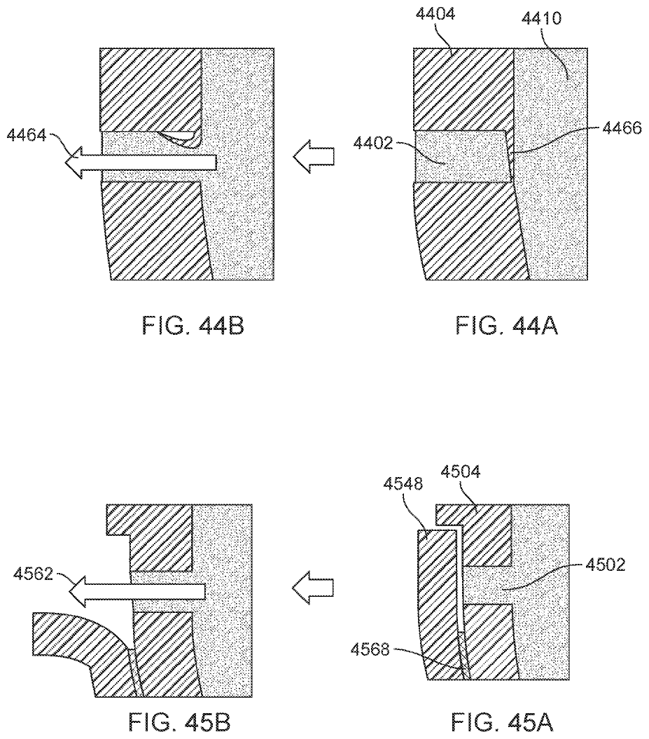

As shown in FIGS. 44A and 44B, a covering member can be a feature, such as, for example, a molded feature 4466 formed on (or adjacent to) an interior end of a nozzle opening 4402 and which forms a barrier between a fluid volume 4410 including a dispensable substance (e.g., drug-containing liquid) retained within the housing 4404 and an environment external to the ingestible device. Internal pressure from a pressurized dispensable substance, e.g., during pressurized dispensable substance release, can cause the dispensable substance to puncture the covering member 4466 fully or partially peel/detach the covering member from the outer surface of the ingestible device to allow dispensable substance-containing jets 4464 to form. The covering member can be composed of various materials, e.g., COC-based films such as COC+LLDPE laminate, and the like. In some embodiments, the covering member 4466 can be composed of material that is intended to be insoluble in gastric media but may break down in the small intestine based on pH (e.g., enteric materials) and/or one or more enzymes, such as, for example, one or more pancreatic enzymes (e.g., lipid-based materials). The covering member 4466 can be composed of material that can hydrate and/or soften when exposed to gastric media without substantially dissolving.

As shown in FIGS. 45A and 45B, a covering member 4548 can be a covering member that is tethered 4568 to the ingestible device, e.g., tethered to an outer portion of the housing 4504. The covering member 4548 can be formed of a flexible material, e.g., an elastomer material. Internal pressure from a pressurized dispensable substance, e.g., during pressurized dispensable substance release, can cause the dispensable substance to detach/displace a portion or all of the covering member from the nozzle opening 4502 of the ingestible device to allow dispensable substance-containing jets 4562 to form.

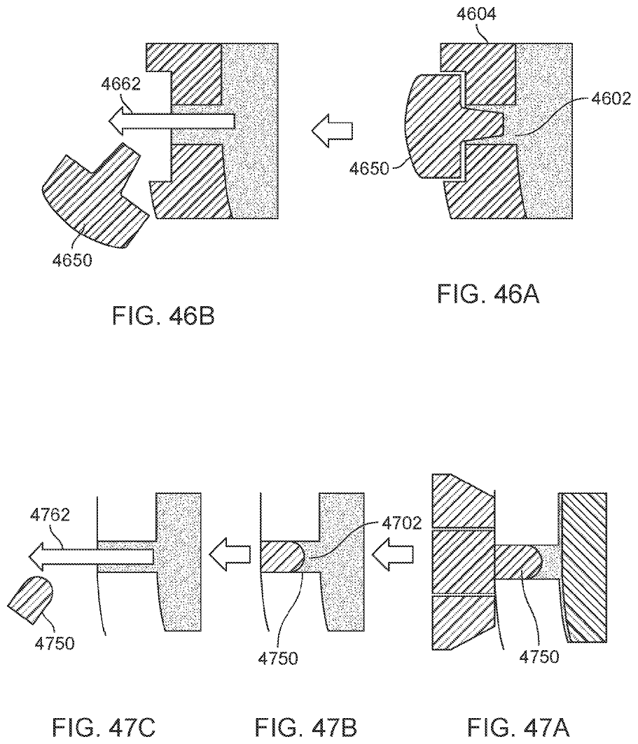

As shown in FIGS. 46A and 46B, a covering member can be a plug 4650, e.g., an elastomer plug, that can block the nozzle opening 4602 from an outside surface of the ingestible device. The plug can be tethered to a housing component 4604 of the ingestible device to prevent dispersion of the released plug into the body. The plug 4650 can be formed of biodegradable materials such that the plug can be processed by the body. Internal pressure from a pressurized dispensable substance, e.g., during pressurized dispensable substance release, can cause the dispensable substance to detach/displace the plug 4650 from the nozzle opening of the ingestible device to allow dispensable substance-containing jets 4662 to form.

As shown in FIGS. 47A-47C, a nozzle opening 4702 can be blocked by a plug 4750 that is formed by applying a liquid-fil gel from an outside apparatus 4770 of the ingestible device, e.g., via a nozzle or rotating mandrel. The liquid-fill gel can harden before a dispensable substance filling process to provide a plug 4750. The gel can be composed of a material that is substantially insoluble in gastric media/dispensable substance, but can break down in small intestine-based pH (e.g., an enteric material) and/or one or more enzymes, such as for example, one or more pancreatic enzymes (e.g., lipid-based material). Internal pressure from a pressurized dispensable substance, e.g., during pressurized dispensable substance release, can cause the dispensable substance to displace the gel from the nozzle opening 4702 of the ingestible device to allow dispensable substance-containing jets 4762 to form.

FIGS. 48A and B depict an embodiment of an ingestible device 4800 utilizing an internal piston. Ingestible device 4800 contains a dispensable substance that is not under pressure when the subject swallows the ingestible device. In FIG. 48A, the nozzles 4802 are shown as covered, and in FIG. 48B the nozzles 4802 are uncovered. The ingestible device 4800 has housing parts 4804 and 4806 connected by a union ring 4808 and with a fluid volume 4810 containing a dispensable substance, a spring 4814, a gas container 4816, a first piston 4818a and a second piston 4818b, a piercer 4820, and an O-ring 4832. The piercer 4820 is held in place with an enteric trigger 4826 that dissolves and used as the triggering mechanism. When the device 4800 is swallowed by the subject, the enteric trigger 4826 prevents the dispensable substance in fluid volume 4810 from being under pressure by holding the spring 4814 and the piercer 4820 in place. When the device 4800 reaches the appropriate location in the GI tract, a triggering event occurs, for example the enteric trigger 4826 degrades and/or dissolves (e.g., due to pH, change in pH, presence of certain enzyme, and/or concentration of certain enzyme) such that the spring 4814 forces the piercer 4820 into the gas container 4816, puncturing the gas container 4816 and causing gas at elevated pressure to leave the container 4816. This causes the gas container 4816 to press against the first piston 4818a and apply pressure to the fluid volume 4810. The pressurized fluid volume 4810 applies pressure to the second piston 4818b cause the second piston 4818b to slide and expose the nozzles 4802 such that the dispensable substance is delivered out of the nozzles 4802 in the form of a jet. The second piston 4818b as shown in FIGS. 48A and B includes an extension portion, which can, for example, help guide the second piston out of a vent or opening in the housing. However, as described herein, XX is not a requirement of the second piston. This can result in trans-epithelial and/or epithelial delivery of the therapeutic agent contained in the dispensable substance.

FIG. 49A-49E show second piston 4919bA-4918bE designs that act as jet covers to prevent the dispensable substance from leaking from the openings prior to a triggering event. Subsequently, when the device reaches the appropriate location in the GI tract, the triggering event occurs, for example, the enteric trigger degrades and/or dissolves (e.g., due to pH, change in pH, presence of certain enzyme, and/or concentration of certain enzyme) such that the spring forces the piercer into the gas cylinder, puncturing the gas cylinder and causing gas at elevated pressure to leave the cylinder. The triggering event causes the gas cylinder to press against the piston and apply pressure to the fluid volume. The pressurized fluid volume 4910 applies pressure to the second piston 4918bA-4918bE causing the second piston 4918bA-4918bE to slide and expose the nozzles 4902 such that the dispensable substance is delivered out of the nozzles 4902 in the form of a jet. This can result in trans-epithelial and/or epithelial delivery of the therapeutic agent contained in the dispensable substance. The second pistons 4918bA-4918bE may have a diameter (DD) that equals the interior diameter of the housing or slightly larger, for example 5-15% larger. For a 000-sized device, the second pistons may have a diameter of about 8.5 to 9.0 mm. The second pistons 4918bA-4918bE may have a thickness (TT) from 0.6 mm to 1.2 mm, with the preferred thickness about 1.0 mm. The second piston 4918bA-4918bE durometer may range from 60 A to 85 A, with the preferred durometer about 70 A. In other embodiments, the openings in the drug module are not placed on the curved surface of the drug module. In FIGS. 49A-49E, this is shown by showing the “nozzle height” at 11.9 mm and 11.3 mm, wherein the 11.3 mm height is clear of the curved surface of the drug module and resulted in better sealing and subsequent jet formation for trans-epithelial delivery (see FIG. 49B).

In other embodiments, one or more vent holes may be introduced to the drug module to allow air between the second piston and the drug module to escape, thereby allowing the second piston 4918bA-4918bE to more easily slide without resistance from trapped air. The second piston 4918bA-4918bE may be shaped to increase the surface area of the portion of the piston in contact with the interior of the drug module, i.e., the flange 4920 of the second piston 4918bA-4918bE may be equal size or larger than the opening to ensure the openings are sealed. This is shown in FIGS. 49D and 49E where the edge of the piston is larger than the edge show in FIG. 49C. The first piston 4918a may be physically coupled to the second piston 4918bA-4918bE by one or more couplers, for example, by arms that are collapsible. The design of the device shown in FIGS. 49A-49E may otherwise be the same as FIGS. 48A and 48B described above.

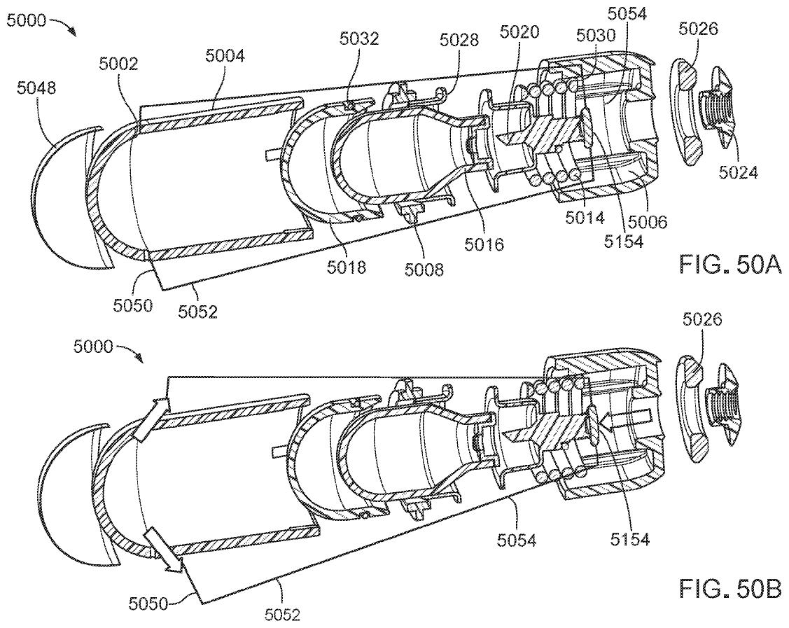

A plug/cover can be fixed over a nozzle opening, where the plug/cover is further connected to a piercer component of the ingestible device via connectors and a ring component. FIGS. 50A and 50B show an embodiment of an ingestible device 5000 including a plug/cover assembly. Ingestible device 5000 includes nozzle opening(s) 5002, a drug container 5004, a drive housing 5006, an O-ring 5032, a retention element 5028, a piercer 5020, a gas seal 5030, a trigger element 5026, a trigger support 5024, a spring 5014, a gas container 5016, a union ring 5008, and a piston 5018. The ingestible device 5000 optionally includes a nozzle cover 5048.

A plug/cover assembly can be a single formed piece, e.g., composed of a plastic material, and fitted externally to the ingestible device such that the plug(s) 5050 cover the nozzle opening(s) 5002 on the ingestible device 5000. The plug/cover assembly can further include connectors 5052 that connect the plug/cover assembly to a ring 5054 component that can be attached to a top of the piercer 5020 and external to a trigger element 5026, such that the ring component 5054 is pulled down by the piercer 5020 when the piercer is released, e.g., after the trigger element 5026 dissolves/degrades, and the plug/cover 5050 are pulled away from the nozzle opening 5002 by the movement of the ring 5054. In some embodiments, the plug/cover 5050 are pulled away from the nozzle opening 5002 by the movement of the ring 5054 in a direction parallel to a length of the ingestible device 5000, e.g., along the outer surface of the ingestible device. The plug/cover 5050 may be pulled away from the nozzle opening 5002 by the movement of the ring 5054 in a direction outwards, e.g., normal, or angled-away, from an outer surface of the ingestible device 5000.

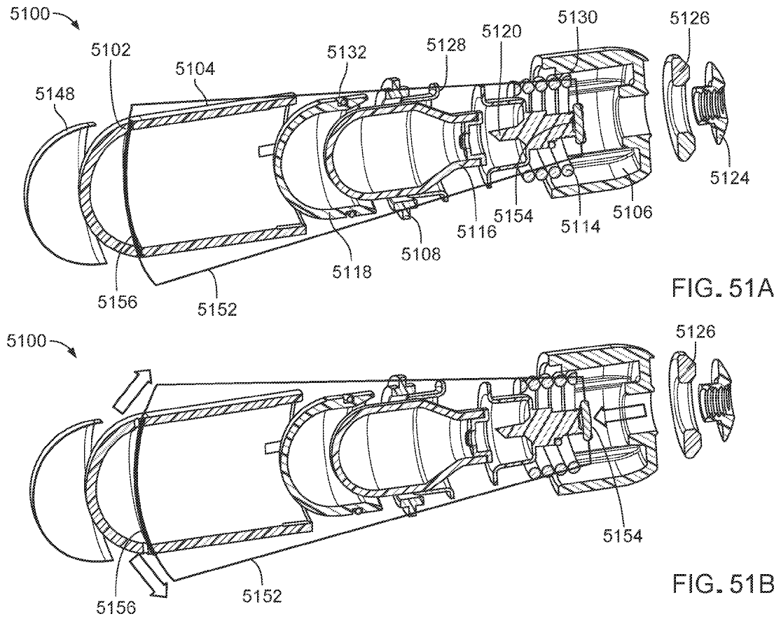

A band can be fixed over one or more nozzle openings, where the band is further connected to a piercer component of the ingestible device via connectors and a ring component. FIGS. 51A and 51B shows an embodiment of an ingestible device 5100 including a band assembly. Ingestible device 5100 includes nozzle opening(s) 5102, a drug container 5104, a drive housing 5106, an O-ring 5132, a retention element 5128, a piercer 5120, a gas seal 5130, a trigger element 5126, a trigger support 5124, a spring 5114, a gas container 5116, a union ring 5108, and a piston 5118. The ingestible device 5100 optionally includes a nozzle cover 5148.

A band assembly can be a single formed piece, e.g., composed of a plastic material, including a band 5156, connectors 5152, and a ring component 5154. A band assembly can be instead multi-piece assembly composed of a band 5156 that is placed around the ingestible device 5100 during a filling process and a connector/ring assembly that are affixed to the band 5156 and piercer component 5120. The band assembly can be connected to the piercer 5120 by a ring component 5154 that can be attached to a top of the piercer component and external to a trigger element 5126, such that the ring component 5154 is pulled down by the piercer 5120 when the piercer is released, e.g., after the trigger element 5126 dissolves/degrades, and the band 5156 is pulled away from the nozzle opening(s) 5102, e.g., along a length of the ingestible device 5100, to expose the nozzle opening(s) 5102 by the movement of the ring 5154 prior or simultaneously to the delivery of the dispensable substance via the nozzle openings 5102.

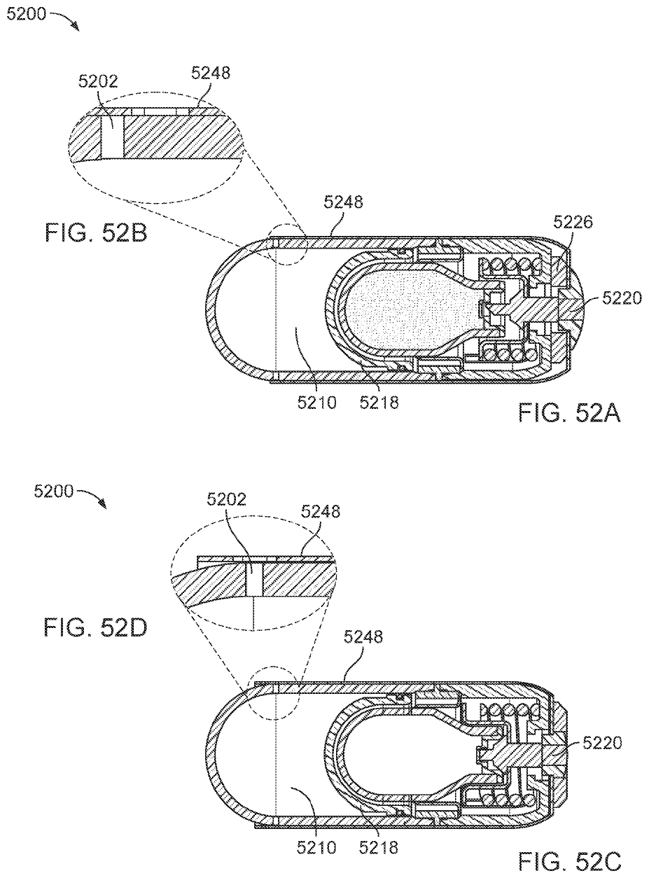

As shown in FIGS. 52A-52D, an ingestible device 5200 includes a sliding cover 5248. The sliding cover 5248 can be a single formed piece, e.g., a sleeve composed of a plastic material, and fitted externally to the ingestible device 5200 such that a portion of the sliding cover covers the nozzle opening(s) 5202 on the ingestible device 5200. The sliding cover 5248 can be attached to a top of the piercer component 5220 and external to a trigger element 5226, such that the sliding cover 5248 is pulled down by the piercer 5220 when the piercer is released, e.g., after the trigger element 5226 dissolves/degrades, and the sliding cover 5248 is pulled away from the nozzle opening 5202 by the movement of the piercer 5220. The sliding cover 5248 can be pulled away from the nozzle openings 5202 by the movement of the sliding cover in a direction parallel to a length of the ingestible device 5200, e.g., along the outer surface of the ingestible device, prior or simultaneously to the delivery of the dispensable substance within fluid volume 5210 via the nozzle openings 5202. In some embodiments, the sliding cover 5248 is fitted internally to the ingestible device 5200 such that a portion of the sliding cover covers the nozzle opening(s) 5202 on the ingestible device 5200. The internal sliding cover can be attached to the piercer component 5220 or the piston 5218.

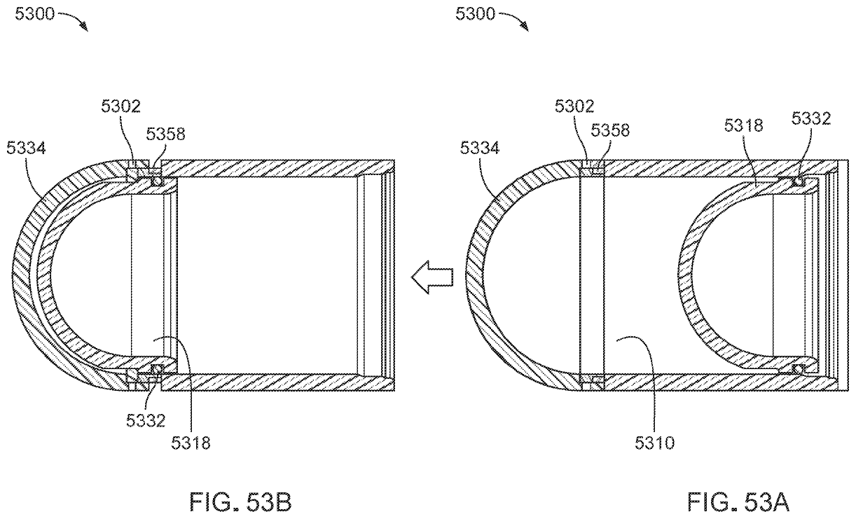

An ingestible device 5300 as shown in FIGS. 53A and 53B includes a cap 5334 affixed over one end of an ingestible device 5300 and partially enclosing a volume 5310. A seal 5358, e.g., an over-molded elastomer-based seal, can be utilized to seal a dispensable substance within the volume 5310 and prevent the dispensing of the dispensable substance while the cap 5334 is affixed over the end of the ingestible device 5300. The seal 5358 can additionally prevent movement of the cap 5334 prior to a delivery of the dispensable substance. When the device 5300 is swallowed by the subject, the enteric trigger prevents the dispensable substance in the fluid volume from being under pressure by holding the spring and the piercer in place. When the device reaches the appropriate location in the GI tract, the enteric trigger degrades and/or dissolves (e.g., due to pH, change in pH, presence of certain enzyme, and/or concentration of certain enzyme) such that the spring forces the piercer into the gas container, puncturing the gas container and causing gas at elevated pressure to leave the container. This causes the gas container to press against the piston and apply pressure to the fluid volume. The pressurized fluid volume applies pressure to the cap and causes the cap to slide open and expose the nozzles such that the dispensable substance is delivered out of the nozzles in the form of a jet. This can result in trans-epithelial and/or epithelial delivery of the therapeutic agent contained in the dispensable substance.

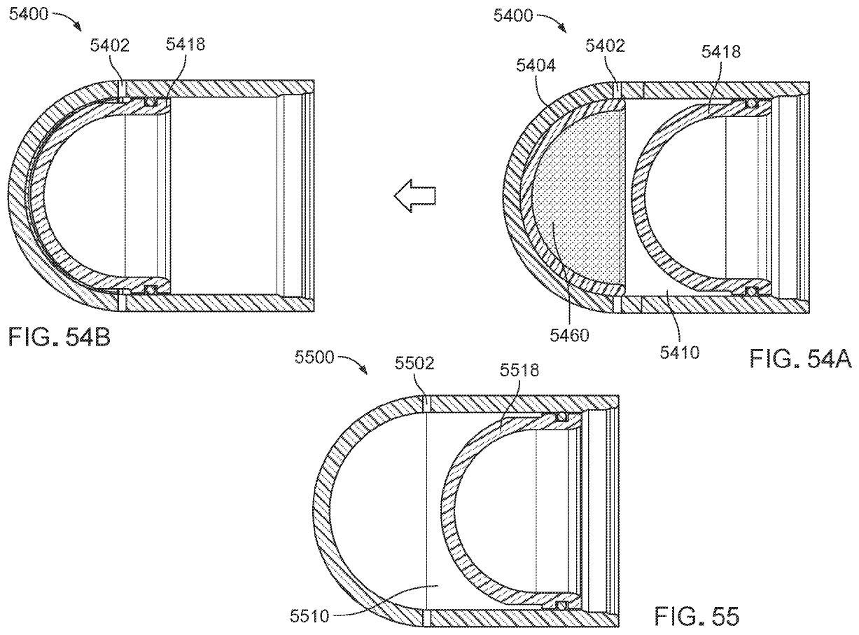

As shown in FIGS. 54A and 54B, an ingestible device 5400 includes an inflated membrane volume 5460, e.g., a gas balloon or similar, located within the volume 5410 including a dispensable substance and arranged to seal the nozzle openings 5402 while the inflated volume 5460 is inflated. In some embodiments, the inflated membrane volume 5460 may conform to one or more contours, e.g., an inner curvature, of the ingestible device housing 5404. The inflated membrane volume 5460 can be composed of a balloon and/or soft material, e.g., a low durometer elastomer. When the device 5400 is swallowed by the subject, the enteric trigger prevents the dispensable substance in the fluid volume from being under pressure by holding the spring and the piercer in place. When the device reaches the appropriate location in the GI tract, the enteric trigger degrades and/or dissolves (e.g., due to pH, change in pH, presence of certain enzyme, and/or concentration of certain enzyme) such that the spring forces the piercer into the gas container, puncturing the gas container and causing gas at elevated pressure to leave the container. This causes the gas container to press against the piston and apply pressure to the fluid volume. The pressurized fluid volume applies pressure to the inflated membrane volume 5460 and causes the inflated membrane volume to deflate or otherwise reposition to expose the nozzles openings 5402 such that the dispensable substance is delivered out of the nozzles in the form of a jet. This can result in trans-epithelial and/or epithelial delivery of the therapeutic agent contained in the dispensable substance.

The ingestible device in FIG. 55 does not include a covering member. For example, the nozzle openings 5502 may be exposed such that when the ingestible device 5500 is swallowed/inserted, an air gap in the nozzle openings 5502 and/or surface tension effects may prevent or deter gastric media from damaging the internal components or dispensable substance (e.g., drug-containing liquid) within the ingestible device. In other words, a differential force may be generated by the movement of the ingestible device within the gastric region between external intestinal forces/pressure and internal forces of the dispensable substance within the volume of the ingestible device. For example, a surface tension of the dispensable substance within a volume 5510 of the ingestible device can be higher than a surrounding environment, e.g., external intestinal forces/pressure within a gastric region in the body, such that a substantial percentage of the dispensable substance is retained within the volume of the ingestible device until a point of delivery of the dispensable substance, e.g., until piston 5518 applies pressure to the volume 5510 to force the dispensable substance retained within the volume 5510 out of the nozzle opening(s) 5502. In one example, at least 75% of the dispensable substance (e.g., at least 85%, at least 95% of the dispensable substance) is retained within the volume of the ingestible device until a point of delivery of the dispensable substance within a gastric region in the body.

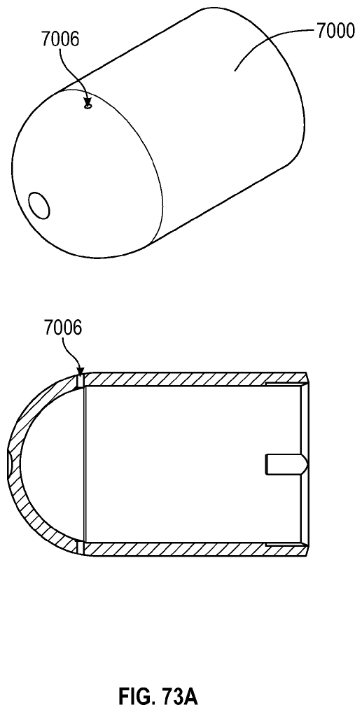

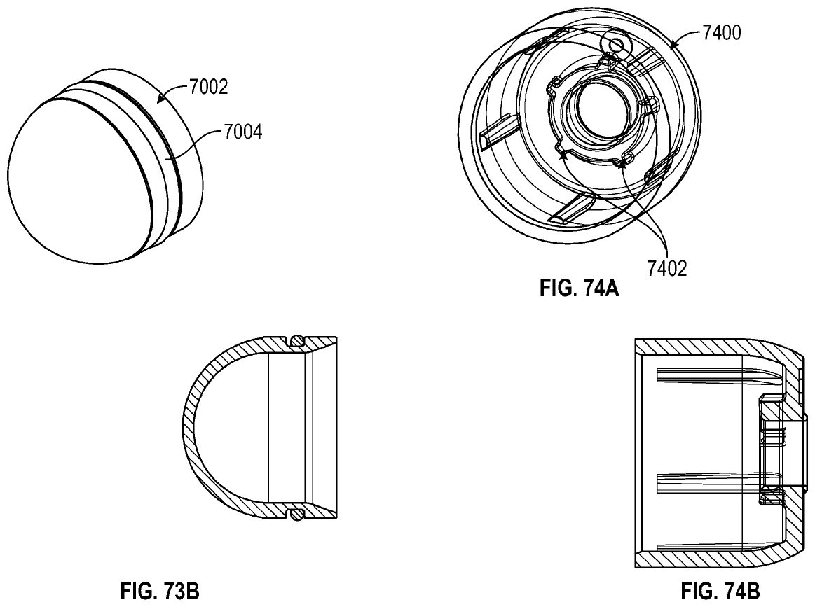

As shown in FIGS. 73A and 73B, the draft of the drug module 7000 (exaggerated for illustration) may be about 0.05 degrees to 0.25 degrees or about 0.10 to 0.15 degrees. A draft less than about 0.25 degrees generally allows for improved sealing between the piston 7002 or piston O-ring 7004 and the drug module housing 7000. The drug module 7000 may include one or more vents 7006 generally located on or near a flatter portion of the drug module 7000. In FIGS. 49A-49E, this is shown as the “nozzle height” at 11.9 mm and 11.3 mm, wherein the 11.3 mm height is clear of the curved surface of the drug module, i.e., at the base or below the dome of the drug module, and results in better sealing, better clearance of the openings, and subsequent jet formation for trans-epithelial delivery.

As shown in FIG. 73B, the draft of the piston 7002 may match the draft of the drug module, for example, about 0.15 degrees. The piston O-ring cross section may be about 0.50 mm to 0.70 mm, or about 0.60 mm. The piston O-ring has a nominal compression of about 14.5% to about 22.0%. For example, at the beginning of piston travel, the O-ring compression may be about 16.5%, and about 20.0% at the end of travel. The O-ring gland width may be about 0.80 mm to 0.90 mm, or about 0.85 mm.

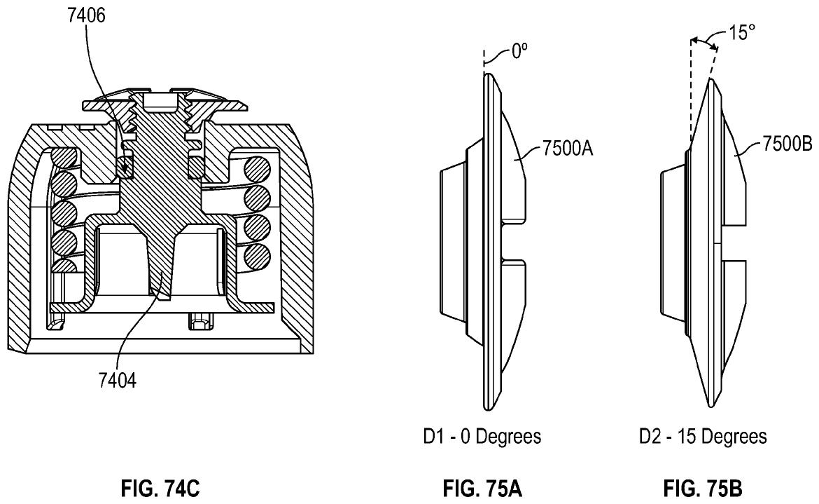

Turning to FIGS. 74A-74C, a trigger housing 7400 includes two to six ribs 7402, e.g., five ribs, which improves spring alignment and location. In this case, the piercer 7404 includes an O-ring 7406 with a gland width of about 0.90 mm to 0.95 mm, and a nominal compression of about 16.5% to about 22.0%. For example, at the beginning of piercer travel, the O-ring compression may be about 20.0% and about 18.7% at the end of travel.

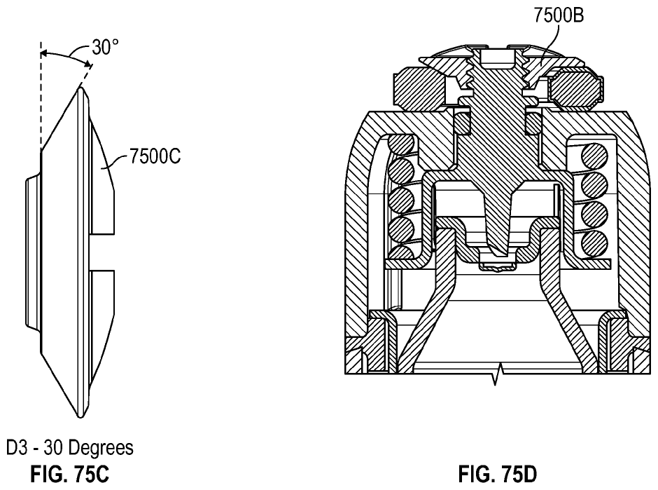

A trigger support 7500A, 7500B or 7500C may be angled to improve triggering of the device. For example, introducing a 15-degree angle to the trigger support, as shown in FIG. 75B, improves trigger degradation of the release component as compared to a trigger support with a 0-degree angle, i.e., flat as in FIG. 75A. Alternatively, a trigger support with an angle of 30-degrees or more, as in FIG. 75C, may not hold against the force of the spring, thereby leading to premature triggering or insufficient “shelf-life”. Hence, the trigger support may be angled from about 10 degrees to 25 degrees, or from about 15 degrees to 20 degrees. The spring that applies a force to the piercer to move the piercer to break the breakable seal of the gas container typically generates a force between about 28 N to 44 N, or about 34 N to 38 N, and may have a wire diameter from 0.85 mm to 0.95 mm or 0.90 mm. The angles above are measured relative to the flat top end surface of the housing shown in FIG. 75D, that is, relative to a plane perpendicular to a central longitudinal axis of the device, and perpendicular to the direction of movement of the piercer.

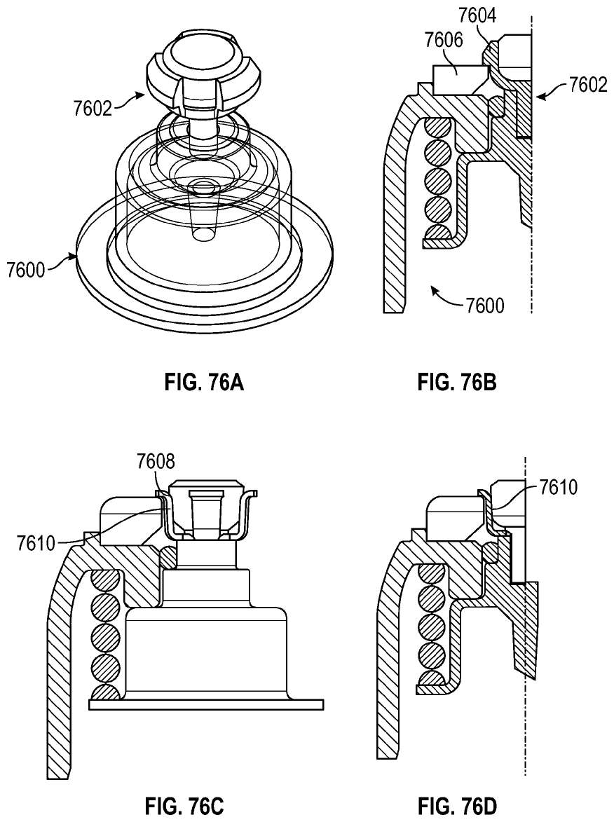

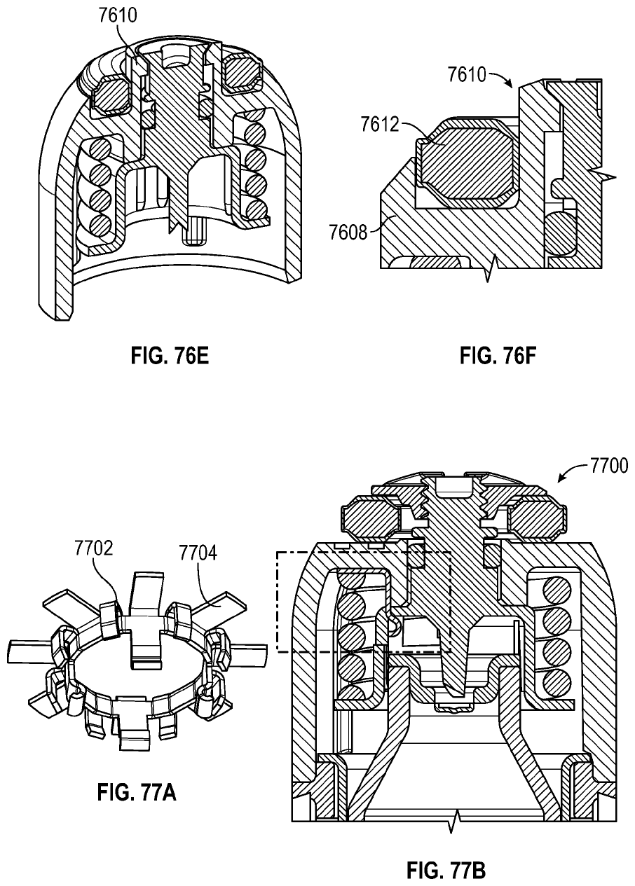

Referring to FIGS. 76A-76F, a piercer 7600 may be incorporated into a collet 7602. More specifically as shown in FIGS. 76A and 76B, the release component is designed as a wedge or other shape, whereby it forces snap arms 7604 of the piercer onto a washer 7606 during assembly. When the release component degrades, the snap arms close, thereby releasing from the washer and allowing the piercer to actuate. Alternatively, as shown in FIGS. 76C and 76D, the release component is held between a circular upstand 7608 and radial snap arms 7610 extend from the trigger housing. When the release component degrades, the radial snap arms 7610 extend outward, thereby releasing from the piercer and allowing the piercer to actuate.

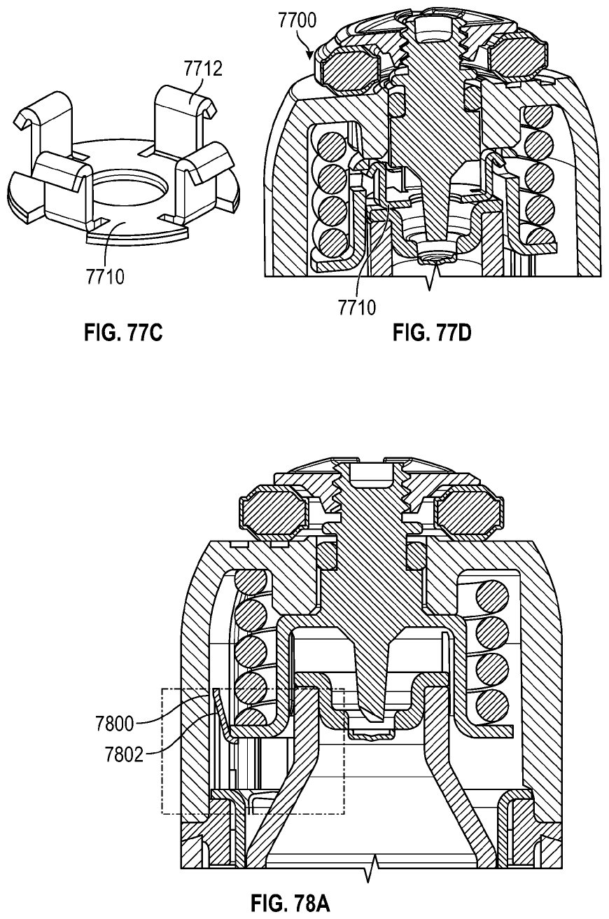

As shown in FIG. 77A-77D, a trigger housing 7700 may include breakaway piercer base 7702 or 7710 designed to improve actuation of the device via piercing of the gas cylinder. Upon degradation of the release component, the breakaway piercer base 7702 or 7710 retains the piercer in place until a threshold force is overcome, for example a threshold force of about 8 N to 30 N or 16N to 30N or the corresponding force applied by a piercer spring, thereafter the piercer extends without interference and pierces the breakable seal of the gas cylinder. The breakaway piercer base 7702 or 7710 is designed to ensure that the piercer moves in a single, swift movement after the release component degrades partially or completely. The breakaway piercer base may be made of any material with appropriate properties to retain the piercer until a threshold force is overcome. For example, the breakaway piercer base may be made of metals, alloys, plastics, fillers, or combinations thereof. The breakaway piercer base may comprise two or more arms. The breakaway piecer base 7702 external radial arms 7704 extending out over the top end of the spring, as shown in FIGS. 77A and 77B. The breakaway piercer base 7710 has internal hook arms 7712.

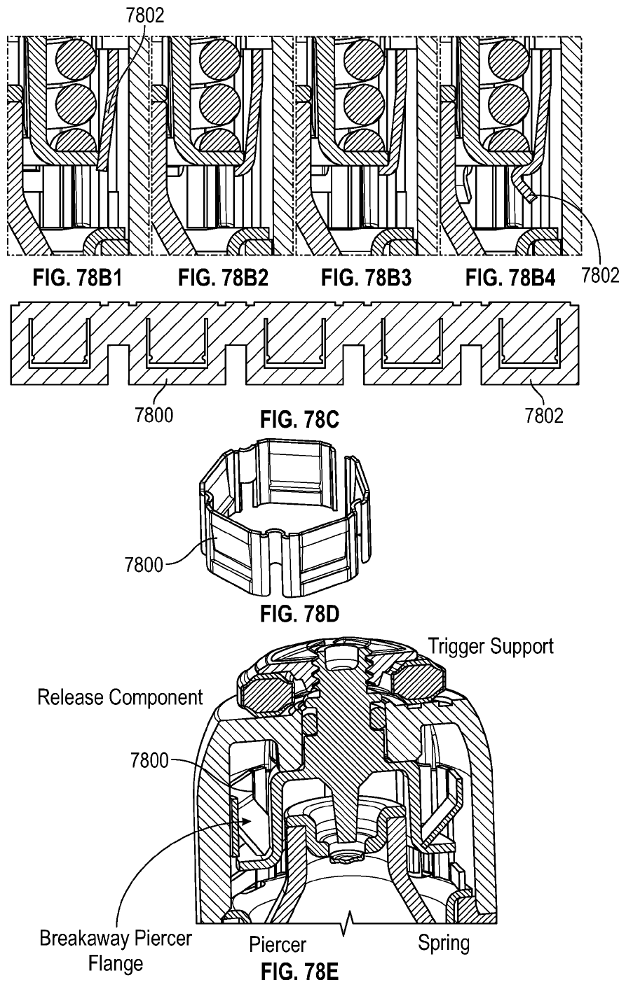

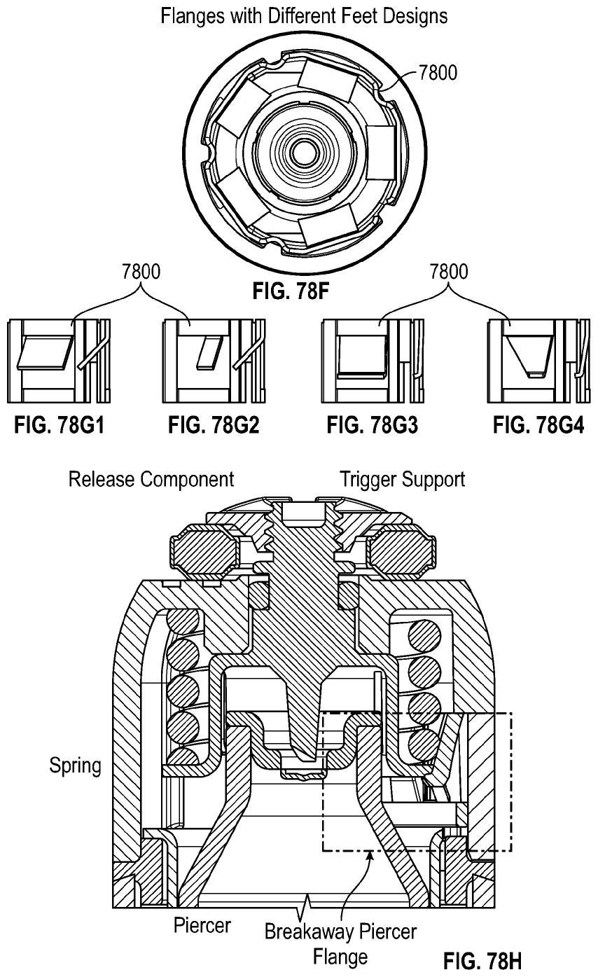

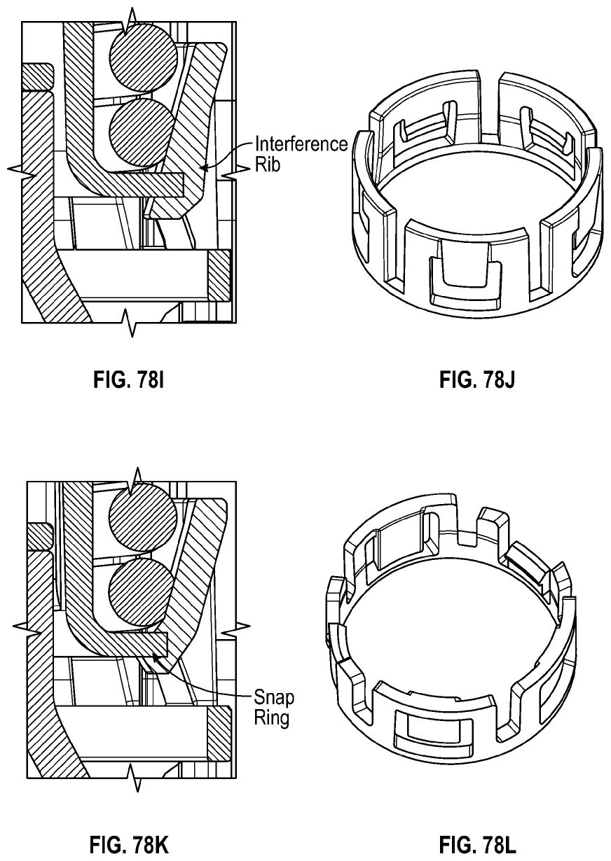

As shown in FIG. 78A-78D, a trigger housing includes a breakaway piercer flange 7800 designed to improve actuation of the device via piercing of the gas cylinder, whereby upon degradation of the release component, the breakaway piercer flange retains the piercer in place until a threshold force is overcome, for example a threshold force of about 20 NN to 30 N or the corresponding force applied by a piercer spring. Thereafter the piercer extends without interference and pierces the breakable seal of the gas cylinder. The breakaway piercer flange is designed to ensure that the piercer moves in a single, swift movement after the release component degrades partially or completely. The breakaway piercer flange may be made of any material with appropriate properties to retain the piercer until a threshold force is overcome. For example, the breakaway piercer flange may be made of metals, alloys, or plastics. The breakaway piercer flange may include feet 7802 that are straight, J-shaped, L-shaped or V-end shaped, as shown in FIGS. 78B1, 78B2, 78B3 and 78B4, respectively, which increases the threshold force of the flange. As shown in FIGS. 78C and 78D, the breakaway piercer flange 7800, if used, may be formed as a flat template and bent into a circular shape.

FIGS. 78E, 78F and 78G1-G4 show a breakaway piercer flange 7800 resting on the internal ribs of the trigger housing and having tabs projecting radially inwardly to engage the piercer. Alternatively, as shown in FIGS. 78H-78L, the breakaway piercer flange arms do not deform outwards during assembly. In this embodiment, the flange arms are preferably made of plastic, the flange arms may include one or both of an interference rib or snap ring as shown in FIG. 78H.

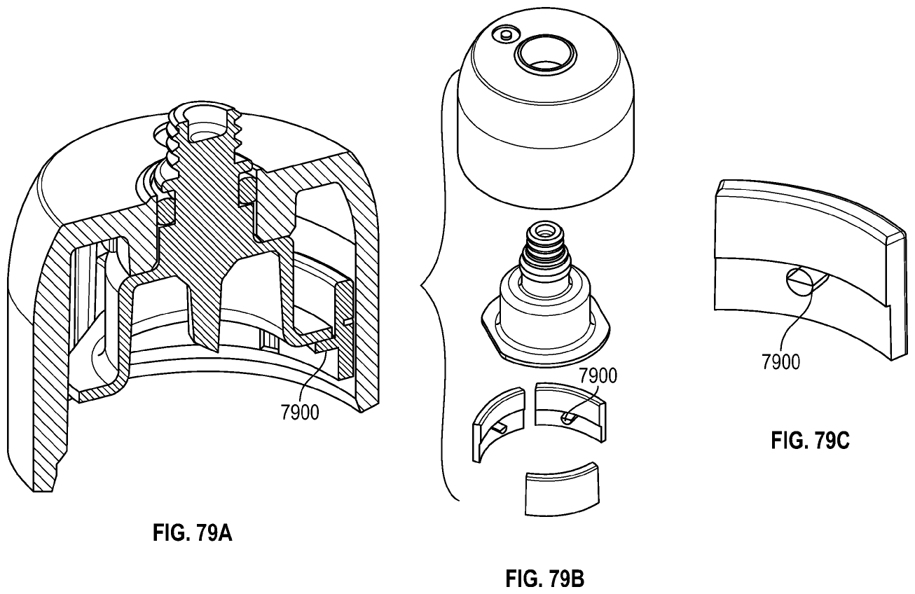

In the example of FIGS. 79A, 79B and 79C, the trigger housing includes two or more shear pins 7900 that retain the piercer in place until a threshold force is overcome, for example a threshold force of about 20 NN to 30 N or the corresponding force applied by a piercer spring, thereafter the piercer extends without interference and pierces the breakable seal of the gas cylinder. The diameter of the shear pins 7900 may range from about 0.6 mm to 1.2 mm.

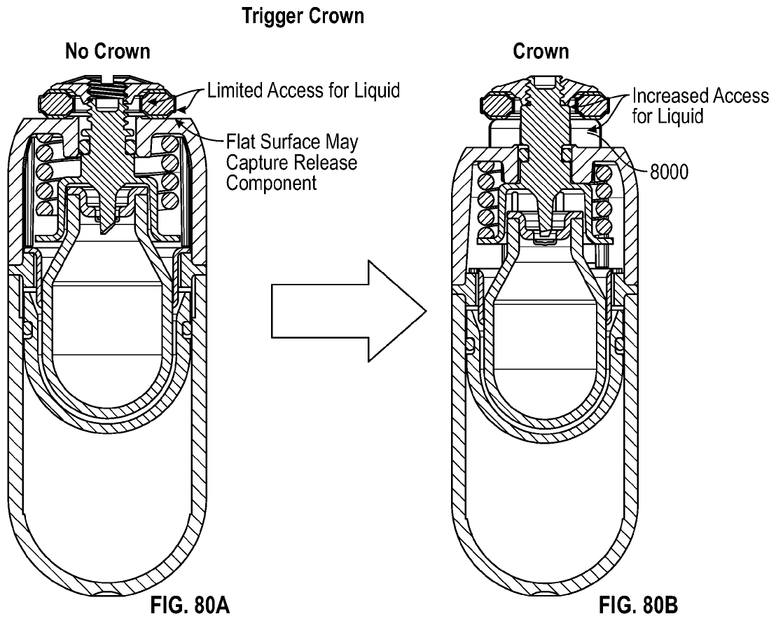

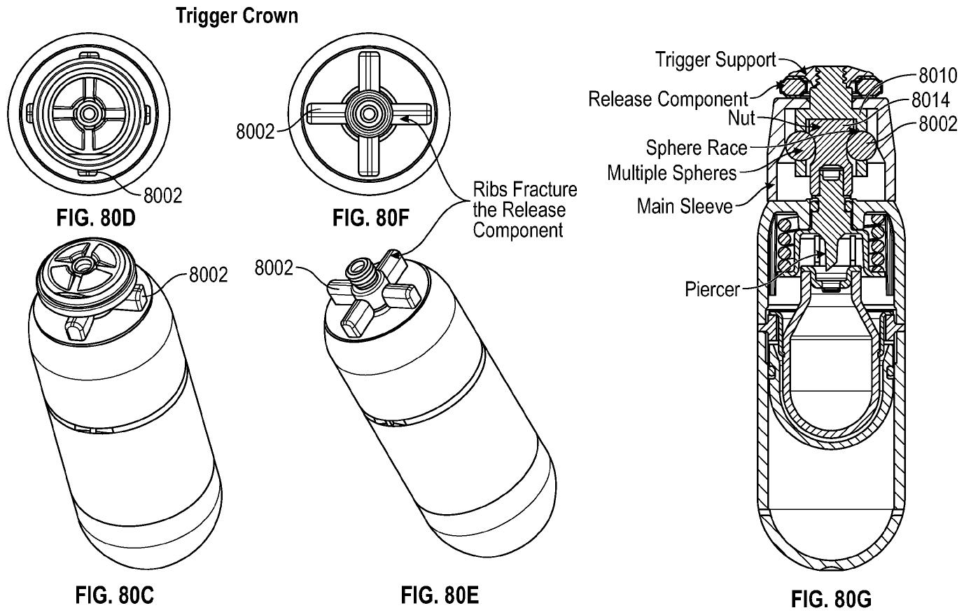

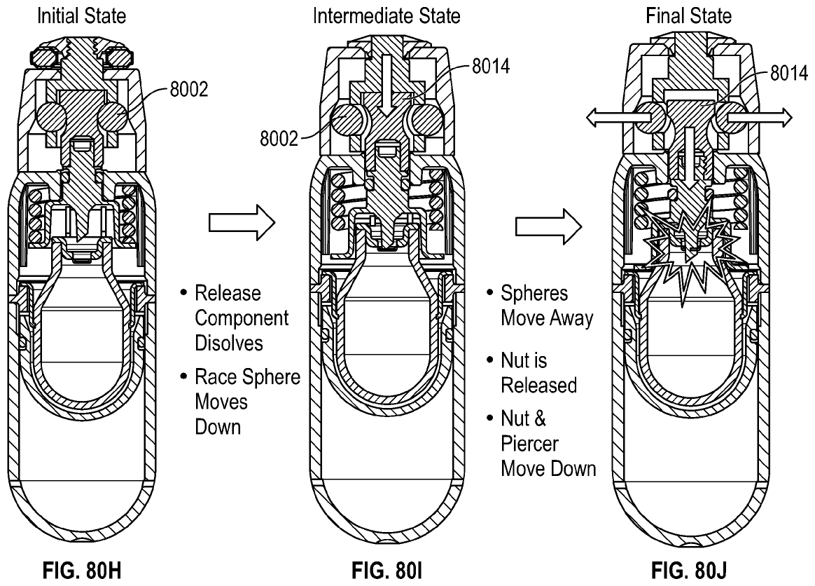

In the embodiment of FIGS. 80A-80F, the trigger housing includes a trigger crown 8000. The crown allows for increased or more uniform hydration of the release component. The crown may include one or more ribs or other features that serve to apply concentrated pressure on the release component, which helps contribute to catastrophic failure of the release component. Further, the crown provides space for debris from the release component to disperse, thereby preventing obstruction of any moving parts on the device such as the trigger support.

The release component may be designed to have greater strength and/or decreased degradation where it interacts with the ribs of the crown, for example, by providing a denser or stronger release component formulation at the site of rib contact. The release component may comprise enteric material, e.g., a polymethacrylate-based copolymers such as Eudragit, at the site of rib contact.

In the design of FIGS. 80G-80J, the trigger housing includes two or more spheres 8002 that are displaced upon partial or complete degradation of the release component. More specifically, when the release component degrades, a sphere race 8010 moves down, allowing the spheres 8002 to move radially thereby releasing the nut. Once the nut 8014 is released, the nut and piercer travel longitudinally and breaks the breakable seal of the gas container.

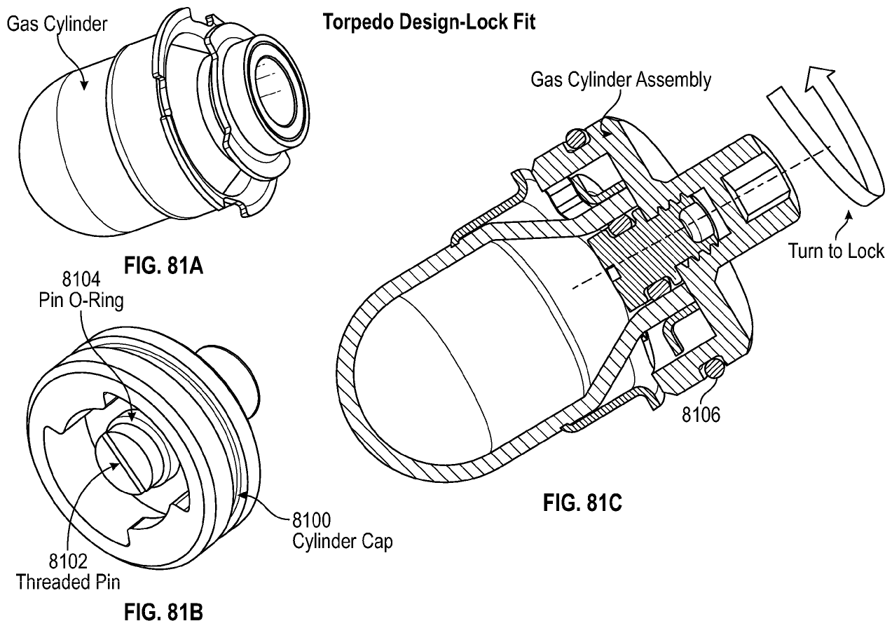

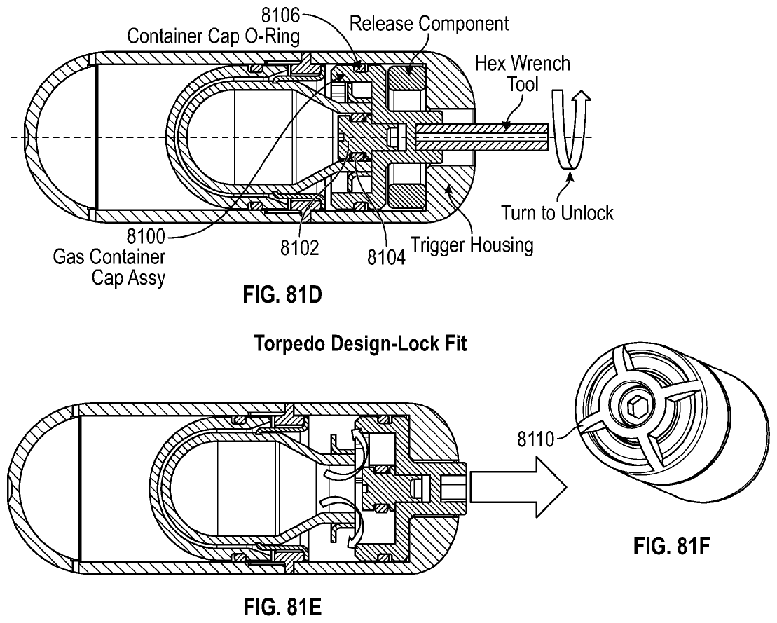

In the embodiment of FIGS. 81A-81C, the trigger housing does not include a spring. Instead, a container cap 8100 is lock fit to the gas container thereby preventing release of the pressurized gas. A threaded pin 8102 and a pin O-ring 8104 are assembled to the container cap 8100. After the gas container is pressurized, the container cap is attached to the gas container and rotated, for example 90 degrees, to lock it in place, as shown in FIG. 81C. A second O-ring 8106 is installed onto the container cap, and both a release component and the gas container assembly are inserted into the trigger housing. The cap is again turned, for example 90 degrees, to unlock it, and now the gas pressure from the gas container pushes the container cap against the release component. After the device is administered, the release component is introduced to biological fluid, where it dissolves or degrades at a predetermined anatomical location based on a physiological condition such as pH. As the release component dissolves or degrades, the container cap is displaced from the gas container, and gas is released from the pressurized container thereby actuating the device. Hence, in this design, no spring and no piercer is used.

Referring to FIGS. 81E and 81F, the trigger housing may include one or more ribs 8110 or other features that serve to apply concentrated pressure on the release component, which helps contribute to catastrophic failure of the release component. In this case, gas from the gas container applies force to the release component, which also helps clear the release component, or pieces of the release component, from the trigger housing. The trigger housing optionally includes openings or “windows” that allow for increased or more uniform hydration of the release component.

The trigger housing may include one or more ribs or other features that serve to apply concentrated pressure on the release component, which helps contribute to catastrophic failure of the release component. Further, the trigger housing may provide space for debris from the release component to disperse, thereby preventing obstruction of any moving parts on the device such as the cylinder cap.

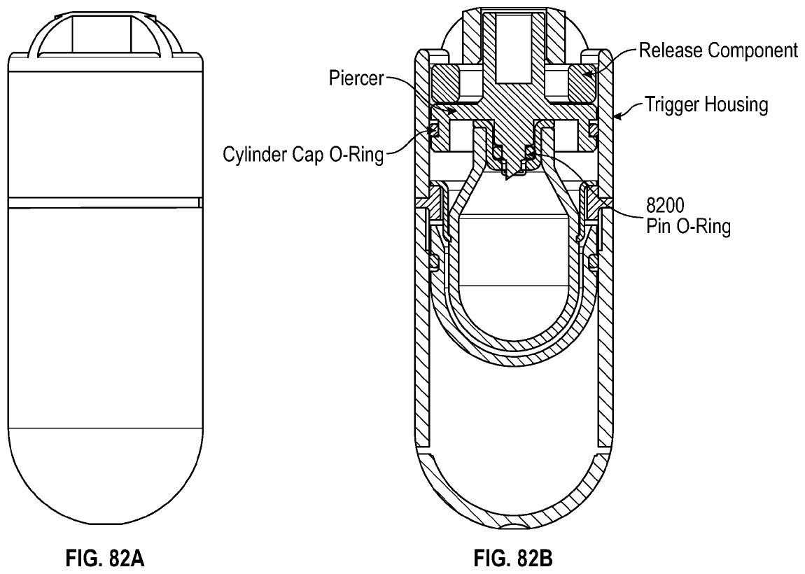

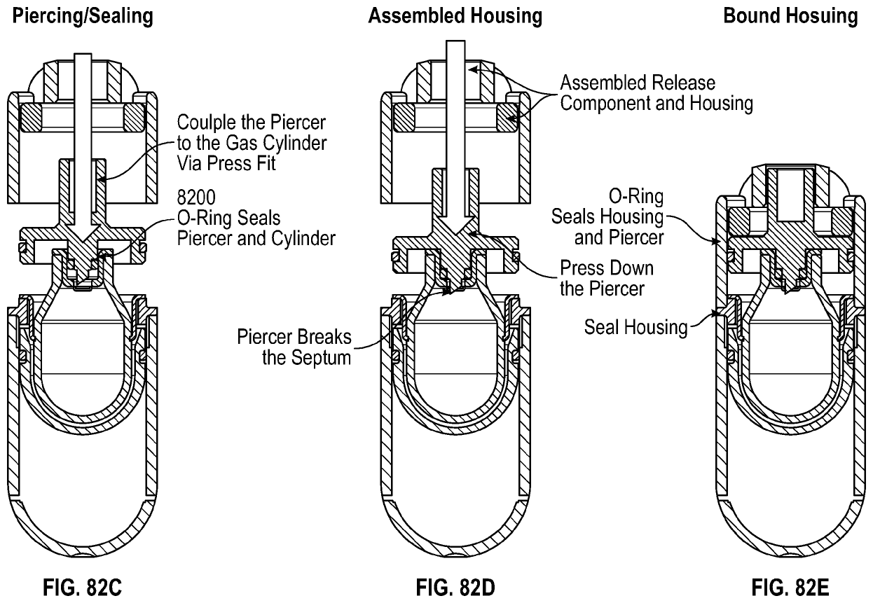

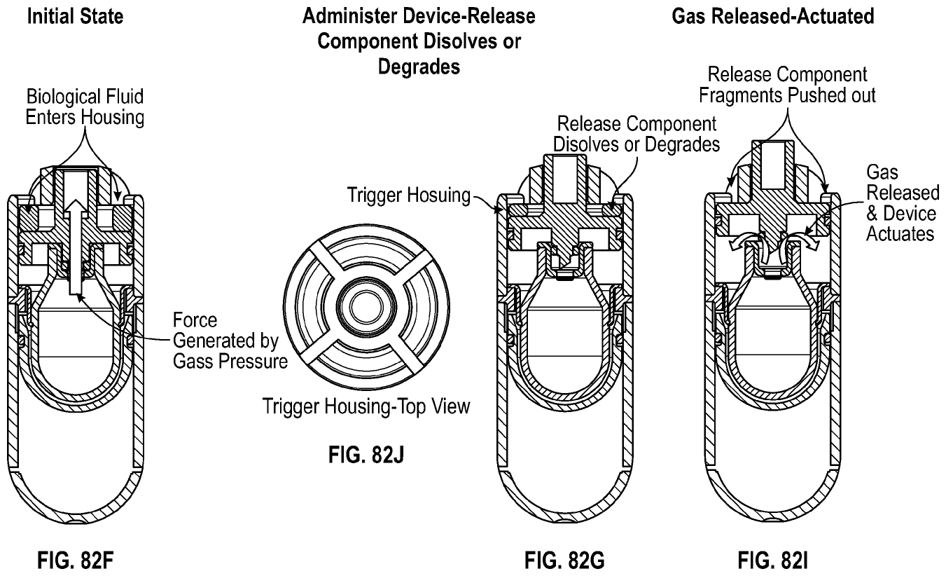

In another embodiment, similar to the device shown in FIGS. 81A-81F, the trigger housing does not include a spring, and instead uses the piercer to seal the gas cylinder—the two components are coupled via a pressure fit as shown in FIGS. 82A-821. The piercer may include an O-ring 8200 to improve the seal. The piercer breaks the breakable seal, or septum, of the gas container. The trigger housing, or drive module housing, is joined to the drug module housing after the piercer is coupled to the gas cylinder. Similar to the device shown in FIGS. 81-81F, the release component helps maintain the seal between the piercer and gas cylinder. When the release component dissolves or degrades in the presence of a biological fluid, the piercer is displaced from the gas cylinder, and gas is released from the pressurized cylinder thereby actuating the device.

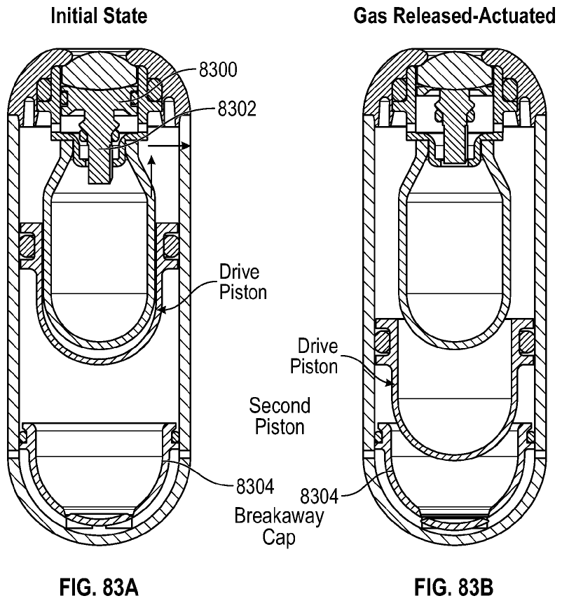

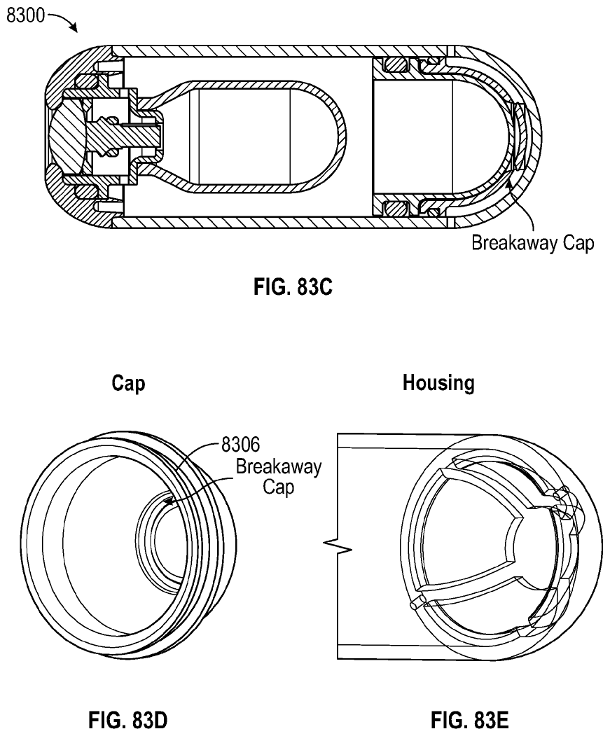

Similar to the devices shown in FIGS. 81A-81F and 82A-821, in the design of FIGS. 83A-83E, the trigger housing does not include a spring, and instead uses a piercer or piercer-like plug 8300 to seal the gas cylinder—the two components are coupled via a spool valve 8302. The shape and position of the release component places the eroding surface in tension to accelerate structural failure or degradation of the release component thereby actuating the device. In another embodiment, the device comprises a second piston 8304 for blocking the openings, wherein the second piston includes an internal breakaway cap 8306. As shown in FIGS. 83C and 83D, the breakaway section lodges into housing recess after it is displaced by the drive piston. The drug payload can flow through channels in the housing and out of the openings. Alternatively, the second piston 8304 can move to uncover the openings as shown in FIGS. 49A and 49B, and the breakaway cap can serve as a vent.

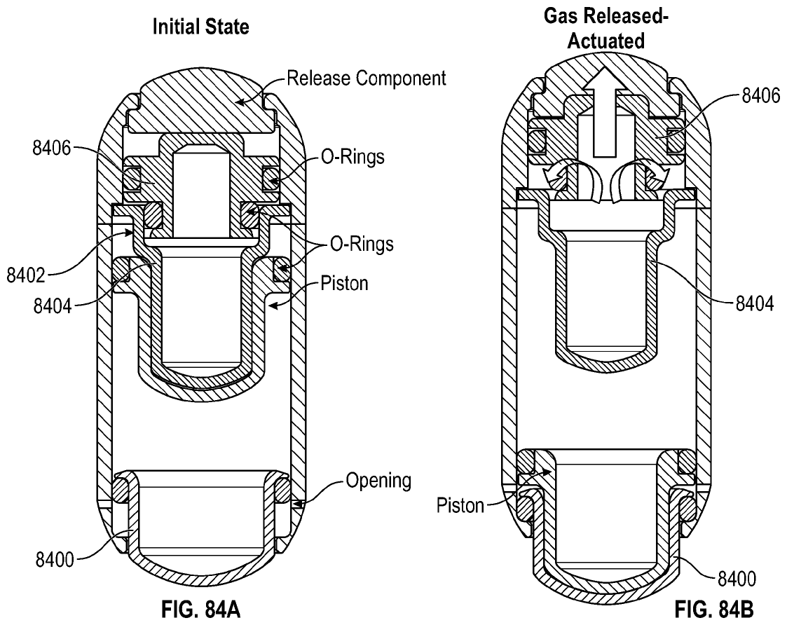

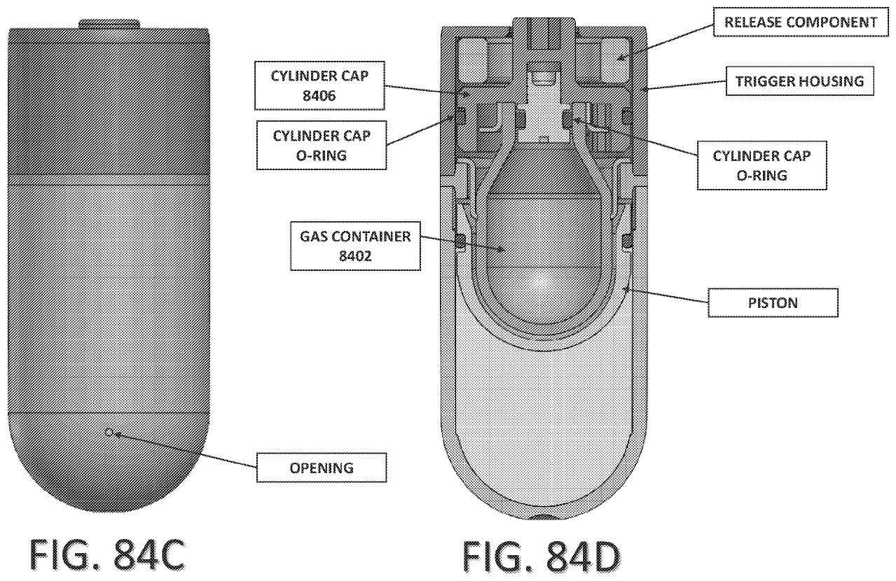

The device shown in FIGS. 84A and 84B is similar to the devices shown in 81A-81F and 82A-821, however, this device includes a second piston 8400 that blocks the one or more openings until the device is actuated. In addition, the gas container 8402 has a first part 8404 sealed with a second part 8406. These two parts separate allowing for gas escape when the release component weakens. The gas cylinder can be reduced to about 80-100 μL total volume, and the drug volume can increase to 500-600 μL. In this embodiment as well, no spring and no piercer is used.

Similar to FIG. 84A and FIG. 84B, the device shown in FIGS. 84C and 84D has a gas container 8402 sealed with a second part such as an end cap 8406. The end cap 8406 may or may not include an inner O-ring on a portion of the end cap extending into the opening of the gas container, and an outer O-ring which slidably seals the end cap against the inner walls of the trigger housing. The end cap 8406 is retained in a sealed position held against the opening of the gas container by the release component. The end cap 8406 prevents gas from escaping from the gas container (while the release component is intact). When the release component partially or fully erodes, degrades, fractures and/or dissolves, the end cap is pushed away from the gas container, allowing gas to escape, which actuates the device. As shown, no piercer and no spring is used. In some embodiments, the device does not include a second piston as shown in FIG. 84D, and in other embodiments there is a second piston as shown in FIGS. 84A and 84B. In some embodiments, the trigger housing includes openings or “windows” that allow for increased or more uniform hydration of the release component and for degraded material from the release component to get displaced away from the housing.

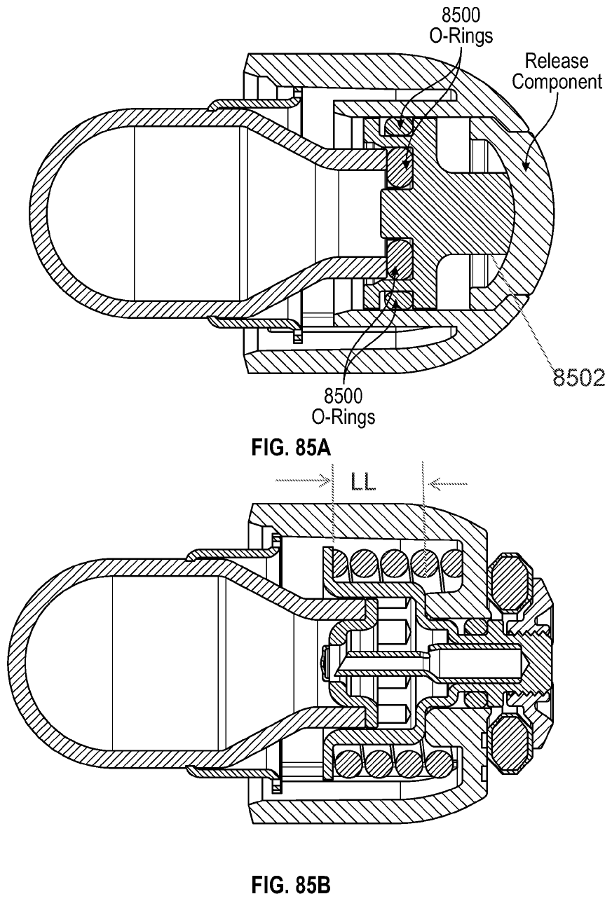

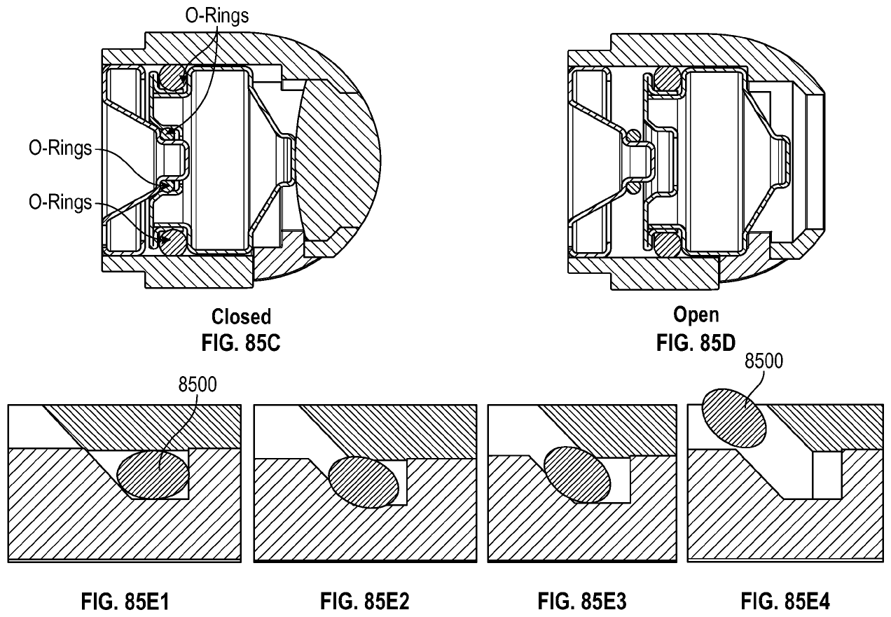

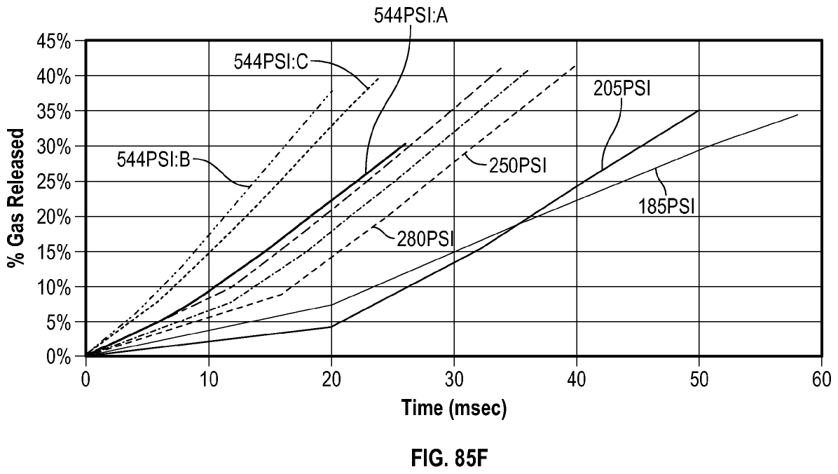

In some embodiments, O-rings are used to seal the gas container as shown in FIGS. 85A to 85E, and the release mechanism uses one or more O-rings 8500 in a receiver 8502 being displaced from the gas pathway as shown in FIGS. 85E1-85E4. FIG. 85F shows test results of displacing O-rings used to seal pressurized gas container. At 250 psi or higher (1.72 MPa) about 35% of gas is released within 35 milliseconds.

FIG. 85B shows an ingestible device 8510 having a gas container having a first or bulb part 8512, a seal plate 8514 joined to the neck or narrow end of the bulb part 8512, and a septum or seal 8516 on the seal plate 8514 which is pierceable by a piercer 8518. The piercer 8518 may have a length measuring from the top of the shroud (which bottoms out against the trigger housing) of about 2-3 mm or about 2.4 to 2.6 mm. In this design, piercer parameters are as shown in Table 2 below.

| | |
|---|---|
| Force at Operating Points (N) | Tensile Stress at Operating Points |

| Description | 4.0 m | 4.65 mm | 5.05 mm | 4.05 mm | 4.65 mm | 5.05 mm |
|---|---|---|---|---|---|---|
| Original 8431 7557 [A] | 34.8 | 30.5 | 27.6 | 51% | 45% | 40% |
| Increased Foree 8431 8350(A] | 45.4 | 39.4 | 35.4 | 55% | 48% | 43% |

| Relaxation after | prestress, |
|---|---|
| Recommended Min | at 500 |
| Len hmm | da s % |
| 4.07 | 1.8 |
| 4.08 | 2.5 |

Thus, the spring may exert a force of about 35 to 45 N.

In some cases the gas container is filled with a liquified gas such as difluoromethane (R-32, CH₂F₂) or argon. Liquified gas provides advantages over non-liquified gas. For example, difluoromethane allows for smaller cylinder volume compared to non-liquified gas, and liquified gas provides a more consistent gas pressure through the piston stroke. Argon has the advantage of being less sensitive to temperature changes than difluoromethane.

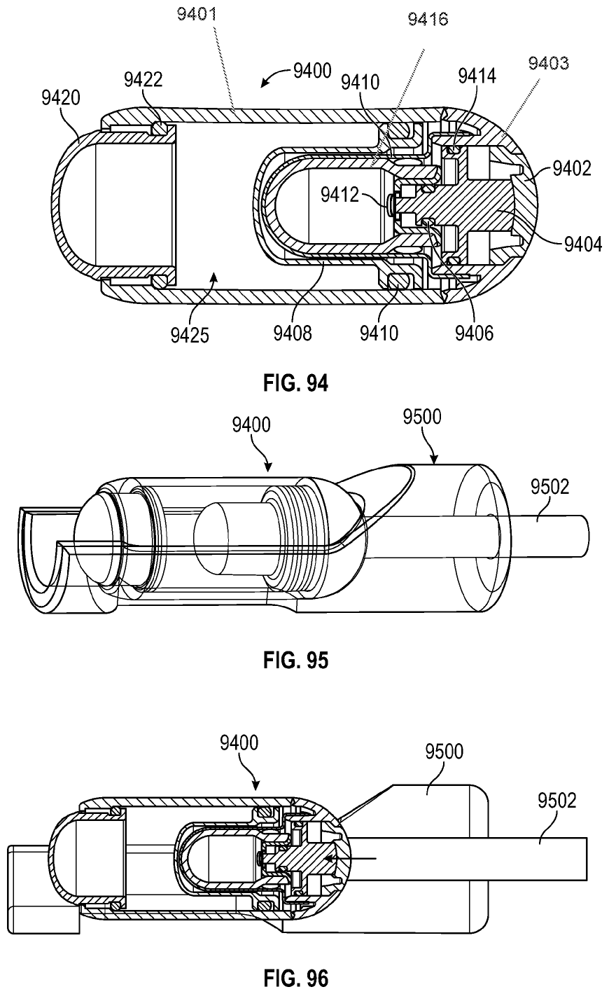

As shown in FIGS. 94-98, an ingestible device 9400 may be designed to provide a two-step gas release. In this design, in a first step, before the device is ingested, gas is released from a gas container by manually breaking or piercing a seal of the gas container using an external element or tool, while also sealing the released gas from the piston, to prevent actual actuation of the device. In a second step, after the device is ingested, a release component dissolves, allowing movement of components within the device to allow the gas to act on the piston and actuate the device. The second step may be performed at the factory, for manufacturing and assembly purposes, or at the point of use, that is immediately before the device is ingested.

The device 9400 includes a first housing 9401 attached to a second housing 9403. A release component 9402 is in the second housing 9403. A piercer 9404 is slidably positioned between the release component 9402 and a seal 9412 on a gas container 9416 containing compressed gas. An O-ring 9406 provides a sliding seal between the piercer 9404 and the second housing 9403. An O-ring 9410 provides a sliding seal between a first piston 9408 and the first housing 9401. An O-ring 9422 provides a sliding seal between a second piston 9420 and the first housing 9401. A dispensable substance. e.g., a liquid, 9425 is contained in a reservoir formed between the first and second pistons, and within the cylindrical sidewalls of the first housing.

Referring to FIGS. 95 and 96, in an example of point-of-use gas release, the device 9400 is placed in a holder 9500 which prevents the device from moving as a plunger 9502 is manually pressed against the release component 9402. This moves the release component 9402 and the piercer 9404 in the direction of the arrow in FIG. 96, with the piercer piercing the seal 9412 on the gas container. The plunger 9502 may slide through a dampening sleeve or element in the holder 9500 to reduce recoil movement resulting from release of the compressed gas.

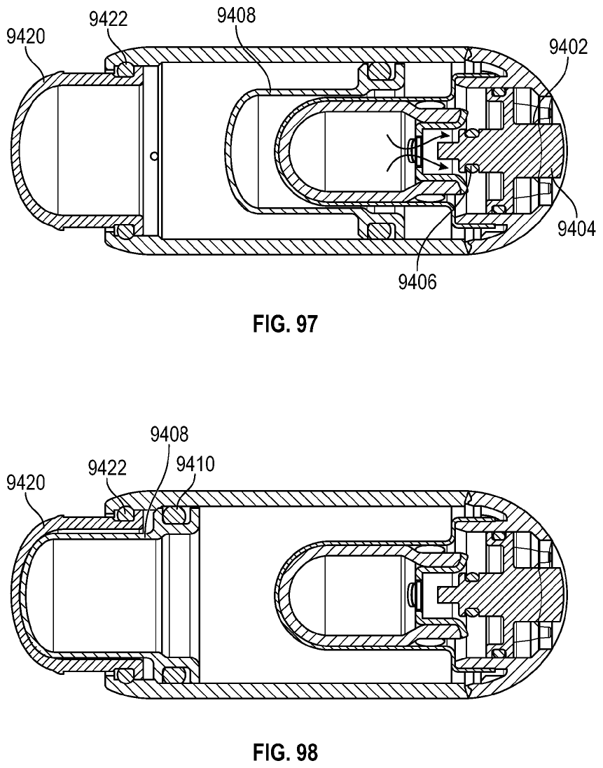

As shown by the arrows in FIG. 97, the compressed gas is released into the second housing 9403, pushing the piercer 9404 and the release component 9402 back to their original positions shown in FIG. 94. The compressed gas is confined into the second housing 9403 by O-ring 9406. The device is then ingested. After it is ingested, the release component partially or fully erodes, degrades, fractures and/or dissolves. The piercer 9404 then moves in the opposite direction (of the arrow in FIG. 96). This allows the compressed gas to flow out of the second housing 9403 and exert force on the first piston 9408. As shown in FIGS. 97 and 98, the first piston 9408 exerts pressure on the dispensable substance, which moves the second piston 9420 (if used) from its original position to the extended position shown in FIG. 98. The dispensable substance is ejected from nozzles or openings in the first housing 9401.

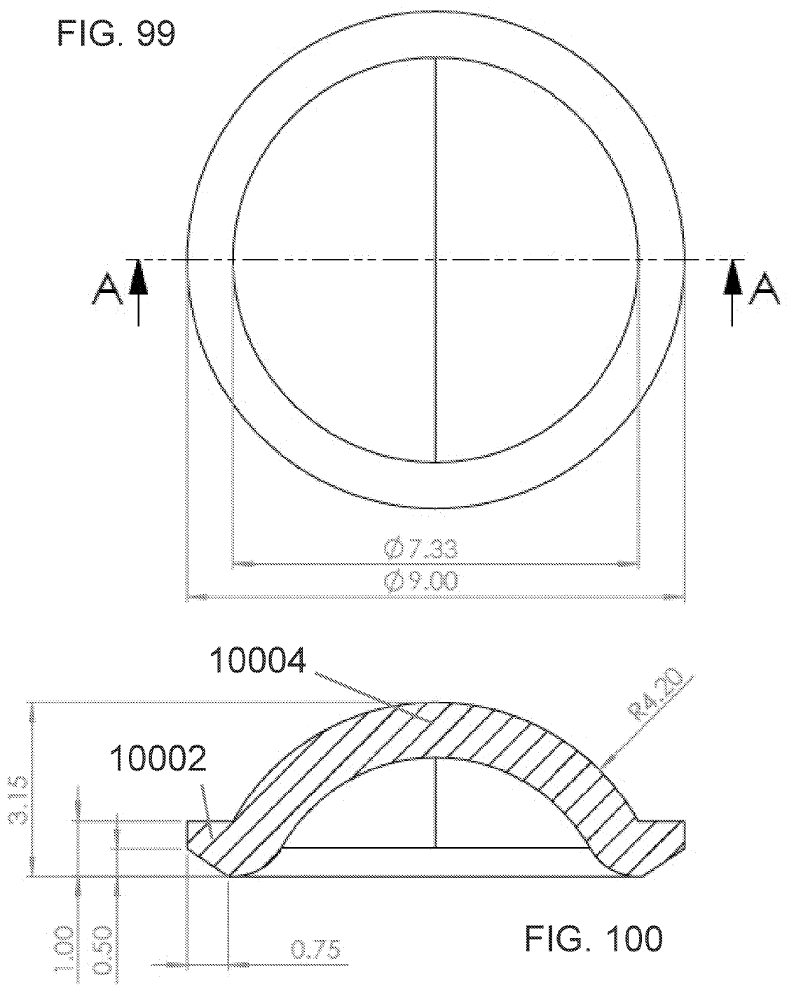

FIGS. 99 and 100 show an alternative second piston configured to make it easier to fill the drug module and provide a higher jet force, which may allow using gas lower pressure (e.g., 320 psi). The dimensions shown are representative examples. As shown the second piston has a perimeter rim 10002 with a flat top surface, a flat perpendicular edge and an angled lower surface. The inner portion or dome 10004 is a spherical shell. Device for Trans-epithelial Delivery

Generally, trans-epithelial delivery can be achieved at any desired location within the GI tract of a subject. In some embodiments, trans-epithelial delivery is achieved in the small intestine of the subject, such as, for example, in the duodenum, the jejunum and/or the ileum. In certain embodiments, trans-epithelial delivery is achieved in the large intestine of the subject, such as, for example, the cecum or the colon.

Trans-epithelial delivery can be achieved using any one of the ingestible devices described above with respect to trans-epithelial delivery. In such embodiments, the relevant parameters are usually modified accordingly. Typically, this modification involves modifying the values for the relevant parameters. Examples are provided in the following paragraphs.

In general, an ingestible device for trans-epithelial delivery is configured to deliver a jet of the dispensable substance having a peak jet power of, in some embodiments, from 6 Watts to 17 Watts, or, in some embodiments, greater than 6.6 Watts to 11 Watts, or, in some embodiments, greater than 6.6 Watts and less than 9 Watts.

Generally, an ingestible device for trans-epithelial delivery is configured to deliver a jet of the dispensable substance having a peak jet pressure of about 2 psig (e.g., about 2.5 psig, about 3 psig, about 3.5 psig, about 4 psig) and/or at most about 10 psig (e.g., at most about 8 psig, at most about 6 psig, at most about 5 psig). In some embodiments, an ingestible device for trans-epithelial delivery is configured to deliver a jet of the dispensable substance having a peak jet pressure of from about 2 psig to about 10 psig (e.g., from about 2.5 psig to about 8 psig, from about 3 psig to about 6 psig, from about 3.5 psig to about 5 psig, from about 4 psig to about 5 psig).

In general, an ingestible device for trans-epithelial delivery is configured to deliver a jet of the dispensable substance having a peak jet force of, in some embodiments, from 0.2 N to 0.6 N, or, in some embodiments, greater than 0.26 N to 0.4 N, or, in some embodiments, from 0.28 N to 0.32 N.

In general, an ingestible device for trans-epithelial delivery is configured to deliver a jet of the dispensable substance: at a minimum jet velocity of from at least about 2 m/s (e.g., at least about 3 m/s, at least about 4 m/s, at least about 5 m/s) and/or at most about 20 m/s (e.g., at most about 15 m/s, at most about 10 m/s, at most about 8 m/s), or with a peak jet velocity of 45 meters per second to 60 meters per second.

In general, an ingestible device for trans-epithelial delivery is configured to provide an internal pressure of, in some embodiments, from 280 psig to 400 psig, or, in some embodiments, from 320 psig to 350 psig.

In general, an ingestible device for trans-epithelial delivery is configured to provide a nozzle pressure of from about 3.62 psig to about 21.76 psig (e.g., from about 3.62 psig to about 18.13 psig, from about 3.62 psig to about 14.50 psig, from about 3.62 psig to about 10.88 psig, from about 3.62 psig to about 7.25 psig, from about 4.35 psig to about 7.25 psig, about 4.35 psig).

Generally, an ingestible device for trans-epithelial delivery is configured to contain a dispensable substance at a peak fluid pressure, in some embodiments, from 270 psig to 390 psig, and in some embodiments, from 310 psig to 340 psig.

In general, an ingestible device for trans-epithelial delivery is configured to directly deliver at least about 50% (e.g., at least about 60%, at least about 70%, at least about 80%, at least about 90%, at least about 95%, at least about 97%) of the dispensable substance from the ingestible device to the mucus.

### Device for Topical Delivery

Generally, topical delivery can be achieved at any desired location within the GI tract of a subject. In some embodiments, topical delivery is achieved in the small intestine of the subject, such as, for example, in the duodenum, the jejunum and/or the ileum. In certain embodiments, topical delivery is achieved in the large intestine of the subject, such as, for example, the cecum, ascending colon, transverse colon, or descending colon.

In general, an ingestible device for topical delivery is configured to provide an internal pressure of, in some embodiments, from 5 psig to 350 psig, or, in some embodiments, from 5 psig to 100 psig, or, in some embodiments, from 5 psig to 50 psig. Unlike an ingestible device for trans-epithelial delivery, an ingestible device for topical delivery does not create a high velocity jet intended to penetrate the epithelial layer of the gastrointestinal; therefore, the internal pressure is generally lower and/or the opening are larger and/or more numerous.

In general, an ingestible device for topical delivery contains the dispensable substance at an initial fluid volume of about 50 μL to about 800 μL (e.g., from about 100 μL to about 600 μL, from about 200 μL to about 400 μL).

In general, an ingestible device for topical delivery is configured to deliver at least about 50% (e.g., at least about 60%, at least about 70%, at least about 80%, at least about 90%, at least about 95%, at least about 97%) of the dispensable substance from the ingestible device into the lumen of the GI tract.

In certain embodiments, an ingestible device for topical delivery is configured as disclosed in the above-discussion regarding trans-epithelial delivery, but with a relatively large number of openings and a relatively large opening diameter such that performance properties for topical delivery (discussed above) can be achieved. As an example, in some embodiments, an ingestible device for topical delivery has from 2 nozzles to 10 nozzles. Each nozzle can have a diameter of from about 0.5 mm to about 3 mm (e.g., from about 1 mm to about 2.5 mm, from about 2 to 2.5 mm).

### Delivery of Therapeutics

Provided herein are ingestible devices and methods that deliver therapeutic agents into the intestinal lumen, mucus, mucosa and/or submucosa by topical, epithelial or trans-epithelial administration to the GI tract of a subject. Current methods of administration for most large molecule therapeutic agents or small molecule therapeutic agents with poor oral bioavailability are subcutaneous (SC), intramuscular (IM), or bolus intravenous (IV) injection targeting the systemic circulation. The devices and methods described herein provide an alternative route of administration to current injectable medications, which can lead to greater convenience and compliance since they minimize or avoid the logistical challenges, patient compliance and adherence challenges, pain, and discomfort associated with traditional routes of administration.

In some embodiments of the devices or methods described herein:
the therapeutic is released at a location in the small intestine of the subject. In some embodiments of any of the devices or methods described herein, the location is in the proximal portion of the small intestine (e.g., duodenum or jejunum);
the location is in the distal portion of the small intestine (e.g., jejunum or ileum). In some embodiments of the devices or methods described herein, the therapeutic is released at a location in the large intestine of the subject. In some embodiments of any of the devices or methods described herein, the location is in the proximal portion of the large intestine (e.g., cecum, ascending colon, or transverse colon); and/or
the location is in the distal portion of the large intestine (e.g., transverse colon or descending colon).

Also, by providing a higher concentration of therapeutic in GI tissue, the devices and methods described herein are particularly well-suited for treatment of diseases and conditions of the endoderm, including the liver.

In some embodiments of any of the devices or methods described herein:
the releasing of the therapeutic is triggered by one or more of: a pH in the jejunum of about 6.1 to about 7.2, a pH in the mid small bowel of about 7.0 to about 7.8, a pH in the ileum of about 7.0 to about 8.0, a pH in the right colon of about 5.7 to about 7.0, a pH in the mid colon of about 5.7 to about 7.4, or a pH in the left colon of about 6.3 to about 7.7;
the releasing of the therapeutic is triggered by degradation of a release component located in the device;
the releasing of the therapeutic is dependent on enzymatic activity at or in the vicinity of the location;
the composition includes a plurality of electrodes including a coating, and releasing the therapeutic is triggered by an electric signal by the electrodes resulting from the interaction of the coating with an intended site of release of the therapeutic;
the release of the therapeutic is triggered by a remote electromagnetic signal. In some embodiments of any of the devices or methods described herein, the release of the therapeutic is triggered by generation in the composition of a gas in an amount sufficient to expel the therapeutic; and/or
the release of the therapeutic is triggered by an electromagnetic signal generated within the device according to a pre-determined drug release profile.

### Therapeutics for Delivery

Therapeutics suitable for use with the devices and methods described herein include both small molecules and large molecules. In some embodiments, the therapeutic agent is a large molecule. Examples of large molecules include, but are not limited to, biologic drugs, proteins including fusion proteins, peptides including cyclic peptides, protein-drug conjugates, cells including stem cells, and nucleic acids such as inhibitory nucleic acids, antisense nucleic acids, siRNA, ribozymes, and the like. In some embodiments, the therapeutic agent is a large molecule with a molecular weight of at least about 60 kilodaltons (kDa), or about 60 kDa to about 200 kDa, about 60 kDa to about 175 kDa, or about 60 kDa to about 150 kDa.

In some other embodiments, the therapeutic agent has a molecular weight of at least about 20 kDa, at least about 30 kDa, at least about 40 kDa, or at least about 50 kDa, or from about 20 kDa to about 200 kDa, about 20 kDa to about 175 kDa or about 20 kDa to about 150 kDa.

In some embodiments, the therapeutic agent is a molecule, e.g., a protein or peptide, with a molecular weight of greater than about 1.5 kDa and less than about 20 kDa, less than about 30 kDa, less than about 40 kDa, less than about 50 kDa or less than about 60 kDa. In some other embodiments, the therapeutic agent has a molecular weight of from about 5 kDa to about 10 kDa, 20 kDa, 30 kDa, 40 kDa or 50 kDa. In some embodiments, the therapeutic agent is a molecule with a molecular weight of about 5 kDa to about 10 kDa, such as about 6 kDa. In some embodiments, the therapeutic agent is a protein or peptide. In some embodiments, the therapeutic agent is a protein-drug conjugate. In some embodiments, the therapeutic agent is insulin.

In some embodiments, the therapeutic agent is a small molecule. A “small molecule,” as used herein, is a compound, typically an organic compound, having a molecular weight of about 50 Da to about 1500 Da, about 60 Da to about 1500 Da, about 500 Da to about 1000 Da, or no more than about 1500 Da, such as about 1000 Da, about 750 Da, or about 500 Da. In some embodiments, the therapeutic agent is a small molecule with a molecular weight of about 50 Da to about 1500 Da. In some embodiments, the therapeutic agent is a small molecule with a molecular weight of about 150 Da to about 1500 Da.

In some embodiments, the therapeutic agent is a non-small molecule. Exemplary non-small molecule therapeutic agents for use in the devices and methods provided herein include, but are not limited to, abatacept, teriparatide, eculizumab, emicizumab, pegfilgrastim, semaglutide, dulaglutide, sargramostim, ustekinumab, secukinumab, tocilizumab, vedolizumab, natalizumab, interferon beta-1a, denosumab, alirocumab, evolocumab, adalimumab, etanercept, trastuzumab, pembrolizumab, pertuzumab, ARO-HBV, glatiramer acetate Copaxone®, LY-3321367, cetuximab (Erbitux®), ipilimumab (Yervoy®), daratumumab (Darzalex®), albumin-bound paclitaxel (Abraxane®), tanezumab, LY-2510924, LCAR-B38M, PF-004518600, TAK-079, PF-06730512, LY-3076226, NOV-13, FAZ-053, LY-3375880, PF-06823859, CNGB3 gene therapy, mosunetuzumab, RG-6147, scAAV/JeT-GAN-based gene therapy, ranibizumab, cofetuzumab pelidotin, SHR-A1201, TAK-671, A-004 (AAV2/5-hRKp.RPGR) gene therapy. NG-HER2 antibody drug conjugate, TAK-164, RG-7861, JNJ-61186372, PF-05206388, NJH-395, PF-05230907, BIIB-059. PF-06688992, ianalumab, TAK-573. PF-06755347, CD200R mAb agonist, cetrelimab, ligelizumab, PF-06801591. JNJ-64407564, polatuzumab vedotin, PF-06817024. NOV-12, BIIB-054, CTL-119, JNJ-61178104, spartalizumab, RNA CART123, LY-3300054, PD-1 mAb agonist, CART-EGFRvIII, NOV-10, TQJ-230, PF-06863135, PCA-062, JNJ-64041757, CNTO-2476, tiragolumab. PF-06946860, elgemtumab, LY-3415244, LKA-651, RG-6109, ECF-843, JNJ-61610588, AAV8-RLBP1 gene therapy, LAG-525, MOR-106, BTLA agonist mAb, AMV-564, JNJ-64041809, MBG-453, CGF-166, brolucizumab, NOV-9, CJM-112, tesidolumab, NIZ-985, MCS-110, BHQ-880, NOV-8, CLR-325, XmAb-13676, huMesoCART, NZV-930, CGM-097, NOV-7, and certolizumab pegol; and biosimilars thereof; and glycosylation variants thereof. Additional exemplary drugs for delivery using any of the devices or methods described herein include those listed in Table 3.

TABLE 3

| | | | | | Drug | |
|---|---|---|---|---|---|---|
| | | | | | Volume | |
| | | | | | and # | |
| | | | | | capsules | |
| | | | | | needed | |
| | | | | | per | |
| Brand Name | Potential | | Drug | | equivalent | Storage |
| (Drug) | Dose | | concentration | | dose^{a} | temperature |
| Humira ® | 40, 80, 160 mg | | ~40 mg/0.4 mL | | 0.8-3.2 mL | 2-8° C. |
| (adalimumab) | | | | | 2-8 Capsules | 25° C. for |
| | | | | | | up to 14 days |
| Remicade ® | 400 | mg | Diluted to ~4 | | NA | 2-8° C. |
| (infliximab) | | | mg/mL | | | 30° C. for |
| | | | | | | up to 12 months |
| Cimzia ® | 400 | mg | ~200 | mg/mL | 4 mL | 2-8° C. |
| (certolizumabpegol) | | | | | 10 Capsules | 2 hrs at room |
| | | | | | | temp |
| Embrel ® | 50 | mg | ~50 | mg/mL | 2 mL | 2-8° C. |
| (etanerecpt) | | | | | 5 Capsules | 25° C. for |
| | | | | | | 14 days |
| Lantis ® (insulin), | sq | | 1 unit ~0.0347 | | | 2-8° C. |
| Novalog ® (insulin) | | | mg, | | | 30° C. for |
| | | | density of | | | up to 28 days |
| | | | crystal is close | | | |
| | | | to 1 g/cm³ | | | |
| Victoza ® | 1.2 | mg | ~6 | mg/mL | 0.4 mL | 2-8° C. |
| (liraglutide) | | | | | 1 Capsules | 30° C. for |
| | | | | | | up to 30 days |
| Bydureon ® | 2 | mg | ~2 mg/0.6 mL | | 1.2 mL | 2-8° C. |
| (exenatide) | | | | | 3 Capsules | 30° C. for |
| | | | | | | up to 28 days |
| (GHIH) | 0.48-2 | mg | | | | |
| (somatostatin) | | | | | | |
| Sandosatin ® | 100-500 | mcg | ~500 | mcg/mL | 0.4-2 mL | 2-8° C. |
| (Octreotide) | | | | | 1-5 Capsules | 30° C. for |
| | | | | | | up to 14 days |
| Avonex ® (interferon | 30 | mcg | ~30 mcg/0.5 mL | | 1 mL | 2-8° C. |
| beta-1a) | | | | | 2.5 Capsules | 30° C. for |
| | | | | | | up to 30 days |
| Tysabri ® | 300 | mg | ~2.69 | mg/mL | NA | 2-8° C. |
| (natalizumab) | | | | | | |
| Avastin ® | 5 | mg | IFU: Do not | | NA | 2-8° C. |
| (bevacizumab) | | | administer as | | | |
| | | | bolus, IV | | | |
| Entyvio ® | 300 | mg | IFU: Do not | | NA | |
| (Vedolizumab) | | | administer as | | | |
| | | | bolus, IV | | | |
| Fragmin ® | 2500-18000 | IU | 5000 | IU | 0.6 mL | Room Temp |
| (Dalteparin) | | | | | 1.5 Capsules | |
| Rocephin ® | 1 | g | ~350 | mg/mL | | |
| (Ceftriaxone)(or | | | | | | |
| other antimicrobials) | | | | | | |
| Interferon alfa-2b | 3-30 million | IU | ~50 | million IU/mL | 0.12-1.2 mL | 2-8° C., up to |
| | | | | | <1-3 Capsules | seven days at |
| | | | | | | room temp |
| Natpara ® | 50-100 | mcg | ~1 | mg/mL | <<1 Capsules | 2-8° C. |
| (Parathyroid | | | | | | |
| Hormone) (PTH) | | | | | | |
| Genotropin ® | 0.2-2 | mg | ~5.3 | mg/mL | <1 Capsule | 2-8° C. |
| Human Growth | | | | | | 4 weeks after |
| Hormone (HGH) | | | | | | reconstitution |

^{a}Number of capsules assumes a drug reservoir of about 400 microliters
sq: subcutaneous
IFU: Instructions for use
IU: International Unit

In some embodiments, the therapeutic agent is a small molecule. Exemplary small molecule therapeutic agents for use in the devices and methods provided herein include, but are not limited to, glasdegib maleate, ibuprofen+paracetamol combination, PF-06873600, LY-3200882, PF-06952229, PF-06821497, LY-3405105, LY-3372689, LY-3023414, enzastaurin, SY-008, taladegib, crenigacestat, merestinib, LY-3214996, ralimetinib, galunisertib, TBA-7371, LY-3381916, LY-2874455, erdafitinib, pimodivir, aprocitentan, JNJ-56136379, BMS-986177, lazertinib, JNJ-64619178, JNJ-55308942, AL-034, JNJ-67670187, JNJ-64264681, JNJ-64417184, JNJ-3534, JNJ-64991524, JNJ-64140284, pimodivir+oseltamivir combination, JNJ-61803534, ipatasertib dihydrochloride, fenebrutinib, RG-6171, belvarafenib, RG-6174, alpelisib, asciminib, leniolisib, clofazimine, siremadlin, capmatinib, PBF-509, LNP-023, UNR-844, ganaplacide, cipargamin, adriforant, LYS-006, QCC-374, MAK-683, LCL-161, BLZ-945, LOU-064, VPM-087. WNT-974, totrombopag, hydroxychloroquine+trametinib combination, LTT-462, NOV-11, LSZ-102, allosteric inhibitors of SHP2 phosphatase, mocravimod dihydrochloride, BCL-201, mivavotinib, DSM-265, sapanisertib, TAK-931, TAK-906, alisertib, TAK-580, pediatric formulation of azilsartan, TAK-418, and vonoprazan fumarate+aspirin combination.

In some embodiments, the therapeutic agent is a monoclonal antibody (mAb). In some embodiments, the mAb is an anti-interleukin-17A (anti-IL-17A) mAb. In some embodiments, the mAb is an anti-interleukin-17A (anti-IL-17A) mAb that can be used to treat inflammatory conditions and/or autoimmune diseases, including, but not limited to, rheumatoid arthritis, plaque psoriasis, active psoriatic arthritis, and ankylosing spondylitis. An exemplary anti-IL-17A mAb is ixekizumab (Taltz®). See, e.g., Genovese et al., Arthritis & Rheumatology, 66.7:1693-1704 (2014). In some embodiments, the mAb is a selective mAb against antiopoietin 2 (Ang2). An exemplary mAb selective against Ang2 is LY3127804. In some embodiments, the mAb is a PD-1 receptor agonist mAb, such as peresolimab (LY3462817), that can be used to treat inflammatory conditions and/or autoimmune diseases.

In some embodiments, the therapeutic suitable for use with the devices and methods described herein is a glucagon-like peptide 1 (GLP-1) receptor agonist. The GLP-1 pathway has been indicated in the treatment of type 2 diabetes mellitus (T2DM) or other metabolic conditions such as obesity. In some embodiments, the GLP-1 receptor agonist is a peptide or a small molecule. In some embodiments, the GLP-1 receptor agonist is formulated with a carrier, or delivery agent, which may be a salt of a medium chain fatty acid derivative.

Exemplary GLP-1 receptor agonists for delivery using any of the devices or methods described herein include those listed in Table 4A.

TABLE 4A
GLP-1 receptor agonists adaptable for delivery via ingestible
device for the treatment of the listed diseases and conditions:

| GLP-1 | | | |
|---|---|---|---|
| Agonist | | Dosage and | |
| (Company) | Tradename | Administration | Comments |
| Albiglutide | Tanzeum | 30 mg/dose (0.82 μmol); up to | GLP-1 (7-36) dimer fused to |
| (GSK) | EU: Eperzan | 50 mg/dose; Once weekly, | recombinant human albumin. |
| | | subcutaneus injection | MWt ~73 kDa. |
| Dulaglutide | Trulicity | 0.75 mg/dose (0.024 μmol); up to | GLP-1(7-37) covalently linked |
| (Eli Lilly) | | 1.5 mg/dose; Once weekly, | to an Fc fragment of human |
| | | subcutaneus injection | IgG4. MWt ~63 kDa |
| Exenatide | Byetta | 5 μg/dose (1.2 nmol); up to | Synthetic form of exendin-4, |
| (Astra | | 10 μg/dose; Twice daily, | a peptide isolated from |
| Zeneca) | | subcutaneus injection | H. suspectum venom. |
| | | | MWt ~4 KDa |
| | Bydureon, | 2 mg/dose (0.48 μmole), Once | Extended release |
| | Bydureon | weekly, subcutaneus injection | microsphere formulations. |
| | Bcise | | |
| Liraglutide | Victoza | 0.6 mg/dose (0.16 μmol); up to | Fatty acylated GLP-1 analog. |
| (Novo | | 1.8 mg/dose; Once daily, | MWt ~4 KDa. |
| Nordisk) | | subcutaneus injection | |
| | Saxenda | 0.6 mg/dose (0.16 μmol); up to | |
| | | 3 mg/dose; Once daily, | |
| | | subcutaneus injection | |
| Lixisenatide | Adlyxin | 10 μg/dose (2.06 nmol); up to | Recombinant DNA-produced |
| (Sanofi- | EU: Lyxumia | 20 μg/dose; Once daily, | GLP-1 analog. |
| Aventis) | | subcutaneus injection | MWt ~5 KDa. |
| Semaglutide | Ozempic | 0.25 mg/dose (0.061 μmol); up to | GLP-1-like peptide-1 analog. |
| (Novo | | 1 mg/dose; Once weekly, | MWt ~4 KDa. |
| Nordisk) | | subcutaneus injection | Longer acting alternative to |
| | | | liraglutide. |
| Semaglutide | Rybelsus | 3 mg oral tablet once daily for first | GLP-1-like peptide-1 analog; |
| (Novo | | 30 days; followed by 7 mg oral | oral formulation with |
| Nordisk) | | tablet once a day | Emisphere Technologies' |
| | | | Eligen SNAC Carrier |
| | | | Technology |

TABLE 4B
GLP-1

| | CAS | | | |
|---|---|---|---|---|
| Drug Name | Number | Company Name | Molecule Type | Mechanism of Action |
| AP-026 | | Chia Tai Tianqing | Protein | Fibroblast Growth |
| | | Pharmaceutical | | Factor 21 (FGF21) |
| | | Group Co Ltd | | Activator; Glucagon |
| | | | | Like Peptide 1 (GLP1) |
| | | | | Activator |
| CA-503 | | Chongqing Chenan | Peptide | Glucagon Like Peptide |
| | | BioPharm Co Ltd | | 1 Receptor (GLP1R) |
| | | | | Agonist |
| DB-081 | | Tonghua Dongbao | | Glucagon Like Peptide |
| | | Pharmaceutical Co | | 1 Receptor (GLP1R) |
| | | Ltd | | Agonist |
| ecnoglutide | 2459531- | Sciwind Biosciences | Biologic | Glucagon Like Peptide |
| | 73-6 | Co Ltd | | 1 (GLP1) Activator |
| exenatide SR | 141758- | Peptron Inc | Synthetic | Glucagon Like Peptide |
| | 74-9 | | Peptide | 1 Receptor (GLP1R) |
| | | | | Agonist |
| GW-002 | | Jiangsu T-mab | Fusion Protein | Glucagon Like Peptide |
| | | BioPharma Co Ltd | | 1 Receptor (GLP1R) |
| | | | | Agonist |
| GXG-6 | | CSPC | Fusion Protein | Glucagon Like Peptide |
| | | Pharmaceutical | | 1 Receptor (GLP1R) |
| | | Group Ltd | | Agonist |
| HZ-010 | | Hangzhou Heze | Synthetic | Gastric Inhibitory |
| | | Pharmaceutical | Peptide | Polypeptide Receptor |
| | | Technology Co Ltd | | (Glucose Dependent |
| | | | | Insulinotropic |
| | | | | Polypeptide Receptor |
| | | | | or GIPR) Agonist; |
| | | | | Glucagon Like Peptide |
| | | | | 1 Receptor (GLP1R) |
| | | | | Agonist |
| HZCX-012 | | Hangzhou Heze | Synthetic | Gastric Inhibitory |
| | | Pharmaceutical | Peptide | Polypeptide Receptor |
| | | Technology Co Ltd | | (Glucose Dependent |
| | | | | Insulinotropic |
| | | | | Polypeptide Receptor |
| | | | | or GIPR) Agonist; |
| | | | | Glucagon Like Peptide |
| | | | | 1 Receptor (GLP1R) |
| | | | | Agonist |
| insulin degludec + | 844439- | Tonghua Dongbao | Recombinant | Glucagon Like Peptide |
| liraglutide | 96-9 & | Pharmaceutical Co | Peptide; | 1 Receptor (GLP1R) |
| | 204656- | Ltd | Recombinant | Agonist; Insulin |
| | 20-2 | | Protein | Receptor (IR or |
| | | | | CD220 or INSR or EC |
| | | | | 2.7.10.1) Agonist |
| KN-056 | | Alphamab Oncology | Fusion Protein | Glucagon Like Peptide |
| | | | | 1 Receptor (GLP1R) |
| | | | | Agonist |
| PB-718 | | PegBio Co Ltd | Peptide | Glucagon Like Peptide |
| | | | | 1 Receptor (GLP1R) |
| | | | | Agonist; Glucagon |
| | | | | Receptor (GL R or |
| | | | | GCGR) Agonist |
| PB-722 | | PegBio Co Ltd | | Glucagon Receptor |
| | | | | (GL R or GCGR) |
| | | | | Agonist |
| pemvidutide | | Altimmune Inc | Synthetic | Glucagon Like Peptide |
| | | | Peptide | 1 Receptor (GLP1R) |
| | | | | Agonist; Glucagon |
| | | | | Receptor (GL R or |
| | | | | GCGR) Agonist |
| THDB-0211 | | Tonghua Dongbao | | Glucagon Like Peptide |
| | | Pharmaceutical Co | | 1 Receptor (GLP1R) |
| | | Ltd | | Agonist |
| THDBH-120 | | Tonghua Dongbao | | Gastric Inhibitory |
| | | Pharmaceutical Co | | Polypeptide Receptor |
| | | Ltd | | (Glucose Dependent |
| | | | | Insulinotropic |
| | | | | Polypeptide Receptor |
| | | | | or GIPR) Agonist; |
| | | | | Glucagon Like Peptide |
| | | | | 1 Receptor (GLP1R) |
| | | | | Agonist |
| THDBH-121 | | Tonghua Dongbao | | Gastric Inhibitory |
| | | Pharmaceutical Co | | Polypeptide Receptor |
| | | Ltd | | (Glucose Dependent |
| | | | | Insulinotropic |
| | | | | Polypeptide Receptor |
| | | | | or GIPR) Agonist; |
| | | | | Glucagon Like Peptide |
| | | | | 1 Receptor (GLP1R) |
| | | | | Agonist |
| dasiglucagon | 1544300- | Zealand Pharma US | Synthetic | Glucagon Receptor |
| | 84-6 | Inc | Peptide | (GL R or GCGR) |
| | | | | Agonist |
| dulaglutide | 923950- | Eli Lilly and Co | Recombinant | Glucagon Like Peptide |
| | 08-7 | | Peptide | 1 Receptor (GLP1R) |
| | | | | Agonist |
| exenatide | 141758- | AstraZeneca AB | Synthetic | Glucagon Like Peptide |
| | 74-9 | | Peptide | 1 Receptor (GLP1R) |
| | | | | Agonist |
| glucagon | 16941- | Novo Nordisk Inc | Recombinant | Glucagon Receptor |
| | 32-5 | | Peptide | (GL R or GCGR) |
| | | | | Agonist |
| insulin glargine + | 160337- | Sanofi-Aventis South | Recombinant | Glucagon Like Peptide |
| lixisenatide | 95-1 & | Africa (Pty) Ltd | Protein | 1 Receptor (GLP1R) |
| | 320367- | | | Agonist; Insulin |
| | 13-3 | | | Receptor (IR or |
| | | | | CD220 or INSR or EC |
| | | | | 2.7.10.1) Agonist |
| liraglutide | 204656- | Novo Nordisk Inc | Recombinant | Glucagon Like Peptide |
| | 20-2 | | Peptide | 1 Receptor (GLP1R) |
| | | | | Agonist |
| lixisenatide | 320367- | Sanofi-Aventis US | Synthetic | Glucagon Like Peptide |
| | 13-3 | LLC | Protein | 1 Receptor (GLP1R) |
| | | | | Agonist |
| pegloxenatide | 2420483- | Jiangsu Hansoh | Synthetic | Glucagon Like Peptide |
| | 82-3 | Pharmaceutical | Peptide | 1 Receptor (GLP1R) |
| | | Group Co Ltd | | Agonist |
| semaglutide | 910463- | Novo Nordisk Inc | Recombinant | Glucagon Like Peptide |
| | 68-2 | | Peptide | 1 Receptor (GLP1R) |
| | | | | Agonist |
| tirzepatide | 2023788- | Eli Lilly and Co | Synthetic | Gastric Inhibitory |
| | 19-2 | | Peptide | Polypeptide Receptor |
| | | | | (Glucose Dependent |
| | | | | Insulinotropic |
| | | | | Polypeptide Receptor |
| | | | | or GIPR) Agonist; |
| | | | | Glucagon Like Peptide |
| | | | | 1 Receptor (GLP1R) |
| | | | | Agonist |
| BI-456906 | | Boehringer Ingelheim | Synthetic | Glucagon Like Peptide |
| | | International GmbH | Peptide | 1 (GLP1) Activator; |
| | | | | Glucagon Receptor |
| | | | | (GL R or GCGR) |
| | | | | Agonist |
| cotadutide | 1686108- | AstraZeneca Plc | Peptide | Glucagon Like Peptide |
| | 82-6 | | | 1 Receptor (GLP1R) |
| | | | | Agonist; Glucagon |
| | | | | Receptor (GL R or |
| | | | | GCGR) Agonist |
| dapiglutide | 2296814- | Zealand Pharma AS | Synthetic | Glucagon Like Peptide |
| | 85-0 | | Peptide | 1 Receptor (GLP1R) |
| | | | | Agonist; Glucagon |
| | | | | Like Peptide 2 |
| | | | | Receptor (GLP2R) |
| | | | | Agonist |
| DD-01 | | D&D Pharmatech Co | Synthetic | Glucagon Like Peptide |
| | | Ltd | Peptide | 1 Receptor (GLP1R) |
| | | | | Agonist; Glucagon |
| | | | | Receptor (GL R or |
| | | | | GCGR) Agonist |
| DR-10624 | | Zhejiang Doer | Fusion Protein | Beta Klotho (Klotho |
| | | Biologics Corp | | Beta Like Protein or |
| | | | | KLB) Activator; |
| | | | | Fibroblast Growth |
| | | | | Factor Receptor 1 |
| | | | | (Basic Fibroblast |
| | | | | Growth Factor |
| | | | | Receptor 1 or Fms |
| | | | | Like Tyrosine Kinase 2 |
| | | | | or N Sam or Proto |
| | | | | Oncogene c Fgr or |
| | | | | CD331 or FLT2 or |
| | | | | FGFR1 or EC |
| | | | | 2.7.10.1) Agonist; |
| | | | | Gastric Inhibitory |
| | | | | Polypeptide Receptor |
| | | | | (Glucose Dependent |
| | | | | Insulinotropic |
| | | | | Polypeptide Receptor |
| | | | | or GIPR) Agonist; |
| | | | | Glucagon Like Peptide |
| | | | | 1 Receptor (GLP1R) |
| | | | | Agonist |
| DR-10627 | | Zhejiang Doer | Fusion Protein | Gastric Inhibitory |
| | | Biologics Corp | | Polypeptide Receptor |
| | | | | (Glucose Dependent |
| | | | | Insulinotropic |
| | | | | Polypeptide Receptor |
| | | | | or GIPR) Agonist; |
| | | | | Glucagon Like Peptide |
| | | | | 1 Receptor (GLP1R) |
| | | | | Agonist |
| efinopegdutide | 2055640- | Merck & Co Inc | Recombinant | Glucagon Like Peptide |
| | 93-0 | | Protein | 1 Receptor (GLP1R) |
| | | | | Agonist; Glucagon |
| | | | | Receptor (GL R or |
| | | | | GCGR) Agonist |
| GMA-105 | | Gmax Biopharm LLC | Monoclonal | Glucagon Like Peptide |
| | | | Antibody | 1 Receptor (GLP1R) |
| | | | | Agonist |
| GMA-106 | | Gmax Biopharm LLC | Fusion Protein | Gastric Inhibitory |
| | | | | Polypeptide Receptor |
| | | | | (Glucose Dependent |
| | | | | Insulinotropic |
| | | | | Polypeptide Receptor |
| | | | | or GIPR) Antagonist; |
| | | | | Glucagon Like Peptide |
| | | | | 1 Receptor (GLP1R) |
| | | | | Agonist |
| HB-1085 | | Wuxi Hebang | Fusion Protein | Glucagon Like Peptide |
| | | Biotechnology Co Ltd | | 1 Receptor (GLP1R) |
| | | | | Agonist |
| HEC-88473 | | HEC Pharma Co Ltd | Fusion Protein | Beta Klotho (Klotho |
| | | | | Beta Like Protein or |
| | | | | KLB) Activator; |
| | | | | Fibroblast Growth |
| | | | | Factor Receptor 1 |
| | | | | (Basic Fibroblast |
| | | | | Growth Factor |
| | | | | Receptor 1 or Fms |
| | | | | Like Tyrosine Kinase 2 |
| | | | | or N Sam or Proto |
| | | | | Oncogene c Fgr or |
| | | | | CD331 or FLT2 or |
| | | | | FGFR1 or EC |
| | | | | 2.7.10.1) Agonist; |
| | | | | Glucagon Like Peptide |
| | | | | 1 Receptor (GLP1R) |
| | | | | Agonist |
| HL-08 | | Hualan Biological | Fusion Protein | Glucagon Like Peptide |
| | | Engineering Inc | | 1 Receptor (GLP1R) |
| | | | | Agonist |
| HR-17031 | | Jiangsu Hengrui | Recombinant | Glucagon Like Peptide |
| | | Medicine Co Ltd | Protein | 1 Receptor (GLP1R) |
| | | | | Agonist; Insulin |
| | | | | Receptor (IR or |
| | | | | CD220 or INSR or EC |
| | | | | 2.7.10.1) Agonist |
| HS-20004 | | Jiangsu Hansoh | Peptide | Glucagon Like Peptide |
| | | Pharmaceutical | | 1 Receptor (GLP1R) |
| | | Group Co Ltd | | Agonist |
| LY-3493269 | | Eli Lilly and Co | Synthetic | Gastric Inhibitory |
| | | | Protein | Polypeptide Receptor |
| | | | | (Glucose Dependent |
| | | | | Insulinotropic |
| | | | | Polypeptide Receptor |
| | | | | or GIPR) Agonist; |
| | | | | Glucagon Like Peptide |
| | | | | 1 Receptor (GLP1R) |
| | | | | Agonist |
| mazdutide | 2259884- | Eli Lilly and Co | Synthetic | Glucagon Like Peptide |
| | 03-0 | | Peptide | 1 Receptor (GLP1R) |
| | | | | Agonist; Glucagon |
| | | | | Receptor (GL R or |
| | | | | GCGR) Agonist |
| NLY-001 | | Neuraly Inc | Synthetic | Glucagon Like Peptide |
| | | | Peptide | 1 Receptor (GLP1R) |
| | | | | Agonist |
| NN-6177 | | Novo Nordisk AS | Synthetic | Glucagon Like Peptide |
| | | | Peptide | 1 Receptor (GLP1R) |
| | | | | Agonist; Glucagon |
| | | | | Receptor (GL R or |
| | | | | GCGR) Agonist |
| PB-119 | | PegBio Co Ltd | Synthetic | Glucagon Like Peptide |
| | | | Peptide | 1 Receptor (GLP1R) |
| | | | | Agonist |
| PF-1801 | 1417579- | ImmunoForge Co Ltd | Fusion Protein | Glucagon Like Peptide |
| | 81-7 | | | 1 Receptor (GLP1R) |
| | | | | Agonist |
| retatrutide | 2381089- | Eli Lilly and Co | Biologic | Gastric Inhibitory |
| | 83-2 | | | Polypeptide Receptor |
| | | | | (Glucose Dependent |
| | | | | Insulinotropic |
| | | | | Polypeptide Receptor |
| | | | | or GIPR) Agonist; |
| | | | | Glucagon Like Peptide |
| | | | | 1 Receptor (GLP1R) |
| | | | | Agonist; Glucagon |
| | | | | Receptor (GL R or |
| | | | | GCGR) Agonist |
| SCO-094 | | Scohia Pharma Inc | Synthetic | Glucagon Like Peptide |
| | | | Peptide | 1 Receptor (GLP1R) |
| | | | | Agonist; Glucose |
| | | | | Dependent |
| | | | | Insulinotropic |
| | | | | Receptor (G Protein |
| | | | | Coupled Receptor 119 |
| | | | | or GPR119) Agonist |
| semaglutide + GIP | 910463- | Novo Nordisk AS | Synthetic | Gastric Inhibitory |
| analogue | 68-2 | | Peptide | Polypeptide Receptor |
| | | | | (Glucose Dependent |
| | | | | Insulinotropic |
| | | | | Polypeptide Receptor |
| | | | | or GIPR) Agonist; |
| | | | | Glucagon Like Peptide |
| | | | | 1 Receptor (GLP1R) |
| | | | | Agonist |
| VK-2735 | | Viking Therapeutics | | Gastric Inhibitory |
| | | Inc | | Polypeptide Receptor |
| | | | | (Glucose Dependent |
| | | | | Insulinotropic |
| | | | | Polypeptide Receptor |
| | | | | or GIPR) Agonist; |
| | | | | Glucagon Like Peptide |
| | | | | 1 Receptor (GLP1R) |
| | | | | Agonist |
| YH-25724 | | Boehringer Ingelheim | Fusion Protein | Fibroblast Growth |
| | | International GmbH | | Factor Receptor 1 |
| | | | | (Basic Fibroblast |
| | | | | Growth Factor |
| | | | | Receptor 1 or Fms |
| | | | | Like Tyrosine Kinase 2 |
| | | | | or N Sam or Proto |
| | | | | Oncogene c Fgr or |
| | | | | CD331 or FLT2 or |
| | | | | FGFR1 or EC |
| | | | | 2.7.10.1) Agonist; |
| | | | | Glucagon Like Peptide |
| | | | | 1 Receptor (GLP1R) |
| | | | | Agonist |
| YN-012 | | Shanghai Innogen | Recombinant | Glucagon Like Peptide |
| | | Pharmaceutical | Protein | 1 Receptor (GLP1R) |
| | | Technology Co Ltd | | Agonist |
| YN-015 | | Shanghai Innogen | Recombinant | Glucagon Like Peptide |
| | | Pharmaceutical | Protein | 1 Receptor (GLP1R) |
| | | Technology Co Ltd | | Agonist |
| AMG-133 | | Amgen Inc | Monoclonal | Gastric Inhibitory |
| | | | Antibody | Polypeptide Receptor |
| | | | Conjugated | (Glucose Dependent |
| | | | | Insulinotropic |
| | | | | Polypeptide Receptor |
| | | | | or GIPR) Antagonist; |
| | | | | Glucagon Like Peptide |
| | | | | 1 Receptor (GLP1R) |
| | | | | Agonist |
| avexitide acetate | 2051593- | Eiger | Synthetic | Glucagon Like Peptide |
| | 46-3 | BioPharmaceuticals | Peptide | 1 Receptor (GLP1R) |
| | | Inc | | Antagonist |
| E-2HSA | | Zhejiang Huayang | Fusion Protein | Glucagon Like Peptide |
| | | Pharmaceutical Co | | 1 Receptor (GLP1R) |
| | | Ltd | | Agonist |
| exendin-9-39 | 133514- | Children's Hospital of | Synthetic | Glucagon Like Peptide |
| | 43-9 | Philadelphia | Peptide | 1 Receptor (GLP1R) |
| | | | | Antagonist |
| GMA-102 | | Gmax Biopharm LLC | Monoclonal | Glucagon Like Peptide |
| | | | Antibody | 1 Receptor (GLP1R) |
| | | | | Agonist |
| GZR-18 | | Gan & Lee | Peptide | Glucagon Like Peptide |
| | | Pharmaceuticals Co | | 1 Receptor (GLP1R) |
| | | Ltd | | Agonist |
| HM-15136 | | Hanmi | Recombinant | Glucagon Receptor |
| | | Pharmaceuticals Co | Peptide | (GL R or GCGR) |
| | | Ltd | | Agonist |
| IONIS-GCGRRx | | Ionis | Antisense | Glucagon Receptor |
| | | Pharmaceuticals Inc | Oligonucleotide | (GL R or GCGR) |
| | | | | Antagonist |
| JY-09 | | Beijing Eastern | Fusion Protein | Glucagon Like Peptide |
| | | Biotech Co Ltd | | 1 Receptor (GLP1R) |
| | | | | Agonist |
| pegapamodutide | 1492924- | OPKO Health Inc | Synthetic | Glucagon Like Peptide |
| | 65-8 | | Peptide | 1 Receptor (GLP1R) |
| | | | | Agonist; Glucagon |
| | | | | Receptor (GL R or |
| | | | | GCGR) Agonist |
| semaglutide + GIP | 910463- | Novo Nordisk AS | Synthetic | Gastric Inhibitory |
| analogue | 68-2 | | Peptide | Polypeptide Receptor |
| | | | | (Glucose Dependent |
| | | | | Insulinotropic |
| | | | | Polypeptide Receptor |
| | | | | or GIPR) Agonist; |
| | | | | Glucagon Like Peptide |
| | | | | 1 Receptor (GLP1R) |
| | | | | Agonist |
| volagidemab | 1233956- | Remd | Monoclonal | Glucagon Receptor |
| | 13-2 | Biotherapeutics Inc | Antibody | (GL R or GCGR) |
| | | | | Antagonist |
| albenatide | 1031700- | ConjuChem LLC | Synthetic | Glucagon Like Peptide |
| | 39-6 | | Peptide | 1 Receptor (GLP1R) |
| | | | | Agonist |
| cagrilintide + | 910463- | Novo Nordisk AS | Synthetic | Calcitonin Receptor |
| semaglutide | 68-2 & | | Peptide | (CALCR) Agonist; |
| | 1415456- | | | Glucagon Like Peptide |
| | 99-3 | | | 1 Receptor (GLP1R) |
| | | | | Agonist; Receptor |
| | | | | Activity Modifying |
| | | | | Protein 1 (Calcitonin |
| | | | | Receptor Like |
| | | | | Receptor Activity |
| | | | | Modifying Protein 1 or |
| | | | | RAMP1) Activator; |
| | | | | Receptor Activity |
| | | | | Modifying Protein 2 |
| | | | | (Calcitonin Receptor |
| | | | | Like Receptor Activity |
| | | | | Modifying Protein 2 or |
| | | | | CRLR Activity |
| | | | | Modifying Protein 2 or |
| | | | | RAMP2) Activator; |
| | | | | Receptor Activity |
| | | | | Modifying Protein 3 |
| | | | | (Calcitonin Receptor |
| | | | | Like Receptor Activity |
| | | | | Modifying Protein 3 or |
| | | | | RAMP3) Activator |
| Diabegone | | Shanghai Innogen | Recombinant | Glucagon Like Peptide |
| | | Pharmaceutical | Protein | 1 Receptor (GLP1R) |
| | | Technology Co Ltd | | Agonist |
| efpeglenatide LA | | Hanmi | Recombinant | Glucagon Like Peptide |
| | | Pharmaceuticals Co | Peptide | 1 Receptor (GLP1R) |
| | | Ltd | | Agonist |
| LAI-287 + | 910463- | Novo Nordisk AS | Recombinant | Glucagon Like Peptide |
| semaglutide | 68-2 | | Protein; | 1 Receptor (GLP1R) |
| | | | Synthetic | Agonist; Insulin |
| | | | Peptide | Receptor (IR or |
| | | | | CD220 or INSR or EC |
| | | | | 2.7.10.1) Agonist |
| SAL-015 | | Shenzhen Salubris | Fusion Protein | Glucagon Like Peptide |
| | | Pharmaceuticals Co | | 1 Receptor (GLP1R) |
| | | Ltd | | Agonist |
| ABG-023 | | AmideBio, LLC | Synthetic | Glucagon Receptor |
| | | | Peptide | (GL R or GCGR) |
| | | | | Agonist |
| ACT-1003 | | Accurus Biosciences | Peptide | Glucagon Like Peptide |
| | | Inc | | 1 Receptor (GLP1R) |
| | | | | Agonist |
| AGM-212 | | AnyGen Co Ltd | Synthetic | Glucagon Like Peptide |
| | | | Peptide | 1 Receptor (GLP1R) |
| | | | | Agonist |
| BZ-043B | | Biozeus | Synthetic | Calcitonin Receptor |
| | | Pharmaceutical SA | Peptide | (CALCR) Agonist; |
| | | | | Glucagon Like Peptide |
| | | | | 1 Receptor (GLP1R) |
| | | | | Agonist; Receptor |
| | | | | Activity Modifying |
| | | | | Protein 1 (Calcitonin |
| | | | | Receptor Like |
| | | | | Receptor Activity |
| | | | | Modifying Protein 1 or |
| | | | | RAMP1) Activator; |
| | | | | Receptor Activity |
| | | | | Modifying Protein 2 |
| | | | | (Calcitonin Receptor |
| | | | | Like Receptor Activity |
| | | | | Modifying Protein 2 or |
| | | | | CRLR Activity |
| | | | | Modifying Protein 2 or |
| | | | | RAMP2) Activator; |
| | | | | Receptor Activity |
| | | | | Modifying Protein 3 |
| | | | | (Calcitonin Receptor |
| | | | | Like Receptor Activity |
| | | | | Modifying Protein 3 or |
| | | | | RAMP3) Activator |
| C-2816 | | AstraZeneca Plc | Recombinant | Cholecystokinin |
| | | | Peptide | Receptor Type A |
| | | | | (Cholecystokinin 1 |
| | | | | Receptor or CCKAR) |
| | | | | Agonist; Glucagon |
| | | | | Like Peptide 1 |
| | | | | Receptor (GLP1R) |
| | | | | Agonist |
| Cbi-Ex4 | | Syracuse University | Synthetic | Glucagon Like Peptide |
| | | | Peptide | 1 Receptor (GLP1R) |
| | | | | Agonist |
| DA-1726 | | NeuroBo | Peptide | Glucagon Like Peptide |
| | | Pharmaceuticals Inc | | 1 Receptor (GLP1R) |
| | | | | Agonist; Glucagon |
| | | | | Receptor (GL R or |
| | | | | GCGR) Agonist |
| DAJC-1 | | Lancaster University | Synthetic | Gastric Inhibitory |
| | | | Peptide | Polypeptide Receptor |
| | | | | (Glucose Dependent |
| | | | | Insulinotropic |
| | | | | Polypeptide Receptor |
| | | | | or GIPR) Agonist; |
| | | | | Glucagon Like Peptide |
| | | | | 1 Receptor (GLP1R) |
| | | | | Agonist |
| DR-10625 | | Zhejiang Doer | Fusion Protein | Beta Klotho (Klotho |
| | | Biologics Corp | | Beta Like Protein or |
| | | | | KLB) Activator; |
| | | | | Fibroblast Growth |
| | | | | Factor Receptor 1 |
| | | | | (Basic Fibroblast |
| | | | | Growth Factor |
| | | | | Receptor 1 or Fms |
| | | | | Like Tyrosine Kinase 2 |
| | | | | or N Sam or Proto |
| | | | | Oncogene c Fgr or |
| | | | | CD331 or FLT2 or |
| | | | | FGFR1 or EC |
| | | | | 2.7.10.1) Agonist; |
| | | | | Gastric Inhibitory |
| | | | | Polypeptide Receptor |
| | | | | (Glucose Dependent |
| | | | | Insulinotropic |
| | | | | Polypeptide Receptor |
| | | | | or GIPR) Agonist; |
| | | | | Glucagon Like Peptide |
| | | | | 1 Receptor (GLP1R) |
| | | | | Agonist |
| DR-10628 | | Zhejiang Doer | Fusion Protein | Gastric Inhibitory |
| | | Biologics Corp | | Polypeptide Receptor |
| | | | | (Glucose Dependent |
| | | | | Insulinotropic |
| | | | | Polypeptide Receptor |
| | | | | or GIPR) Agonist; |
| | | | | Glucagon Like Peptide |
| | | | | 1 Receptor (GLP1R) |
| | | | | Agonist |
| E-6 | | California Institute for | Synthetic | Glucagon Like Peptide |
| | | Biomedical Research | Peptide | 1 Receptor (GLP1R) |
| | | | | Agonist |
| efpeglenatide + | 1296200- | Hanmi | Recombinant | Glucagon Like Peptide |
| HM-12470 | 77-5 | Pharmaceuticals Co | Peptide; | 1 Receptor (GLP1R) |
| | | Ltd | Synthetic | Agonist; Insulin |
| | | | Peptide | Receptor (IR or |
| | | | | CD220 or INSR or EC |
| | | | | 2.7.10.1) Agonist |
| efpeglenatide + | 1296200- | Hanmi | Synthetic | Glucagon Receptor |
| HM-15136 | 77-5 | Pharmaceuticals Co | Peptide | (GL R or GCGR) |
| | | Ltd | | Agonist |
| exenatide + | 141758- | Adocia SAS | Recombinant | Glucagon Like Peptide |
| glucagon + | 74-9 & | | Peptide; | 1 Receptor (GLP1R) |
| pramlintide | 16941- | | Synthetic | Agonist; Glucagon |
| | 32-5 & | | Peptide | Receptor (GL R or |
| | 151126- | | | GCGR) Agonist; Islet |
| | 32-8 | | | Amyloid Polypeptide |
| | | | | (Amylin or Diabetes |
| | | | | Associated Peptide or |
| | | | | Insulinoma Amyloid |
| | | | | Peptide or IAPP) |
| | | | | Activator |
| exenatide + | 141758- | Adocia SAS | Recombinant | Glucagon Like Peptide |
| pramlintide | 74-9 & | | Peptide; | 1 Receptor (GLP1R) |
| | 151126- | | Synthetic | Agonist |
| | 32-8 | | Peptide | |
| Extendin-Fc | | Uni-Bio Science | Recombinant | Glucagon Like Peptide |
| | | Group Ltd | Peptide | 1 Receptor (GLP1R) |
| | | | | Agonist |
| G-49 | | AstraZeneca Plc | | Glucagon Like Peptide |
| | | | | 1 Receptor (GLP1R) |
| | | | | Agonist; Glucagon |
| | | | | Receptor (GL R or |
| | | | | GCGR) Agonist |
| KP-405 | | Kariya | | Gastric Inhibitory |
| | | Pharmaceuticals IVS | | Polypeptide Receptor |
| | | | | (Glucose Dependent |
| | | | | Insulinotropic |
| | | | | Polypeptide Receptor |
| | | | | or GIPR) Agonist; |
| | | | | Glucagon Like Peptide |
| | | | | 1 (GLP1) Activator |
| LA-EX | | Shandong Luye | Synthetic | Glucagon Like Peptide |
| | | Pharmaceutical Co | Peptide | 1 Receptor (GLP1R) |
| | | Ltd | | Agonist |
| MK-1462 | | Merck & Co Inc | Synthetic | Glucagon Like Peptide |
| | | | Peptide | 1 Receptor (GLP1R) |
| | | | | Agonist; Glucagon |
| | | | | Receptor (GL R or |
| | | | | GCGR) Agonist |
| OGB-21502 | | Onegene | Fusion Protein | Beta Klotho (Klotho |
| | | Biotechnology Inc | | Beta Like Protein or |
| | | | | KLB) Activator; |
| | | | | Fibroblast Growth |
| | | | | Factor Receptor 1 |
| | | | | (Basic Fibroblast |
| | | | | Growth Factor |
| | | | | Receptor 1 or Fms |
| | | | | Like Tyrosine Kinase 2 |
| | | | | or N Sam or Proto |
| | | | | Oncogene c Fgr or |
| | | | | CD331 or FLT2 or |
| | | | | FGFR1 or EC |
| | | | | 2.7.10.1) Agonist; |
| | | | | Glucagon Like Peptide |
| | | | | 1 Receptor (GLP1R) |
| | | | | Agonist; Glucagon |
| | | | | Receptor (GL R or |
| | | | | GCGR) Agonist; |
| | | | | Interleukin 1 Receptor |
| | | | | Type 1 (CD121 |
| | | | | Antigen Like Family |
| | | | | Member A or |
| | | | | Interleukin 1 Receptor |
| | | | | Alpha or p80 or |
| | | | | CD121a or IL1R1) |
| | | | | Agonist |
| P-11 | | Protheragen Inc | Fusion Protein | Glucagon Like Peptide |
| | | | | 1 Receptor (GLP1R) |
| | | | | Agonist |
| PGLP-1 | | China | Synthetic | Glucagon Like Peptide |
| | | Pharmaceutical | Peptide | 1 Receptor (GLP1R) |
| | | University | | Agonist |
| REMD-2.59 | | Remd | Monoclonal | Glucagon Receptor |
| | | Biotherapeutics Inc | Antibody | (GL R or GCGR) |
| | | | | Antagonist |
| REMD-524 | | Remd | Monoclonal | Glucagon Receptor |
| | | Biotherapeutics Inc | Antibody | (GL R or GCGR) |
| | | | | Antagonist |
| SFR-9458 | | FortuneRock (China) | Fusion Protein | Glucagon Like Peptide |
| | | Ltd | | 1 Receptor (GLP1R) |
| | | | | Agonist |
| SL-209 | | Beijing SL | Recombinant | Glucagon Like Peptide |
| | | Pharmaceutical Co | Peptide | 1 Receptor (GLP1R) |
| | | Ltd | | Agonist |
| TB-013 | | Twist Bioscience | Monoclonal | Glucagon Like Peptide |
| | | Corp | Antibody | 1 Receptor (GLP1R) |
| | | | | Antagonist |
| TB-592 | | Twist Bioscience | Fusion Protein | Glucagon Like Peptide |
| | | Corp | | 1 Receptor (GLP1R) |
| | | | | Agonist |
| TE-8105 | | Immunwork Inc | Peptide | Glucagon Like Peptide |
| | | | | 1 Receptor (GLP1R) |
| | | | | Agonist |
| THDBH-110 | | Tonghua Dongbao | | Glucagon Like Peptide |
| | | Pharmaceutical Co | | 1 Receptor (GLP1R) |
| | | Ltd | | Agonist |
| THDBH-111 | | Tonghua Dongbao | | Glucagon Like Peptide |
| | | Pharmaceutical Co | | 1 Receptor (GLP1R) |
| | | Ltd | | Agonist |
| XL-110 | | XL-protein GmbH | Fusion Protein | Glucagon Like Peptide |
| | | | | 1 Receptor (GLP1R) |
| | | | | Agonist |
| XL-310 | | XL-protein GmbH | Fusion Protein | Glucagon Like Peptide |
| | | | | 1 Receptor (GLP1R) |
| | | | | Agonist; Neuropeptide |
| | | | | Y Receptor Type 2 |
| | | | | (NPY Y2 Receptor or |
| | | | | NPY2R) Agonist |
| XW-003 + XW-017 | | Sciwind Biosciences | Peptide | Gastric Inhibitory |
| | | Co Ltd | | Polypeptide Receptor |
| | | | | (Glucose Dependent |
| | | | | Insulinotropic |
| | | | | Polypeptide Receptor |
| | | | | or GIPR) Antagonist; |
| | | | | Glucagon Like Peptide |
| | | | | 1 Receptor (GLP1R) |
| | | | | Agonist |
| ACP-003 | | Sanofi | Synthetic | Glucagon Like Peptide |
| | | | Peptide | 1 Receptor (GLP1R) |
| | | | | Agonist |
| exenatide + | 141758- | Adocia SAS | Recombinant | Glucagon Like Peptide |
| glucagon | 74-9 & | | Peptide; | 1 Receptor (GLP1R) |
| | 16941- | | Synthetic | Agonist; Glucagon |
| | 32-5 | | Peptide | Receptor (GL R or |
| | | | | GCGR) Agonist |
| albiglutide | 782500- | GlaxoSmithKline Inc | Recombinant | Glucagon Like Peptide |
| | 75-8 | | Protein | 1 Receptor (GLP1R) |
| | | | | Agonist |

As yet a further example, in some embodiments the surface of an ingestible device is very smooth. However, in certain embodiments, the outer surface of an ingestible device has a non-zero degree of roughness. In such embodiments, having a non-zero degree of roughness for the outer surface of an ingestible device may result in a relatively desirable navigation of the ingestible device navigate through the GI tract of a subject. As an example, an ingestible device having an outer surface with a non-zero rugosity may pass through one or more regions of the GI tract in a relatively slow manner. In some embodiments, an ingestible device can have an outer surface with a non-zero rugosity can include, for example, one or more regions that are grooved. An outer surface with a non-zero rugosity can, for example, allow for more time and opportunity to deliver one or more dispensable substances when the ingestible device is disposed within an appropriate region of the GI tract. Such a device can be used for delivery as desired, including, for example, trans-epithelial delivery, epithelial delivery or topical delivery. Generally, the parameters for such delivery are similar to those described elsewhere herein.

As still a further example, while various embodiments of ingestible devices having one or more nozzles have been described in which the exit(s) of the nozzle(s) is flush with an exterior surface of the ingestible devices, the disclosure is not limited to such embodiments. For example, an ingestible device having one or more nozzles may be configured so that the nozzle exit(s) extend outwardly from one or more regions of the outer surface of the ingestible device. In some embodiments in which an ingestible device includes one or more nozzles that extend outwardly from one or more regions of the outer surface of the device, the nozzle(s) are disposed on a longer axis of the device. In another embodiments in which an ingestible device includes one or more nozzles that extend outwardly from one or more regions of the outer surface of the device, the nozzle(s) are disposed on the radial axis of the device. Such an arrangement can allow for enhanced alignment with the surface of the GI tract, e.g., the one or more nozzles are in closer proximity to the mucosal tissue of the GI tract. More generally, any ingestible device described herein having one or more nozzles can be configured such one or more of the nozzles extend outwardly from one or more regions of the outer surface of the ingestible device.

As an additional example, while certain capsule shapes have been disclosed, the disclosure is not limited to such shapes. For example, in some embodiments, the diameter of the capsule adjacent one end that is substantially different (e.g., substantially smaller) from the diameter of the capsule adjacent to the opposite end of the capsule. An example of such a capsule is a droplet-shaped capsule. In certain embodiments, a capsule have a shape described in the present paragraph may allow for the capsule to get relatively close to the mucus (and, as a result, relatively close to the epithelial layer) of the GI tract.

As another example, in some embodiments, an ingestible device is less dense than the fluid present in one or more (e.g., all) regions of the GI tract that are of interest in using the ingestible device to delivery one or more dispensable substances. Such an ingestible device can pass through one or more regions of the GI tract in a relatively predictable fashion, which can enhance the ability to deliver the one or more dispensable substances to one or more desired locations in a relatively controlled and/or predictable fashion. In some situations, an ingestible device is less dense than the fluid present in one or more (e.g., all) regions of the GI tract that are of interest in using the ingestible device to delivery one or more dispensable substances can be referred to as not being buoyant.

As another example, while certain examples of embodiments of a seal (e.g., a foil seal) for a nozzle exit have been described, the disclosure is not limited to such seals. More generally, a seal for a nozzle exit can have any shape and be formed of any material such that it resists breakage until breakage is desired. In addition, a seal for a nozzle exit can be in the interior of the device (e.g., on the surface of the drug reservoir) whereby the entrance to the nozzle is sealed. In some embodiments, a seal for a nozzle exit can be formed of ethyl cellulose (e.g., EthoCel) or polyvinyl acetate (e.g., Kollicoat® a BASF coating polymer). In certain embodiments, a seal can in the form of a film, such as, for example, a film having a thickness of from about 10 μm to about 50 μm (e.g., from about 20 μm to about 40 μm, such as about 30 μm). In some embodiments, a seal for a nozzle exit can be formed of a coating that covers some or all of the exterior surface of the capsule material, including the nozzle exit(s) to be sealed. Such coatings can be a monolayer coating or a multilayer coating. Materials that can be used in a monolayer coating include cyclic olefin copolymer (COC), polytetrafluoroethylene (PTFE), thermopolymers, and cellulose acetate. The thickness of such a monolayer can be, for example, from about 25 μm to about 200 μm (e.g., about 35 μm, about 75 μm, about 140 μm, about 200 μm). In some embodiments, the coating is a 140 μm COC monolayer. COC is commercially available from, for example, TekniPlex. In certain embodiments, the coating is a 75 μm PTFE monolayer. In some embodiments, the coating is a 200 μm PTFE monolayer. In certain embodiments, the coating is a 35 μm cellulose acetate monolayer. In some embodiments, the coating is a 75 μm cellulose acetate monolayer. Cellulose acetate is commercially available from, for example, Agar Scientific. A multilayer coating can be formed of, for example, a layer of COC and a polymer layer (e.g., polychlorotrifluoroethylene (PCTFE)) with a tie layer therebetween. Such a multilayer coating can have a thickness of from about 25 μm to about 75 μm (e.g., about 50 μm). For example, a multilayer coating be formed of a layer of COC (e.g., 20 μm), a tie layer (e.g., 16 μm) and a layer of polychlorotrifluoroethylene (e.g., 15 μm). An example of such a commercially available multilayer material is Tekniflex CTA160 (TekniPlex). As noted above, in some embodiments, the seal for a nozzle can be a polyolefin material, for example, having a thickness of from about 40 μm to about 60 μm (e.g., about 50 μm). An example, a commercially available polyolefin Trans-Pharma TRA-150 (Tanscendia). In certain embodiments, a seal can be composed of LDPE, for example, having a thickness of from about 10 μm to about 100 μm (e.g., from about 20 μm to about 80 μm, about 25 μm, about 50 μm, about 100 μm). An example of a commercial supplier of such LDPE is Goodfellows. In some embodiments, the seal for a nozzle can be composed of polyethylene terephthalate (PET), for example having a thickness of from about 5 μm to about 15 μm (e.g., about 13 μm), such as can be acquired from Nordsen Medical, Salem, N.H., USA.

In certain embodiments, the seal for a nozzle can be composed of PTFE, for example having a thickness of from about 50 μm to about 250 μm (e.g., about 75 μm, about 75 μm), such as can be acquired from RS Components. In certain embodiments, the seal for a nozzle can be composed of fluorinated ethylene propylene (FEP) or nylon (e.g., nylon 12). In some embodiments, the seal for a nozzle can be formed of a metal (e.g., a metal foil). An example of such a metal is aluminum, such as household aluminum foil. In some embodiments, a film containing a metal may have a multilayer construction.

In some embodiments, one or more components of an ingestible device can include one or more starch based polymers, such as one or more thermoplastic starch polymers. In some embodiments, the starch based polymer may be starch as such or a polymer having a high starch content selected from more than 70% starch, more than 80% starch, or more than 90% starch. Examples of molecules in starch include amylose and amylopectin. In some embodiments, a starch-based polymer can be general fully biodegradable. In some embodiments, a starch based polymer may be maize starch, such as, for example, Cornpack. A starch based polymers may be decomposable. In some cases, a starch based polymer can be relatively stable and relatively inert in solid dosage forms.

In some embodiments, one or more of the components of an ingestible device can include one or more cellulose based polymers.

In some embodiments, one or more components of an ingestible device include one or more biodegradable polymers.

As a further example, in some embodiments, the formulation is deposited in the submucosa and/or the mucosa (e.g., into the lamina propria) of the small intestine of the subject. In some embodiments, the formulation is deposited in the submucosa and/or the mucosa (e.g., into the lamina propria) of the duodenum of the subject. In some embodiments, the formulation is deposited in the submucosa and/or the mucosa (e.g., into the lamina propria) of the jejunum or the ileum of the subject.

As yet another example, a first portion of the pharmaceutical formulation released from the device is deposited in the submucosa and a second portion is deposited in the mucosa (such as the lamina propria), and/or is released into the lumen, and may subsequently adhere to the mucus of the gastrointestinal tract. In some embodiments, the first portion of the pharmaceutical formulation deposited into the submucosa contains at least about 99% of the total pharmaceutical formulation released from the device, wherein the % is a w/w %, a w/v %, or a v/v % of the pharmaceutical formulation. In other embodiments, the first portion of the pharmaceutical formulation deposited into the submucosa contains at least about 95%, about 90%, about 85%, about 80%, about 75%, about 70%, about 75%, about 70%, about 65%, about 60%, about 55% or about 50% of the pharmaceutical formulation, wherein the % is a w/w %, a w/v %, or a v/v % of the pharmaceutical formulation. In yet other embodiments, the first portion of the pharmaceutical formulation deposited into the submucosa contains at least about 45%, at least about 40%, at least about 35%, at least about 30%, at least about 25%, at least about 20%, at least about 15%, at least about 10%, or at least about 5% of the pharmaceutical formulation, wherein the % is a w/w %, a w/v %, or a v/v % of the pharmaceutical formulation.

As an additional example, the formulation is topically delivered to the small intestine, the duodenum or the jejunum of the subject. In some embodiments, the formulation is topically delivered to the ileum of the subject. The topical delivery of the formulation to the small intestine may be for use in treating ileal Crohn's disease.

As another example, in some embodiments, the formulation is topically delivered to the large intestine, the cecum, the colon or to the rectum of the subject. Topical delivery of the formulation to the large intestine may be used for treating an inflammatory bowel disease (IBD), where the IBD is Crohn's disease or ulcerative colitis.

Releasing of the therapeutic may be triggered by one or more of: a pH in the jejunum of about 6.1 to about 7.2, a pH in the mid small bowel of about 7.0 to about 7.8, a pH in the ileum of about 7.0 to about 8.0, a pH in the right colon of about 5.7 to about 7.0, a pH in the mid colon of about 5.7 to about 7.4, or a pH in the left colon of about 6.3 to about 7.7, such as about 7.0.

Releasing of the therapeutic may be triggered by degradation of a release component located in the device, and may be dependent on enzymatic activity at or in the vicinity of the location. In some embodiments of any of the devices or methods described herein:
the composition includes a plurality of electrodes including a coating, and releasing the therapeutic is triggered by an electric signal by the electrodes resulting from the interaction of the coating with an intended site of release of the therapeutic; the release of the therapeutic is triggered by a remote electromagnetic signal; the release of the therapeutic is triggered by generation in the composition of a gas in an amount sufficient to expel the therapeutic; and/or the release of the therapeutic is triggered by an electromagnetic signal generated within the device according to a pre-determined drug release profile.

As a further example, an ingestible device includes one or more safety mechanisms, e.g., to reduce/eliminate the possibility of an undesirably high pressure building within the ingestible device. Such a safety mechanism can be configured, for example, as a disc that opens (e.g., bursts) when the pressure within the ingestible device reaches or exceeds a certain value. Optionally, a safety mechanism can be configured as a valve that opens when the pressure within the ingestible device reaches or exceeds a certain value. In some embodiments, a safety mechanism can be configured as one or more recess channels, e.g., in the interior wall of the device.

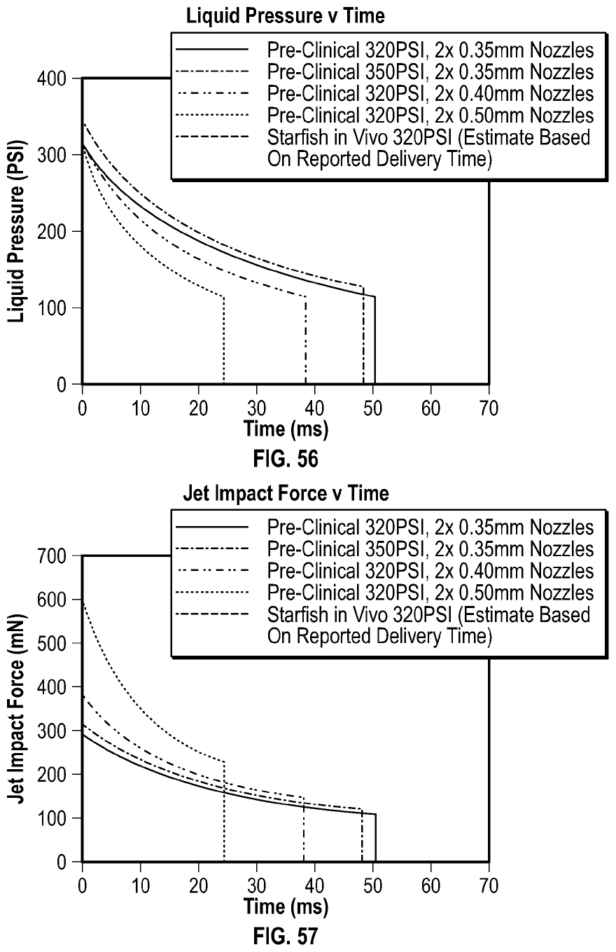

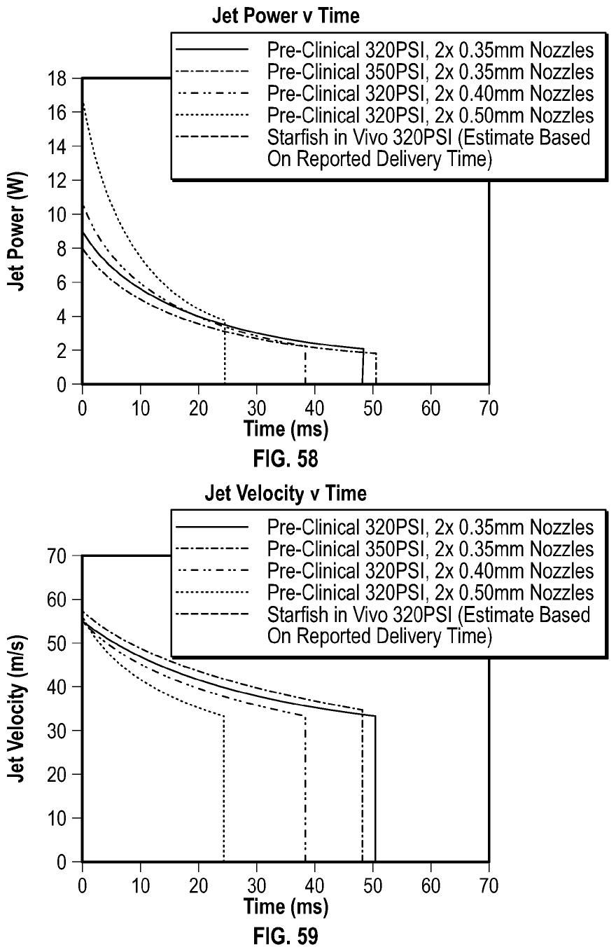

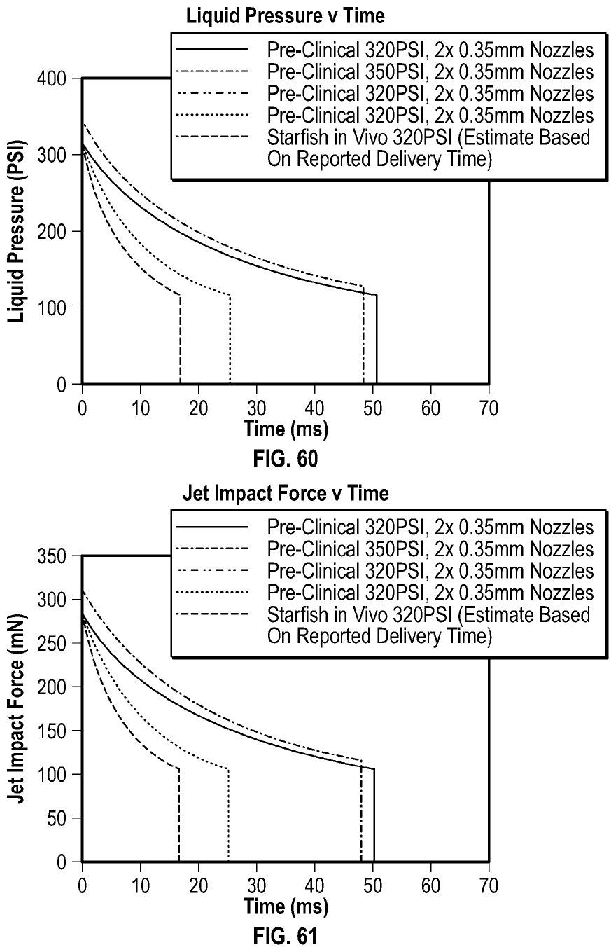

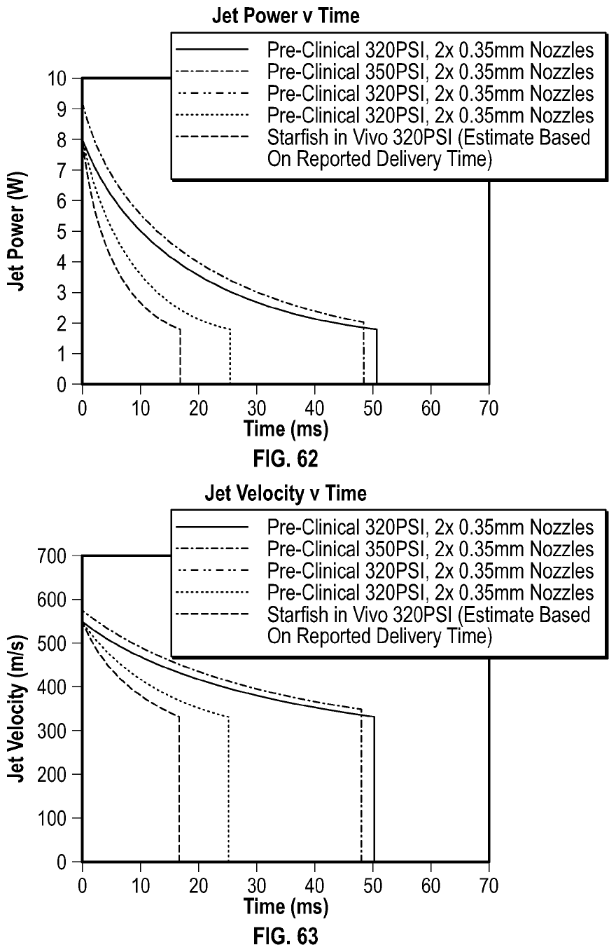

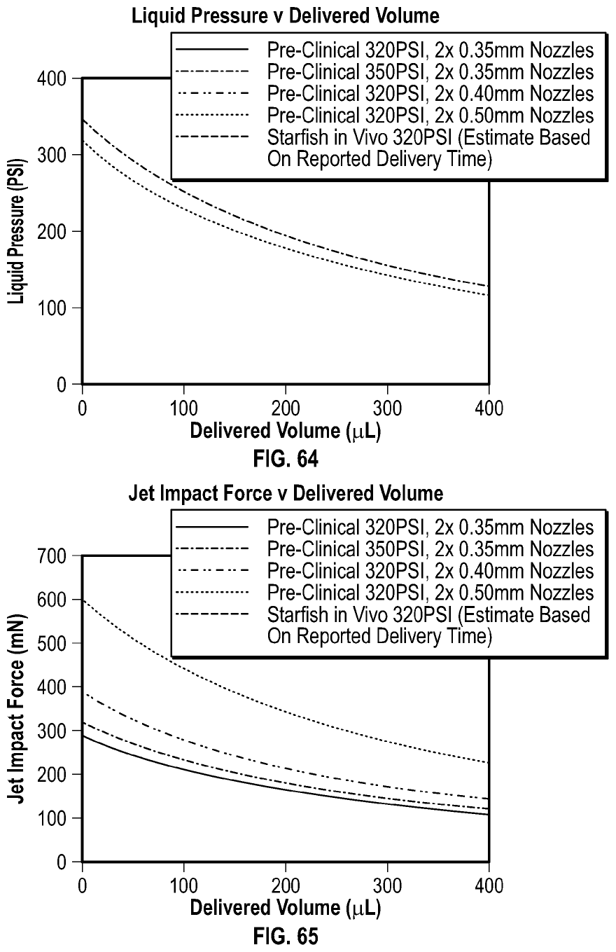

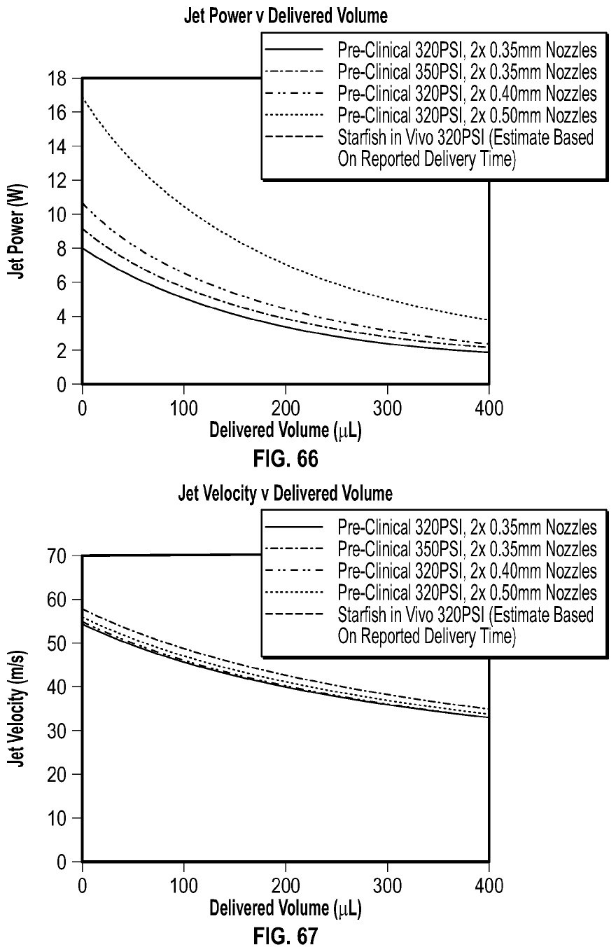

With reference to FIGS. 56-72B and the below tables, a number of additional ingestible device examples are described in more detail.

### Example 1—Modelling Device Performance

In this Example, modelling was used to determine the performance parameters of an ingestible device for delivering a dispensable substance.

### Model

The driving pressure, for a given point in the dose delivery, is related to the delivered liquid volume, and the resulting increase in gas volume, by equations of state for adiabatic expansion. The velocity (e.g., peak jet velocity, average jet velocity, or minimum jet velocity) through the orifice (or nozzle) is in turn given by the driving pressure. This is a steady state approximation in which transient effects of fluid acceleration/deceleration are ignored. In other words, the gas expansion is rapid allowing little time for heat transfer/thermal equilibration to the surroundings. Thus, this is treated as an adiabatic (no energy loss).

For an adiabatic process, Pressure P and Volume V of a gas are related as follows, assuming that fluid (liquid) pressure is equal to gas pressure (frictionless piston).

$$\frac{{P}_{2}}{{P}_{1}} = {( \frac{{V}_{1}}{{V}_{2}} )}^{γ} ,$$

where P is pressure. V is volume, and y is the ratio of specific heats.

Pipe shear pressure is given by the Darcy-Weisbach equation:

$${P}_{pipe} = f \frac{ρ {Lv}_{o}^{2}}{2 {d}_{o}} ,$$

where ρ is the density of a liquid, L is the nozzle length, uₒ is the velocity through the nozzle orifice, dₒ is the diameter of an nozzle orifice, and f is the Darcy friction factor for pipe flow.

The friction factor for rough pipes is given by:

$$\frac{1}{{f}^{1 / 2}} = - 2. log ( \frac{ϵ / {d}_{o}}{3.7} + \frac{2.51}{{Ref}^{1 / 2}} )$$

where ϵ is the pipe surface roughness, and Re is the Reynolds number for the fluid.

Haaland proposed the following explicit approximation, which differs by less than 2% from Colebrook:

$${f}^{- 1 / 2} = \frac{1}{{f}^{1 / 2}} = - 1.8 log ( {( \frac{ϵ / {d}_{o}}{3.7} )}^{1.11} + \frac{6.9}{Re} )$$

$$f = \frac{1}{{( - 1.8 log ( {( \frac{ϵ / {d}_{o}}{3.7} )}^{1.11} + \frac{6.9}{Re} ) )}^{2}}$$

Therefore, pipe pressure is:

$${P}_{pipe} = \frac{ρ {Lv}_{o}^{2}}{2 {{d}_{o} ( - 1.8 log ( {( \frac{ϵ / {d}_{o}}{3.7} )}^{1.11} + \frac{6.9}{Re} ) )}^{2}}$$

This explicit approximation requires iterative solution if Re is unknown.

Pipe exit and entry losses are assumed as being given by:

$${P}_{Entry} = \frac{1}{2} {C}_{Entry} {ρ}_{l} {v}^{2}$$

$${P}_{Exit} = \frac{1}{2} {C}_{Exit} {ρ}_{l} {v}^{2}$$

Thus, overall pressure drop across the orifice is:

$${P}_{Orifice} = f \frac{ρ {Lv}_{o}^{2}}{2 {d}_{o}} + \frac{1}{2} ( {C}_{Entry} + {C}_{Exit} ) ρ {Lv}_{o}^{2} ,$$

Where C_{entry} is the coefficient of discharge on entry, and Cₑₓᵢₜ is the coefficient of discharge on exit.

The total flowrate is:

Q=Nπdₒ²vₒ,

where Q is the volumetric flow rate through a single orifice.

Accounting for piston friction, the liquid delivery pressure through the orifice is related to the gas pressure as follows. The force balance on the piston is given by:

$$\frac{π}{4} {d}_{Piston}^{2} ( {P}_{gas} - {P}_{Liquid} ) - {F}_{Friction} = {a}_{Piston} {m}_{Piston}$$

Applying a steady state assumption yields:

$$\frac{π}{4} {d}_{Piston}^{2} ( {P}_{gas} - {P}_{Liquid} ) - {F}_{Friction}$$

Rearranging, results in:

$${P}_{Orifice} = {P}_{Gas} = \frac{{F}_{Friction}}{\frac{π}{4} {d}_{Piston}^{2}}$$

The jet impact force is given by the rate of change of the jet momentum at the impact surface:

$$F = dp / dt = d ( mv ) / dt = v ( dm / dt ) + m ( dv / dt ) ,$$

Where p is momentum, v is velocity and m is mass.

Assuming constant jet velocity for a given time step, the Dv/dt term goes to zero, yielding:

$$F = V ( dm / dt ) , or F = velocity * mass flow rate$$

$$F = density * area * velocity \hat{} 2$$

$$F = 1 / 4 * pi * density * diameter \hat{} 2 * velocity \hat{} 2$$

The jet power has been shown to correlate with needle-free penetration and dispersion by:

$$Power = 1 / 8 * pi * density * diameter \hat{} 2 * velocity \hat{} 3$$

### Example 2—Modelling Device Performance

In this Example, modelling was used to determine the performance parameters of different ingestible device configurations for delivering a dispensable substance. The model was the same as described in Example 1.

### Device and Fluid Properties

Number of nozzles=2
Nozzle throat geometry=circular, sharp-edged orifice
Piston diameter=7 mm
Piston friction=3.7 N
Friction pressure loss=14 psig
Dispensable substance (fluid)=Water.
Fluid density=1,000 kg/m3.
Fluid viscosity=1centiPoise
Ratios of specific heat (air)=1.4.
Initial dose volume of dispensable substance=300 μL
Initial gas volume=400 μL

With these different ingestible device configurations, the modelling yielded the results shown in Table 5. Note that liquid pressure is the same as fluid pressure.

TABLE 5

| | | Initial | Peak | | | |
|---|---|---|---|---|---|---|
| Nozzle | Nozzle | Internal | Fluid | Peak Jet | Peak Jet | Peak Jet |
| Diameter | Length | Pressure | Pressure | Velocity | Force | Power |
| (mm) | (mm) | (psig) | (psig) | (m/s) | (mN) | (W) |
| 0.35 | 2.0 | 320 | 306 | 47.7 | 231 | 5.5 |
| 0.35 | 0.70 | 320 | 306 | 50.6 | 260 | 6.6 |
| 0.20 | 0.70 | 320 | 306 | 48.4 | 74 | 1.8 |
| 0.10 | 0.70 | 320 | 306 | 43.0 | 15 | 0.31 |
| 0.20 | 0.25 | 320 | 306 | 50.8 | 81 | 2.1 |
| 0.10 | 0.25 | 320 | 306 | 48.4 | 18 | 0.44 |
| 0.20 | 0.70 | 20 | 6 | 6.7 | 1.4 | 0.0048 |
| 0.10 | 0.70 | 20 | 6 | 6 | 0.27 | 0.0008 |
| 0.20 | 0.25 | 20 | 6 | 7.1 | 1.6 | 0.0057 |
| 0.10 | 0.25 | 20 | 6 | 7 | 0.35 | 0.0012 |
| 0.20 | 0.70 | 660 | 646 | 70.4 | 156 | 5.5 |
| 0.10 | 0.70 | 2070 | 2056 | 111.9 | 98 | 5.5 |
| 0.20 | 0.25 | 600 | 586 | 70.4 | 156 | 5.5 |
| 0.10 | 0.25 | 1650 | 1636 | 112.0 | 98 | 5.5 |
| 0.10 | 0.25 | 34 | 20 | 12.3 | 1.2 | 0.007 |
| 0.10 | 0.25 | 320 | 306 | 48.4 | 18.4 | 0.44 |
| 0.10 | 0.25 | 175 | 161 | 35.0 | 9.6 | 0.17 |

### Example 3—Jet Measurements

High-speed video was used to measure jet parameters of a dispensable substance (water) delivered from devices having different nozzle diameters and nozzle lengths. The parameters for the devices were the same as those listed above in Example 2. The receiving medium (external environment) was air with gelatin (4% gelatin solution) located 5 mm from the nozzle exit. The nozzles were made of machined aluminum. The nozzle shape was a circular, sharp-edged orifice.

The results are shown in the last two columns of Table 6. The first eight columns of Table 6 provide data for the nozzles based on modelling (see Table 5 above).

TABLE 6

| | | | | | | | | Test | Test |
|---|---|---|---|---|---|---|---|---|---|
| | | Initial | Peak | Peak | Peak | Peak | Dose | Peak | Dose |
| Nozzle | Nozzle | Internal | Fluid | Jet | Jet | Jet | Delivery | Jet | Delivery |
| Diameter | Length | Pressure | Pressure | Velocity | Force | Power | Time | Force | Time |
| (mm) | (mm) | (psig) | (psig) | (m/s) | (mN) | (W) | (ms) | (mN) | (ms) |
| 0.35 | 2.0 | 320 | 306 | 47.7 | 231 | 5.5 | 57 | 184 | 62 |
| 0.35 | 0.70 | 320 | 306 | 50.6 | 260 | 6.6 | 53 | 228 | 51 |
| 0.10 | 0.25 | 34 | 20 | 12.3 | 1.2 | 0.0007 | 3060 | 8 | 3600 |
| 0.10 | 0.25 | 320 | 306 | 48.4 | 18.4 | 0.44 | 725 | 15 | 600 |
| 0.10 | 0.25 | 175 | 161 | 35.0 | 9.6 | 0.17 | 1022 | 12 | 830 |

The experimental peak force values show some agreement with the experimental peak force values from modelling. The experimental dose delivery times also show good correlation with the dose delivery times from modelling.

The ability of the nozzles in Table 6 to deliver the dispensable substance into the gelatin was also investigated. At a gas pressure of 320 PSIG, the power of the high-speed liquid jet and its ability to penetrate the gelatin was significantly reduced for the 0.1 mm diameter nozzles compared to the 0.35 mm diameter nozzle. Decreasing the gas pressure below 320 PSIG further reduced the power of the jet and extended the dose delivery time. For the nozzle with a diameter of 0.1 mm and a length of 0.25, there was no penetration into the gelatin for a gas pressure of 34 PSIG.

From Examples 2 and 3, it appears that peak jet power is a particularly significant parameter in determining whether an ingestible device will be able to provide trans-epithelial delivery of a dispensable substance. For example, with the relatively smaller nozzle diameters but relatively high peak jet velocity, a relatively high peak jet force could be achieved, but the peak jet power could be insufficient to achieve trans-epithelial delivery of a dispensable substance. It is believed that it is the combination of both high peak jet pressure and a relatively large nozzle diameter (with a corresponding relatively large jet diameter) are involved in successfully providing trans-epithelial delivery.

### Example 4—Evaluation of the Performance of an Ingestible Multi-Nozzle Jet Delivery Device in Beagle Dogs and Female Yorkshire Pigs

### Introduction

Studies were performed using molded ingestible devices designed and constructed for jet delivery to the GI tract. The ingestible devices operate by the triggered release of a laminar flow of fluid with sufficient energy to deposit the drug payload into gastrointestinal tissue initiated by the disintegration of a trigger module at a desired location. After deposition, the drug may be absorbed into the systemic circulation. In this Example, the performance of each iteration of the ingestible device in both ex-vivo tissue and in vivo animal studies is described, including, inter alia, enteric trigger location confirmation, nozzle cover performance, jet parameter characterization, and submucosal injection pharmacokinetics.

### Fluoroscopy Confirmation of Deployment and Location in Beagle Dogs

The beagle dog has similar GI motility to humans and administering ingestible devices to canines is relatively easy compared to other animals. In the present study, 10 out of 12 ingestible devices successfully deployed in the small intestine as confirmed via C-arm imaging of an lohexol payload. Overall triggering time was consistent in each group, with 25% enteric triggers (i.e., a 25% enteric coating weight-see Example 5 below) having an average deployment of 1 h and 8 min±5 min post gastric emptying, approximately 14 minutes faster than 30% enteric triggers.

### Ex-Vivo Tissue Deposition and GI Anatomy Comparison in Canines, Swine, and Human.

Benchtop studies of ex vivo intestinal tissue ink deposition showed the canine model was not an ideal model for drug disposition in the gastric tissue. Ingestible devices with black India Ink were wrapped with either swine or canine jejunum tissue in a wet chamber at room temperature. A small amount of fluid was then added to the tissue to assist in triggering the device via trigger degradation. Less injection volume was observed in the beagle dog gastric tissue compared to swine or human tissue.

A comparison of the gastrointestinal anatomy and physiology between swine, canine and humans is provided in Tables 7 and 8 below. It was determined that the Yucatan minipig model provides a viable animal model for evaluating injection gastrointestinal tissue. However, due to the variable gastric residence times observed in swine (0.9 hours to 20 days) (see Table 5) and the difference in stomach anatomy compared to canines and humans, a semi-autonomous ingestible device with an uncoated trigger was intraduodenally placed by endoscopy in some studies.

In terminal necropsy tests in swine, variable gas and water pockets in the small intestine between animals suggested variable motility and enlarged intestinal diameter may affect submucosal injection efficiency in the swine model. In humans, the literature has also shown that variation of water pockets is present in the small intestine. After ingestion of water, fluid is distributed in fluid pockets that are variable in both volume and number throughout the small intestine. Therefore, triggering of the ingestible device in the proximal portion of the small intestine, where there is less fluid and gas variability, may be preferred.

TABLE 7
Physiological condition comparison

| | | | GET | GET | Small | Small |
|---|---|---|---|---|---|---|
| | | Duodenum | (Fasted) | (Fed) | Intestine | Intestine |
| | Gastric pH | pH | (hr) | (hr) | Transit (hr) | Vol (ml) |
| Human | 0.4-4 | 5-7 | 0.66-1 h | ~2-5 h | ~2-4 h | 212 ± 110 |
| | (fasted) | | | | | |
| | 2-4.5 (fed) | | | | | |
| Swine | 1.4-4 | 6.0 | Variable. 4- | Variable | 3-4 h; 1-2 d; | 476 ± 253 |
| | (Fasted) | | 24 h, and up | | variable | |
| | 4.4 (Fed) | | to 20 days | | | |
| Canine | 1.5(fasted) | 6.2 | 0.4-1 h | Variable, | 2-3 h | 300 |
| | 3-5 (fed) | | | ~12-13 h | | |

TABLE 8
GI anatomy and motility comparison

| | Human | Dog | Pig |
|---|---|---|---|
| Duodenum* | 4% | 6% | 4-4.5% |
| Jejunum* | 38% | 90% | 88-91% |
| Ileum* | 58% | 4% | 4-5% |
| Villi Shape | Finger | Tongue | Finger |
| | Shaped | Shaped | Shaped |
| Mucus | 15.5 μm (D) | N/A | 25.6 μm (D) |
| Thickness | | | 35.6 μm (J) |

*% Length of Small Intestine

### Ingestible Device Injection and Pharmacokinetics in a Swine Model

To evaluate the injection efficiency of the ingestible jet delivery device, semi-autonomous devices with uncoated trigger were administrated by intraduodenal endoscopic placement (ID) and released into the proximal small intestines of female Yucatan swine. In a first study (Q19), the devices were filled with PGN-001 (a variant of adalimumab) and included nozzle covers to prevent premature drug loss. The nozzle covers, which consist of 40% RTV (room-temperature-vulcanizing silicone, commercially available as Permatex 81730) and 60% Mineral Spirits, were applied using a dip coating machine before filling with 0.415 mL PGN-001.

### Results

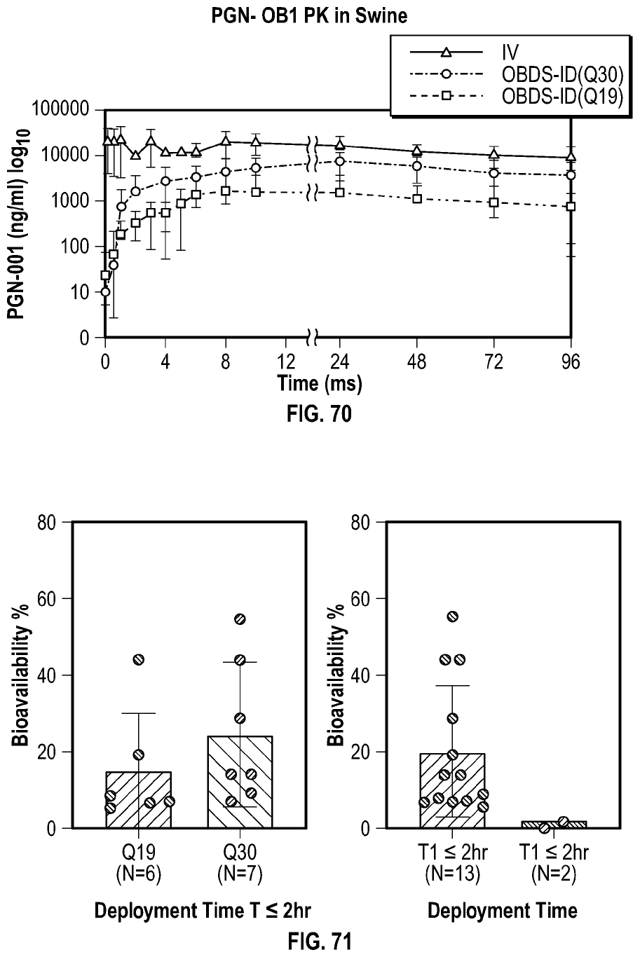

First, in the ex-vivo bench testing, it was found that the nozzle cover did not interfere with the tissue drug deposition. Second, in the in vivo portion of the study, 44% bioavailability of PGN-001 was achieved in 7 out of 12 animals (See Table 8 and FIG. 70). The animal-to-animal variation of oral bioavailability ranged from 6-44% (N=6; an average of 15%) and appeared to be a function of one or more of time to triggering, differences in GI motility, the presence of pockets, intestinal distension due to gas, or variation in device efficiency or function. Also, it should be noted that the devices with 350 PSI initial gas pressure showed higher bioavailability compared to the 320 PSI devices, which aligns with the results described in Examples 2 and 3. For example, it was observed that O-ring seal quality improves device performance as demonstrated in a follow-on study (Q28) with devices that yielded up to 62% bioavailability in vivo.

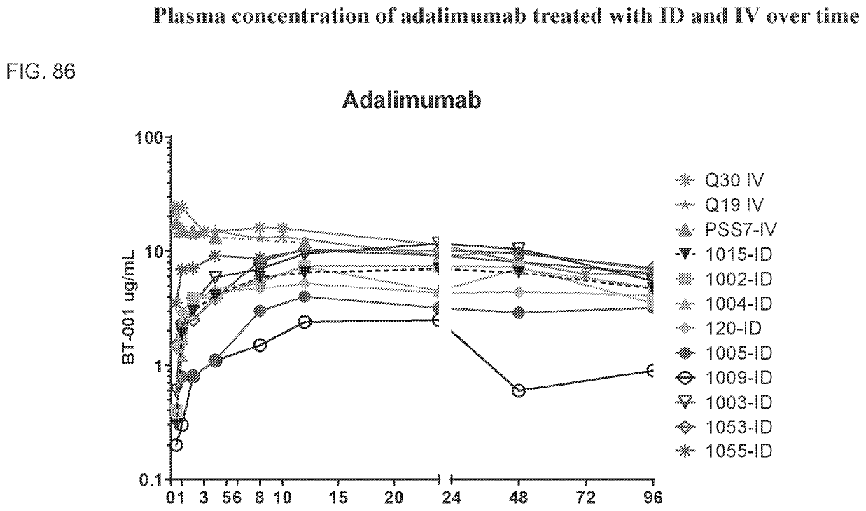

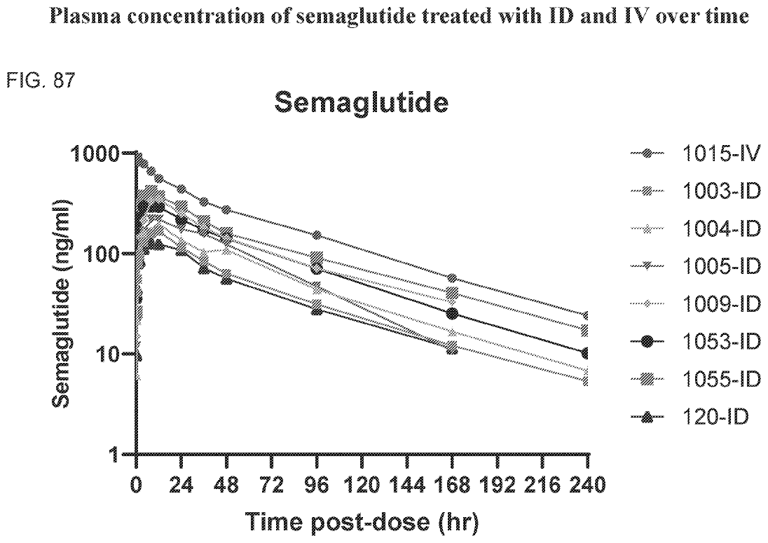

In the Q30 study, additional updates to the ingestible device assembly process were implemented to further improve device efficiency. Ex-vivo tissue testing prior to the in vivo study showed consistent tissue ink deposition. In addition, the ingestible devices were pre-soaked in a simulated gastric fluid for up to 13 min to pre-hydrate the trigger prior to administration in the small intestine. Eight out of 13 animals showed detectable drugs in the blood with earlier triggering ($2 hours) associated with higher bioavailability. An average bioavailability of 25% was seen in animals with a trigger time (T1)≤2 hr (N=7), while a trigger time of 72 hr post-dose yielded a bioavailability of only 1.2% (N=1). See Tables 9 and 10, and FIGS. 86 and 87.

TABLE 9
Device characteristics across different in vivo studies

| Study | Animal | Study | Test | | Nozzle | | Jet | Piercer | Piston- |
|---|---|---|---|---|---|---|---|---|---|
| number | model | type | Articles | Trigger | number | PSI | cover | O-ring | O-ring |
| Q5 | Beagle | C-arm | Iohexol | 30% | 2 | 350 | 5% | Silicone | Silicone |
| | dog | images | | & 25% | | | HPMC | | |
| Q19 | Minipig | PK | PGN-001 | Uncoated | 2 | 320 | Jet | Silicone | Teflon |
| | | | | | | | Cover | | |
| Q28 | Minipig | PK | PGN-001 | Uncoated | 2 | 350 | Jet | More | Silicone |
| | | | | | | | Cover | Silicone | |
| Q30 | Minipig | PK | PGN-001 | Uncoated | 2 & 6 | 350 | Jet | More | DM |
| | | | | | | | Cover | Silicone | |

TABLE 10
Submucosal Injection PK Results

| | # of | | | Average | Average | |
|---|---|---|---|---|---|---|
| Study | capsule | Animals with | Average | bioavailability | bioavailability | Maximum |
| number | dosed | drug in blood | bioavailability | with T1 ≤ 2 | with T1 > 2 | Bioavailability |
| Q19 | 12 | 7 | 13% | 15%; N = 6 | 0.42% | 44% |
| Q28 | 8 | 4 | 16% | 62%; N = 1 | 0.47% | 62% |
| Q30 | 13 | 8 | 22% | 25%; N = 7 | 1.2% | 55% |

### Monoclonal Antibody Pharmacokinetics in a Swine Model Using Externally-Triggered Device

A swine study similar to those described above was repeated using an externally-triggered device. The triggering mechanism is similar to the triggering mechanism described in FIG. 80A; however, the device is coupled to an endoscope, which is used to mechanically displace the sphere and thereby actuate the device.

Similar to the above pre-clinical experiments, the present study was performed using molded ingestible devices designed and constructed for jet delivery to the GI tract. The ingestible devices operate by the triggered release of a laminar flow of fluid with sufficient energy to deposit the drug payload into gastrointestinal tissue initiated by the triggering at a desired location-in the present study using an endoscopic to trigger the device. After deposition, the drug may be absorbed into the systemic circulation.

The externally-triggered study was performed using adalimumab, a TNFα inhibitor that binds specifically to TNF and neutralizes the biological function of TNF by blocking its interaction with both TNFR1 and TNFR2 receptors. Devices filled with ˜56 mg of BT-001, a variant of adalimumab, were administrated by intraduodenal (ID) endoscopic placement and were externally triggered in the proximal small intestine of the female Yucatan swine. Serum was collected from 0-96 hours post-dose and compared with the averaged IV control from four different studies with dose adjusted by weights.

### Results

Nine out of 9 (9/9) devices were successfully advanced through the pyloric sphincter via endoscopic placement and triggered in the proximal small intestine. All nine animals showed detectable drug levels up to 96 hours post-dosing (FIG. 86) and had an oral bioavailability average of 51/4%+24% (CV: 46%), ranging from 10%-90% compared to the IV control. No significant clinical signs were observed in the animals before or after dosing for up to 10 days.

### Conclusion

This study demonstrated that oral administration of the oral systemic device has the potential to achieve an average of 51% and as high as 90% bioavailability of an adalimumab variant in animals.

### Peptide Pharmacokinetics in a Swine Model Using Externally-Triggered Device

The above externally-triggered study was also performed using semaglutide, a glucagon-like-peptide-1 (GLP-1) receptor agonist that stimulates insulin secretion and suppress glucagon release. Devices filled with ˜0.96 mg of semaglutide were administrated by intraduodenal (ID) endoscopic placement and were externally triggered in the proximal small intestine of the female Yucatan swine. Plasma samples were then collected from 0 to 240 hours post-dose. Systemic concentrations of semaglutide were measured using LC-MS-MS to evaluate the PK of semaglutide with the OBDS device via ID administration (animals 1003-ID; 1004-ID; 1005-ID; 1009-ID; 10053-ID and 1055-ID in FIG. 87) compared with the IV control (animal 1015-IV in FIG. 87).

### Results

Seven out of eight (7/8) devices were successfully advanced through the pyloric sphincter via endoscopic placement and triggered in the proximal small intestine. One animal had a procedure error and was excluded from PK analysis. All seven animals showed detectable drug levels up to 10 days post-dosing (FIG. 87) and had an oral bioavailability average of 37%±15% (CV: 40%), ranging from 19%-60% compared to the IV control. No significant clinical signs were observed in the animals before or after dosing for up to 10 days.

### Conclusion

This study demonstrated that oral administration of the OBDS device has the potential to achieve an average of 37% and as high as 60% bioavailability of a GLP-1 receptor agonist in animals. This is a magnitude higher than the currently marketed oral tablet, Rybelsus®, which has less than 1% bioavailability estimated in human trials.

### Summary

These results demonstrate that an ingestible delivery system can deliver a monoclonal antibody (e.g., adalimumab) at bioavailability levels close to the subcutaneous route of administration (64% over IV, at 40 mg bi-weekly dose) estimated in human trials, and a peptide (e.g., semaglutide) at average bioavailability levels of 37% and as high as 60%. With increased dosing frequency and dose per device, the ingestible devices described herein may offer a new, non-invasive route of administration that can lead to improved patient adherence and compliance.

### Example 5—Pre-Clinical Ingestible Device Characterization

The preclinical ingestible devices used in Example 4 were tested to characterize the resulting jet parameters. These results were compared to modeled jet parameters as described in Examples 1-3.

### Dose Delivery Math Model: Pre-Clinical Devices

The modelling used to determine the performance parameters of the preclinical ingestible devices are described in Example 1. A summary of the device inputs for the different preclinical devices (e.g., 320 PSI vs 350 PSI devices) is provided below, as well as the test apparatus parameters and empirically derived factors (e.g., empirical loss coefficient as obtained from tuning to experimental outputs).

### Pre-Clinical 320PSI, 2×0.35 mm Nozzles

| | Units | Values |
|---|---|---|
| Fixed Inputs | | |
| Nozzle Diameter | mm | 0.35 |
| Nozzle Length | mm | 0.60 |
| Number of Nozzles | | 2 |
| Piston Friction | N | 2.0 |
| Piston Diameter | mm | 8.50 |
| Friction Press Loss | bar | 0.35 |
| Friction Press Loss | PSI | 5.11 |
| Liquid Density | kg/m3 | 1000 |
| Liquid Viscosity | Pas | 0.001 |
| Ratio of Specific Heat | (Air) | 1.4 |
| Initial Gas Pressure | PSI | 320 |
| Initial Gas Pressure | bar | 22.06 |
| Test Apparatus | | |
| Initial Dose Volume | μl | 400 |
| Dose Ullage | μl | 0 |
| Delivered Dose | μl | 400 |
| Gas to Dose Ratio | | 1.00 |
| Initial Gas Volume | μl | 400 |
| Final Gas Volume | μl | 800 |

Empirical Factors for Tuning

| | |
|---|---|
| Entrance Loss Coefficient | 0.35 |
| Exit Loss Coefficient | 1.00 |

For the above device features and parameters (e.g., a 320PSI preclinical device with two 0.35×0.60 mm nozzles), the modelling yielded the results shown in Table 11 and FIG. 56-67, namely a device that delivered 400 μl of liquid with a peak liquid pressure of 315 PSI (liquid pressure is the same as fluid pressure) at a peak velocity of 55 m/s and an average velocity 38 m/s. The ingestible device can create a liquid laminar jet with a peak jet impact force of 288 mN and a peak jet power of 7.9 W. The total delivery time is modeled to be 50.6 ms.

TABLE 11
Pre-Clinical 320 PSI, 2x 0.35 mm Nozzles: Device and Jet Parameters

| | | | | | | | | | | | | | Time |
|---|---|---|---|---|---|---|---|---|---|---|---|---|---|
| | | | | | | Est. | Jet | Jet | | Flowrate | | Time for | from |
| Delivered | Gas | Gas | Liquid | Liquid | | Pressure | Impact | Impact | Jet | Per | Total | Delivered | Start of |
| Dose | Volume | Pressure | Pressure | Pressure | Velocity | Drop | Force | Pressure | Power | Orifice | Flowrate | Vol | Dose |
| μl | μl | bar | bar | PSI | m/s | bar | mN | PSI | W | m3/s | μl/s | ms | ms |
| 0 | 400 | 22.1 | 21.7 | 314.9 | 54.7 | 21.71 | 288.3 | 434.6 | 7.89 | 5.27E−06 | 10533.4 | | 0.0 |
| 50 | 450 | 18.7 | 18.4 | 266.2 | 50.3 | 18.36 | 243.7 | 367.4 | 6.13 | 4.84E−06 | 9684.8 | 4.95 | 4.9 |
| 100 | 500 | 16.1 | 15.8 | 229.0 | 46.7 | 15.79 | 209.6 | 316.0 | 4.89 | 4.49E−06 | 8981.8 | 5.36 | 10.3 |
| 150 | 550 | 14.1 | 13.8 | 199.8 | 43.6 | 13.77 | 182.8 | 275.6 | 3.98 | 4.19E−06 | 8388.1 | 5.76 | 16.1 |
| 200 | 600 | 12.5 | 12.2 | 176.3 | 40.9 | 12.15 | 161.3 | 243.2 | 3.30 | 3.94E−06 | 7878.7 | 6.15 | 22.2 |
| 250 | 650 | 11.2 | 10.8 | 157.1 | 38.6 | 10.83 | 143.7 | 216.6 | 2.78 | 3.72E−06 | 7436.1 | 6.53 | 28.7 |
| 300 | 700 | 10.1 | 9.7 | 141.1 | 36.6 | 9.73 | 129.0 | 194.5 | 2.36 | 3.52E−06 | 7047.1 | 6.90 | 35.6 |
| 350 | 750 | 9.2 | 8.8 | 127.6 | 34.8 | 8.80 | 116.7 | 175.9 | 2.03 | 3.35E−06 | 6702.0 | 7.27 | 42.9 |
| 400 | 800 | 8.4 | 8.0 | 116.1 | 33.2 | 8.0 | 106.2 | 160.1 | 1.76 | 3.20E−06 | 6393.3 | 7.64 | 50.6 |

| | Units | Values |
|---|---|---|
| Fixed Inputs | | |
| Nozzle Diameter | mm | 0.35 |
| Nozzle Length | mm | 0.60 |
| Number of Nozzles | | 2 |
| Piston Friction | N | 2.0 |
| Piston Diameter | mm | 8.50 |
| Friction Press Loss | bar | 0.35 |
| Friction Press Loss | PSI | 5.11 |
| Liquid Density | kg/m3 | 1000 |
| Liquid Viscosity | Pas | 0.001 |
| Ratio of Specific Heat | (Air) | 1.4 |
| Initial Gas Pressure | PSI | 350 |
| Initial Gas Pressure | bar | 22.06 |
| Test Apparatus | | |
| Initial Dose Volume | μl | 400 |
| Dose Ullage | μl | 0 |
| Delivered Dose | μl | 400 |
| Gas to Dose Ratio | | 1.00 |
| Initial Gas Volume | μl | 400 |
| Final Gas Volume | μl | 800 |

Empirical Factors for Tuning

| | |
|---|---|
| Entrance Loss Coefficient | 0.35 |
| Exit Loss Coefficient | 1.00 |

For the above device features and parameters (e.g., a 350PSI preclinical device with two 0.35×0.60 mm nozzles), the modelling yielded the results shown in Table 12 and FIG. 56-67, namely a device that delivered 400 μl of liquid with a peak liquid pressure of 345 PSI (liquid pressure is the same as fluid pressure) at a peak velocity of 57 m/s and an average velocity 44 m/s. The ingestible device can create a liquid laminar jet with a peak jet impact force of 316 mN and a peak jet power of 9.1 W. The total delivery time is modeled to be 48.3 ms.

TABLE 12
Pre-Clinical 350 PSI, 2x 0.35 mm Nozzles: Device and Jet Parameters

| | | | | | | | | | | | | | Time |
|---|---|---|---|---|---|---|---|---|---|---|---|---|---|
| | | | | | | Est. | Jet | Jet | | Flowrate | | Time for | from |
| Delivered | Gas | Gas | Liquid | Liquid | | Pressure | Impact | Impact | Jet | Per | Total | Delivered | Start of |
| Dose | Volume | Pressure | Pressure | Pressure | Velocity | Drop | Force | Pressure | Power | Orifice | Flowrate | Vol | Dose |
| μl | μl | bar | bar | PSI | m/s | bar | mN | PSI | W | m3/s | μl/s | ms | ms |
| 0 | 400 | 24.1 | 23.8 | 344.9 | 57.3 | 23.78 | 315.8 | 476.1 | 9.05 | 5.51E−06 | 11024.2 | | 0.0 |
| 50 | 450 | 20.5 | 20.1 | 291.7 | 52.7 | 20.11 | 267.0 | 402.6 | 7.03 | 5.07E−06 | 10137.5 | 4.73 | 4.7 |
| 100 | 500 | 17.7 | 17.3 | 251.0 | 48.9 | 17.30 | 229.7 | 346.3 | 5.61 | 4.70E−06 | 9402.9 | 5.12 | 9.8 |
| 150 | 550 | 15.5 | 15.1 | 219.0 | 45.6 | 15.10 | 200.4 | 302.1 | 4.57 | 4.39E−06 | 8782.5 | 5.50 | 15.3 |
| 200 | 600 | 13.7 | 13.3 | 193.3 | 42.9 | 13.33 | 176.9 | 266.6 | 3.79 | 4.13E−06 | 8250.5 | 5.87 | 21. |
| 250 | 650 | 12.2 | 11.9 | 172.3 | 40.5 | 11.88 | 157.6 | 237.6 | 3.19 | 3.89E−06 | 7788.1 | 6.23 | 27.4 |
| 300 | 700 | 11.0 | 10.7 | 154.8 | 38.4 | 10.67 | 141.6 | 213.5 | 2.72 | 3.69E−06 | 7381.9 | 6.59 | 34.0 |
| 350 | 750 | 10.0 | 9.7 | 140.1 | 36.5 | 9.66 | 128.1 | 193.1 | 2.34 | 3.51E−06 | 7021.6 | 6.94 | 41.0 |
| 400 | 800 | 9.1 | 8.8 | 127.5 | 34.8 | 8.79 | 116.6 | 175.8 | 2.03 | 3.35E−06 | 6699.4 | 7.29 | 48.3 |

As described in Examples 2 and 3, a relatively large nozzle diameter (and the corresponding relatively large jet diameter) can significantly increase the jet power. Below are the device and jet parameters for 320 PSI ingestible devices with 2×0.40 mm nozzles and 2×0.50 mm nozzles.

Pre-Clinical 320 PSI, 2×0.40 mm Nozzles and 320 PSI, 2×0.50 mm Nozzles

| | Units | Values |
|---|---|---|
| Fixed Inputs | | |
| Nozzle Diameter | mm | 0.40/0.50 |
| Nozzle Length | mm | 0.60 |
| Number of Nozzles | | 2 |
| Piston Friction | N | 2.0 |
| Piston Diameter | mm | 8.50 |
| Friction Press Loss | bar | 0.35 |
| Friction Press Loss | PSI | 5.11 |
| Liquid Density | kg/m3 | 1000 |
| Liquid Viscosity | Pas | 0.001 |
| Ratio of Specific Heat | (Air) | 1.4 |
| Initial Gas Pressure | PSI | 320 |
| Initial Gas Pressure | bar | 22.06 |
| Test Apparatus | | |
| Initial Dose Volume | μl | 400 |
| Dose Ullage | μl | 0 |
| Delivered Dose | μl | 400 |
| Gas to Dose Ratio | | 1.00 |
| Initial Gas Volume | μl | 400 |
| Final Gas Volume | μl | 800 |

Empirical Factors for Tuning

| | |
|---|---|
| Entrance Loss Coefficient | 0.35 |
| Exit Loss Coefficient | 1.00 |

For the above device features and parameters (e.g., a 320PSI preclinical device with two 0.40×0.60 mm nozzles and a 320PSI preclinical device with two 0.50×0.60 mm nozzles), the modelling yielded the results shown in Table 13 and 14, respectively. Also, see FIG. 56-67.

The 320PSI device with two 0.40 mm diameter nozzles delivered 400 μl of liquid with a peak liquid pressure of 315 PSI (liquid pressure is the same as fluid pressure) at a peak velocity of 55 m/s and an average velocity 42 m/s. The ingestible device can create a liquid laminar jet with a peak jet impact force of 381 mN and a peak jet power of 10.5 W. The total delivery time is modeled to be 38.5 ms. See Table 13 and FIG. 56-67.

The 320PSI device with two 0.50 mm diameter nozzles delivered 400 μl of liquid with a peak liquid pressure of 315 PSI (liquid pressure is the same as fluid pressure) at a peak velocity of 56 m/s and an average velocity 43 m/s. The ingestible device can create a liquid laminar jet with a peak jet impact force of 605 mN and a peak jet power of 16.8 W. The total delivery time is modeled to be 24.4 ms. See Table 14 and FIG. 56-67.

TABLE 13
Pre-Clinical 320 PSI, 2x 0.40 mm Nozzles: Device and Jet Parameters

| | | | | | | | | | | | | | Time |
|---|---|---|---|---|---|---|---|---|---|---|---|---|---|
| | | | | | | Est. | Jet | Jet | | Flowrate | | Time for | from |
| Delivered | Gas | Gas | Liquid | Liquid | | Pressure | Impact | Impact | Jet | Per | Total | Delivered | Start of |
| Dose | Volume | Pressure | Pressure | Pressure | Velocity | Drop | Force | Pressure | Power | Orifice | Flowrate | Vol | Dose |
| μl | μl | bar | bar | PSI | m/s | bar | mN | PSI | W | m3/s | μl/s | ms | ms |
| 0 | 400 | 22.1 | 21.7 | 314.9 | 55.1 | 21.71 | 381.0 | 439.8 | 10.49 | 6.92E−06 | 13839.3 | | 0.0 |
| 50 | 450 | 18.7 | 18.4 | 266.2 | 50.6 | 18.36 | 322.1 | 371.8 | 8.15 | 6.36E−06 | 12724.6 | 3.76 | 3.8 |
| 100 | 500 | 16.1 | 15.8 | 229.0 | 47.0 | 15.79 | 277.1 | 319.8 | 6.50 | 5.90E−06 | 11801.1 | 4.08 | 7.8 |
| 150 | 550 | 14.1 | 13.8 | 199.8 | 43.9 | 13.77 | 241.6 | 278.9 | 5.30 | 5.51E−06 | 11021.1 | 4.38 | 12.2 |
| 200 | 600 | 12.5 | 12.2 | 176.3 | 41.2 | 12.15 | 213.2 | 246.1 | 4.39 | 5.18E−06 | 10352.0 | 4.68 | 16.9 |
| 250 | 650 | 11.2 | 10.8 | 157.1 | 38.9 | 10.83 | 189.9 | 219.2 | 3.69 | 4.89E−06 | 9770.5 | 4.97 | 21.9 |
| 300 | 700 | 10.1 | 9.7 | 141.1 | 36.8 | 9.73 | 170.6 | 196.9 | 3.14 | 4.63E−06 | 9259.5 | 5.25 | 27.1 |
| 350 | 750 | 9.2 | 8.8 | 127.6 | 35.0 | 8.80 | 154.3 | 178.1 | 2.70 | 4.40E−06 | 8806.2 | 5.54 | 32.7 |
| 400 | 800 | 8.4 | 8.0 | 116.1 | 33.4 | 8.01 | 140.4 | 162.0 | 2.35 | 4.20E−06 | 8400.8 | 5.81 | 38.5 |

TABLE 14
Pre-Clinical 320 PSI, 2x 0.50 mm Nozzles: Device and Jet Parameters

| | | | | | | | | | | | | | Time |
|---|---|---|---|---|---|---|---|---|---|---|---|---|---|
| | | | | | | Est. | Jet | Jet | | Flowrate | | Time for | from |
| Delivered | Gas | Gas | Liquid | Liquid | | Pressure | Impact | Impact | Jet | Per | Total | Delivered | Start of |
| Dose | Volume | Pressure | Pressure | Pressure | Velocity | Drop | Force | Pressure | Power | Orifice | Flowrate | Vol | Dose |
| μl | μl | bar | bar | PSI | m/s | bar | mN | PSI | W | m3/s | μl/s | ms | ms |
| 0 | 400 | 22.1 | 21.7 | 314.9 | 55.5 | 21.71 | 604.6 | 446.6 | 16.78 | 1.09E−05 | 21791.5 | | 0.0 |
| 50 | 450 | 18.7 | 18.4 | 266.2 | 51.0 | 18.36 | 511.2 | 377.6 | 13.04 | 1.00E−05 | 20036.7 | 2.39 | 2.4 |
| 100 | 500 | 16.1 | 15.8 | 229.0 | 47.3 | 15.79 | 439.7 | 324.8 | 10.40 | 9.29E−06 | 18582.8 | 2.59 | 5.0 |
| 150 | 550 | 14.1 | 13.8 | 199.8 | 44.2 | 13.77 | 383.5 | 283.3 | 8.47 | 8.68E−06 | 17355.0 | 2.78 | 7.8 |
| 200 | 600 | 12.5 | 12.2 | 176.3 | 41.5 | 12.15 | 338.4 | 249.9 | 7.02 | 8.15E−06 | 16301.6 | 2.97 | 10.7 |
| 250 | 650 | 11.2 | 10.8 | 157.1 | 39.2 | 10.83 | 301.4 | 222.7 | 5.90 | 7.69E−06 | 15386.2 | 3.16 | 13.9 |
| 300 | 700 | 10.1 | 9.7 | 141.1 | 37.1 | 9.73 | 270.7 | 200.0 | 5.03 | 7.29E−06 | 14581.7 | 3.34 | 17.2 |
| 350 | 750 | 9.2 | 8.8 | 127.6 | 35.3 | 8.80 | 244.9 | 180.9 | 4.32 | 6.93E−06 | 13868.1 | 3.51 | 20.7 |
| 400 | 800 | 8.4 | 8.0 | 116.1 | 33.7 | 8.01 | 222.9 | 164.6 | 3.75 | 6.61E−06 | 13229.8 | 3.69 | 24.4 |

### Jet Performance of Pre-Clinical Devices

In this section, the results of engineering testing carried out on the ingestible devices manufactured for preclinical studies are provided. The tested devices were made of plastic parts (e.g., polycarbonate) connected with UV adhesive. The trigger element included an enteric coating (e.g., 35% weight gain Acryl-EZE) and in some cases the trigger element was uncoated to facilitate faster triggering during testing. The nominal gas container fill was 15.7 mg, which produces a drive pressure of approximately 320 PSI. Additional testing was done on devices with a nominal gas container fill was 17.2 mg, which produces a drive pressure of approximately 350 PSI. During this round of testing, the devices did not include a nozzle covers, which are envisaged in the final device to seal the nozzles during storage and stomach transit and later displace to allow jet delivery after triggering. However, testing with jet covers is described in a subsequent section herein.

### Jet Rig Methods

Below is a summary of the jet force rig methods used to measure jet performance:
1. Mount the device onto the Jet Force Rig
a. Obtain Gelatin test sample and mount onto the Jet Force Rig with a stand-off distance of 5 mm from the outlet of a device nozzle; test should begin within 5 minutes of removing the Gelatin sample from the fridge.
b. Ensure the force sensor of the Jet Force Rig is positioned with a stand-off distance of 5 mm from the outlet of a device nozzle.
2. Start HSV Camera recording
3. Fill the Pre-Clinical Prototype Mount with tap water to initiate the process of device triggering. Observe the device triggering. Record the following data as per Jet Force Rig operating procedure:
a. Jet impact force profile
b. Peak Force (from force profile)
c. Time To Peak Force (from force profile)
d. Jet Delivery Time (from force profile)
e. Jet penetration depth, width and start of bolus in Gelatin test sample
4. Once device activation is complete, turn off HSV Camera and save captured video.

### Jet Rig Results

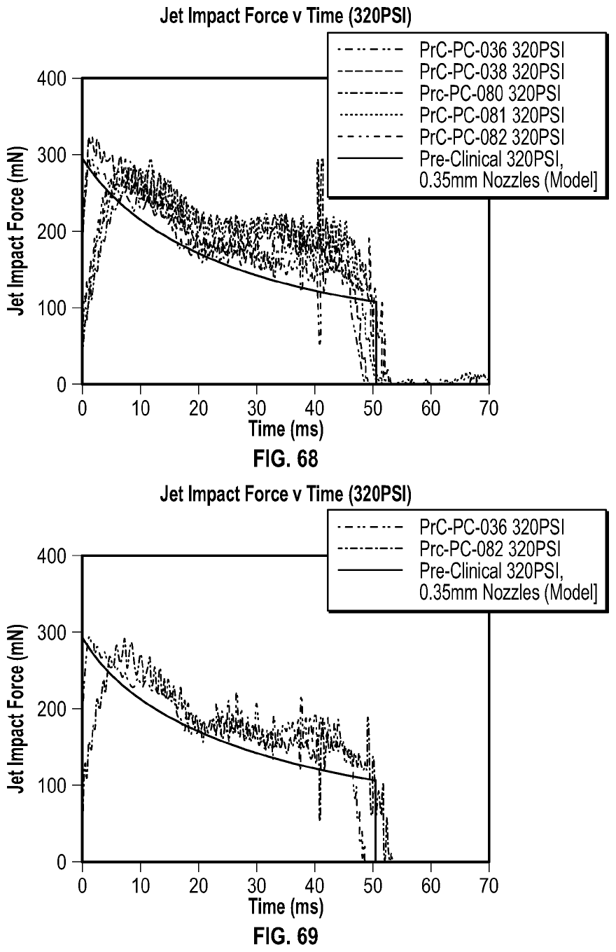

Jet performance profile results are summarized in Table 15. Note these values are derived from the raw force profile graphs. Examples of raw force profile graphs versus modeled force profiles are shown in FIG. 68-69.

TABLE 15
Summary of Jet Performance Profile metrics

| | 15.7 mg gas fill (320 psi) | | 17.2 mg gas fill (350 psi) | |
|---|---|---|---|---|
| | (N = 7) | | (N = 3) | |
| | Mean | Range | Mean | Range |
| Peak Force | 298 | 283-324 | 321 | 306-333 |
| (mN) | | | | |
| Time To Peak | 4.9 | 1.0-9.4 | 2.6 | 0.3-4.0 |
| Force (ms) | | | | |
| Jet Delivery | 50.4 | 48.2-52.8 | 47.1 | 46.8-47.4 |
| Time (ms) | | | | |

Testing with Nozzle Covers

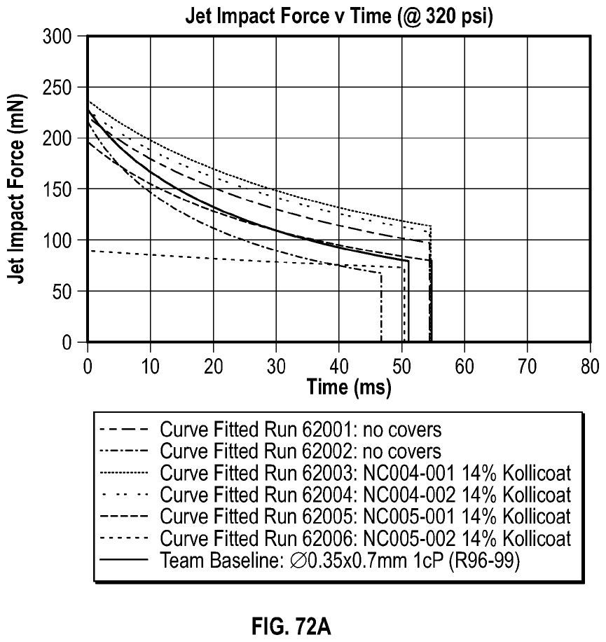

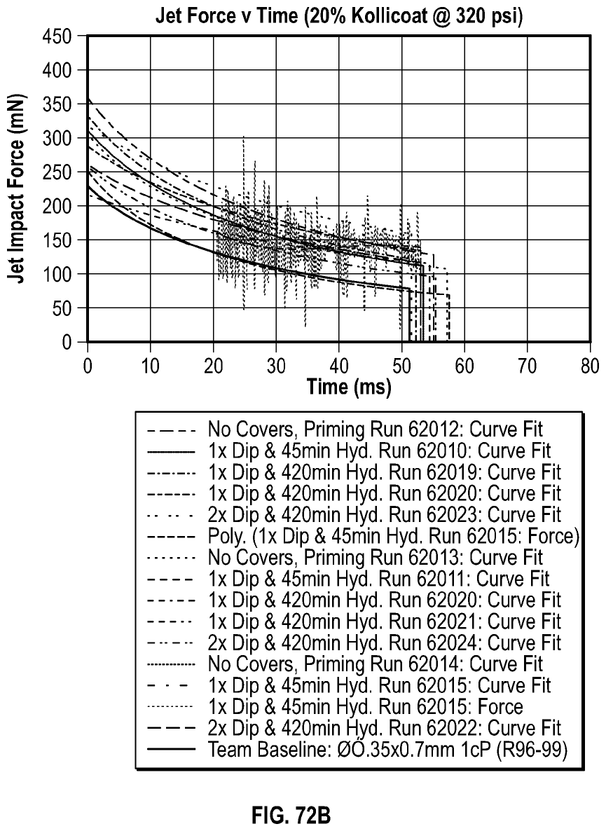

This section provides results from jet force rig testing of preclinical ingestible devices with nozzle covers. The same jet force rig described above was used to measure the jet characteristics of devices with nozzle covers. Cyclic olefin copolymer (COC) drug containers were dip coated with a formulation of 14% or 20% Kollicoat MAE 100 P in 97% IPA, before curing to evaporate the IPA and leave a 100% Kollicoat MAE 100 P nozzle cover coating. The 320PSI devices with nozzle covers produced repeatable drug delivery times ˜55 ms (N=4) and jet forces similar to devices without nozzle covers. See FIGS. 72A and B. For the 20% Kollicoat covers, longer conditioning (3 hours versus 30 minutes) produced force plots closer to the baseline. Conditioning is done to mimic device residence in the human gastrointestinal tract.

Testing with Second Piston as Nozzle Cover

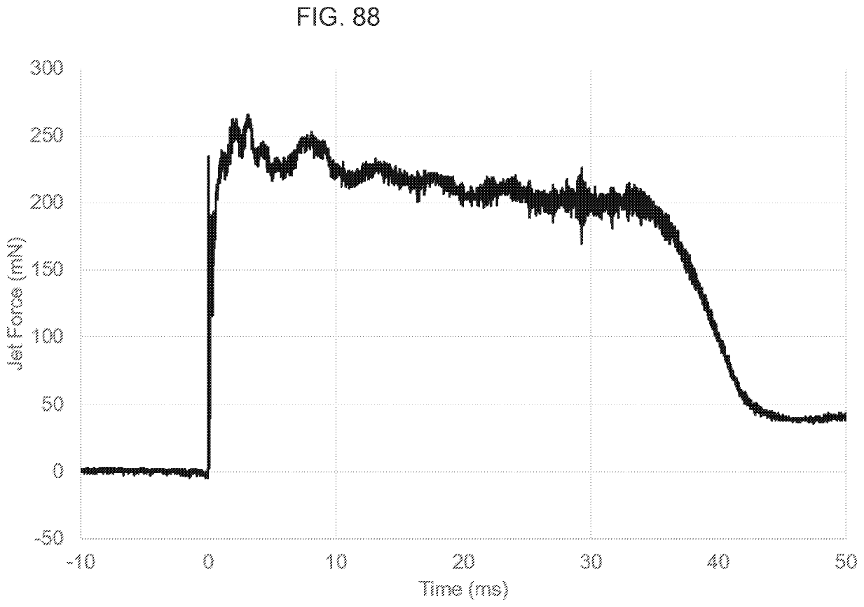

This section provides results from jet force rig testing of ingestible devices with mechanical nozzle covers, for example, like the second pistons shown in FIG. 49. The same jet force rig described above was used to measure the jet characteristics of devices that incorporated second piston “D” from FIG. 49, and the jet forces were measured over time. As shown in FIG. 88, the average jet force of eight devices, all with an internal pressure of 350 psi and incorporating second piston D to block the two openings prior to triggering, produced uniform jet force profiles (standard deviation of the peak force was calculated to be ˜33.4 mN) with the peak jet forces all exceeding 200 mN and an average peak force of 292.4 mN. One of the devices was excluded from the analysis because its total deployment time was about 30 ms, representing a significant outlier.

### Testing at Different Viscosities

This section provides results from jet force rig testing of ingestible devices with payloads of different viscosities. The same jet force rig described above was used to measure the jet characteristics of devices delivering a 50 cP liquid formulation vs water (1 cP). See Table 16 and FIG. 93.

TABLE 16
50 cP Formulation Observations

| Capsule | Viscosity | Observation |
|---|---|---|
| Water | 1 | Uniform jet formation from both nozzles |
| 1 | 50 | Uniform jet formation from both nozzles |
| 2 | 50 | Uniform jet formation from both nozzles |
| 3 | 50 | Uniform jet formation from both nozzles |
| 4 | 50 | Uniform jet formation from both nozzles |

As shown in FIG. 93, the 50 cP jet profiles (an average of the four 50 cP devices in Table 16) are similar to water, albeit at a slightly lower force but with peak forces still exceeding 200 mN.

### Recalibrated Model Results

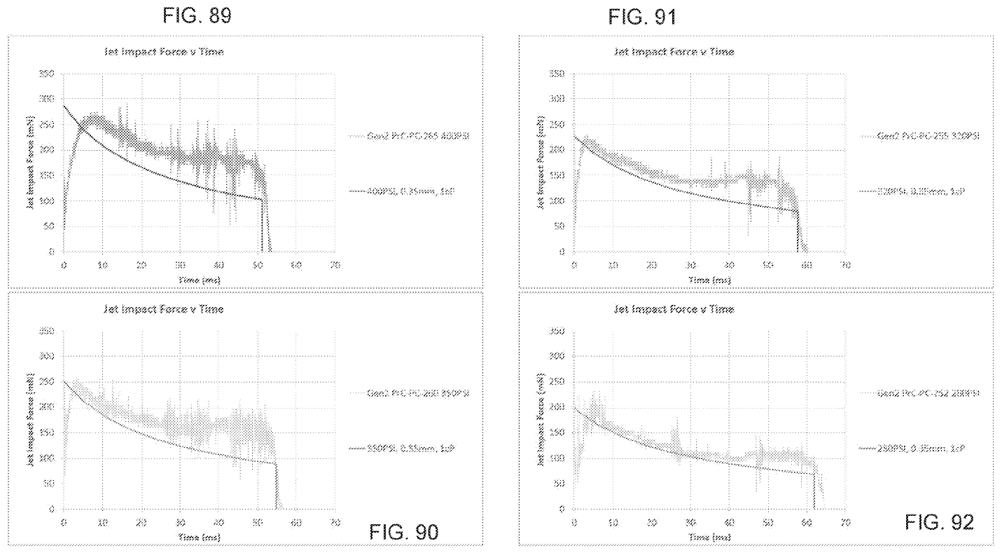

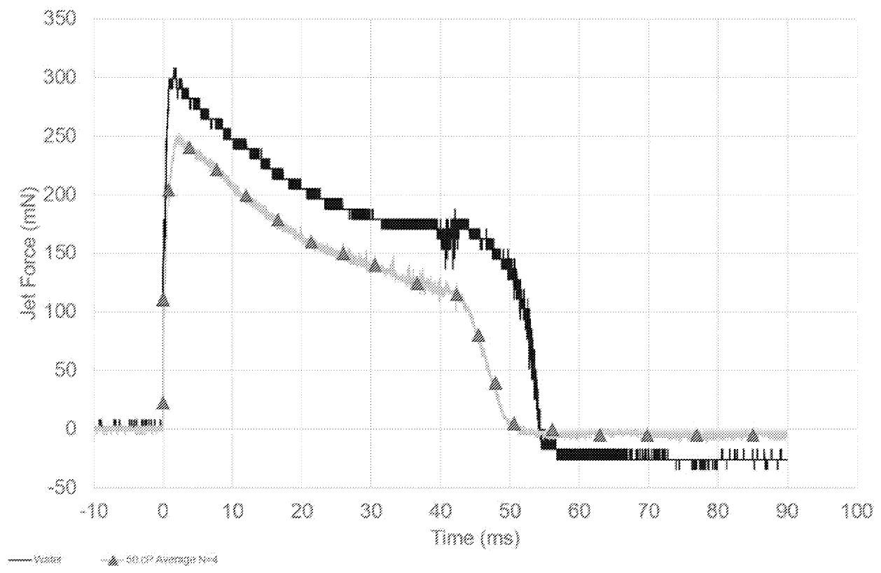

Additional jet force rig testing (as described above) was performed to measure force and the average peak force for devices with no nozzle covers at 280, 320, 350 & 400 psi internal pressure (or “drive pressure”). The average delivery time was also measured. See Table 17 and FIGS. 89-92.

TABLE 17
Results summary for peak force and jet delivery time

| Internal | Average | Average Jet |
|---|---|---|
| Pressure | Peak Jet | Delivery Time |
| (psi) | Force (mN) | (ms) |
| 280 | 196 | 60.0 |
| 320 | 225 | 60.3 |
| 350 | 251 | 56.6 |
| 400 | 282 | 52.7 |

Using the jet force rig data shown in Table 18, the device performance models described in Examples 1 and 2 were recalibrated and the calculated peak jet power, peak jet force, and average jet velocities at different internal pressures and viscosities are provided in Table 18 below. In FIGS. 89-92, the predicted jet forces and delivery times are shown using the smooth, thick line, while the experimental results summarized in Table 17 are shown using the noisier, light line. Re-calibration involves tuning the “Entrance Loss Coefficient” in the model (between 0 and 1) to a best fit with the experimental jet force rig results.

TABLE 18
Device Performance at Different Viscosities using Updated Model Calibration

| | | | | | | Previous Model | |
|---|---|---|---|---|---|---|---|
| | | | Updated Model Calibration | | | Calibration | |
| | Gas | | Calculated | Calculated | Calculated | Calculated | Calculated |
| Nozzle | Drive | Viscosity | Peak Jet | Peak Jet | Avg jet | Peak Jet | Peak Jet |
| diameter | Pressure | (mPas) | Power | Force | velocity | Power | Force |
| (mm) | (psi) | or (cP) | (W) | (mN) | (m/s) | (W) | (mN) |
| 0.350 | 280 | 1 | 4.5 | 197 | 34-35 | 6.4 | 252 |
| 0.350 | 320 | 1 | 5.5 | 227 | 33-36 | 7.9 | 288 |
| 0.350 | 350 | 1 | 6.3 | 249 | 38-39 | 9.0 | 316 |
| 0.350 | 400 | 1 | 7.8 | 286 | 41-42 | 11.1 | 362 |
| 0.350 | 280 | 50 | 4.1 | 185 | 32-33 | 5.8 | 235 |
| 0.350 | 320 | 50 | 5.0 | 214 | 32-35 | 7.1 | 270 |
| 0.350 | 350 | 50 | 5.8 | 235 | 37-38 | 8.2 | 296 |
| 0.350 | 400 | 50 | 7.2 | 271 | 39-40 | 10.1 | 341 |
| 0.350 | 280 | 100 | 3.8 | 176 | 31-32 | 5.3 | 222 |
| 0.350 | 320 | 100 | 4.7 | 204 | 31-34 | 6.6 | 256 |
| 0.350 | 350 | 100 | 5.4 | 225 | 35-37 | 7.6 | 282 |
| 0.350 | 400 | 100 | 6.7 | 260 | 38-39 | 9.4 | 325 |

### Example 6—Trigger Development and Testing

Further characterization and development of the uncoated trigger formulation was carried out to meet objectives of strength, disintegration speed and disintegration consistency:

Strength: Trigger material (i.e., formed restraining element herein referred to as “tablet”) tensile strength required ˜≥1.0 MPa (calculated using part diameter approximation to take account of central hole) and low friability required for subsequent use in coating machinery.

Fast Disintegration: Fast uncoated tablet disintegration required in 9 ml pH6.5 FASSIF 37° C. (no stirring) when tested in sprung loaded device. Disintegration can be used interchangeably with degradation or erosion as used herein.

Consistent Disintegration: Uncoated tablet to provide consistent disintegration across batches and after storage for up to approx. 6 months.

Different uncoated trigger (i.e., tablet) formulations were created and tested for breaking force, disintegration time, friability and dimensional stability. Restraining elements (tablets) were made with 7.6 kN compression force (284 MPa) to a height of 1.45 mm. Faster disintegration (e.g., reduced average time to trigger from [1 min 43s] to [0 min 48s]) was seen with a decrease in tablet mass (56.0 mg vs 42.2 mg) and also small decrease in the ratio of mass to exposed surface area (54.9 mg/cm2 vs 49.4 mg/cm2). Examples of trigger materials with their corresponding breaking force, disintegration times, and friability assessments are provided below in Table 19.

TABLE 19
Uncoated Trigger Formulations: Breaking Force, Disintegration Time, and Friability

| | Cohort 1% w/w | | | | | Cohort 2% w/w | |
|---|---|---|---|---|---|---|---|
| | 141#019A | 141#019B | 141#019C | 141#019D | 141#019E | 141#020A | 141#020B |
| Material | C1-A | C1-B | C1-C | C1-D | C1-E | C2-A | C2-B |
| StarTab | 49.6 | 59.3 | 48.8 | 48.8 | 47.8 | 45.3 | 40.3 |
| MCC 102 | 49.6 | 39.3 | 48.8 | 48.8 | 47.8 | 45.3 | 40.3 |
| Ac-di-Sol | 0.50 | 1.0 | 2.0 | 1.0 | 1.0 | 1.0 | 1.0 |
| Mg S | 0.25 | 0.25 | 0.25 | 0.25 | 0.25 | 0.25 | 0.25 |
| Crospovidone | | | | 1.0 | 3.0 | 3.0 | 3.0 |
| Crosslinked | | | | | | 5.0 | 15 |
| Starch | | | | | | | |
| Explotab | | | | | | | |
| Mannitol | | | | | | | |

Results

| | | | | | | | |
|---|---|---|---|---|---|---|---|
| Breaking | 0.87 | 0.81 | 1.01 | 1.04 | 1.08 | 0.88 | 0.92 |
| force | | | | | | | |
| (kg) n = 3 | | | | | | | |
| Disintegration | <30 s | <30 s | <30 s | <30 s | <30 s | <30 s | <30 s |
| time (s) | | | | | | | |
| Friability | Good | Good | Good | Good | Good | Good | Good |

| | Cohort 2% w/w | | | | |
|---|---|---|---|---|---|
| | 141#020C | 141#020D | 141#020E | 141#020F | 141#020G |
| Material | C2-C | C2-D | C2-E | C2-F | C2-G |
| StarTab | 46.3 | 42.8 | 46.8 | 44.3 | 40.3 |
| MCC 102 | 46.3 | 42.8 | 46.8 | 44.3 | 40.3 |
| Ac-di-Sol | 2.0 | 1.0 | 1.0 | 1.0 | 1.0 |
| Mg S | 0.25 | 0.25 | 0.25 | 0.25 | 0.25 |
| Crospovidone | | 3.0 | | | 3.0 |
| Crosslinked | | | | | |
| Starch | | | | | |
| Explotab | | | 5.0 | 10 | 10 |
| Mannitol | 5.0 | 10 | | | 5.0 |
| | Results | | | | |
| Breaking | 0.96 | 0.88 | 0.83 | 0.89 | 0.73 |
| force | | | | | |
| (kg) n = 3 | | | | | |
| Disintegration | <30 s | <30 s | <30 s | <30 s | <30 s |
| time (s) | | | | | |
| Friability | Good | Good | Good | Good | Good |

Preferred uncoated trigger formulations include C1-A (StarTab+MCC 102+Ac-di-Sol+Mg S), C1-E (StarTab+MCC 102+Ac-di-Sol+Mg S+Crospovidone), C2-B (StarTab+MCC 102+Ac-di-Sol+Mg S+Crospovidone+Cross linked+Starch), C2-D (StarTab+MCC 102+Ac-di-Sol+Mg S+Crospovidone+Mannitol), and C2-F (StarTab+MCC 102+Ac-di-Sol+Mg S+Explotab). The trigger formulations may further comprise an enteric material (e.g., Opadry 5% w/g+Acryl-EZE 35% w/g) coated on the outside of the trigger formulations provided in Table 19. The enteric material can be coated at different weights (e.g., 5%, 10%, 15%, 20%, 25%, 30%, 35%, 40%, 45%, 50% and values in between) depending on the desired triggering location in the gastrointestinal tract.

### Mechanical Trigger Modifications—Testing Results

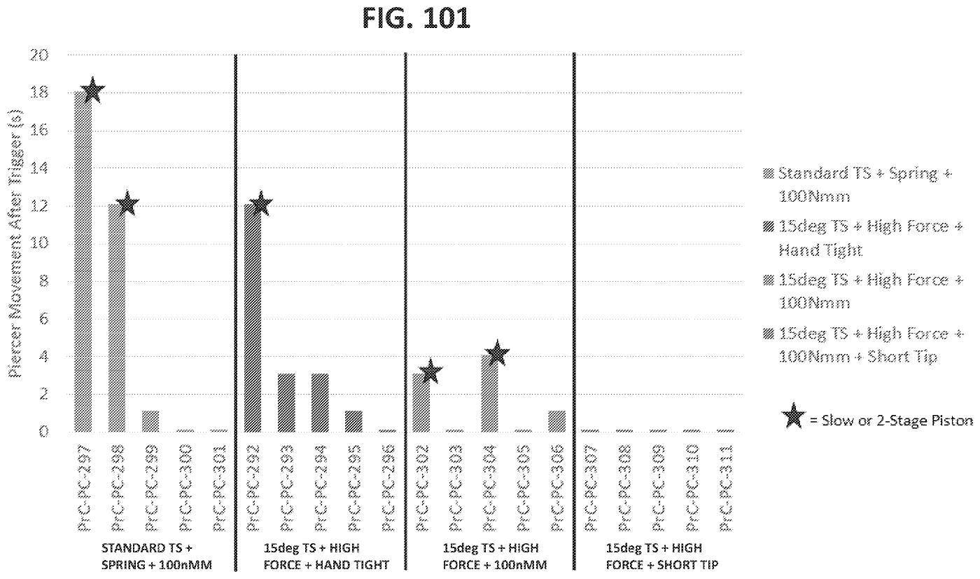

The angle supports shown in shown in FIGS. 75A-D were tested for their ability to actuate the device via piercing of the gas cannister as intended (i.e., the septum of the gas container fails in a single, catastrophic event, which results in a single, rapid movement of the piston). When five of the 15-degree angled supports were tested with high force springs (about 35-45 N) and shorter piercers (about 2.55 mm datum plane length), all five devices actuated immediately upon release component dissolution and there were no slow or two-stage pistons (i.e., the piston moves too slowly or non-uniformly to create liquid jets). This is shown in FIG. 101, which shows the time to piercer movement after dissolution or degradation of the release component or “trigger”. Suspected slow or two-stage pistons are marked with a star. Devices without the angled support were slower to actuate and resulted in some slow or two-stage pistons. In FIG. 101, “TS” means trigger support, “High Force” refers to the force of the spring, and “Hand Tight” and “100Nmm” refers to the force applied to tighten trigger support to the release component.

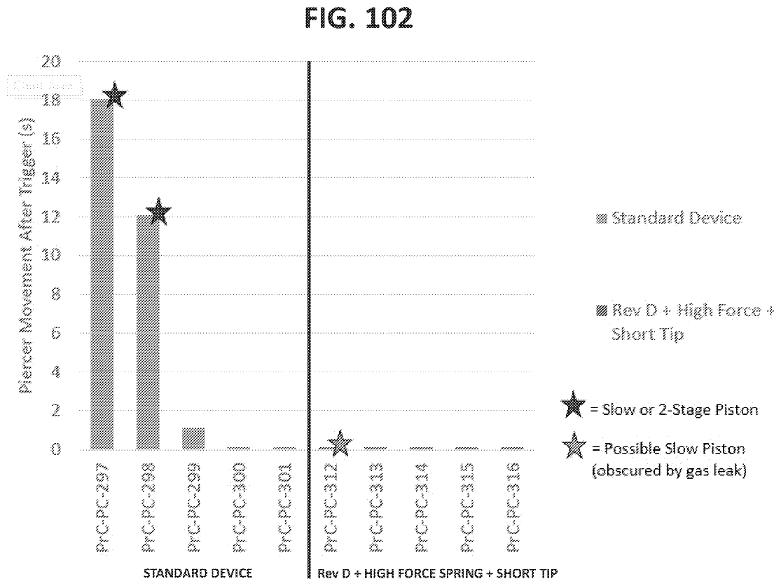

The breakaway piercer flanges shown in FIGS. 78A-L were also tested for their ability to actuate the device via piercing of the gas cannister as intended (i.e., the septum of the gas container fails in a single, catastrophic event, which results in a single, rapid movement of the piston). See FIG. 102. All five of the devices with breakaway piercer flanges actuated immediately upon release component dissolution; however, one of the devices had a suspected slow piston. Video imaging showed a potential gas leak at the glue joint suggesting the cause of the slow piston was not trigger related. “Standard Devices” did not have the breakaway piercer flanges. As described above, “High Force” refers to the force of the spring (about 35-45 N) and “Short Tip” refers to the piercer dimensions (about 2.55 mm datum plane length). “Rev D” refers to the design of the breakaway piercer flange.

From the additional testing and new data discussed above, the following observations can be made:

In general, an ingestible device for trans-epithelial delivery is configured to deliver a jet of the dispensable substance having one or more of:
a peak jet power of from about 3 Watts to about 8 Watts, or from about 4.2 Watts to about 7.8 Watts. or from about 4.6 Watts to about 7.0 Watts, or from about 5.0 Watts to about 6.3 Watts;
a minimum jet power of from about 1.7 W to about 0.8 W (e.g., from about 1.5 W to about 0.9 W, or from about 1.3 W to about 1.0 W);
an average jet power of at least about 1.9 W (e.g., about 2.2 W, about 2.4 W) and/or at most about 3.8 W (e.g., at most about 3.5 W, at most about 3.1 W);
a peak jet force of from about 150 mN to about 310 mN, or, from about 195 mN to about 285 mN, or, from about 215 mN to about 250 mN;
a minimum jet force of from about 63 mN to about 102 mN (e.g., from about 70 mN to about 92 mN, or from about 76 mN to about 86 mN);
an average jet force of from about 110 mN to about 172 mN (e.g., from about 118 mN to about 162 mN, from about 126 mN to about 158 mN);
a peak jet velocity of about 43 meters per second to about 55 meters per second, or about 45 meters per second to about 53 meters per second, or about 47 meters per second to about 51 meters per second;
an average jet velocity of from about 32 m/s to about 42 m/s (e.g., about 33 m/s, about 34 m/s, about 35 m/s, about 36 m/s, about 37 m/s, about 38 m/s, about 39 m/s, about 40 m/s, about 41 m/s);
a jet stable length of at least about 5 millimeters, or from about 5-15 mm, or from about 5-10 mm, or from about 5-8 mm;
a jet diameter of from about 0.25 mm to about 0.40 mm, or, in some embodiments from about 0.30 mm to about 0.35 mm, or, in some embodiments about 0.35 mm;
an internal pressure of, in some embodiments, from about 250 psi to about 400 psi, or from about 280 psi to about 370 psi, or from about 300 psi to about 350 psi;
a peak fluid pressure of from 280 psi to 320 psi or from 290 psi to 310 psi; and/or
an entrance loss coefficient of about 0.50 to about 0.90, or about 0.60 to about 0.80, or about 0.70.

In one example, an ingestible device with two nozzles each having a nozzle diameter of 0.35 mm diameter and a nozzle length of 0.60 mm, and containing a 1 cP dispensable substance at a peak fluid pressure of 308 psi can deliver a jet of the dispensable substance at a peak jet velocity of about 49 m/s and at a peak jet impact pressure of about 342 psi and a peak jet power of about 5.5 Watts with a total time of delivery of about 58 milliseconds (ms).

In another example, an ingestible device with two nozzles each having a nozzle diameter of 0.35 mm diameter and a nozzle length of 0.60 mm, and containing a 1 cP dispensable substance at a peak fluid pressure of 268 psi can deliver a jet of the dispensable substance at a peak jet velocity of about 45 m/s and at a peak jet impact pressure of about 298 psi and a peak jet power of about 4.5 Watts with a total time of delivery of about 62 milliseconds (ms).

In another example, an ingestible device with two nozzles each having a nozzle diameter of 0.35 mm diameter and a nozzle length of 0.60 mm, and containing a 50 cP dispensable substance at a peak fluid pressure of 308 psi can deliver a jet of the dispensable substance at a peak jet velocity of about 47 m/s and at a peak jet impact pressure of about 323 psi and a peak jet power of about 5.0 Watts with a total time of delivery of about 60 milliseconds (ms).

In another example, an ingestible device with two nozzles each having a nozzle diameter of 0.35 mm diameter and a nozzle length of 0.60 mm, and containing a 50 cP dispensable substance at a peak fluid pressure of 338 psi can deliver a jet of the dispensable substance at a peak jet velocity of about 50 m/s and at a peak jet impact pressure of about 355 psi and a peak jet power of about 5.8 Watts with a total time of delivery of about 57 milliseconds (ms).

A number of embodiments have been described. Nevertheless, various modifications may be made without departing from the spirit and scope of the disclosure. Accordingly, other embodiments are within the scope of the following claims.

### U.S. National Phase Only: Incorporation by Reference

The following patent applications are incorporated by reference. The citations to each of these applications in U.S. Provisional Application 63/321,537 are also incorporated herein by reference.
WO2020106704;
62/948,082;
63/027,427;
63/086,630;
63/321,537;
WO2021119482;
WO2022034041;
WO2022033949;
WO2019178071;
US 2017/0258583.

1-101. (canceled)

102. An ingestible device, comprising:
one or more openings in a housing;
a gas container in the housing containing a compressed gas;
an end cap held into engagement with the gas container by a release component;
a reservoir of dispensable substance in the housing;
wherein after the ingestible device is ingested, actuation of the release component allows displacement of the end cap to release compressed gas from the gas container to drive a jet of the dispensable substance out through the openings.

103. The ingestible device of claim 102 having no spring and no piercer.

104. The ingestible device of claim 102 with the end cap having an inner O-ring on a portion of the end cap extending into an opening of the gas container, and an outer O-ring which slidably seals the end cap against inner walls of the housing.

105. The ingestible device of claim 102 further including a first piston in the housing between the gas container and the openings.

106. The ingestible device of claim 105 wherein the housing comprises a first section and a second section, with the first piston in the first section and the release component and the end cap in the second section.

107. The ingestible device of claim 105 wherein the reservoir is between the first piston and a second piston.

108. The ingestible device of claim 107 wherein the first piston conforms to the shape of the gas container and the second piston conforms to the shape of the first piston.

109. The ingestible device of claim 102 having a peak jet force of about 150 mN to 310 mN and a peak jet velocity of about 45 m/s to 60 m/s.

110. The ingestible device of claim 102 wherein the release component has an annular shoulder having a first diameter retained by a ring at an end of the housing, the ring having a second diameter less than the first diameter.

111. The ingestible device of claim 102 wherein a back surface of the end cap is held against a front surface of the release component.

112. An ingestible device, comprising:
a storage reservoir in a housing;
one or more openings in the housing;
the openings in the housing connecting into the storage reservoir;
a first piston slidable longitudinally within the housing;
a second piston slidable longitudinally within the housing; and
the storage reservoir between the first piston and the second piston.

113. The ingestible device of claim 112 further including a gas container in the housing, the gas container containing a compressed gas, and the first piston between the gas container and the storage reservoir.

114. The ingestible device of claim 113 further including an end cap secured into the gas container by a release component, wherein upon actuation of the device after ingestion, the end cap moves in a first direction releasing the compressed gas from the gas container, the compressed gas driving the first piston and the second piston to move in a second direction opposite from the first direction.

115. The ingestible device of claim 113 wherein the second piston is movable from a first position, wherein the second piston closes off the openings, to a second position wherein the second piston is displaced from the openings.

116. The ingestible device of claim 112 wherein the storage reservoir has cylindrical sidewalls converging towards the openings at a draft angle of 0.3 to 0.1 degrees.

117. The ingestible device of claim 112 having a peak jet force of about 150 mN to 310 mN and a peak jet velocity of about 45 m/s to 60 m/s.

118. An ingestible device, comprising:
at least one opening in a housing;
a gas container in the housing containing a compressed gas;
an end cap having one or more O-rings sealing the gas container; and
a release component;
wherein after the ingestible device is ingested, actuation of the release component allows displacement of the end cap to release the compressed gas from the gas container to drive a jet of a dispensable substance out through the opening.

119. The ingestible device of claim 118 further including a first piston and a second piston in the housing, the first piston between the gas container and the opening, and the second piston movable from a first position, wherein the second piston closes off the opening, to a second position wherein the second piston is displaced from the opening.

120. The ingestible device of claim 119 wherein the first piston is movable from a first position overlying the gas container, to a second position away from the gas container, the first piston substantially within the second piston when in the second position.
